(19) United States
(12) Patent Application Publication
(10) Pub. No.: US 2011/0052501 A1
(43) Pub. Date: Mar. 3, 2011

(54) POLYPEPTIDES AND POLYNUCLEOTIDES, AND USES THEREOF AS A DRUG TARGET FOR PRODUCING DRUGS AND BIOLOGICS

(76) Inventors: Liat Dassa, Tel-Aviv (IL); Galit Rotman, Herzeliya (IL); Eve Montia, Rehovot (IL); Avi Yeshah Rosenberg, Kfar-Saba (IL); Anat Cohen-Dayag, Rehovot (IL); Amir Toporik, Parde hana Kab (IL); Zurit Levine, Herzliya (IL); Shira Walach, Hod Ha Sharon (IL); Shirley Sameach-Greenwald, Kfar-Saba (IL); Yaron Kinar, Tel-Aviv (IL); Ofer Levy, Jerusalem (IL); Sergey Nemzar, Raanana (IL)

(21) Appl. No.: 12/863,189

(22) PCT Filed: Feb. 1, 2009
(86) PCT No.: PCT/IL09/00123

§ 371 Date: Jul. 28, 2010

Related U.S. Application Data
(60) Provisional application No. 61/025,054, filed on Jan. 31, 2008.
(60) Provisional application No. 61/035,168, filed on Mar. 10, 2008.
(60) Provisional application No. 61/043,599, filed on Apr. 9, 2008.

(51) Int. Cl. A61K 49/00; C07K 14/47; C07K 16/18; A61K 39/395; C12N 9/96; G01N 33/68; A61P 35/00; A61P 35/02; A61P 35/04; A61P 37/02; A61P 19/02; A61P 29/00; A61P 25/00; A61P 17/06; A61P 37/06; G01N 1/00

(52) U.S. Cl. 424/9.2; 530/350; 530/387.9; 424/139.1; 530/387.3; 530/391.3; 435/188; 530/391.7; 436/501; 424/9.1; 73/863

(57) ABSTRACT

This invention relates to a novel target for production of immune and non-immune based therapeutics and for disease diagnosis. More particularly, the invention provides therapeutic antibodies against KIAA0746, CD20 or CD55 antigens, which are differentially expressed in cancer and in specific blood cells, and diagnostic and therapeutic usages. This invention further relates to the discovery of extracellular domains of KIAA0746 and its variants, CD20 and its variants, CD55 and its variants, which are suitable targets for immunotherapy, cancer therapy, treatment of inflammatory, allergic and autoimmune disorders, and drug development.

### RELATED APPLICATIONS

This invention claims benefit of priority to and incorporates by reference in their entireties U.S. Provisional Application Nos. 61/025,054, filed on Jan. 31, 2008; 61/035,168, filed on Mar. 10, 2008; and 61/043,599, filed on Apr. 9, 2008.

### FIELD OF THE INVENTION

This invention relates to the discovery of certain proteins that are differentially expressed in specific tissues and their use as therapeutic and diagnostic targets.

### BACKGROUND OF THE INVENTION

Tumor antigens are ideally positioned as biomarkers and drug targets, and they play a critical role in the development of novel strategies for active and passive immunotherapy agents, to be used as stand-alone therapies or in conjunction with conventional therapies for cancer. Tumor antigens can be classified as either tumor-specific antigens (TSAs) where the antigens are expressed only in tumor cells and not in normal tissues, or tumor-associated antigens (TAAs) where the antigens are overexpressed in tumor cells but nonetheless also present at low levels in normal tissues.

TAAs and TSAs are validated as targets for passive (antibody) therapy as well as active immunotherapy using strategies to break immune tolerance and stimulate the immune system. The antigenic epitopes that are targeted by these therapeutic approaches are present at the cell surface, overexpressed in tumor cells compared to non-tumor cells, and are targeted by antibodies that block functional activity, inhibit cell proliferation, or induce cell death.

There are a growing number of tumor-associated antigens against which monoclonal antibodies have been tested or are in use as treatment for cancer. The identification and molecular characterization of novel tumor antigens expressed by human malignancies is an active field in tumor immunology. Several approaches have been used to identify tumor-associated antigens as target candidates for immunotherapy, including high throughput bioinformatic approaches, based on genomics and proteomics. The identification of novel TAAs or TSAs expands the spectrum of tumor antigen targets available for immune recognition and provides new target molecules for the development of therapeutic agents for passive immunotherapy, including monoclonal antibodies, whether unmodified or otherwise linked to or combined with an active agent. Such novel antigens may also point the way to more effective therapeutic vaccines for active or adoptive immunotherapy.

Cancer vaccination involves the administration of tumor antigens and is used to break immune tolerance and induce an active T-cell response to the tumor. Vaccine therapy includes the use of naked DNA, peptides, recombinant protein, and whole cell therapy, where the patient's own tumor cells are used as the source of the vaccine. With the identification of specific tumor antigens, vaccinations are more often carried out by dendritic cell therapy, whereby dendritic cells are loaded with the relevant protein or peptide, or transfected with vector DNA or RNA.

The major applications of anti-TAA antibodies for treatment of cancer are therapy with a naked antibody, therapy with a drug-conjugated antibody, and fusion therapy with cellular immunity. Ever since their discovery, antibodies were envisioned as “magic bullets” that would deliver toxic agents, such as drugs, toxins, enzymes and radioisotopes, specifically to the diseased site and leaving the non-target normal tissues unaffected. Indeed, antibodies, and in particular antibody fragments, can function as carriers of cytotoxic substances such as radioisotopes, drugs and toxins. Immunotherapy with such immunoconjugates is more effective than with the naked antibody.

In contrast to the overwhelming success of naked (such as Rituxan and Campath) and conjugated antibodies (such as Bexxar and Zevalin) in treating hematological malignancies, only modest success has been achieved in the immunotherapy of solid tumors. One of the major limitations in successful application of immunotherapy to solid tumors is the large molecular size of the intact immunoglobulin that results in prolonged serum half-life but in poor tumor penetration and uptake. Indeed, only a very small amount of administered antibody (as low as 0.01%) reaches the tumor. In addition to their size, antibodies encounter other impediments before reaching their target antigens expressed on the cell surface of solid tumors. Some of the barriers include poor blood flow in large tumors, permeability of vascular endothelium, elevated interstitial fluid pressure of tumor stroma, and heterogeneous antigen expression.

With the advent of antibody engineering, small molecular weight antibody fragments exhibiting improved tumor penetration have been generated. Such antibody fragments are often conjugated to specific cytotoxic molecules and are designed to selectively deliver them to cancer cells. Still, solid tumors remain a formidable challenge for therapy, even with immunoconjugated antibody fragments.

The new wave of optimization strategies involves the use of biological modifiers to modulate the impediments posed by solid tumors. Thus, in combination to antibodies or their conjugated antibody fragments, various agents are being used to improve the tumor blood flow, enhance vascular permeability, lower tumor interstitial fluid pressure by modulating stromal cells and extracellular matrix components, upregulate expression of target antigens and improve penetration and retention of therapeutic agent.

Immunotherapy with antibodies represents an exciting opportunity for combinations with standard modalities, such as chemotherapy, as well as combinations with diverse biological agents to obtain synergistic activity. Indeed, unconjugated mAbs are more effective when used in combination with other therapeutic agents, including other antibodies.

Another component of the immune system response to immunotherapy is the cellular response, specifically—the T cell response and activation of cytotoxic T cells (CTLs). The efficiency of the immune system in mediating tumor regression depends on the induction of antigen-specific T-cell responses through physiologic immune surveillance, priming by vaccination, or following adoptive transfer of T-cells. Although a variety of tumor-associated antigens have been identified and many immunotherapeutic strategies have been tested, objective clinical responses are rare. The reasons for this include the inability of current immunotherapy approaches to generate efficient T-cell responses, the presence of regulatory cells that inhibit T-cell responses, and other escape mechanisms that tumors develop, such as inactivation of cytolytic T-cells through expression of negative costimulatory molecules. Effective immunotherapy for cancer will require the use of appropriate tumor-specific antigens; the optimization of the interaction between the antigenic peptide, the APC and the T cell; and the simultaneous blockade of negative regulatory mechanisms that impede immunotherapeutic effects.

Harnessing the immune system to treat chronic diseases is a major goal of immunotherapy. Active and passive immunotherapies are proving themselves as effective therapeutic strategies. Passive immunotherapy, using monoclonal antibodies or receptor Fc-fusion proteins, has come of age and has shown great clinical success. A growing number of such therapeutic agents have been approved or are in clinical trials to prevent allograft rejection or to treat autoimmune diseases and cancer. Active immunotherapy (i.e. vaccines) has been effective against agents that normally cause acute self-limiting infectious diseases followed by immunity and has been at the forefront of efforts to prevent the infectious diseases that plague humankind. However, active immunotherapy has been much less effective against cancer or chronic infectious diseases primarily because these have developed strategies to escape normal immune responses. Among these are negative costimulators of the B7 family, such as B7-H1 and B7-H4, which are highly expressed in certain tumors, and afford local protection from immune cells-mediated attack.

The efficiency of the immune system in mediating tumor regression depends on the induction of antigen-specific T-cell responses through physiologic immune surveillance, priming by vaccination, or following adoptive transfer of T-cells. Although a variety of tumor-associated antigens have been identified and many immunotherapeutic strategies have been tested, objective clinical responses are rare. The reasons for this include the inability of current immunotherapy approaches to generate efficient T-cell responses, the presence of regulatory cells that inhibit T-cell responses, and other escape mechanisms that tumors develop, such as inactivation of cytolytic T-cells through expression of negative costimulatory molecules. Effective immunotherapy for cancer will require the use of appropriate tumor-specific antigens; the optimization of the interaction between the antigenic peptide, the APC and the T cell; and the simultaneous blockade of negative regulatory mechanisms that impede immunotherapeutic effects.

Passive tumor immunotherapy uses the exquisite specificity and lytic capability of the immune system to target tumor specific antigens and treat malignant disease with a minimum of damage to normal tissue. Several approaches have been used to identify tumor-associated antigens as target candidates for immunotherapy. The identification of novel tumor specific antigens expands the spectrum of tumor antigen targets available for immune recognition and provides new target molecules for the development of therapeutic agents for passive immunotherapy, including monoclonal antibodies, whether unmodified or armed. Such novel antigens may also point the way to more effective therapeutic vaccines for active or adoptive immunotherapy.

Despite recent progress in the understanding of cancer biology and cancer treatment, as well as better understanding of the molecules involved in immune responses, the success rate for cancer therapy and for the treatment of autoimmune diseases remains low. Therefore, there is an unmet need for new therapies which can successfully treat cancer and/or autoimmune disorders.

### BRIEF SUMMARY OF THE INVENTION

Without wishing to be limited in any way, in some embodiments the present invention relates to a protein KIAA0746 and its variants, variants of CD20, variants of CD55, which are differentially expressed by some cancers and specific blood cells, and therefore are suitable targets for immunotherapy, cancer therapy, treatment of inflammatory and autoimmune disorders, and drug development. In other embodiments, this invention further relates to the discovery of extracellular domains of KIAA0746 and its variants, variants of CD20, and variants of CD55 which are suitable targets for immunotherapy including treatment and prevention of inflammatory, allergic and autoimmune disorders, cancer therapy, and drug development.

In still other embodiments, the present invention relates to therapeutic and diagnostic antibodies and therapies and diagnostic methods using said antibodies and antibody fragments that specifically bind to proteins according to the invention or a soluble or secreted portion thereof, especially the ectodomain.

According to some embodiments of the present invention there is provided novel therapeutic and diagnostic compositions containing at least one KIAA0746, CD20 or CD55 protein or one of the novel splice variants disclosed herein as well as to provide these novel KIAA0746, CD20 or CD55 splice variants, and nucleic acid sequences encoding for same or fragments thereof, especially the ectodomain or secreted forms of KIAA0746, CD20 or CD55 proteins and/or splice variants.

According to other embodiments of the present invention such proteins, splice variants and nucleic acid sequences are used as novel targets for development of drugs which specifically bind to the KIAA0746, CD20 or CD55 proteins and/or splice variants, and/or drugs which agonize or antagonize the binding of other moieties to the KIAA0746, CD20 or CD55 proteins and/or splice variants.

According to other embodiments of the present invention there is provided drugs which modulate (agonize or antagonize) at least one KIAA0746, CD20 or CD55 related biological activity. Such drugs include by way of example antibodies, small molecules, peptides, ribozymes, antisense molecules, siRNA's and the like. These molecules may directly bind or modulate an activity elicited by the KIAA0746, CD20 or CD55 proteins or KIAA0746, CD20 or CD55 DNA or portions or variants thereof or may indirectly modulate a KIAA0746, CD20 or CD55KIAA0746, CD20, CD55 associated activity or binding of molecules to KIAA0746, CD20, CD55, and portions and variants thereof such as by modulating the binding of KIAA0746, CD20 or CD55 to its counterreceptor or endogenous ligand.

Optionally there is provided novel splice variants of a known KIAA0746 protein (SwissProt accession identifier NP_{—}056002; LOC23231; (SEQ ID NO:14)) or a polynucleotide encoding same, which optionally may be used as diagnostic markers and/or therapeutic agents which agonize or antagonize the binding of other moieties to the KIAA0746 proteins and/or which modulate (agonize or antagonize) at least one KIAA0746 related biological activity.

According to one more specific embodiment, the novel splice variant is an isolated polynucleotide comprising a nucleic acid having a nucleic acid sequence as set forth in any one of Z43375_{—}1_T3 (SEQ ID NO:2), Z43375_{—}1_T6 (SEQ ID NO:3), Z43375_{—}1_T7 (SEQ ID NO:4), Z43375_{—}1_T14 (SEQ ID NO:5), Z43375_{—}1_T16 (SEQ ID NO:6), Z43375_{—}1_T20 (SEQ ID NO:7), Z43375_{—}1_T22 (SEQ ID NO:8), Z43375_{—}1_T23 (SEQ ID NO:9), Z43375_{—}1_T28 (SEQ ID NO:10), Z43375_{—}1_T30 (SEQ ID NO:11), Z43375_{—}1_T31 (SEQ ID NO:12), Z43375_{—}1_T33 (SEQ ID NO:13), or a sequence homologous thereto. According to another embodiment, the isolated polynucleotide is at least 95% homologous to any one of Z43375_{—}1_T3 (SEQ ID NO:2), Z43375_{—}1_T6 (SEQ ID NO:3), Z43375_{—}1_T7 (SEQ ID NO:4), Z43375_{—}1_T14 (SEQ ID NO:5), Z43375_{—}1_T16 (SEQ ID NO:6), Z43375_{—}1_T20 (SEQ ID NO:7), Z43375_{—}1_T22 (SEQ ID NO:8), Z43375_{—}1_T23 (SEQ ID NO:9), Z43375_{—}1_T28 (SEQ ID NO:10), Z43375_{—}1_T30 (SEQ ID NO:11), Z43375_{—}1_T31 (SEQ ID NO:12), and Z43375_{—}1_T33 (SEQ ID NO:13).

According to yet another more specific embodiment, the novel KIAA00746 splice variant is an isolated protein or polypeptide having an amino acid sequence as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), or a sequence homologous thereto. According to another embodiment, the isolated polypeptide is at least 95% homologous to any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30). According to other embodiments of the present invention there is provided molecules and isolated polypeptides comprising the soluble ectodomain (ECD) of the KIAA0746 proteins and fragments thereof as well as nucleic acid sequences encoding said soluble ectodomain, as well as fragments thereof and conjugates and the use thereof as therapeutics.

In more specific embodiments the present invention provides discrete portions of the KIAA0746 proteins including different portions of the extracellular domain corresponding to residues 33-1023 of Z43375_{—}1_P4 (SEQ ID NO:18), or residues 17-1049 of Z43375_{—}1_P8 (SEQ ID NO:19), or residues 33-887 of Z43375_{—}1_P40 (SEQ ID NO:20), or residues 33-995 of Z43375_{—}1_P46 (SEQ ID NO:21), or residues 33-1022 of Z43375_{—}1_P47 (SEQ ID NO:22), or residues 33-977 of Z43375_{—}1_P50 (SEQ ID NO:23), or residues 33-792 of Z43375_{—}1_P51 (SEQ ID NO:24), or residues 33-1010 of Z43375_{—}1_P52 (SEQ ID NO:25), or residues 33-839 of Z43375_{—}1_P53 (SEQ ID NO:26), or residues 33-833 of Z43375_{—}1_P54 (SEQ ID NO:27), or residues 33-867 of Z43375_{—}1_P55 (SEQ ID NO:28), or residues 33-714 of Z43375_{—}1_P56 (SEQ ID NO:29), or residues 21-770 of Z43375_{—}1_P60 (SEQ ID NO:30), or variants thereof possessing at least 80% sequence identity, more preferably at least 90% sequence identity therewith and even more preferably at least 95, 96, 97, 98 or 99% sequence identity therewith.

Proteins Z43375_{—}1_P40 (SEQ ID NO:20), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28) and Z43375_{—}1_P56 (SEQ ID NO:29) are predicted to be secreted proteins, and therefore the entire length of these mature proteins is predicted to be extracellular.

According to other more specific embodiments, the present invention provides a novel splice variant of a known CD20 protein (SwissProt accession identifier CD20 HUMAN (SEQ ID NO:32); known also according to the synonyms B-lymphocyte surface antigen B1; Leu-16; Bp35) or a polynucleotide encoding same, which optionally may be used as diagnostic markers and/or therapeutic agents which agonize or antagonize the binding of other moieties to the CD20-variant proteins and/or which modulate (agonize or antagonize) at least one CD20-variant related biological activity.

It is another embodiment of the invention to provide molecules and isolated polypeptides comprising the soluble ectodomain (ECD) of the CD20 proteins and fragments thereof as well as nucleic acid sequences encoding said soluble ectodomain, as well as fragments thereof and conjugates and the use thereof as therapeutics.

According to one embodiment, the novel CD20 splice variant is an isolated polynucleotide comprising a nucleic acid having a nucleic acid sequence as set forth in HSCD20B_{—}1_T12 (SEQ ID NO:31), or a sequence homologous thereto. According to another embodiment, the isolated polynucleotide is at least 95% homologous to any one of HSCD20B_{—}1_T12 (SEQ ID NO:31).

According to yet another embodiment, the novel splice variant is an isolated protein or polypeptide having an amino acid sequence as set forth in any one of HSCD20B_{—}1_P5 (SEQ ID NO:33), or a sequence homologous thereto. According to another embodiment, the isolated polypeptide is at least 95% homologous to any one of HSCD20B_{—}1_P5 (SEQ ID NO:33).

According to some embodiments of the present invention there is provided molecules and isolated polypeptides comprising the soluble ectodomain (ECD) of the CD20-variant proteins and fragments thereof as well as nucleic acid sequences encoding said soluble ectodomain, as well as fragments thereof and conjugates and the use thereof as therapeutics including their use in immunotherapy (by depleting or modulating the activity of B-cells or other immune cells).

According to yet further embodiments of the present invention there are discrete portions of the CD20-variant proteins including different portions of the extracellular domain corresponding to residues 87-109 or residues 1-63 of HSCD20B_{—}1_P5 (SEQ ID NO:33) sequence disclosed herein, or variants thereof possessing at least 80% sequence identity, more preferably at least 90% sequence identity therewith and even more preferably at least 95, 96, 97, 98 or 99% sequence identity therewith.

According to certain embodiments, the present invention provides novel splice variants of a known CD55 protein (SwissProt accession identifier DAF_HUMAN (SEQ ID NO:42); known also according to the synonym CD55 antigen) or a polynucleotide encoding same, which optionally may be used as diagnostic markers and/or therapeutic agents which agonize or antagonize the binding of other moieties to the CD55 variant proteins and/or which modulate (agonize or antagonize) at least one CD55 variant related biological activity.

According to one embodiment, the novel CD55 splice variant is an isolated polynucleotide comprising a nucleic acid having a nucleic acid sequence as set forth in HUMDAF_T10 (SEQ ID NO:34), HUMDAF_T11 (SEQ ID NO:35), HUMDAF_T17 (SEQ ID NO:36), HUMDAF_T24 (SEQ ID NO:38), HUMDAF_T30 (SEQ ID NO:39), HUMDAF_T31 (SEQ ID NO:40), HUMDAF_T32 (SEQ ID NO:41), or a sequence homologous thereto. According to another embodiment, the isolated polynucleotide is at least 95% homologous to HUMDAF_T10 (SEQ ID NO:34), HUMDAF_T11 (SEQ ID NO:35), HUMDAF_T17 (SEQ ID NO:36), HUMDAF_T24 (SEQ ID NO:38), HUMDAF_T30 (SEQ ID NO:39), HUMDAF_T31 (SEQ ID NO:40), and HUMDAF_T32 (SEQ ID NO:41).

According to yet another embodiment, the novel CD55 splice variant is an isolated protein or polypeptide having an amino acid sequence as set forth in, HUMDAF_P20 (SEQ ID NO:53), HUMDAF_P29 (SEQ ID NO:55), HUMDAF_P30 (SEQ ID NO:56), HUMDAF_P31 (SEQ ID NO:57) or a sequence homologous thereto. According to another embodiment, the isolated polypeptide is at least 95, 96, 97, 98 or 99% homologous to HUMDAF_P20 (SEQ ID NO:53), HUMDAF_P29 (SEQ ID NO:55), HUMDAF_P30 (SEQ ID NO:56), and HUMDAF_P31 (SEQ ID NO:57).

According to some embodiments of the present invention there is provided molecules and isolated polypeptides comprising the soluble ectodomain (ECD) of the CD55 proteins and fragments thereof as well as nucleic acid sequences encoding said soluble ectodomain, as well as fragments thereof and conjugates and the use thereof as therapeutics.

According to yet further embodiments of the present invention there are discrete portions of the CD55 proteins including different portions of the extracellular domain corresponding to residues 35-497 of HUMDAF_P14 (SEQ ID NO:51), or residues 35-523 of HUMDAF_P15 (SEQ ID NO:52), or residues 35-497 of HUMDAF_P20 (SEQ ID NO:53), or residues 36-371 of HUMDAF_P26 (SEQ ID NO:54), or residues 35-328 of HUMDAF_P29 (SEQ ID NO:55), or residues 35-497 of HUMDAF_P30 (SEQ ID NO:56), or residues 35-523 of HUMDAF_P31 (SEQ ID NO:57), or variants thereof possessing at least 80% sequence identity, more preferably at least 90% sequence identity therewith and even more preferably at least 95, 96, 97, 98 or 99% sequence identity therewith.

According to some embodiments of the present invention there is provided an isolated or purified soluble protein or nucleic acid sequence encoding having or encoding the extracellular domain of any one of the KIAA0746, CD20, or CD55 proteins which optionally may be directly or indirectly attached to a non-KIAA0746, non-CD20, or non-CD55 protein or nucleic acid sequence, respectively, such as a soluble immunoglobulin domain or fragment.

According to some embodiments of the present invention there is provided molecules and isolated polypeptides comprising edge portion, tail or head portion, of any one of the KIAA0746, CD20, CD55 novel variants of the invention, or a homologue or a fragment thereof as well as nucleic acid sequences encoding said edge portion, tail or head portion, as well as fragments thereof and conjugates and the use thereof as therapeutics and/or for diagnostics.

According to other embodiments of the present invention there is provided molecules and isolated polypeptides comprising a bridge, edge portion, tail or head portion, as depicted in any one of SEQ ID NOs: 176-218, or a homologue or a fragment thereof as well as nucleic acid sequences encoding said edge portion, tail or head portion, as well as fragments thereof and conjugates and the use thereof as therapeutics and/or for diagnostics.

According to some embodiments of the present invention there is provided vectors such as plasmids and recombinant viral vectors and host cells containing the vectors that express any one of KIAA0746, CD20, CD55, its secreted or soluble form and/or the ECD of the KIAA0746, CD20, or CD55 protein and variants thereof or polypeptide conjugates containing any of the foregoing.

According to still other embodiments there is provided use of these vectors such as plasmids and recombinant viral vectors and host cells containing that express any one of KIAA0746, CD20, CD55, its secreted or soluble form and/or the ECD of the KIAA0746, CD20, CD55 protein and variants thereof or polypeptide conjugates containing any of the foregoing to produce said KIAA0746, CD20, CD55 protein, fragments or variants thereof and/or conjugates containing any one of the foregoing.

According to some embodiments of the present invention there is provided pharmaceutical or diagnostic compositions containing any of the foregoing.

According to some embodiments of the present invention there is provided compounds and use thereof including KIAA0746, CD20, or CD55 variant proteins, and fragments thereof, and KIAA0746, CD20, or CD55 ectodomain or fragments or variants thereof, which are suitable for immunotherapy, treatment, prevention or diagnosis of cancer, inflammatory or autoimmune disorders, transplant rejection, graft versus host disease, and/or for blocking or promoting immune costimulation mediated by the KIAA0746, CD20, or CD55 polypeptide.

According to some embodiments of the present invention there are provided compounds and use thereof including KIAA0746, CD20, or CD55 variant proteins, and fragments thereof, and KIAA0746, CD20, or CD55 ectodomain or fragments or variants thereof, which are suitable for treatment, prevention, or diagnosis of cancer, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

According to some embodiments of the present invention there are provided compounds and use thereof including KIAA0746 or CD55 variant proteins, and fragments thereof, and KIAA0746 or CD55 ectodomain or fragments or variants thereof, which are suitable for treatment, prevention or diagnosis of cancer, wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

According to some embodiments of the present invention there are provided compounds and use thereof including CD20 variant proteins, and fragments thereof, and CD20 ectodomain or fragments or variants thereof, which are suitable for treatment, prevention, or diagnosis of cancer, wherein the cancer is a hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

According to some embodiments of the present invention there are provided compounds and use thereof including KIAA0746, CD20, or CD55 variant proteins, and fragments thereof, and KIAA0746, CD20, or CD55 ectodomain or fragments or variants thereof, which are suitable for treatment, prevention or diagnosis of immune related condition, wherein the immune related condition is inflammatory or autoimmune disease, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

According to some embodiments of the present invention there are provided compounds and use thereof including KIAA0746 or CD20 variant proteins, and fragments thereof, and KIAA0746 or CD20 ectodomain or fragments or variants thereof, which are suitable for treatment, prevention or diagnosis of immune related condition, wherein the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

According to some embodiments of the present invention there are provided compounds and use thereof including CD55 variant proteins, and fragments thereof, and CD55 ectodomain or fragments or variants thereof, which are suitable for treatment, prevention or diagnosis of immune related condition, wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

According to other embodiments of the present invention, there is provided compounds (and the use thereof) including CD20 or CD55 variant proteins and fragments thereof and CD20 or CD55 variant ectodomain or fragments or variants thereof, which are suitable for treatment, prevention or diagnosis of acute and chronic rejection of organ transplantation, allogenic stem cell transplantation, autologous stem cell transplantation, bone marrow transplantation, and graft versus host disease.

According to other embodiments of the present invention, there is provided compounds (and the use thereof) including CD55 variant transcripts, proteins and fragments thereof and CD55 variant ectodomain or fragments or variants thereof, which are suitable for transgenic animals generation and the use of these CD55 variant-transgenic animals for xenotransplantation.

According to other embodiments of the present invention, there is provided compounds (and the use thereof) including CD55 variant proteins and fragments thereof and CD55 variant ectodomain or fragments or variants thereof, which are suitable for treatment, prevention or diagnosis of the ischemia-reperfusion injury related disorders, selected from the group including but not limited to ischemia-reperfusion injury related disorder associated with ischemic and post-ischemic events in organs and tissues, thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from angiography, cardiopulmonary and cerebral resuscitation, cardiac surgery, organ surgery, organ transplantation, and systemic and intragraft inflammatory responses after cold ischemia-reperfusion in the setting of organ transplantation.

According to some embodiments of the present invention there are provided compounds and use thereof including CD55 variant proteins and fragments thereof and CD55 variant ectodomain or fragments or variants thereof, which are suitable for treatment, prevention or diagnosis of inflammation of the respiratory tract disorders, selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

According to some embodiments of the present invention there are provided compounds and use thereof including KIAA0746 or CD20 variant proteins, and fragments thereof, and KIAA0746 or CD20 ectodomain or fragments or variants thereof, which are suitable for treatment, prevention or diagnosis of lymphoproliferative disorders. According to the invention the lymphoproliferative disorder is selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

According to some embodiments of the present invention there are provided compounds and use thereof including CD55 variant proteins and fragments thereof and CD55 variant ectodomain or fragments or variants thereof, which are suitable for treatment, prevention or diagnosis of disease states in which complement activation and deposition is involved in pathogenesis.

According to other embodiments of the present invention, there is provided monoclonal or polyclonal antibodies and antibody fragments and conjugates containing such, that specifically bind the full length KIAA0746, CD20 or CD55 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), its secreted form and/or the ECD thereof or conjugates or fragments thereof. These antibodies are potentially useful as therapeutics and/or diagnostic agents (both in vitro and in vivo diagnostic methods). Included in particular are antibodies and fragments that are immune activating or immune suppressing such as antibodies or fragments that target cells via ADCC (antibody dependent cellular cytotoxicity) or CDC (complement dependent cytotoxicity) activities. In addition these antibodies are useful for generating and selecting for anti-idiotypic antibodies specific thereto which also are potentially useful as therapeutics and/or diagnostic agents (both in vitro and in vivo diagnostic methods).

According to some embodiments of the present invention there is provided diagnostic methods that include the use of any of the foregoing including by way of example immunohistochemical assay, radioimaging assays, in-vivo imaging, radioimmunoassay (RIA), ELISA, slot blot, competitive binding assays, fluorimetric imaging assays, Western blot, FACS, and the like. In particular this includes assays which use chimeric or non-human antibodies or fragments that specifically bind the intact KIAA0746, CD20 or CD55 protein, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), its soluble form, its ECD, and or conjugates, fragments or variants thereof.

According to other embodiments of the present invention, there is provided therapeutically effective polyclonal or monoclonal antibodies against any one of the KIAA0746, CD20 or CD55 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates, and variants thereof or anti-idiotypic antibodies specific to any of the foregoing for treating, preventing or diagnosing conditions wherein the KIAA0746, CD20 or CD55 antigen or its secreted or soluble form or ECD and/or portions or variants thereof are differentially expressed, including various cancers and malignancies, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

According to other embodiments, there is provided use of novel therapeutically effective polyclonal or monoclonal antibodies against any one of the KIAA0746 or CD55 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates, and variants thereof or anti-idiotypic antibodies specific to any of the foregoing for treating, preventing or diagnosing conditions wherein the KIAA0746 or CD55 antigen or its secreted or soluble form or ECD and/or portions or variants thereof are differentially expressed, including various cancers and malignancies, wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic. According to still other embodiments there is provided use of novel therapeutically effective polyclonal or monoclonal antibodies against CD20 antigen, selected from the group consisting of HSCD20B_{—}1_P5 (SEQ ID NO:33), and fragments, conjugates, and variants thereof for treating, preventing or diagnosing conditions wherein the CD20 antigen or its secreted or soluble form or ECD and/or portions or variants thereof are differentially expressed, including various cancers and malignancies, wherein the cancer is selected from the group consisting of hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

According to still other embodiments there is provided use of novel therapeutically effective polyclonal or monoclonal antibodies against any one of the KIAA0746, CD20 or CD55 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates and variants thereof or anti-idiotypic antibodies specific to any of the foregoing for treating, preventing or diagnosing of non-malignant disorders such as immune related condition, wherein the immune related condition is inflammatory or autoimmune disease, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

According to still other embodiments there is provided use of novel therapeutically effective polyclonal or monoclonal antibodies against any one of the KIAA0746 or CD20 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), and fragments, conjugates and variants thereof or anti-idiotypic antibodies specific to any of the foregoing for treating, preventing or diagnosing of immune related condition, wherein the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

According to still other embodiments there is provided use of novel therapeutically effective polyclonal or monoclonal antibodies against any one of the CD55 antigen, selected from the group consisting of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates and variants thereof or anti-idiotypic antibodies specific to any of the foregoing for treating, preventing or diagnosing of immune related condition, wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

According to still other embodiments there is provided use of novel therapeutically effective polyclonal or monoclonal antibodies against any one of the CD55 antigen, selected from the group consisting of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates and variants thereof or anti-idiotypic antibodies specific to any of the foregoing for treating, preventing or diagnosing of ischemia-reperfusion injury, wherein the ischemia-reperfusion injury is selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

According to still other embodiments there is provided use of novel therapeutically effective polyclonal or monoclonal antibodies against any one of the CD55 antigen, selected from the group consisting of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates and variants thereof or anti-idiotypic antibodies specific to any of the foregoing for treating, preventing or diagnosing of inflammation of the respiratory tract disorder, wherein the inflammation of the respiratory tract disorder is selected from the group consisting of chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

According to still other embodiments there is provided use of novel therapeutically effective polyclonal or monoclonal antibodies against any one of the KIAA0746 or CD20 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), and fragments, conjugates and variants thereof or anti-idiotypic antibodies specific to any of the foregoing for treating, preventing or diagnosing of lymphoproliferative disorder, wherein the lymphoproliferative disorder is selected from the group consisting of EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

According to still other embodiments there is provided use of novel therapeutically effective polyclonal or monoclonal antibodies against CD55 antigen, selected from the group consisting of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and fragments, conjugates and variants thereof or anti-idiotypic antibodies specific to any of the foregoing for treating, preventing or diagnosing of disease states in which complement activation and deposition is involved in pathogenesis.

According to still other embodiments there is provided use of novel therapeutically effective polyclonal or monoclonal antibodies against any one of the CD20 or CD55 antigen, selected from the group consisting of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), HSCD20B_{—}1_P5 (SEQ ID NO:33), and fragments, conjugates and variants thereof or anti-idiotypic antibodies specific to any of the foregoing for treating, preventing or diagnosing transplant rejection disorders, selected from the group including but not limited to acute and chronic rejection of organ transplantation and/or of allogeneic stem cell transplantation, autologous stem cell transplantation, bone marrow transplantation, Graft Versus Host Disease (GVHD), rejection in xenotransplantation.

According to still other embodiments there is provided use of antibodies and antibody fragments, and conjugates thereof, against the KIAA0746, CD20 or CD55 antigen, selected from the group consisting of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57) in modulating (enhancing or inhibiting) immunity. It is another embodiment of the invention to produce antibodies and antibody fragments against discrete portions of the KIAA0746 proteins including different portions of the extracellular domain corresponding to residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105. According to other embodiments there is provided a method to produce or select for anti-idiotypic antibodies specific to any of the foregoing.

It is another specific embodiment of the invention to produce antibodies and antibody fragments against discrete portions of the CD20 proteins including different portions of the extracellular domain corresponding to residues 87-109 or residues 1-63 of HSCD20B_{—}1_P5 (SEQ ID NO:33), corresponding to amino acid sequence depicted in SEQ ID NO: 106 or SEQ ID NO:107, respectively. According to other embodiments there is provided a method to produce or select for anti-idiotypic antibodies specific to any of the foregoing.

According to other embodiments there is provided a method to produce antibodies and antibody fragments against discrete portions of the CD55 proteins including different portions of the extracellular domain corresponding to residues 35-497 of HUMDAF_P14 (SEQ ID NO:51), corresponding to amino acid sequence depicted in SEQ ID NO:108, or residues 35-523 of HUMDAF_P15(SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112. According to still other embodiments there is provided a method to produce or select for anti-idiotypic antibodies specific to any of the foregoing.

It is a embodiment of the invention to provide polyclonal and monoclonal antibodies and fragments thereof or an antigen binding fragment thereof comprising an antigen binding site that binds specifically to the KIAA0746, CD20 or CD55 proteins, its variants, its soluble forms, the ECD thereof and/or variants and fragments thereof. According to still other embodiments there is provided a method to produce or select for anti-idiotypic antibodies specific to any of the foregoing.

According to still other embodiments there is provided a method to use such antibodies and fragments thereof for treatment or prevention of cancer and/or for modulating (activating or blocking) the activity of the target in the immune co-stimulatory system.

According to still other embodiments there is provided a method to select monoclonal and polyclonal antibodies and fragments thereof against KIAA0746, CD20 or CD55 which are suitable for treatment or prevention of cancer, immune related condition, and/or for blocking or enhancing immune costimulation mediated by the KIAA0746, CD20 or CD55 polypeptide.

According to still other embodiments there is provided a method to use antibodies against any one of the KIAA0746, CD20 or CD55 antigen, soluble form, ECD or fragment or variant thereof for the treatment and diagnosis of cancers wherein the cancer is selected from the group consisting of hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

According to some embodiments of the present invention there is provided use of antibodies and antibody fragments against any one of the KIAA0746, CD20 or CD55 antigen, its soluble form, or ECD and variants or fragments thereof as well as soluble polypeptides containing the ectodomain of the KIAA0746, CD20 or CD55 antigen or a portion thereof which are useful for immune modulation, including treatment of immune related conditions, wherein the immune related conditions are inflammatory and/or autoimmune diseases, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

According to some embodiments of the present invention there are provided compounds and use thereof including drugs such as small molecules, aptamers, peptides, antibodies and fragments that bind any one of the KIAA0746, CD20 or CD55 antigen, as well as ribozymes or antisense or siRNAs which target the KIAA0746, CD20 or CD55 nucleic acid sequence or fragments or variants thereof which are useful for treatment or prevention of immune related conditions, and/or for blocking or enhancing immune costimulation mediated by the KIAA0746, CD20 or CD55 polypeptide.

According to some embodiments of the present invention there are provided compounds and use thereof including drugs such as small molecules, aptamers, peptides, antibodies and fragments that bind the KIAA0746 or CD20 antigen, as well as ribozymes or antisense or siRNAs which target the KIAA0746 or CD20 nucleic acid sequence or fragments or variants thereof which are useful for treatment or prevention of immune related conditions, selected from the group including but not limited to rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

According to some embodiments of the present invention there are provided compounds and use thereof including drugs such as small molecules, aptamers, peptides, antibodies and fragments that bind the CD55 antigen, as well as ribozymes or antisense or siRNAs which target the CD55 nucleic acid sequence or fragments or variants thereof which are useful for treatment or prevention of immune related conditions, selected from the group including but not limited to rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis. According to some embodiments of the present invention there are provided compounds and use thereof including drugs such as small molecules, aptamers, peptides, antibodies and fragments that bind the KIAA0746, CD20 or CD55 antigen, as well as ribozymes or antisense or siRNAs which target the KIAA0746, CD20 or CD55 nucleic acid sequence or fragments or variants thereof which are useful for treatment or prevention of cancer.

According to some embodiments of the present invention there are provided compounds and use thereof including drugs such as small molecules, aptamers, peptides, antibodies and fragments that bind the KIAA0746 or CD55 antigen, as well as ribozymes or antisense or siRNAs which target the KIAA0746 or CD55 nucleic acid sequence or fragments or variants thereof which are useful for treatment or prevention of cancer, selected from the group including but not limited to colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

According to some embodiments of the present invention there are provided compounds and use thereof including drugs such as small molecules, aptamers, peptides, antibodies and fragments that bind the CD20 antigen, as well as ribozymes or antisense or siRNAs which target the CD20 nucleic acid sequence or fragments or variants thereof which are useful for treatment or prevention of cancer, selected from the group including but not limited to hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

According to some embodiments of the present invention there are provided compounds and use thereof including drugs such as small molecules, aptamers, peptides, antibodies and fragments that bind the KIAA0746 or CD20 antigen, as well as ribozymes or antisense or siRNAs which target the KIAA0746 or CD20 nucleic acid sequence or fragments or variants thereof which are useful for treatment or prevention of lymphoproliferative disorders.

According to some embodiments of the present invention there are provided compounds and use thereof including drugs such as small molecules, aptamers, peptides, antibodies and fragments that bind the CD55 antigen, as well as ribozymes or antisense or siRNAs which target the CD55 nucleic acid sequence or fragments or variants thereof which are useful for treatment or prevention of inflammation of the respiratory tract disorders or ischemia-reperfusion injury related disorders.

According to still other embodiments there is provided therapeutic and diagnostic antibodies and fragments and conjugates thereof useful in treating or diagnosing any of the foregoing that specifically bind to amino-acids residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105.

It is a preferred embodiment to provide therapeutic and diagnostic antibodies and fragments and conjugates thereof useful in treating or diagnosing any of the foregoing that specifically bind to amino-acids residues 87-109 or residues 1-63 of HSCD20B_{—}1_P5 (SEQ ID NO:33), corresponding to amino acid sequence depicted in SEQ ID NO: 106 or SEQ ID NO: 107, respectively.

It is a preferred embodiment to provide therapeutic and diagnostic antibodies and fragments and conjugates thereof useful in treating or diagnosing any of the foregoing that specifically bind to amino-acids residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112.

It is also a preferred embodiment to provide antibodies and fragments thereof that bind to KIAA0746, CD20 or CD55 and the specific residues above-identified and fragments thereof, wherein the antibody is a chimeric, humanized, fully human antibody and/or is an antibody or antibody fragment having CDC or ADCC activities on target cells.

It is also a preferred embodiment to provide chimeric and human antibodies and fragments thereof and conjugates thereof that bind to KIAA0746, CD20 or CD55 and the specific residues above-identified and fragments thereof.

According to other embodiments of the present invention there is provided antibody fragments and conjugates thereof useful in the foregoing therapies and related diagnostic methods including but not limited to Fab, F(ab′)₂, Fv or scFv fragment.

It is also an embodiment of the invention to directly or indirectly attach the subject antibodies and fragments to markers and other effector moieties such as a detectable marker, or to an effector moiety such as an enzyme, a toxin, a therapeutic agent, or a chemotherapeutic agent.

In a preferred embodiment the inventive antibodies or fragments may be attached directly or indirectly to a radioisotope, a metal chelator, an enzyme, a fluorescent compound, a bioluminescent compound or a chemiluminescent compound.

According to other embodiments of the present invention there is provided pharmaceutical and diagnostic compositions that comprise a therapeutically or diagnostically effective form of an antibody or antibody fragment.

According to other embodiments of the present invention there is provided a method for inhibiting the growth of cells that express KIAA0746 in a subject, comprising: administering to said subject an antibody that specifically binds to the antigen referred to herein as Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30) or KIAA0746.

According to other embodiments of the present invention there is provided methods for treating, or preventing cancer, comprising administering to a patient an effective amount of a monoclonal antibody that specifically bind Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30) or KIAA0746.

Preferably these antibodies are used for treating or preventing cancer selected from the group including but not limited to ovarian cancer, lung cancer, colorectal cancer, prostate cancer, pancreas cancer, liver cancer, melanoma, kidney cancer, head and neck cancer, and wherein the ovarian cancer, lung cancer, colorectal cancer, prostate cancer, pancreas cancer, liver cancer, melanoma, kidney cancer, head and neck cancer is non-metastatic, invasive or metastatic, wherein preferably the antibody has an antigen-binding region specific for the extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30).

According to some embodiments of the present invention there is provided methods for treating, or preventing immune related conditions, comprising administering to a patient an effective amount of a polyclonal or monoclonal antibody or fragment or a conjugate containing that specifically bind Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), and Z43375_{—}1_P60 (SEQ ID NO:30).

It is a more preferred embodiment of the invention to use these antibodies for treating or preventing immune related condition selected from the group including but not limited to rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders (such as pemphigus, pemphigoid), atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy, wherein preferably the antibody has an antigen-binding region specific for the extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), and Z43375_{—}1_P60 (SEQ ID NO:30).

According to some embodiments of the present invention there is provided methods for treating or preventing lymphoproliferative disorder, comprising administering to a patient an effective amount of a polyclonal or monoclonal antibody or fragment or a conjugate containing that specifically bind Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), and Z43375_{—}1_P60 (SEQ ID NO:30).

It is a more preferred embodiment of the invention to use these antibodies for treating or preventing lymphoproliferative disorder selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS), wherein preferably the antibody has an antigen-binding region specific for the extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), and Z43375_{—}1_P60 (SEQ ID NO:30).

It is another specific embodiment of the invention to inhibit the growth of cells that express CD20 in a subject, comprising: administering to said subject an antibody that specifically binds to the antigen referred to herein as HSCD20B_{—}1_P5 (SEQ ID NO:33), or CD20.

It is another specific embodiment of the invention to provide methods for treating or preventing cancer, comprising administering to a patient an effective amount of a monoclonal antibody that specifically binds to HSCD20B_{—}1_P5 (SEQ ID NO:33) or CD20.

It is a more preferred embodiment of the invention to use these antibodies for treating or preventing hematological malignancy, selected from the group including but not limited to acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy is non-metastatic, invasive or metastatic, and wherein preferably the antibody has an antigen-binding region specific for the extracellular domain of HSCD20B_{—}1_P5 (SEQ ID NO:33) or CD20.

It is another embodiment of the invention to provide methods for treating or preventing immune related conditions, comprising administering to a patient an effective amount of a polyclonal or monoclonal antibody or fragment that specifically binds HSCD20B_{—}1_P5 (SEQ ID NO:33) or CD20.

It is a more preferred embodiment of the invention to use these antibodies for treating or preventing immune related condition, selected from the group including but not limited to rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders (such as pemphigus, pemphigoid), atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy, and wherein preferably the antibody has an antigen-binding region specific for the extracellular domain of HSCD20B_{—}1_P5 (SEQ ID NO:33) or CD20.

It is another preferred embodiment of the invention to use these antibodies for treating or preventing immune related condition, selected from the group including but not limited to acute and chronic rejection of organ transplantation, allogeneic stem cell transplantation, autologous stem cell transplantation, bone marrow transplantation, and treatment of Graft Versus Host Disease (GVHD), and wherein preferably the antibody has an antigen-binding region specific for the extracellular domain of HSCD20B_{—}1_P5 (SEQ ID NO:33) or CD20.

According to some embodiments of the present invention there is provided methods for treating or preventing lymphoproliferative disorder, comprising administering to a patient an effective amount of a polyclonal or monoclonal antibody or fragment or a conjugate containing that specifically bind HSCD20B_{—}1_P5 (SEQ ID NO:33) or CD20.

It is a more preferred embodiment of the invention to use these antibodies for treating or preventing lymphoproliferative disorder selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS), wherein preferably the antibody has an antigen-binding region specific for the extracellular domain of HSCD20B_{—}1_P5 (SEQ ID NO:33) or CD20.

It is another embodiment of the invention to inhibit the growth of cells that express CD55 in a subject, comprising: administering to said subject an antibody that specifically binds to the antigen referred to herein as HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is another embodiment of the invention to provide methods for treating or preventing cancer, comprising administering to a patient an effective amount of a monoclonal antibody that specifically bind HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is a more preferred embodiment of the invention to use these antibodies for treating cancers selected from the group including but not limited to colorectal cancer, lung cancer, prostate cancer, pancreas cancer, ovarian cancer, gastric cancer and liver cancer, and wherein the colorectal cancer, lung cancer, prostate cancer, pancreas cancer, ovarian cancer, gastric cancer and liver cancer is non-metastatic, invasive or metastatic, and wherein preferably the antibody has an antigen-binding region specific for the extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

According to some embodiments of the present invention there is provided methods for treating or preventing immune related condition, comprising administering to a patient an effective amount of a polyclonal or monoclonal antibody or fragment that specifically bind HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is a more preferred embodiment of the invention to use these antibodies for treating immune related condition selected from the group including but not limited to rheumatoid arthritis (RA), systemic lupus erythematosus (SLE), lupus nephtirits and multiple sclerosis (MS), inflammatory bowel disease (IBD), ulcerative colitis and psoriasis, and or for therapy of disease states in which complement activation and deposition is involved in pathogenesis, and wherein preferably the antibody has an antigen-binding region specific for the extracellular domain of HUMDAF_P14 (SEQ ID NO:51), HUMDAF_P15 (SEQ ID NO:52), HUMDAF_P20 (SEQ ID NO: 53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is another preferred embodiment of the invention to use these antibodies for treating immune related condition selected from the group including but not limited to acute and chronic rejection of organ transplantation and of allogeneic stem cell transplantation, autologous stem cell transplantation, bone marrow transplantation, treatment of Graft Versus Host Disease (GVHD), rejection in xenotransplantation, and wherein preferably the antibody has an antigen-binding region specific for the extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

According to some embodiments of the present invention there is provided methods for treating or preventing inflammation of the respiratory tract disorders, comprising administering to a patient an effective amount of a polyclonal or monoclonal antibody or fragment that specifically bind HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is a more preferred embodiment of the invention to use these antibodies for treating inflammation of the respiratory tract disorder, selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis, and wherein preferably the antibody has an antigen-binding region specific for the extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

According to some embodiments of the present invention there is provided methods for treating or preventing ischemia-reperfusion injury disorders, comprising administering to a patient an effective amount of a polyclonal or monoclonal antibody or fragment that specifically bind HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is a more preferred embodiment of the invention to use these antibodies for treating ischemia-reperfusion injury disorder, selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation, and wherein preferably the antibody has an antigen-binding region specific for the extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or CD55.

It is another embodiment of the invention to use part or all of the ectodomain of KIAA0746, CD20, CD55 or its variants and conjugates thereof for administration as an anti-cancer vaccine, for immunotherapy of cancer, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

It is another embodiment of the invention to use part or all of the ectodomain of KIAA0746 or its variants and conjugates thereof for administration as an anti-cancer vaccine, for immunotherapy of cancer, selected from but not limited to ovarian cancer, lung cancer, colorectal cancer, prostate cancer, pancreas cancer, liver cancer, melanoma, kidney cancer, head and neck cancer.

It is another embodiment of the invention to use part or all of the ectodomain of CD20, or its variants and conjugates thereof for administration as an anti-cancer vaccine, for immunotherapy of cancer, selected from but not limited to acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia.

It is another embodiment of the invention to use part or all of the ectodomain of CD55 or its variants and conjugates thereof for administration as an anti-cancer vaccine, for immunotherapy of cancer, selected from but not limited to colorectal cancer, lung cancer, prostate cancer, pancreas cancer, ovarian cancer, gastric cancer and liver cancer.

According to the present invention, each one of the following: the KIAA0746 ectodomain, CD20 ectodomain, CD55 ectodomain, antibodies and fragments that bind the KIAA0746, CD20 or CD55 antigen, the compounds including drugs such as small molecules, aptamers, peptides, as well as ribozymes or antisense or siRNAs which target the KIAA0746, CD20 or CD55 nucleic acid sequence or fragments or variants thereof which are useful for treatment or prevention of cancer, immune related conditions, and/or for blocking or enhancing immune co-stimulation mediated by the KIAA0746, CD20 or CD55 polypeptide, optionally may be used with simultaneous blockade of several co-stimulatory pathways or in combination therapy with conventional drugs, such as immunosuppressants or cytotoxic drugs for cancer.

According to some embodiments of the present invention there is provided assays for detecting the presence of at least one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57) protein in vitro or in vivo in a biological sample or individual comprising contacting the sample with an antibody having specificity for at least one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57) polypeptides, or a combination thereof, and detecting the binding of at least one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57) protein in the sample to said antibody.

According to some embodiments of the present invention there is provided methods for detecting a disease, diagnosing a disease, monitoring disease progression or treatment efficacy or relapse of a disease, or selecting a therapy for a disease, comprising detecting the expression of at least one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57).

In a related embodiment the detected diseases will include cancers wherein the cancer is selected from the group consisting of hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

With regard to lung cancer, the disease is selected from the group consisting of non-metastatic, invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affecting treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

With regard to ovarian cancer, the compounds of the present invention optionally may be used in the diagnosis, treatment or prognostic assessment of non-metastatic, invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

With regard to breast cancer, the compounds of the invention are useful in determining a probable outcome in breast cancer; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercalcemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

With regard to renal cancer, the compounds of this invention may be used for the diagnosis, treatment selection and monitoring, or assessment of prognosis of primary and/or metastatic cancer of the kidney, including but not limited to renal cell carcinoma (i.e. renal adenocarcinoma), as well as other non-epithelial neoplasms of the ovary, including nephroblastoma (i.e. Wilm's tumor), transitional cell neoplasms of the renal pelvis, and various sarcomas of renal origin. With regard to liver cancer, the compounds of this invention may be used for the diagnosis, treatment selection and monitoring, or assessment of prognosis of primary and metastatic cancer of the liver and intrahepatic bile duct, including hepatocellular carcinoma, cholangiocarcinoma, hepatic angiosarcoma and hepatoblastoma.

With regard to pancreatic cancer, the compounds of this invention may be used for the diagnosis, treatment selection and monitoring, or assessment of prognosis of primary and/or metastatic cancer of the exocrine pancreas, including but not limited to adenocarcinoma, serous and mucinous cystadenocarcinomas, acinar cell carcinoma, undifferentiated carcinoma, pancreatoblastoma and neuroendocrine tumors such as insulinoma.

With regard to prostate cancer, the compounds of this invention may be used for the diagnosis, treatment selection and monitoring, or assessment of prognosis of primary and/or metastatic cancer of the prostate, including but not limited to prostatic intraepithelial neoplasia, atypical adenomatous hyperplasia, prostate adenocarcinoma, mucinous or signet ring tumor, adenoid cystic carcinoma, prostatic duct carcinoma, carcinoid and small-cell undifferentiated cancer. In some embodiments the polypeptides/polynucleotides of this invention are useful in the diagnosis of prostate cancer, which includes, inter alia, the differential diagnosis between prostate cancer and BPH, prostatitis and/or prostatism.

With regard to melanoma, the compounds of this invention may be used for the diagnosis, treatment selection and monitoring, or assessment of prognosis of primary and/or metastatic malignant melanoma, including but not limited to cutaneous melanoma such as superficial spreading melanoma, nodular melanoma, acral lentiginous melanoma and lentigo maligna melanoma, as well as mucosal melanoma, intraocular melanoma, desmoplastic/neurotropic melanoma and melanoma of soft parts (clear cell sarcoma).

With regard to gastric cancer, the compounds of this invention may be used for the diagnosis, treatment selection and monitoring, or assessment of prognosis of primary and/or metastatic gastric cancer, including but not limited to gastric carcinoma, gastric adenocarcinoma (Intestinal or Diffused). With regard to liver cancer, the compounds of this invention may be used for the diagnosis, treatment selection and monitoring, or assessment of prognosis of primary and/or metastatic liver cancer, including but not limited to hepatocellular carcinoma (HCC), hepatocellular cancer, intrahepatic cholangiocarcinomas (bile duct cancers), angiosarcomas and hemangiosarcomas.

With regard to head and neck cancer, the compounds of this invention may be used for the diagnosis, treatment selection and monitoring, or assessment of prognosis of primary and/or metastatic head and neck cancer, including but not limited to squamous cell carcinoma, verrucous carcinoma, carcinoid of the head and neck.

In another related embodiment the detected diseases will include immune related conditions, wherein the immune related conditions are inflammatory and autoimmune diseases, selected from the group consisting of multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease. In a related aspect the foregoing assays will detect cells affected by the disease using an antibody that binds specifically to at least one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57) protein wherein the assays may be effected in vitro or in vivo, and include RIA, ELISA, fluorimetric assays, FACS, slot blot, Western blot, immunohistochemical assays, radioimaging assays and the like. In some embodiments, this invention provides a method for diagnosing a disease in a subject, comprising detecting in the subject or in a sample obtained from said subject at least one polypeptide or polynucleotide selected from the group consisting of: a polypeptide comprising an amino acid sequence as set forth in any one of SEQ ID NOs: 18-30, 33, 51-57, 93-114;

a polypeptide comprising a bridge, edge portion, tail or head portion, of any one of SEQ ID NOs: 176-218, or a homologue or a fragment thereof;

a polynucleotide comprising a nucleic acid sequence as set forth in any one of SEQ ID NOs: 1-13, 31, 34-41;

a polynucleotide comprising a nucleic acid sequence encoding a polypeptide comprising a bridge, edge portion, tail or head portion, of any one of SEQ ID NOs: 176-218;

an oligonucleotide having a nucleic acid sequence as set forth in SEQ ID NOs: 81, 84, 87, 90, 92.

According to further embodiment, the method of detecting a polypeptide according to the invention comprises employing an antibody capable of specifically binding to at least one epitope of a polypeptide comprising an amino acid sequence of a polypeptide comprising a bridge, edge portion, tail, or head portion of any one of SEQ ID NOs: 176-218, and/or antibody capable of specifically binding to at least one epitope of a polypeptide comprising an amino acid sequence of a polypeptide comprising an extracellular domain of any one of KIAA0746, CD20 or CD55, particularly as depicted in any one of SEQ ID NOs:93-114.

According to one embodiment, detecting the presence of the polypeptide or polynucleotide is indicative of the presence of the disease and/or its severity and/or its progress. According to another embodiment, a change in the expression and/or the level of the polynucleotide or polypeptide compared to its expression and/or level in a healthy subject or a sample obtained therefrom is indicative of the presence of the disease and/or its severity and/or its progress. According to a further embodiment, a change in the expression and/or level of the polynucleotide or polypeptide compared to its level and/or expression in said subject or in a sample obtained therefrom at earlier stage is indicative of the progress of the disease. According to still further embodiment, detecting the presence and/or relative change in the expression and/or level of the polynucleotide or polypeptide is useful for selecting a treatment and/or monitoring a treatment of the disease.

According to one embodiment, detecting a polynucleotide of the invention comprises employing a primer pair, comprising a pair of isolated oligonucleotides capable of specifically hybridizing to at least a portion of a polynucleotide having a nucleic acid sequence as set forth in SEQ ID NOs: 1-13, 31, 34-41, 71, 72, 81, 84, 87, 90, 92, or polynucleotides homologous thereto.

According to another embodiment, detecting a polynucleotide of the invention comprises employing a primer pair, comprising a pair of isolated oligonucleotides as set forth in SEQ ID NOs:58-65, 79-80, 82-83, 85-86, 88-89, 91, 115-121.

The invention also includes the following specific embodiments.

In one embodiment the invention includes an isolated polypeptide selected from Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P20 (SEQ ID NO:53), HUMDAF_P29 (SEQ ID NO:55), HUMDAF_P30 (SEQ ID NO:56), HUMDAF_P31 (SEQ ID NO:57), or a fragment or variant thereof that possesses at least 95, 96, 97, 98 or 99% sequence identity therewith.

In another embodiment the invention includes a fragment or conjugate comprising any one of the foregoing polypeptides.

In another embodiment the invention includes any one of the foregoing polypeptides fused to an immunoglobulin domain.

In another embodiment the invention includes any of the foregoing polypeptides attached to a detectable or therapeutic moiety.

In another embodiment the invention includes a nucleic acid sequence encoding any of the foregoing polypeptides.

In another embodiment the invention includes any of the nucleic acid sequences selected from Z43375_{—}1_T3 (SEQ ID NO:2), Z43375_{—}1_T6 (SEQ ID NO:3), Z43375_{—}1_T7 (SEQ ID NO:4), Z43375_{—}1_T14 (SEQ ID NO:5), Z43375_{—}1_T16 (SEQ ID NO:6), Z43375_{—}1_T20 (SEQ ID NO:7), Z43375_{—}1_T22 (SEQ ID NO:8), Z43375_{—}1_T23 (SEQ ID NO:9), Z43375_{—}1_T28 (SEQ ID NO:10), Z43375_{—}1_T30 (SEQ ID NO:11), Z43375_{—}1_T31 (SEQ ID NO:12), Z43375_{—}1_T33 (SEQ ID NO:13), HSCD20B_{—}1_T12 (SEQ ID NO:31), HUMDAF_T10 (SEQ ID NO:34), HUMDAF_T11 (SEQ ID NO:35), HUMDAF_T17 (SEQ ID NO:36), HUMDAF_T24 (SEQ ID NO:38), HUMDAF_T30 (SEQ ID NO:39), HUMDAF_T31 (SEQ ID NO:40), HUMDAF_T32 (SEQ ID NO:41), or a fragment or variant and conjugates thereof that possesses at least 95, 96, 97, 98 or 99% sequence identity therewith.

In another embodiment the invention includes an isolated KIAA00746, CD20 or CD55 ectodomain polypeptide, or a fragment or conjugate thereof.

In another embodiment the invention includes any of the foregoing polypeptides, comprising a sequence of amino acid residues having at least 95, 96, 97, 98 or 99% sequence identity with amino acid residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, residues 87-109 of HSCD20B_{—}1_P5 (SEQ ID NO:33), corresponding to amino acid sequence depicted in SEQ ID NO:106, or residues 1-63 of HSCD20B_{—}1_P5 (SEQ ID NO:33), corresponding to amino acid sequence depicted in SEQ ID NO:107, or residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:109, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112.

In another embodiment the invention includes any of the foregoing polypeptides, comprising the extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57).

In another embodiment the invention includes any of the foregoing polypeptides, attached to a detectable or therapeutic moiety.

In another embodiment the invention includes any of the foregoing nucleic acid sequences encoding any one of the KIAA0746, CD20, CD55 ectodomain polypeptides and conjugates thereof.

In another embodiment the invention includes an expression vector containing any of the foregoing nucleic acid sequences.

In another embodiment the invention includes a host cell comprising the foregoing expression vector or a virus containing a nucleic acid sequence encoding the KIAA0746, CD20, CD55 ectodomain polypeptide, or fragment or conjugate thereof, wherein the cell expresses the polypeptide encoded by the DNA segment.

In another embodiment the invention includes a method of producing any one of the KIAA0746, CD20, CD55 ectodomain polypeptides, or fragment or conjugate thereof, comprising culturing the foregoing host cell, wherein the cell expresses the polypeptide encoded by the DNA segment or nucleic acid and recovering said polypeptide.

In another embodiment the invention includes any of the foregoing isolated soluble KIAA0746, CD20, CD55 ectodomain wherein said polypeptide blocks or inhibits the interaction of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof with a corresponding functional ligand.

In another embodiment the invention includes the foregoing isolated soluble KIAA0746, CD20, CD55 ectodomains, wherein said polypeptide replaces or augments the interaction of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant or conjugate thereof with a corresponding functional ligand.

In another embodiment the invention includes a fusion protein comprising any of the foregoing isolated soluble KIAA0746, CD20, CD55 ectodomain joined to a non-KIAA0746, non-CD20, non-CD55 protein sequence, correspondingly.

In another embodiment the invention includes any of the foregoing fusion proteins, wherein the non-KIAA0746, non-CD20, non-CD55, protein is at least a portion of an immunoglobulin molecule.

In another embodiment the invention includes any of the foregoing fusion proteins, wherein a polyalkyl oxide moiety such as polyethylene glycol is attached to the polypeptide.

In another embodiment the invention includes any of the foregoing fusion proteins, wherein the immunoglobulin heavy chain constant region is an Fc fragment.

In another embodiment the invention includes any one of the protein sequences of the KIAA0746, CD20, CD55 ECDs fused to mouse Fc, or nucleic acid sequences encoding the KIAA0746, CD20, CD55 ECDs fused to mouse Fc.

In another embodiment the invention includes any of the foregoing fusion proteins wherein the immunoglobulin heavy chain constant region is an isotype selected from the group consisting of an IgG1, IgG2, IgG3, IgG4, IgM, IgE, IgA and IgD.

In another embodiment the invention includes any of the foregoing fusion proteins, wherein the polypeptide is fused to a VASP domain.

In another embodiment the invention includes any of the foregoing fusion proteins, wherein the fusion protein modulates lymphocyte activation.

In another embodiment the invention includes a pharmaceutical composition comprising any of the foregoing polynucleotide sequences and further comprising a pharmaceutically acceptable diluent or carrier.

In another embodiment the invention includes a pharmaceutical composition comprising the foregoing vector and further comprising a pharmaceutically acceptable diluent or carrier.

In another embodiment the invention includes a pharmaceutical composition comprising the foregoing host cell and further comprising a pharmaceutically acceptable diluent or carrier.

In another embodiment the invention includes a pharmaceutical composition comprising any of the foregoing KIAA0746, CD20, CD55 ectodomains and further comprising a pharmaceutically acceptable diluent or carrier.

In another embodiment the invention includes a pharmaceutical composition comprising any of the foregoing polypeptides and further comprising a pharmaceutically acceptable diluent or carrier.

In another embodiment the invention includes a pharmaceutical composition comprising the foregoing fusion protein and further comprising a pharmaceutically acceptable diluent or carrier.

In another embodiment the invention includes a method for treating or preventing cancer, comprising administering to a subject in need thereof a pharmaceutical composition comprising: a soluble molecule having the extracellular domain of KIAA0746, CD20, CD55 polypeptide, or fragment or conjugate thereof; or polypeptide, comprising a sequence of amino acid residues having at least 95, 96, 97, 98 or 99% sequence identity with amino acid residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, or residues 87-109 of HSCD20B_{—}1_P5 (SEQ ID NO:33), corresponding to amino acid sequence depicted in SEQ ID NO:106, or residues 1-63, of HSCD20B_{—}1_P5 (SEQ ID NO:33), corresponding to amino acid sequence depicted in SEQ ID NO:107, or residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same.

In another embodiment the invention includes the foregoing method, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention includes the foregoing method wherein the pharmaceutical composition comprises: a soluble molecule having the extracellular domain of KIAA0746, CD55 polypeptide, or a fragment or conjugate thereof; or polypeptide, comprising a sequence of amino acid residues having at least 95% sequence identity with amino acid residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, or residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same, and wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention includes the foregoing method wherein the pharmaceutical composition comprises a soluble molecule having the extracellular domain of CD20 polypeptide, or a fragment or conjugate thereof; or polypeptide, comprising a sequence of amino acid residues having at least 95% sequence identity with amino acid residues 87-109 of HSCD20B_{—}1_P5 (SEQ ID NO:33), corresponding to amino acid sequence depicted in SEQ ID NO:106, or amino acid residues 1-63, of HSCD20B_{—}1_P5 (SEQ ID NO:33), corresponding to amino acid sequence depicted in SEQ ID NO:107, or polypeptide, comprising an extracellular domain of HSCD20B_{—}1_P5 (SEQ ID NO:33), or a nucleic acid sequence encoding the same, and wherein the cancer is a hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

In another embodiment the invention includes the foregoing method for treating or preventing cancer, used in combination therapy with other treatment methods known in the art selected from the group consisting of radiation therapy, antibody therapy, chemotherapy, surgery, or in combination therapy with other biological agents, conventional drugs, anti-cancer agents, immunosuppressants, cytotoxic drugs for cancer, chemotherapeutic agents, or in combination with therapeutic agents targeting other complement regulatory proteins (CRPs).

In another embodiment the invention includes the foregoing method for treating or preventing cancer, used in combination therapy with other treatment methods known in the art, wherein the cancer is previously untreated follicular, CD20-positive, B-cell NHL, and wherein the treatment comprises using a pharmaceutical composition comprising any of a soluble molecule having the extracellular domain of CD20 polypeptide, or a fragment or conjugate thereof; or polypeptide, comprising a sequence of amino acid residues having at least 95% sequence identity with amino acid residues 87-109 of HSCD20B_{—}1_P5 (SEQ ID NO:33), corresponding to amino acid sequence depicted in SEQ ID NO:106, or amino acid residues 1-63 of HSCD20B_{—}1_P5 (SEQ ID NO:33), corresponding to amino acid sequence depicted in SEQ ID NO:107, or polypeptide, comprising an extracellular domain of HSCD20B_{—}1_P5 (SEQ ID NO:33), or a nucleic acid sequence encoding the same, in combination with CVP chemotherapy (cyclophosphamide, vincristine and prednisolone).

In another embodiment the invention includes the foregoing method for treating or preventing cancer, used in combination therapy with other treatment methods known in the art, wherein the cancer is previously untreated diffuse large B-cell, CD20-positive NHL, and wherein the treatment comprises using a pharmaceutical composition comprising any of a soluble molecule having the extracellular domain of CD20 polypeptide, or a fragment or conjugate thereof; or polypeptide, comprising a sequence of amino acid residues having at least 95% sequence identity with amino acid residues 87-109 of HSCD20B_{—}1_P5 (SEQ ID NO:33), corresponding to amino acid sequence depicted in SEQ ID NO:06, or amino acid residues 1-63 of HSCD20B_{—}1_P5 (SEQ ID NO:33), corresponding to amino acid sequence depicted in SEQ ID NO:107, or polypeptide, comprising an extracellular domain of HSCD20B_{—}1_P5 (SEQ ID NO:33), or a nucleic acid sequence encoding the same, in combination with CHOP (cyclophosphamide, doxorubicin, vincristine and prednisolone) or other anthracycline-based chemotherapy regimens.

In another embodiment the invention includes the foregoing method for treating or preventing cancer, used in combination therapy with other treatment methods known in the art, wherein the cancer is previously untreated diffuse NHL mantle cell lymphoma, and wherein the treatment comprises using a pharmaceutical composition comprising any of a soluble molecule having the extracellular domain of CD20 polypeptide, or a fragment or conjugate thereof; or polypeptide, comprising a sequence of amino acid residues having at least 95% sequence identity with amino acid residues 87-109 of HSCD20B_{—}1_P5 (SEQ ID NO:33), corresponding to amino acid sequence depicted in SEQ ID NO:106, or amino acid residues 1-63 of HSCD20B_{—}1_P5 (SEQ ID NO:33), corresponding to amino acid sequence depicted in SEQ ID NO:107, or polypeptide, comprising an extracellular domain of HSCD20B_{—}1_P5 (SEQ ID NO:33), or a nucleic acid sequence encoding the same, in combination with CHOP (cyclophosphamide, doxorubicin, vincristine and prednisolone) or other anthracycline-based chemotherapy regimens. In another embodiment the invention includes a method for treating or preventing immune related conditions, comprising administering to a subject in need thereof a pharmaceutical composition comprising: a soluble molecule having the extracellular domain of KIAA0746, CD20, CD55 polypeptide, or fragment or conjugate thereof; or polypeptide, comprising a sequence of amino acid residues having at least 95, 96, 97, 98 or 99% sequence identity with amino acid residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, or residues 87-109 of HSCD20B_{—}1_P5 (SEQ ID NO:33), corresponding to amino acid sequence depicted in SEQ ID NO:106, or residues 1-63, of HSCD20B_{—}1_P5 (SEQ ID NO:33), corresponding to amino acid sequence depicted in SEQ ID NO:107, or residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same.

In another embodiment the invention includes the foregoing method, wherein the immune related conditions are inflammatory, allergic or autoimmune diseases, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In another embodiment the invention includes the foregoing method, wherein the pharmaceutical composition comprises a soluble molecule having the extracellular domain of KIAA0746, CD20 polypeptide, or fragment or conjugate thereof; or polypeptide, comprising a sequence of amino acid residues having at least 95% sequence identity with amino acid residues 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, or residues 87-109 of HSCD20B_{—}1_P5 (SEQ ID NO:33), corresponding to amino acid sequence depicted in SEQ ID NO:106, or residues 1-63 of HSCD20B_{—}1_P5 (SEQ ID NO:33), corresponding to amino acid sequence depicted in SEQ ID NO:107; or polypeptide, comprising an extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33); or a nucleic acid sequence encoding the same, and the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

In another embodiment the invention includes the foregoing method, wherein the pharmaceutical composition comprises a soluble molecule having the extracellular domain of CD55 polypeptide, or fragment or conjugate thereof; or polypeptide, comprising a sequence of amino acid residues having at least 95% sequence identity with amino acid residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same, and wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In another embodiment the invention includes a method for treating or preventing ischemia-reperfusion injury, comprising administering to a subject in need thereof a pharmaceutical composition comprising: a soluble molecule having the extracellular domain of CD55 polypeptide, or fragment or conjugate thereof; or polypeptide, comprising a sequence of amino acid residues having at least 95% sequence identity with amino acid residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same.

In another embodiment the invention includes the foregoing method, wherein the ischemia-reperfusion injury is selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In another embodiment the invention a method for treating or preventing inflammation of the respiratory tract disorder, comprising administering to a subject in need thereof a pharmaceutical composition comprising: a soluble molecule having the extracellular domain of CD55 polypeptide, or fragment or conjugate thereof; or polypeptide, comprising a sequence of amino acid residues having at least 95% sequence identity with amino acid residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112; or polypeptide, comprising an extracellular domain of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57); or a nucleic acid sequence encoding the same.

In another embodiment the invention includes the foregoing method, wherein the inflammation of the respiratory tract disorder is selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

In another embodiment the invention includes the foregoing method for treating or preventing immune related conditions, used in combination therapy with other treatment methods known in the art selected from the group consisting of antibody therapy, biological agents, conventional drugs, immunosuppressants, cytotoxic drugs, or in combination with therapeutic agents targeting other complement regulatory proteins (CRPs).

In another embodiment the invention includes a method for treating or preventing lymphoproliferative disorders, selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS), comprising administering to a subject in need thereof a pharmaceutical composition comprising: a soluble molecule having the extracellular domain of any one of KIAA0746 or CD20 polypeptide, or fragment or conjugate thereof; or polypeptide, comprising a sequence of amino acid residues having at least 95% sequence identity with amino acid residues 33-1023 of Z43375_{—}1_P4 (SEQ ID NO:18), or residues 17-1049 of Z43375_{—}1_P8 (SEQ ID NO:19), or residues 33-887 of Z43375_{—}1_P40 (SEQ ID NO:20), or residues 33-995 of Z43375_{—}1_P46 (SEQ ID NO:21), or residues 33-1022 of Z43375_{—}1_P47 (SEQ ID NO:22), or residues 33-977 of Z43375_{—}1_P50 (SEQ ID NO:23), or residues 33-792 of Z43375_{—}1_P51 (SEQ ID NO:24), or residues 33-1010 of Z43375_{—}1_P52 (SEQ ID NO:25), or residues 33-839 of Z43375_{—}1_P53 (SEQ ID NO:26), or residues 33-833 of Z43375_{—}1_P54 (SEQ ID NO:27), or residues 33-867 of Z43375_{—}1_P55 (SEQ ID NO:28), or residues 33-714 of Z43375_{—}1_P56 (SEQ ID NO:29), or residues 21-770 of Z43375_{—}1_P60 (SEQ ID NO:30), or residues 87-109 of HSCD20B_{—}1_P5 (SEQ ID NO:33), or residues 1-63 of HSCD20B_{—}1_P5 (SEQ ID NO:33); or polypeptide, comprising an extracellular domain of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33); or a nucleic acid sequence encoding the same.

In another embodiment the invention includes an siRNA, antisense RNA, or ribozyme that binds the transcript encoding any one of the KIAA0746, CD20, CD55 polypeptides, selected from Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), or a fragment or a variant thereof, and inhibits its expression.

In another embodiment the invention includes a polyclonal or monoclonal antibody that specifically binds and/or modulates an activity elicited by any one of the KIAA0746, CD20, CD55 polypeptides, selected from Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or a variant thereof and conjugates thereof, and anti-idiotypic antibodies specific to any of the foregoing.

In another embodiment the invention includes a monoclonal or polyclonal antibody or an antigen binding fragment thereof comprising an antigen binding site that binds specifically to any one of the KIAA0746, CD20, CD55 polypeptides comprised in Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or fragment or variant thereof that is at least 80% identical thereto and anti-idiotypic antibodies specific to any of the foregoing.

In another embodiment the invention includes a monoclonal or polyclonal antibody or an antigen binding fragment thereof comprising an antigen binding site that binds specifically to any one of the SEQ ID NOs: 70; 77; 78; 126-129.

In another embodiment the invention includes any of the foregoing antibodies or fragments thereof, wherein said antibody blocks or inhibits the interaction of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof or anti-idiotypic antibody with a counterpart or cell component or tissue structure promoting an opposite activity or function.

In another embodiment the invention includes any of the foregoing antibodies or fragments wherein said antibody replaces or augments the interaction of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof or anti-idiotypic antibody with a counterpart or cell component or tissue structure promoting an opposite function or activity.

In another embodiment the invention includes a method for modulating lymphocyte activity, comprising contacting a Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57) positive lymphocyte with a bioactive agent capable of modulating KIAA0746-mediated, CD20-mediated, or CD55-mediated, signaling in an amount effective to modulate at least one lymphocyte activity.

In another embodiment the invention includes the foregoing method, wherein said agent comprises an antagonist of KIAA0746-mediated, CD20-mediated, or CD55-mediated signaling, and wherein said contacting inhibits the attenuation of lymphocyte activity mediated by such signaling.

In another embodiment the invention includes the foregoing method, wherein said contacting increases lymphocyte activity.

In another embodiment the invention includes the foregoing method wherein said antagonist comprises a blocking agent capable of interfering with the functional interaction of KIAA0746, CD20, or CD55 antigen and its counterpart.

In another embodiment the invention includes the foregoing antibody or antibody fragment which is suitable for treatment or prevention of cancer.

In another embodiment the invention includes the foregoing method wherein the administered antibody or fragment inhibits negative stimulation of T cell activity against cancer cells.

In another embodiment the invention includes any of the foregoing antibodies or fragments, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention includes any of the foregoing antibodies or fragments, wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention includes any of the foregoing antibodies or fragments, wherein the cancer is selected from the group consisting of hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy, and wherein the cancer is non-metastatic, invasive or metastatic.

In another embodiment the invention includes any of the foregoing antibodies or fragments, which are suitable for treatment or prevention of immune related disorders, by modulating the activity of any one of the KIAA0746, CD20 or CD55 proteins.

In another embodiment the invention includes any of the foregoing antibodies or fragments, which are suitable for treating an immune related condition, wherein the immune related conditions are inflammatory and autoimmune diseases, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In another embodiment the invention includes any of the foregoing antibodies or fragments, which is suitable for treatment or prevention of immune related disorders, by modulating the activity of any one of the KIAA0746 or CD20 proteins, wherein the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

In another embodiment the invention includes any of the foregoing antibodies or fragments, which is suitable for treatment or prevention of immune related disorders, by modulating the activity of CD55 protein, wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In another embodiment the invention includes any of the foregoing antibodies or fragments, which is suitable for treatment or prevention of ischemia-reperfusion injury, by modulating the activity of CD55 protein.

In another embodiment the invention includes any of the foregoing antibodies or fragments, which is suitable for treatment or prevention of ischemia-reperfusion injury, wherein the ischemia-reperfusion injury is selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In another embodiment the invention includes any of the foregoing antibodies or fragments, which is suitable for treatment or prevention of inflammation of the respiratory tract disorder, by modulating the activity of CD55 protein.

In another embodiment the invention includes any of the foregoing antibodies or fragments, which is suitable for treatment or prevention of inflammation of the respiratory tract disorder, wherein the inflammation of the respiratory tract disorder is selected from the group including but not limited to chronic obstructive pulmonary disease (COPD), acute respiratory distress syndrome (ARDS), severe acute respiratory syndrome (SARS), asthma, allergy, pulmonary emphysema, pulmonary inflammation, environmental airway disease, airway hyper-responsiveness, chronic bronchitis, acute lung injury, bronchial disease, lung diseases, and cystic fibrosis.

In another embodiment the invention includes any of the foregoing antibodies or fragments, which are suitable for treatment or prevention of lymphoproliferative disorders, by modulating the activity of any one of the KIAA0746 and CD20 proteins.

In another embodiment the invention includes any of the foregoing antibodies or fragments, which are suitable for treatment or prevention of lymphoproliferative disorders, wherein the lymphoproliferative disorder is selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

In another embodiment the invention includes any of the foregoing antibodies or antibody fragments, that specifically binds to amino-acids: 33-1023 of Z43375_{—}1_P4 (SEQ ID NO:18), corresponding to amino acid sequence depicted in SEQ ID NO:93, or residues 17-1049 of Z43375_{—}1_P8 (SEQ ID NO:19), corresponding to amino acid sequence depicted in SEQ ID NO:94, or residues 33-887 of Z43375_{—}1_P40 (SEQ ID NO:20), corresponding to amino acid sequence depicted in SEQ ID NO:95, or residues 33-995 of Z43375_{—}1_P46 (SEQ ID NO:21), corresponding to amino acid sequence depicted in SEQ ID NO:96, or residues 33-1022 of Z43375_{—}1_P47 (SEQ ID NO:22), corresponding to amino acid sequence depicted in SEQ ID NO:97, or residues 33-977 of Z43375_{—}1_P50 (SEQ ID NO:23), corresponding to amino acid sequence depicted in SEQ ID NO:98, or residues 33-792 of Z43375_{—}1_P51 (SEQ ID NO:24), corresponding to amino acid sequence depicted in SEQ ID NO:99, or residues 33-1010 of Z43375_{—}1_P52 (SEQ ID NO:25), corresponding to amino acid sequence depicted in SEQ ID NO:100, or residues 33-839 of Z43375_{—}1_P53 (SEQ ID NO:26), corresponding to amino acid sequence depicted in SEQ ID NO:101, or residues 33-833 of Z43375_{—}1_P54 (SEQ ID NO:27), corresponding to amino acid sequence depicted in SEQ ID NO:102, or residues 33-867 of Z43375_{—}1_P55 (SEQ ID NO:28), corresponding to amino acid sequence depicted in SEQ ID NO:103, or residues 33-714 of Z43375_{—}1_P56 (SEQ ID NO:29), corresponding to amino acid sequence depicted in SEQ ID NO:104, or residues 21-770 of Z43375_{—}1_P60 (SEQ ID NO:30), corresponding to amino acid sequence depicted in SEQ ID NO:105, or residues 87-109 of HSCD20B_{—}1_P5 (SEQ ID NO:33), corresponding to amino acid sequence depicted in SEQ ID NO:106, or residues 1-63, of HSCD20B_{—}1_P5 (SEQ ID NO:33), corresponding to amino acid sequence depicted in SEQ ID NO:107, or residues 35-497 of HUMDAF_P14 (SEQ ID NO:51), corresponding to amino acid sequence depicted in SEQ ID NO:108, or residues 35-523 of HUMDAF_P15 (SEQ ID NO:52), corresponding to amino acid sequence depicted in SEQ ID NO:109, or residues 35-497 of HUMDAF_P20 (SEQ ID NO:53), corresponding to amino acid sequence depicted in SEQ ID NO:108, or residues 36-371 of HUMDAF_P26 (SEQ ID NO:54), corresponding to amino acid sequence depicted in SEQ ID NO:110, or residues 35-328 of HUMDAF_P29 (SEQ ID NO:55), corresponding to amino acid sequence depicted in SEQ ID NO:111, or residues 35-497 of HUMDAF_P30 (SEQ ID NO:56), corresponding to amino acid sequence depicted in SEQ ID NO:108, or residues 35-523 of HUMDAF_P31 (SEQ ID NO:57), corresponding to amino acid sequence depicted in SEQ ID NO:112, or a variant or fragment or an epitope thereof.

In another embodiment the invention includes any of the foregoing antibodies or fragments, wherein the antigen binding site contains from about 3-7 contiguous or non-contiguous amino acids, more typically at least 5 contiguous or non-contiguous amino acids. These binding sites include conformational and non-conformational epitopes.

In another embodiment the invention includes any of the foregoing antibodies or fragments, wherein the antibody is a fully human antibody.

In another embodiment the invention includes any of the foregoing antibodies or fragments, wherein the antibody is a chimeric antibody.

In another embodiment the invention includes the foregoing antibodies or fragments wherein the antibody is a humanized or primatized antibody.

In another embodiment the invention includes any of the foregoing antibodies or fragments, wherein the fragment is selected from the group consisting of Fab, Fab′, F(ab′)2, F(ab′), F(ab), Fv or scFv fragment and minimal recognition unit.

In another embodiment the invention includes any of the foregoing antibodies or fragments, wherein the antibody or fragment is coupled to a detectable marker, or to an effector moiety.

In another embodiment the invention includes any of the foregoing antibodies or fragments, wherein the effector moiety is an enzyme, a toxin, a therapeutic agent, or a chemotherapeutic agent.

In another embodiment the invention includes any of the foregoing antibodies or fragments, wherein the detectable marker is a radioisotope, a metal chelator, an enzyme, a fluorescent compound, a bioluminescent compound or a chemiluminescent compound.

In another embodiment the invention includes a pharmaceutical composition that comprises any of the foregoing antibodies or a fragment thereof.

In another embodiment the invention includes a pharmaceutical composition that comprises the foregoing antibodies or a fragment thereof.

In another embodiment the invention includes a method of inducing or enhancing an immune response, comprising administering to a patient in need thereof any of the foregoing antibodies or fragments and detecting induction or enhancement of said immune response.

In another embodiment the invention includes a method for potentiating a secondary immune response to an antigen in a patient, which method comprises administering effective amounts any of the foregoing antibodies or fragments.

In another embodiment the invention includes the foregoing method, wherein the antigen is preferably a cancer antigen, a viral antigen or a bacterial antigen, and the patient has preferably received treatment with an anticancer vaccine or a viral vaccine.

In another embodiment the invention includes a method of treating a patient with a KIAA0746, CD20, or CD55 positive malignancy, comprising administering to the patient an effective amount of any of the foregoing antibodies or fragments.

In another embodiment the invention includes the foregoing method, used in combination therapy with other treatment methods known in the art selected from the group consisting of radiation therapy, antibody therapy, chemotherapy, surgery, or in combination therapy with conventional drugs, anti-cancer agents, immunosuppressants, cytotoxic drugs for cancer, chemotherapeutic agents, or in combination with therapeutic agents targeting other complement regulatory proteins (CRPs).

In another embodiment the invention includes the foregoing method further comprising co-administering a chemotherapeutic agent.

In another embodiment the invention includes the foregoing method for treating or preventing cancer, used in combination therapy with other treatment methods known in the art, wherein the cancer is previously untreated follicular, CD20-positive, B-cell NHL, and wherein the treatment comprises administering to the patient an effective amount of any of the foregoing antibodies or fragments specific to CD20, in combination with CVP chemotherapy (cyclophosphamide, vincristine and prednisolone).

In another embodiment the invention includes the foregoing method for treating or preventing cancer, used in combination therapy with other treatment methods known in the art, wherein the cancer is previously untreated diffuse large B-cell, CD20-positive NHL, and wherein the treatment comprises administering to the patient an effective amount of any of the foregoing antibodies or fragments specific to CD20, in combination with CHOP (cyclophosphamide, doxorubicin, vincristine and prednisolone) or other anthracycline-based chemotherapy regimens.

In another embodiment the invention includes the foregoing method for treating or preventing cancer, used in combination therapy with other treatment methods known in the art, wherein the cancer is previously untreated diffuse NHL mantle cell lymphoma, and wherein the treatment comprises administering to the patient an effective amount of any of the foregoing antibodies or fragments specific to CD20, in combination with CHOP (cyclophosphamide, doxorubicin, vincristine and prednisolone) or other anthracycline-based chemotherapy regimens.

In another embodiment the invention includes the foregoing method, wherein said malignancy is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention includes the foregoing method, wherein said malignancy is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention includes the foregoing method, wherein said malignancy is selected from the group consisting of hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy, and wherein the cancer is non-metastatic, invasive or metastatic.

In another embodiment the invention includes a method of inhibiting growth of cells that express a polypeptide selected from Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof in a subject, comprising: administering to said subject any of the foregoing antibodies or fragments.

In another embodiment the invention includes a method of treating or preventing cancer comprising the administration of a therapeutically effective amount of an antibody or binding fragment that specifically binds the Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof that possesses at least 80% sequence identity therewith.

In another embodiment the invention includes the foregoing method, wherein the cancer is selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is e non-metastatic, invasive or metastatic.

In another embodiment the invention includes the foregoing method, wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention includes the foregoing method, wherein the cancer is selected from the group consisting of hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy, and wherein the cancer is non-metastatic, invasive or metastatic.

In another embodiment the invention includes the foregoing method, carried out using combination therapy with other treatment methods known in the art selected from the group consisting of radiation therapy, antibody therapy, chemotherapy, surgery, or in combination therapy with other biological agents, conventional drugs, anti-cancer agents, immunosuppressants, cytotoxic drugs for cancer, chemotherapeutic agents, or in combination with therapeutic agents targeting other complement regulatory proteins (CRPs).

In another embodiment the invention includes the foregoing method wherein the antibody is a human, humanized or chimeric antibody or antigen binding fragment.

In another embodiment the invention includes the foregoing method wherein the antibody or fragment is attached directly or indirectly to an effector moiety.

In another embodiment the invention includes the foregoing method, wherein the effector is selected from a drug, toxin, radionuclide, fluorophore and an enzyme.

In another embodiment the invention includes a method for treating or preventing an immune related condition, comprising administering to a patient a therapeutically effective amount of an antibody that specifically binds to Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33) HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof that possesses at least 80% sequence identity therewith or an anti-idiotypic antibody specific to any of the foregoing.

In another embodiment the invention includes the foregoing method, wherein the immune related condition comprises one or more of an inflammatory or an autoimmune disease selected from the group consisting of multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In another embodiment the invention includes the foregoing method, wherein the antibody specifically binds to Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), and HSCD20B_{—}1_P5 (SEQ ID NO:33), or a fragment or variant thereof that possesses at least 80% sequence identity therewith wherein, or an anti-idiotypic antibody specific to any of the foregoing and the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

In another embodiment the invention includes the foregoing method, wherein the antibody specifically binds to HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof that possesses at least 80% sequence identity therewith or an anti-idiotypic antibody specific to any of the foregoing, and the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In another embodiment the invention includes the a method for treating or preventing an ischemia-reperfusion injury, comprising administering to a patient a therapeutically effective amount of an antibody that specifically binds to HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof that possesses at least 80% sequence identity therewith or an anti-idiotypic antibody specific to any of the foregoing, wherein the ischemia-reperfusion injury is selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In another embodiment the invention includes a method for treating or preventing an inflammation of the respiratory tract disorder, comprising administering to a patient a therapeutically effective amount of an antibody that specifically binds to HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof that possesses at least 80% sequence identity therewith or an anti-idiotypic antibody specific to any of the foregoing, wherein the inflammation of the respiratory tract disorder is selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

In another embodiment the invention includes a method for treating or preventing a lymphoproliferative disorder, comprising administering to a patient a therapeutically effective amount of an antibody that specifically binds to Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), or a fragment or variant thereof that possesses at least 80% sequence identity therewith, wherein the lymphoproliferative disorder is selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS) In another embodiment the invention includes the foregoing method, wherein the antibody has an antigen-binding region specific for the extracellular domain of any one of said KIAA0746, CD20, CD55 polypeptides.

In another embodiment the invention includes the foregoing method, wherein the treatment is combined with a moiety useful for treating immune related condition.

In another embodiment the invention includes the foregoing method, wherein the moiety is a cytokine antibody, cytokine receptor antibody, drug, or another immunomodulatory agent.

In another embodiment the invention includes an assay for detecting the presence of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof in a biological sample comprising contacting the sample with an antibody of any one of the foregoing, and detecting the binding of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof in the sample.

In another embodiment the invention includes a method for any one of screening for a disease, detecting a presence or a severity of a disease, diagnosing a disease, prognosis of a disease, monitoring disease progression or treatment efficacy or relapse of a disease, or selecting a therapy for a disease, comprising detecting expression and/or presence in a subject or in a sample obtained from the subject a polypeptide having a sequence at least 85% homologous to the amino acid sequence as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or with a polypeptide having a sequence comprising the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57).

In another embodiment the invention includes the foregoing method, wherein the polypeptide having the amino acid sequence as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), as set forth in SEQ ID NOs: 93-114, or a fragment or variant thereof.

In another embodiment the invention includes the foregoing method, wherein detecting the expression and/or the presence of the polypeptide is performed in vivo or in vitro.

In another embodiment the invention includes the foregoing method, wherein the disease is selected from cancer, selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention includes the foregoing method, which comprises detecting the polypeptide as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), or the polypeptide having the sequence comprising the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), and HSCD20B_{—}1_P5 (SEQ ID NO:33), and wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention includes the foregoing method, which comprises detecting the polypeptide as set forth in any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or the polypeptide having the sequence comprising the extracellular domain of any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and wherein the cancer is hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

In another embodiment the invention includes the foregoing method, wherein the disease is an immune related condition.

In another embodiment the invention includes the foregoing method, wherein the immune related condition is an inflammatory and/or an autoimmune disease, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In another embodiment the invention includes the foregoing method, which comprises detecting the polypeptide as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), or the polypeptide having the sequence comprising the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), and HSCD20B_{—}1_P5 (SEQ ID NO:33), and wherein the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

In another embodiment the invention includes the foregoing method, which comprises detecting the polypeptide as set forth in any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or the polypeptide having the sequence comprising the extracellular domain of any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In another embodiment the invention includes the foregoing method, which comprises detecting the polypeptide as set forth in any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or the polypeptide having the sequence comprising the extracellular domain of any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and wherein the disease is ischemia-reperfusion injury, selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In another embodiment the invention includes the foregoing method, which comprises detecting the polypeptide as set forth in any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or the polypeptide having the sequence comprising the extracellular domain of any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and wherein the disease is respiratory tract disorder, selected from the group including but not limited to chronic obstructive pulmonary disease (COPD), acute respiratory distress syndrome (ARDS), severe acute respiratory syndrome (SARS), asthma, allergy, pulmonary emphysema, pulmonary inflammation, environmental airway disease, airway hyper-responsiveness, chronic bronchitis, acute lung injury, bronchial disease, lung diseases, and cystic fibrosis.

In another embodiment the invention includes the foregoing method, which comprises detecting the polypeptide as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), and HSCD20B_{—}1_P5 (SEQ ID NO:33), or the polypeptide having the sequence comprising the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), and wherein the disease is lymphoproliferative disorder, selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

In another embodiment the invention includes a method of using an antibody or antigen binding fragment that specifically binds Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), or a fragment or variant thereof for in vivo imaging of tumors or inflammatory sites characterized by the differential expression of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof.

In another embodiment the invention includes the foregoing method, wherein the detection is conducted by immunoassay.

In another embodiment the invention includes the foregoing method, wherein the immunoassay utilizes an antibody which specifically interacts with the polypeptide having a sequence at least 85% homologous to the amino acid sequence as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or with a polypeptide having a sequence comprising the extracellular domain of any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57).

In another embodiment the invention includes an antibody specific to Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), or a fragment or variant thereof that elicits apoptosis or lysis of cancer cells that express said protein.

In another embodiment the invention includes any of the foregoing antibodies or fragments, wherein said apoptosis or lysis activity involves CDC or ADCC activity of the antibody.

In another embodiment the invention includes any of the foregoing antibodies or fragments, wherein the cancer cells are selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention includes any of the foregoing antibodies or fragments, wherein the antibody or fragment is specific to any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), and HSCD20B_{—}1_P5 (SEQ ID NO:33), and wherein the cancer cells are colorectal cancer, lung cancer, prostate cancer, pancreas cancer, ovarian cancer, gastric cancer, liver cancer, melanoma, kidney cancer, head and neck cancer cells.

In another embodiment the invention includes any of the foregoing antibodies or fragments, wherein the antibody or fragment is specific to any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and wherein the cancer cells are hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia cancer cells.

In another embodiment the invention relates to any of the foregoing isolated soluble KIAA0746, CD20, CD55 ectodomain polypeptides, wherein said polypeptide or a fragment or variant thereof is used as an anti-cancer vaccine for cancer immunotherapy.

In another embodiment the invention relates to any isolated polypeptide comprising an amino acid sequence having at least 80%, 85%, 90%, 95, 96, 97, 98 or 99%, 100% homologous to the sequence as that set forth in any one of SEQ ID NOs: 176-218, or a fragment thereof.

In another embodiment the invention relates to any isolated polynucleotide, comprising an amplicon having a nucleic acid sequence selected from the group consisting of SEQ ID NOs:81, 84, 87, 90, 92, or polynucleotides homologous thereto.

In another embodiment the invention relates to any primer pair, comprising a pair of isolated oligonucleotides capable of amplifying the above mentioned amplicon.

In another embodiment the invention relates to the primer pair, comprising a pair of isolated oligonucleotides having a sequence selected from the group consisting of SEQ ID NOs: 58-65, 79-80, 82-83, 85-86, 88-89, 91, 115-121.

In another embodiment the invention relates to a method for screening for a disease, disorder or condition in a subject, comprising detecting in the subject or in a sample obtained from said subject a polynucleotide having a sequence at least 85% homologous to the nucleic acid sequence as set forth in any one of SEQ ID NOs: 1-13, 31, 34-41, 71, 72, 81, 84, 87, 90, 92.

In another embodiment the invention relates to the method for any one of screening for a disease, detecting a presence or a severity of a disease, diagnosing a disease, prognosis of a disease, monitoring disease progression or treatment efficacy or relapse of a disease, or selecting a therapy for a disease, comprising detecting in a subject or in a sample obtained from the subject comprising detecting in the subject or in a sample obtained from said subject a polynucleotide having a sequence at least 85%, 90%, 95%, 100% homologous to the nucleic acid sequence as set forth in any one of SEQ ID NOs: 1-13, 31, 34-41, 71, 72, 81, 84, 87, 90, 92.

In another embodiment the invention relates to the method as above, wherein the disease is a cancer, selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In another embodiment the invention relates to the method as above, which comprises detecting the nucleic acid sequence as set forth in any one of SEQ ID NOs: 1-13, 31, 81, 84, 87, and wherein the cancer is selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In another embodiment the invention relates to the method as above, which comprises detecting the nucleic acid sequence as set forth in any one of SEQ ID NOs: 34-41, 90, 92, and wherein the cancer is the cancer is hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

In another embodiment the invention relates to the method as above, wherein the disease is immune related condition.

In another embodiment the invention includes the foregoing method, wherein the immune related condition is an inflammatory and/or an autoimmune disease, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In another embodiment the invention includes the foregoing method, which comprises detecting the nucleic acid sequence as set forth in any one of SEQ ID NOs: 1-13, 31, 81, 84, 87, and wherein the immune related condition is selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy.

In another embodiment the invention includes the foregoing method, which comprises detecting the nucleic acid sequence as set forth in any one of SEQ ID NOs: 34-41, 90, 92, and wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In another embodiment the invention includes the foregoing method, which comprises detecting the nucleic acid sequence as set forth in any one of SEQ ID NOs: 34-41, 90, 92, and wherein the disease is ischemia-reperfusion injury, selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In another embodiment the invention includes the foregoing method, which comprises detecting the nucleic acid sequence as set forth in any one of SEQ ID NOs: 34-41, 90, 92, and wherein the disease is respiratory tract disorder, selected from the group including but not limited to chronic obstructive pulmonary disease (COPD), acute respiratory distress syndrome (ARDS), severe acute respiratory syndrome (SARS), asthma, allergy, pulmonary emphysema, pulmonary inflammation, environmental airway disease, airway hyper-responsiveness, chronic bronchitis, acute lung injury, bronchial disease, lung diseases, and cystic fibrosis.

In another embodiment the invention includes the foregoing method, which comprises detecting the nucleic acid sequence as set forth in any one of SEQ ID NOs: 1-13, 31, 81, 84, 87, and wherein the disease is lymphoproliferative disorder, selected from the group consisting of EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

In another embodiment the invention relates to the method as above, wherein the detection is performed using an oligonucleotide pair capable of hybridizing to at least a portion of a nucleic acid sequence at least 85% homologous to the nucleic acid sequence set forth in SEQ ID NO: 1-13, 31, 34-41, 71, 72, 81, 84, 87, 90, or 92.

In another embodiment the invention relates to the method as above wherein the detection is performed using an oligonucleotide pair as set forth in any one of SEQ ID NOs: 58-65, 79-80, 82-83, 85-86, 88-89, 91, or 115-121.

In another embodiment the invention relates to any polypeptide consisting essentially of amino acid sequences as set forth in any one of SEQ ID NOs: 70; 77; 78; or 126-129.

Unless otherwise defined, all technical and scientific terms used herein have the same meaning as commonly understood by one of ordinary skill in the art to which this invention belongs. Although methods and materials similar or equivalent to those described herein optionally may be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. Other features and advantages of the invention will be apparent from the following detailed description and claims.

### BRIEF DESCRIPTION OF THE FIGURES

FIG. 1 shows schematic summary of quantitative real-time PCR analysis.

FIG. 2 shows alignment comparison of the KIAA0746 variant proteins to the known KIAA0746 proteins. FIG. 2A shows alignment of Z43375_{—}1_P4 (SEQ ID NO:18) to Q68CR1_HUMAN (SEQ ID NO:16). FIG. 2B shows alignment of Z43375_{—}1_P8 (SEQ ID NO:19) to O94847_HUMAN (SEQ ID NO:17). FIG. 2C shows alignment of Z43375_{—}1_P40 (SEQ ID NO:20) to Q68CR1_HUMAN (SEQ ID NO:16). FIG. 2D shows alignment of Z43375_{—}1_P46 (SEQ ID NO:21) to Q68CR1_HUMAN (SEQ ID NO:16). FIG. 2E shows alignment of Z43375_{—}1_P47 (SEQ ID NO:22) to Q68CR1_HUMAN (SEQ ID NO:16). FIG. 2F shows alignment of Z43375_{—}1_P50 (SEQ ID NO:23) to Q68CR1_HUMAN (SEQ ID NO:16). FIG. 2G shows alignment of Z43375_{—}1_P51 (SEQ ID NO:24) to Q68CR1_HUMAN (SEQ ID NO:16). FIG. 2H shows alignment of Z43375_{—}1_P52 (SEQ ID NO:25) to Q68CR1_HUMAN (SEQ ID NO:16). FIG. 2AI shows alignment of Z43375_{—}1_P53 (SEQ ID NO:26) to Q68CR1_HUMAN (SEQ ID NO:16) FIG. 2J shows alignment of Z43375_{—}1_P54 (SEQ ID NO:27) to Q68CR1_HUMAN (SEQ ID NO:16). FIG. 2K shows alignment of Z43375_{—}1_P55 (SEQ ID NO:28) to Q68CR1_HUMAN (SEQ ID NO:16). FIG. 2L shows alignment of Z43375_{—}1_P56 (SEQ ID NO:29) to Q68CR1_HUMAN (SEQ ID NO:16).

FIG. 3 shows scatter plot, demonstrating the expression of KIAA0746 transcripts on a virtual panel of all tissues and conditions using MED discovery engine.

FIG. 4 is a histogram showing expression of KIAA0746 transcripts which are detectable by primers as depicted in sequence name CGEN-790_seg33-34-36F1 (SEQ ID NO:79) and CGEN-790_seg33-34-36R1 (SEQ ID NO:80) in various tissue, as listed in Table 1 herein. The sample numbers are marked on line X of the graph, according to Table 1, and appear once at every three time (eg: 25, 28, 31, . . . ).

FIG. 5 is a histogram showing expression of KIAA0746 transcripts which are detectable by primers as depicted in sequence name CGEN-790_seg33-34-36F2 (SEQ ID NO:82) and CGEN-790_seg33-34-36R2 (SEQ ID NO:83) in various tissue, as listed in Table 1 herein. The sample numbers are marked on line X of the graph, according to Table 1, and appear once at every three time (eg: 25, 28, 31, . . . ).

FIG. 6A is a histogram showing expression of KIAA0746 transcripts which are detectable by primers as depicted in sequence name CGEN-790_seg33-34-36F1 (SEQ ID NO:79) and CGEN-790_seg33-34-36R1 (SEQ ID NO:80) on blood panel, as described in Table 2.

FIG. 6B—is a histogram showing expression of KIAA0746 transcripts which are detectable by primers as depicted in sequence name CGEN-790_seg33-34-36F1 (SEQ ID NO:79) and CGEN-790_seg33-34-36R1 (SEQ ID NO:80) on ovary panel, as described in Table 4.

FIG. 7 presents nucleic acid sequences of the KIAA0746_T0_P4 ECD_mFc ORFs (SEQ ID NO:122-125). Gene specific sequence correspond to the ECD sequence is marked in bold faced, TEV cleavage site sequence is underlined, mFc sequence is Italic and IL6 signal peptide sequence is bold Italic. FIG. 7A shows the KIAA0746_(aa 34-305) ECD_mFc DNA sequence (1647 bp) (SEQ ID NO:122); FIG. 7B shows the KIAA0746_(a.a 306-508) ECD-_mFc DNA sequence (1446 bp) (SEQ ID NO:123), FIG. 7C shows the KIAA0746_(a.a 509-765) ECD_mFc DNA sequence (1602 bp) (SEQ ID NO:124); FIG. 7D shows the KIAA0746_(a.a 766-1023) ECD_mFc DNA sequence (1611 bp) (SEQ ID NO:125).

FIG. 8 presents amino acid sequences of the KIAA0746_T0_P4 ECD_mFc ORFs (SEQ ID NO:126-129). Gene specific sequence correspond to the ECD sequence is marked in bold faced, TEV cleavage site sequence is underlined, mFc sequence is Italic and IL6 signal peptide sequence is bold Italic. FIG. 8A shows the KIAA0746_(a.a 34-305) ECD_mFc amino acid sequence (SEQ ID NO:126); FIG. 8B shows the KIAA0746_(a.a 306-508) ECD_mFc amino acid sequence (SEQ ID NO:127), FIG. 8C shows the KIAA0746_(a.a 509-765) ECD_mFc amino acid sequence (SEQ ID NO:128); FIG. 8D shows the KIAA0746_(a.a 766-1023) ECD_mFc amino acid sequence (SEQ ID NO:129).

FIG. 9 shows the results of a Western blot analysis of KIAA0746_(aa 34-305) ECD_mFc (SEQ ID NO:126), KIAA0746_(aa 306-508) ECD_mFc (SEQ ID NO:127), KIAA0746_(aa 509-765) ECD_mFc (SEQ ID NO:128) and KIAA0746_(aa 766-1023) ECD_mFc (SEQ ID NO:129)—constructs in the medium of HEK-293T stably transfected cells. The lanes are as follows: Molecular weight marker (Amersham, full range rainbow, catalog number RPN800) are marked; lane 1—KIAA0746_(aa 34-305) ECD_mFc (SEQ ID NO: 126); lane 2—KIAA0746_(aa 306-508) ECD_mFc (SEQ ID NO: 127); lane 3—KIAA0746_(aa 509-765) ECD_mFc (SEQ ID NO: 128); lane 4—KIAA0746_(aa 766-1023) ECD_mFc (SEQ ID NO: 129); lane 5—pIRES puro3 empty vector.

FIG. 10 alignment of HSCD20B_{—}1_P5 (SEQ ID NO:33) and known protein CD20 HUMAN (SEQ ID NO:32).

FIG. 11A is a histogram showing expression of CD20-variant transcripts which are detectable by amplicon as depicted in sequence name seg00-12F2R2 (SEQ ID NO:87) in blood-specific panel relative to median of the normal samples, described in Table 2.

FIG. 11B is a histogram showing expression of CD20-variant transcripts which are detectable by amplicon as depicted in sequence name seg10-12F2R2 (SEQ ID NO:87) in blood-specific panel relative to median of the kidney normal samples described in Table 2.

FIG. 12 is a histogram showing expression of CD20-variant transcripts which are detectable by amplicon as depicted in sequence name seg10-12F2R2 (SEQ ID NO:87) in normal panel, described in Table 3.

FIG. 13 is a histogram showing expression of CD20-variant transcripts which are detectable by amplicon as depicted in sequence name seg10-12F2R2 (SEQ ID NO:87) in a combined panel, described in Table 5.

FIG. 14 shows the DNA sequence of CD20_T12_FLAG (SEQ ID NO:73). Gene specific sequence corresponding to CD20_T12 ORF sequence is marked in bold faced, FLAG sequence is in italics.

FIG. 15 shows the amino acid sequence of CD20_P5_FLAG (SEQ ID NO:74). The amino acid sequence corresponding to CD20_P5 ORF is marked in bold faced, FLAG sequence is in italics.

FIG. 16 shows the DNA sequence of CD20_T12 (amino acids 66-109)_FLAG (SEQ ID NO:75). Gene specific sequence corresponding to CD20_T12 (amino acids 66-109) sequence is marked in bold faced, GST sequence is in italics and underlined and FLAG sequence is in italics.

FIG. 17 shows the amino acid sequence of GST_CD20_P5 (amino acids 66-109)_FLAG (SEQ ID NO:76). amino acid sequences corresponding to CD20_P5 (amino acids 66-109) sequence is marked in bold faced, GST sequence is in italics and underlined and FLAG sequence is in italics.

FIG. 18 shows western blot analysis of cell lysates of E. coli bacteria DH5a transformed with either GST_CD20_P5 (amino acids 66-109)_FLAG pGEX-6P-1 or with the empty vector pGEX-P6-1, using rabbit anti CD20_SV95 (SEQ ID NO:78) antibodies. In FIG. 18A anti CD20_SV_{—}95 antibodies from rabbit 5359 were used. In FIG. 18B anti CD20_SV95 (SEQ ID NO:78) antibodies from rabbit 5360 were used. Lane 1: pGEX-6P-1, T0; Lane 2: pGEX-6P-1, T3, Lane 3: GST_CD20_SV95 (SEQ ID NO:78), T0; Lane 4: GST_CD20_P5, T3. TO represents zero time. T3 represents time equals 3 hours.

FIG. 19 shows alignment comparison of the HUMDAF variant proteins to the known CD55 proteins. FIGS. 19A, 19B and 19C show the alignment comparison of the HUMDAF_P14 (SEQ ID NO:51) to proteins DAF_HUMAN (SEQ ID NO:42), Q8TD13_HUMAN (SEQ ID NO:50) and Q8TD14 HUMAN (SEQ ID NO:48), respectively. FIGS. 19D, 19E and 19F show the alignment comparison of the HUMDAF_P15 (SEQ ID NO:52) to proteins DAF_HUMAN (SEQ ID NO:42), Q8TD13_HUMAN (SEQ ID NO:50) and Q8TD14 HUMAN (SEQ ID NO:48), respectively. FIGS. 19G, 19H and 19I show the alignment comparison of the HUMDAF_P20 (SEQ ID NO:53) to proteins DAF_HUMAN (SEQ ID NO:42), Q8TD13_HUMAN (SEQ ID NO:50) and Q8TD14 HUMAN (SEQ ID NO:48), respectively. FIGS. 19J and 19K show the alignment comparison of the HUMDAF_P26 (SEQ ID NO:54) to proteins DAF_HUMAN (SEQ ID NO:42), and Q8TD13_HUMAN (SEQ ID NO:50), respectively. FIGS. 19L and 19M show the alignment comparison of the HUMDAF_P29 (SEQ ID NO:55) to proteins DAF_HUMAN (SEQ ID NO:42), and Q8TD13_HUMAN (SEQ ID NO:50), respectively. FIGS. 19N, 19O and 19P show the alignment comparison of the HUMDAF_P30 (SEQ ID NO:56) to proteins DAF_HUMAN (SEQ ID NO:42), Q8TD13_HUMAN (SEQ ID NO:50) and Q8TD14 HUMAN (SEQ ID NO:48), respectively. FIGS. 19Q, 19R and 19S show the alignment comparison of the HUMDAF_P31 (SEQ ID NO:57) to proteins DAF_HUMAN (SEQ ID NO:42), Q8TD13_HUMAN (SEQ ID NO:50) and Q8TD14 HUMAN (SEQ ID NO:48), respectively.

FIG. 20 is a schematic presentation of the CD55 and CD55 splice variants gene structure.

FIGS. 21-22 show scatter plots, demonstrating the expression of HUMDAF transcripts on a virtual panel of all tissues and conditions using MED discovery engine. FIG. 21 shows overexpression of CD55 transcripts in liver cancer. FIG. 22 shows overexpression of CD55 transcripts in pancreatic cancer.

FIG. 23 is a schematic presentation of amplicons used in the experimental assessment of the expression of CD55 and CD55 splice variants.

FIG. 24 is a histogram showing expression of CD55 transcripts which are detectable by the amplicon as depicted in sequence name HUMDAF_DB7_seg24-28_F1R1 (SEQ ID NO:90) on colon panel, described in Table 6.

FIG. 25 is a histogram showing expression of CD55 wild type transcripts which are detectable by the amplicon as depicted in sequence name HUMDAF_DB7_seg24junc27-30_F2R2 (SEQ ID NO:92) on colon panel, described in Table 6.

FIG. 26 presents the ratio of the expression quantity of the wild type CD55, detectable by the amplicon as depicted in sequence name HUMDAF_DB7_seg24junc27-30_F2R2 (SEQ ID NO:92), versus the expression of CD55 variants, detectable by the amplicon as depicted in sequence name HUMDAF_DB7_seg24-28_F1R1 (SEQ ID NO:90), on colon panel, described in Table 6.

FIG. 27 is a histogram showing expression of CD55 transcripts which are detectable by the amplicon as depicted in sequence name HUMDAF_DB7_seg24-28_F1R1 (SEQ ID NO:90) on normal panel, described in Table 3.

FIG. 28 is a histogram showing expression of CD55 wild type transcripts which are detectable by the amplicon as depicted in sequence name HUMDAF_DB7_seg24junc27-30_F2R2 (SEQ ID NO:92) on normal panel, described in Table 3.

FIG. 29 presents the ratio of the expression quantity of the wild type CD55, detectable by the amplicon as depicted in sequence name HUMDAF_DB7_seg24junc27-30_F2R2 (SEQ ID NO:92), versus the expression of CD55 variants, detectable by the amplicon as depicted in sequence name HUMDAF_DB7_seg24-28_F1R1 (SEQ ID NO:90), on normal panel, described in Table 6.

FIGS. 30A and 30B are histograms showing expression of CD55 transcripts which are detectable by the amplicon as depicted in sequence name HUMDAF_DB7_seg24-28_F1R1 (SEQ ID NO:90) on panel of primary immune cells and lymphomas (Table 2). FIG. 30A presents relative expression of each sample relative to median of the normal samples. FIG. 30B presents relative expression of each sample relative to median of the kidney samples.

FIGS. 31A and 31B are histograms showing expression of CD55 wild type transcripts which are detectable by the amplicon as depicted in sequence name HUMDAF_DB7_seg24junc27-30_F2R2 (SEQ ID NO:92) on panel of primary immune cells and lymphomas (Table 2). FIG. 31A presents relative expression of each sample relative to median of the normal samples. FIG. 31B presents relative expression of each sample relative to median of the kidney samples.

FIG. 32 presents the ratio of the expression quantity of the wild type CD55, detectable by the amplicon as depicted in sequence name HUMDAF_DB7_seg24junc27-30_F2R2 (SEQ ID NO:92), versus the expression of CD55 variants, detectable by the amplicon as depicted in sequence name HUMDAF_DB7_seg24-28_F1R1 (SEQ ID NO:90), on panel of primary immune cells and lymphomas (Table 2).

FIG. 33 demonstrates ethidium bromide agarose gel analysis of the CD55 PCR products. Lanes 1 and 9 represent 100 bp DNA marker (Fermentas, Catalog number SM0244) lanes 2-8 represent the PCR products as follows, lane 2-Ovary borderline tumor 38-GC-SIA-BRD; lane 3—Ovary cancer 30-GC-SIC-MUC; lane 4—Lung cancer 17-(89)-Bc-Adeno; lane 5—Lung cancer 18-(76)-Bc-Adeno; lane 6—Colon cancer 24-(14)-Ic-AdenoSIII; lane 7—Colon cancer 25-(23)-Ic-AdenoSIII; lane 8—Colon cancer 27-GC-AdenoSIII.

FIG. 34 shows the DNA sequence of the CD55 transcript HUMDAF TO FLAG (SEQ ID NO:66). Gene specific sequence corresponding to CD55_T0 ORF sequence is marked in bold faced, FLAG tag sequence is in italics, silent mutation is underlined.

FIG. 35 shows the amino acid sequence of CD55_P0_FLAG (SEQ ID NO:67); amino acid sequence corresponding to CD55 ORF is marked in bold faced, FLAG sequence is in italics.

FIG. 36 shows the DNA sequence of the CD55 transcript CD55_T11_P15(1-523)_FLAG (SEQ ID NO:68). Gene specific sequence corresponding to CD55_T11 ORF sequence is marked in bold faced, FLAG tag sequence is in italics, point mutation is underlined.

FIG. 37 shows the amino acid sequence of CD55_T11_P15(1-523)_FLAG (SEQ ID NO:69). FLAG sequence is in italics.

FIGS. 38A-C demonstrate immuno-precipitation of CD55 followed by western blot analysis. FIG. 38A presents the results of immuno-precipitation with mouse anti CD55 (NaM16-4D3) antibody, followed by western blot with commercial mouse anti CD55 (ab54595). FIGS. 38B and 38C present immuno-precipitation with mouse anti CD55 (NaM16-4D3) antibody, followed by western blot with rabbit anti CD55_P15 sera, 5619 and 5620, respectively. Lane 1 represents un-transfected CHO-K1 cells; lane 2 represents CHO-K1 stably transfected with CD55_P15_S523FLAG pIRESpuro3; lane 3 represents CHO-K1 stably transfected with CD55_P0_FLAG (SEQ ID NO:66). A cross reactive band is marked by *.

FIG. 39 demonstrates the immuno-fluorescence analysis, demonstrating the specific binding of the anti-CD55_antibodies specific to CD55 variants (SEQ ID NOs: 51, 52, 53, 56 and 57) to CD55 variant proteins in colon cells.

### DETAILED DESCRIPTION OF THE INVENTION

The present invention, in some embodiments, relates to any one of the antigens referred to as KIAA0746, CD20, CD55, and its corresponding amino acid and nucleic acid sequence, and portions and variants thereof and conjugates thereof and the use thereof as a therapeutic or diagnostic target. In particular the invention, in some embodiments, uses this antigen and discrete portions thereof as a drug target for therapeutic small molecules, peptides, antibodies, antisense RNAs, siRNAs, ribozymes, and the like. More particularly the invention relates to diagnostic and therapeutic polyclonal and monoclonal antibodies and fragments thereof that bind KIAA0746, CD20, CD55 and portions and variants thereof, especially those that target the ectodomain or portions or variants thereof particularly human or chimeric monoclonal antibodies, that bind specifically to the antigen Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), and HUMDAF_P31 (SEQ ID NO:57), and variants thereof and anti-idiotypic antibodies specific thereto including those that promote or inhibit activities elicited by KIAA0746, CD20, CD55.

In certain embodiments, the antibodies of the invention are derived from particular heavy and light chain germline sequences and/or comprise particular structural features such as CDR regions comprising particular amino acid sequences. The invention provides isolated antibodies, methods of making such antibodies, immunoconjugates and bispecific molecules comprising such antibodies and pharmaceutical and diagnostic compositions containing the antibodies, immunoconjugates or bispecific molecules of the invention.

The invention, in other embodiments, also relates to in vitro and in vivo methods of using the antibodies and fragments, to detect KIAA0746, CD20, CD55, as well as to treat diseases associated with expression of KIAA0746, CD20, or CD55, such as malignancies that differentially express KIAA0746, CD20, or CD55. The invention, in other embodiments, further relates to methods of using the antibodies and fragments, specific for KIAA0746, CD20, CD55 to treat immune related conditions. The invention, in other embodiments, further relates to methods of using the antibodies and fragments, specific for KIAA0746 or CD20, to treat lymphoproliferative disorder. The invention, in other embodiments, further relates to methods of using the antibodies and fragments, specific for CD55, to treat diseases in which complement activation and deposition is involved in pathogenesis, inflammation of the respiratory tract disorders and ischemia-reperfusion injury related disorders. Preferably these antibodies will possess ADCC or CDC activity against target cells such as cancer cells.

Also, the invention, in other embodiments, relates to the KIAA0746, CD20, CD55 antigen and portions thereof including soluble polypeptide conjugates containing the ectodomain of KIAA0746, CD20, CD55 and/or the corresponding DNAs or vectors or cells expressing same for use in immunotherapy. Further the invention, in other embodiments, provides vectors, cells containing and use thereof for the expression of the KIAA0746, CD20, CD55 antigen, as well as discrete portions and variants thereof. Also, the invention, in other embodiments, provides non-antibody based KIAA0746, CD20, CD55 modulatory agents such as peptides, antisense RNAs, siRNAs, carbohydrates, and other small molecules that specifically bind and/or modulate a KIAA0746, CD20, CD55 related activity.

In order that the present invention may be more readily understood, certain terms are first defined. Additional definitions are set forth throughout the detailed description.

The term KIAA0746 refers to the protein encoded by any one of the Z43375_{—}1_T0 (SEQ ID NO:1), Z43375_{—}1_T3 (SEQ ID NO:2), Z43375_{—}1_T6 (SEQ ID NO:3), Z43375_{—}1_T7 (SEQ ID NO:4), Z43375_{—}1_T14 (SEQ ID NO:5), Z43375_{—}1_T16 (SEQ ID NO:6), Z43375_{—}1_T20 (SEQ ID NO:7), Z43375_{—}1_T22 (SEQ ID NO:8), Z43375_{—}1_T23 (SEQ ID NO:9), Z43375_{—}1_T28 (SEQ ID NO:10), Z43375_{—}1_T30 (SEQ ID NO:11), Z43375_{—}1_T31 (SEQ ID NO:12), Z43375_{—}1_T33 (SEQ ID NO:13) transcripts reported herein, particularly to proteins as set forth in any one of Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), and Z43375_{—}1_P60 (SEQ ID NO:30), and variants thereof, especially those possessing at least 80, 85, 90, 95 or higher sequence identity therewith. According to some embodiments of the present invention, KIAA0746 transcripts and/or proteins are differentially expressed in cancer, particularly in prostate cancer, pancreas cancer, ovary cancer, lung cancer, liver cancer, colon cancer, kidney cancer, melanoma, head and neck cancer, wherein the cancer is non-metastatic, invasive or metastatic; as well as in non-malignant disorders such as immune related conditions, particularly in rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders (such as pemphigus, pemphigoid), atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy; and in lymphoproliferative disorders.

The term CD20 refers to the protein encoded by HSCD20B_{—}1_T12 (SEQ ID NO:31) transcripts reported herein, particularly to protein as set forth in HSCD20B_{—}1_P5 (SEQ ID NO:33), and variants thereof especially those possessing at least 80, 85, 90, 95 or higher sequence identity therewith. According to some embodiments of the present invention, CD20 transcripts and/or proteins are differentially expressed in cancer, particularly in hematological malignancies, primarily B-cell derived, such as acute lymphocytic leukemia, chronic lymphocytic leukemia, acute myelogenous leukemia, chronic myelogenous leukemia, multiple myeloma, and B-cell lymphoma, selected from the group consisting of, but not limited to non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the cancer is invasive or metastatic; as well as in non-malignant disorders such as immune related conditions, particularly rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders (such as pemphigus, pemphigoid), atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy; acute and chronic rejection of organ transplantation, allogeneic stem cell transplantation, autologous stem cell transplantation, bone marrow transplantation, and treatment of Graft Versus Host Disease (GVHD), as well as in lymphoproliferative disorders.

The term CD55 refers to the protein encoded by any one of the HUMDAF_T10 (SEQ ID NO:34), HUMDAF_T11 (SEQ ID NO:35), HUMDAF_T17 (SEQ ID NO:36), HUMDAF_T19 (SEQ ID NO:37), HUMDAF_T24 (SEQ ID NO:38), HUMDAF_T30 (SEQ ID NO:39), HUMDAF_T31 (SEQ ID NO:40), HUMDAF_T32 (SEQ ID NO:41) transcripts reported herein, particularly to proteins as set forth in any one of HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57), and variants thereof especially those possessing at least 80, 85, 90, 95 or higher sequence identity therewith. According to some embodiments of the present invention, the CD55 transcripts and/or proteins are differentially expressed in cancer, particularly in colorectal cancer, lung cancer, prostate cancer, pancreas cancer, ovarian cancer, gastric cancer, liver cancer, wherein the cancer is non-metastatic, invasive or metastatic; as well as non-malignant disorders such as immune related conditions, particularly rheumatoid arthritis (RA), systemic lupus erythematosus (SLE), lupus nephtirits and multiple sclerosis (MS), inflammatory bowel disease (IBD), ulcerative colitis, psoriasis, disease states in which complement activation and deposition is involved in pathogenesis, inflammation of the respiratory tract disorders, ischemia-reperfusion injury related disorders, transplant rejection and graft versus host disease.

Preferably such KIAA0746, CD20, CD55 variants will possess at least 80% sequence identity therewith, more preferably at least 90% sequence identity therewith and even more preferably at least 95% sequence identity therewith.

The term the “soluble ectodomain (ECD)” or “ectodomain” of KIAA0746 refers to the polypeptide sequences below or the corresponding nucleic acid sequences (which does not comprise the signal peptide and the TM of KIAA0746 protein):

>Z43375_1_P4 (SEQ ID NO: 18) amino acid residues from 33 to 1023 (SEQ ID NO: 93)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVWAKHVAEKNGYLGHVIRKGLNAYLEGSWHEALLYYVLAA
ETGIEVSQTNLAHICEERPDLARRYLGVNCVWRYYNFSVFQIDAPSFAYLKMGDLYYYGH
QNQSQDLELSVQMYAQAALDGDSQGFFNLALLIEEGTIIPHHILDFLEIDSTLHSNNISI
LQELYERCWSHSNEESFSPCSLAWLYLHLRL
>Z43375_1_P8 (SEQ ID NO: 19) amino acid residues from 17 to 1049 (SEQ ID NO: 94)
KHPERAANQPAGWGAARLQTCQQGGSPNPAGGQVENVVPSLGRQTSLTTSVIPKAEQSVA
YKDFIYFTVFEGNVRNVSEVSVEYLCSQPCVVNLEAVVSSEFRSSIPVYKKRWKNEKHLH
TSRTQIVHVKFPSIMVYRDDYFIRHSISVSAVIVRAWITHKYSGRDWNVKWEENLLHAVA
KNYTLLQTIPPFERPFKDHQVCLEWNMGYIWNLRANRIPQCPLENDVVALLGFPYASSGE
NTGIVKKFPRFRNRELEATRRQRMDYPVFTVSLWLYLLHYCKANLCGILYFVDSNEMYGT
PSVFLTEEGYLHIQMHLVKGEDLAVKTKFIIPLKEWFRLDISFNGGQIVVTTSIGQDLKS
YHNQTISFREDFHYNDTAGYFIIGGSRYVAGIEGFFGPLKYYRLRSLHPAQIFNPLLEKQ
LAEQIKLYYERCAEVQEIVSVYASAAKHGGERQEACHLHNSYLDLQRRYGRPSMCRAFPW
EKELKDKHPSLFQALLEMDLLTVPRNQNESVSEIGGKIFEKAVKRLSSIDGLHQISSIVP
FLTDSSCCGYHKASYYLAVFYETGLNVPRDQLQGMLYSLVGGQGSERLSSMNLGYKHYQG
IDNYPLDWELSYAYYSNIATKTPLDQHTLQGDQAYVETIRLKDDEILKVQTKEDGDVFMW
LKHEATRGNAAAQQRLAQMLFWGQQGVAKNPEAAIEWYAKGALETEDPALIYDYAIVLFK
GQGVKKNRRLALELMKKAASKGLHQAVNGLGWYYHKFKKNYAKAAKYWLKAEEMGNPDAS
YNLGVLHLDGIFPGVPGRNQTLAGEYFHKAAQGGHMEGTLWCSLYYITGNLETFPRDPEK
AVVWAKHVAEKNGYLGHVIRKGLNAYLEGSWHEALLYYVLAAETGIEVSQTNLAHICEER
PDLARRYLGVNCVWRYYNFSVFQIDAPSFAYLKMGDLYYYGHQNQSQDLELSVQMYAQAA
LDGDSQGFFNLALLIEEGTIIPHHILDFLEIDSTLHSNNISILQELYERCWSHSNEESFS
PCSLAWLYLHLRL
>Z43375_1_P40 (SEQ ID NO: 20) amino acid residues from 33 to 887 (SEQ ID NO: 95)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVWAKHVAEKNGYLGHVIRKGLNAYLEGSWPQKVQNFYLVP
SKKRDQCLRFRPPLP
>Z43375_1_P46 (SEQ ID NO: 21) amino acid residues from 33 to 995 (SEQ ID NO: 96)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYAS SGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVHEALLYYVLAAETGIEVSQTNLAHICEERPDLARRYLGV
NCVWRYYNFSVFQIDAPSFAYLKMGDLYYYGHQNQSQDLELSVQMYAQAALDGDSQGFFN
LALLIEEGTIIPHHILDFLEIDSTLHSNNISILQELYERCWSHSNEESFSPCSLAWLYLH
LRL
>Z43375_1_P47 (SEQ ID NO: 22) amino acid residues from 33 To 1022 (SEQ ID NO: 97)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYAS SGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVWAKHVAEKNGYLGHVIRKGLNAYLEGSWHEALLYYVLAA
ETGIEVSQTNLAHICEERPDLARRYLGVNCVWRYYNFSVFQIDAPSFAYLKMGDLYYYGH
QNQSQDLELSVQMYAQAALDGDSQGFFNLALLIEEGTIIPHHILDFLEIDSTLHSNNISI
LQELYERCWSHSNEESFSPCSLAWLYLHLR
>Z43375_1_P50 (SEQ ID NO: 23) amino acid residues from 33 To 977 (SEQ ID NO: 98)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVWAKHVAEKNGYLGHVIRKGLNAYLEGSWHEALLYYVLAA
ETGIEVSQTNLAHICEERPDLARRYLGVNCVWRYYNFSVFQIDAPSFAYLKMGDLYYYGH
QNQSQDLELSVQMYAQAALDGDSQGFFNLALLIEEGTVRKVLEPQ
>Z43375_1_P51 (SEQ ID NO: 24) amino acid residues from 33 To 792 (SEQ ID NO: 99)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQHI
>Z43375_1_P52 (SEQ ID NO: 25) amino acid residues from 33 To 1010 (SEQ ID NO: 100)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVWAKHVAEKNGYLGHVIRKGLNAYLEGSWHEALLYYVLAA
ETGIEVSQTNLAHICEERPDLARRYLGVNCVWRYYNFSVFQIDAPSFAYLKMGDLYYYGH
QNQSQDLELSVQMYAQAALDGDSQGFFNLALLIEEGTIIPHHILDFLEIDSTLHSNNISI
LQELYERSTFWEPFCYPY
>Z43375_1_P53 (SEQ ID NO: 26) amino acid residues from 33 To 839 (SEQ ID NO: 101)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGLPRHCHVHCKSSCDSSCRCL
>Z43375_1_P54 (SEQ ID NO: 27) amino acid residues from 33 To 833 (SEQ ID NO: 102)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVV
>Z43375_1_P55 (SEQ ID NO: 28) amino acid residues from 33 To 867 (SEQ ID NO: 103)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKGQGVKKNRRLALELMKKAASKGLHQAVNGLGWYYHKFKKNYA
KAAKYWLKAEEMGNPDASYNLGVLHLDGIFPGVPGRNQTLAGEYFHKAAQGGHMEGTLWC
SLYYITGNLETFPRDPEKAVVKSLSTSVLGHPHTDTLALQKIVLHNTFGFKFNLT
>Z43375_1_P56 (SEQ ID NO: 29) amino acid residues from 33 To 714 (SEQ ID NO: 104)
RQTSLTTSVIPKAEQSVAYKDFIYFTVFEGNVRNVSEVSVEYLCSQPCVVNLEAVVSSEF
RSSIPVYKKRWKNEKHLHTSRTQIVHVKFPSIMVYRDDYFIRHSISVSAVIVRAWITHKY
SGRDWNVKWEENLLHAVAKNYTLLQTIPPFERPFKDHQVCLEWNMGYIWNLRANRIPQCP
LENDVVALLGFPYASSGENTGIVKKFPRFRNRELEATRRQRMDYPVFTVSLWLYLLHYCK
ANLCGILYFVDSNEMYGTPSVFLTEEGYLHIQMHLVKGEDLAVKTKFIIPLKEWFRLDIS
FNGGQIVVTTSIGQDLKSYHNQTISFREDFHYNDTAGYFIIGGSRYVAGIEGFFGPLKYY
RLRSLHPAQIFNPLLEKQLAEQIKLYYERCAEVQEIVSVYASAAKHGGERQEACHLHNSY
LDLQRRYGRPSMCRAFPWEKELKDKHPSLFQALLEMDLLTVPRNQNESVSEIGGKIFEKA
VKRLSSIDGLHQISSIVPFLTDSSCCGYHKASYYLAVFYETGLNVPRDQLQGMLYSLVGG
QGSERLSSMNLGYKHYQGIDNYPLDWELSYAYYSNIATKTPLDQHTLQGDQAYVETIRLK
DDEILKVQTKEDGDVFMWLKHEATRGNAAAQQRLAQMLFWGQQGVAKNPEAAIEWYAKGA
LETEDPALIYDYAIVLFKVRIT
>Z43375_1_P60 (SEQ ID NO: 30) amino acid residues from 21 To 770 (SEQ ID NO: 105)
NLCGILYFVDSNEMYGTPSVFLTEEGYLHIQMHLVKGEDLAVKTKFIIPLKEWFRLDISF
NGGQIVVTTSIGQDLKSYHNQTISFREDFHYNDTAGYFIIGGSRYVAGIEGFFGPLKYYR
LRSLHPAQIFNPLLEKQLAEQIKLYYERCAEVQEIVSVYASAAKHGGERQEACHLHNSYL
DLQRRYGRPSMCRAFPWEKELKDKHPSLFQALLEMDLLTVPRNQNESVSEIGGKIFEKAV
KRLSSIDGLHQISSIVPFLTDSSCCGYHKASYYLAVFYETGLNVPRDQLQGMLYSLVGGQ
GSERLSSMNLGYKHYQGIDNYPLDWELSYAYYSNIATKTPLDQHTLQGDQAYVETIRLKD
DEILKVQTKEDGDVFMWLKHEATRGNAAAQQRLAQMLFWGQQGVAKNPEAAIEWYAKGAL
ETEDPALIYDYAIVLFKGQGVKKNRRLALELMKKAASKGLHQAVNGLGWYYHKFKKNYAK
AAKYWLKAEEMGNPDASYNLGVLHLDGIFPGVPGRNQTLAGEYFHKAAQGGHMEGTLWCS
LYYITGNLETFPRDPEKAVVWAKHVAEKNGYLGHVIRKGLNAYLEGSWHEALLYYVLAAE
TGIEVSQTNLAHICEERPDLARRYLGVNCVWRYYNFSVFQIDAPSFAYLKMGDLYYYGHQ
NQSQDLELSVQMYAQAALDGDSQGFFNLALLIEEGTIIPHHILDFLEIDSTLHSNNISIL
QELYERCWSHSNEESFSPCSLAWLYLHLRL;

and variants thereof possessing at least 80% sequence identity, more preferably at least 90% sequence identity therewith and even more preferably at least 95, 96, 97, 98 or 99% sequence identity therewith. The term the “soluble ectodomain (ECD)” or “ectodomain” of CD20 refers to the polypeptide sequences below or the corresponding nucleic acid sequences (which does not comprise the signal peptide and the TM of CD20 protein: HSCD20B_{—}1_P5 (SEQ ID NO:33) amino acid residues 87-109 (SEQ ID NO:106) PLWGGIMPECEKRKMSNSHHHFL; or HSCD20B_{—}1_P5 (SEQ ID NO:33) amino acid residues 1-63 (SEQ ID NO:107)

MTTPRNSVNGTFPAEPMKGPIAMQSGPKPLFRRMSSLVGPTQSFFMRESK
TLGAVQIMNGLFH,

and variants thereof possessing at least 80% sequence identity, more preferably at least 90% sequence identity therewith and even more preferably at least 95, 96, 97, 98 or 99% sequence identity therewith.

The term the “soluble ectodomain (ECD)” or “ectodomain” of CD55 refers to the polypeptide sequences below or the corresponding nucleic acid sequences (which does not comprise the signal peptide and the TM of CD55 protein):

>HUMDAF_P14 (SEQ ID NO: 51), amino acid residues 35-497 (SEQ ID NO: 108)
DCGLPPDVPNAQPALEGRTSFPEDTVITYKCEESFVKIPGEKDSVICLKGSQWSDIEEF
CNRSCEVPTRLNSASLKQPYITQNYFPVGTVVEYECRPGYRREPSLSPKLTCLQNLKW
STAVEFCKKKSCPNPGEIRNGQIDVPGGILFGATISFSCNTGYKLFGSTSSFCLISGSSVQ
WSDPLPECREIYCPAPPQIDNGIIQGERDHYGYRQSVTYACNKGFTMIGEHSIYCTVN
NDEGEWSGPPPECRGKSLTSKVPPTVQKPTTVNVPTTEVSPTSQKTTTKTTTPNAQGT
ETPSVLQKHTTENVSATRTPPTPQKPTTVNVPATIVTPTPQKPTTINVPATGVSSTPQR
HTIVNVSATGTLPTLQKPTRANDSATKSPAAAQTSFISKTLSTKTPSAAQNPMMTNAS
ATQATLTAQRFTTAKVAFTQSPSAAPTRSTPVSRTTKHFHETTPNKGSGTTS
>HUMDAF_P15 (SEQ ID NO: 52), amino acid residues 35-523 (SEQ ID NO: 109)
DCGLPPDVPNAQPALEGRTSFPEDTVITYKCEESFVKIPGEKDSVICLKGSQWSDIEEF
CNRSCEVPTRLNSASLKQPYITQNYFPVGTVVEYECRPGYRREPSLSPKLTCLQNLKW
STAVEFCKKKSCPNPGEIRNGQIDVPGGILFGATISFSCNTGYKLFGSTSSFCLISGSSVQ
WSDPLPECREIYCPAPPQIDNGIIQGERDHYGYRQSVTYACNKGFTMIGEHSIYCTVN
NDEGEWSGPPPECRGKSLTSKVPPTVQKPTTVNVPTTEVSPTSQKTTTKTTTPNAQGT
ETPSVLQKHTTENVSATRTPPTPQKPTTVNVPATIVTPTPQKPTTINVPATGVSSTPQR
HTIVNVSATGTLPTLQKPTRANDSATKSPAAAQTSFISKTLSTKTPSAAQNPMMTNAS
ATQATLTAQRFTTAKVAFTQSPSAAHKSTNVHSPVTNGLKSTQRFPSAHITATRSTPV
SRTTKHFHETTPNKGSGTTS
>HUMDAF_P20 (SEQ ID NO: 53), amino acid residues 35-497 (SEQ ID NO: 108)
DCGLPPDVPNAQPALEGRTSFPEDTVITYKCEESFVKIPGEKDSVICLKGSQWSDIEEF
CNRSCEVPTRLNSASLKQPYITQNYFPVGTVVEYECRPGYRREPSLSPKLTCLQNLKW
STAVEFCKKKSCPNPGEIRNGQIDVPGGILFGATISFSCNTGYKLFGSTSSFCLISGSSVQ
WSDPLPECREIYCPAPPQIDNGIIQGERDHYGYRQSVTYACNKGFTMIGEHSIYCTVN
NDEGEWSGPPPECRGKSLTSKVPPTVQKPTTVNVPTTEVSPTSQKTTTKTTTPNAQGT
ETPSVLQKHTTENVSATRTPPTPQKPTTVNVPATIVTPTPQKPTTINVPATGVSSTPQR
HTIVNVSATGTLPTLQKPTRANDSATKSPAAAQTSFISKTLSTKTPSAAQNPMMTNAS
ATQATLTAQRFTTAKVAFTQSPSAAPTRSTPVSRTTKHFHETTPNKGSGTTS
>HUMDAF_P26(SEQ ID NO: 54), amino acid residues 36-371 (SEQ ID NO: 110)
SRVEHTMLQTCMSSLSGDCGLPPDVPNAQPALEGRTSFPEDTVITYKCEESFVKIPGE
KDSVICLKGSQWSDIEEFCNRSCEVPTRLNSASLKQPYITQNYFPVGTVVEYECRPGY
RREPSLSPKLTCLQNLKWSTAVEFCKKKSCPNPGEIRNGQIDVPGGILFGATISFSCNT
GYKLFGSTSSFCLISGSSVQWSDPLPECREIYCPAPPQIDNGIIQGERDHYGYRQSVTY
ACNKGFTMIGEHSIYCTVNNDEGEWSGPPPECRGKSLTSKVPPTVQKPTTVNVPTTEV
SPTSQKTTTKTTTPNAQATRSTPVSRTTKHFHETTPNKGSGTTS
>HUMDAF_P29(SEQ ID NO: 55), amino acid residues 35-328 (SEQ ID NO: 111)
DCGLPPDVPNAQPALEGRTSFPEDTVITYKCEESFVKIPGEKDSVICLKGSQWSDIEEF
CNLGTVVEYECRPGYRREPSLSPKLTCLQNLKWSTAVEFCKKKSCPNPGEIRNGQIDV
PGGILFGATISFSCNTGYKLFGSTSSFCLISGSSVQWSDPLPECREIYCPAPPQIDNGIIQG
ERDHYGYRQSVTYACNKGFTMIGEHSIYCTVNNDEGEWSGPPPECRGKSLTSKVPPT
VQKPTTVNVPTTEVSPTSQKTTTKTTTPNAQATRSTPVSRTTKHFHETTPNKGSGTTS
>HUMDAF_P30(SEQ ID NO: 56), amino acid residues 35-497 (SEQ ID NO: 108)
DCGLPPDVPNAQPALEGRTSFPEDTVITYKCEESFVKIPGEKDSVICLKGSQWSDIEEF
CNRSCEVPTRLNSASLKQPYITQNYFPVGTVVEYECRPGYRREPSLSPKLTCLQNLKW
STAVEFCKKKSCPNPGEIRNGQIDVPGGILFGATISFSCNTGYKLFGSTSSFCLISGSSVQ
WSDPLPECREIYCPAPPQIDNGIIQGERDHYGYRQSVTYACNKGFTMIGEHSIYCTVN
NDEGEWSGPPPECRGKSLTSKVPPTVQKPTTVNVPTTEVSPTSQKTTTKTTTPNAQGT
ETPSVLQKHTTENVSATRTPPTPQKPTTVNVPATIVTPTPQKPTTINVPATGVSSTPQR
HTIVNVSATGTLPTLQKPTRANDSATKSPAAAQTSFISKTLSTKTPSAAQNPMMTNAS
ATQATLTAQRFTTAKVAFTQSPSAAPTRSTPVSRTTKHFHETTPNKGSGTTS
>HUMDAF_P31, (SEQ ID NO: 57), amino acid residues 35-523 (SEQ ID NO: 112)
DCGLPPDVPNAQPALEGRTSFPEDTVITYKCEESFVKIPGEKDSVICLKGSQWSDIEEF
CNRSCEVPTRLNSASLKQPYITQNYFPVGTVVEYECRPGYRREPSLSPKLTCLQNLKW
STAVEFCKKKSCPNPGEIRNGQIDVPGGILFGATISFSCNTGYKLFGSTSSFCLISGSSVQ
WSDPLPECREIYCPAPPQIDNGIIQGERDHYGYRQSVTYACNKGFTMIGEHSIYCTVN
NDEGEWSGPPPECRGKSLTSKVPPTVQKPTTVNVPTTEVSPTSQKTTTKTTTPNAQGT
ETPSVLQKHTTENVSATRTPPTPQKPTTVNVPATIVTPTPQKPTTINVPATGVSSTPQR
HTIVNVSATGTLPTLQKPTRANDSATKSPAAAQTSFISKTLSTKTPSAAQNPMMTNAS
ATQATLTAQRFTTAKVAFTQSPSAAHKSTNVHSPVTNGLKSTQRFPSAHITATRSTPV
SRTTKHFHETTPNKGSGTTS,

and variants thereof possessing at least 80% sequence identity, more preferably at least 90% sequence identity therewith and even more preferably at least 95, 96, 97, 98 or 99% sequence identity therewith.

The term “immune response” refers to the action of, for example, lymphocytes, antigen presenting cells, phagocytic cells, granulocytes, and soluble macromolecules produced by the above cells or cells produced by the liver or spleen (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell.

As used herein, the phrase “cell surface receptor” includes, for example, molecules and complexes of molecules capable of receiving a signal and the transmission of such a signal across the plasma membrane of a cell and/or within a cell.

The term “antibody” as referred to herein includes whole polyclonal and monoclonal antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. In addition the term “antibody” optionally includes anti-idiotypic antibodies generated against or specific to any of the antibodies and fragments according to the invention.

The term “antigen-binding portion” of an antibody (or simply “antibody portion”), as used herein, refers to one or more fragments of an antibody that retain the ability to specifically bind to an antigen (e.g., KIAA0746, CD20, CD55 proteins). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V Light, V Heavy, Constant light (CL) and CH1 domains; (ii) a F(ab′).2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds KIAA0746, CD20, CD55 proteins or KIAA0746, CD20, CD55 is substantially free of antibodies that specifically bind antigens other than KIAA0746, CD20, CD55 proteins, respectively. An isolated antibody that specifically binds KIAA0746, CD20, CD55 proteins or may, however, have cross-reactivity to other antigens, such as KIAA0746, CD20, CD55 proteins or KIAA0746, CD20, CD55 molecules from other species, respectively. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human KIAA0746, CD20, CD55 proteins is intended to refer to an antibody that binds to human KIAA0746, CD20, CD55 proteins, respectively, preferably one with a KD of 5×10−8 M or less, more preferably 3×10−8 M or less, and even more preferably 1×.10−9 M or less.

The term “K-assoc” or “Ka”, as used herein, is intended to refer to the association rate of a particular antibody-antigen interaction, whereas the term “Kdiss” or “Kd,” as used herein, is intended to refer to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface Plasmon resonance, preferably using a biosensor system such as a Biacore® system.

As used herein, the term “high affinity” for an IgG antibody refers to an antibody having a KD of 10−8 M or less, more preferably 10−9 M or less and even more preferably 10−10 M or less for a target antigen. However, “high affinity” binding can vary for other antibody isotypes. For example, “high affinity” binding for an IgM isotype refers to an antibody having a KD of 10−7 M or less, more preferably 10−8 M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the term “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein, the term “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein, the term “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It will be noted that a bridge cannot be extended beyond the length of the sequence in either direction, and it will be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it will also be read as including bridges in which n is any number of amino acids between 10-50 amino acids in length. Furthermore, the bridge polypeptide cannot extend beyond the sequence, so it will be read such that 49−x (for example) is not less than 1, nor 50+((n−2)−x) (for example) greater than the total sequence length.

The term “cancer” as used herein will encompass any disease disorder or condition selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain is non-metastatic, invasive or metastatic.

With regard to lung cancer, the disease is selected from the group including but not limited to squamous cell lung carcinoma, lung adenocarcinoma, carcinoid, small cell lung cancer or non-small cell lung cancer.

With regard to breast cancer, the disease is selected from the group including but not limited to primary and metastatic cancer of the breast, including mammary carcinomas such as Infiltrating Ductal carcinoma, Ductal carcinoma in-situ, Infiltrating Lobular carcinoma, Lobular carcinoma in-situ, Inflammatory breast cancer, Paget's disease of the breast, and other non-epithelial neoplasms of the breast, including fibrosarcomas, leiomyosarcomas, rhapdomyosarcomas, angiosarcomas, cystosarcoma phyllodes.

With regard to ovarian cancer, the disease is selected from the group including but not limited to primary and metastatic cancer of the ovary, including epithelial ovarian cancer such as serous, mucinous, endometroid, clear cell, mixed epithelial, undifferentiated carcinomas and Brenner tumor, as well as other non-epithelial neoplasms of the ovary, including germ cell malignancies.

With regard to liver cancer, the disease is selected from the group including but not limited to primary and metastatic cancers of the liver and intrahepatic bile duct, including hepatocellular carcinoma, cholangiocarcinoma, hepatic angiosarcoma and hepatoblastoma.

With regard to renal cancer, the disease is selected from the group including but not limited to primary and metastatic cancer of the kidney, including renal cell carcinoma (i.e. renal adenocarcinoma), as well as other non-epithelial neoplasms of the ovary, including nephroblastoma (i.e. Wilm's tumor), transitional cell neoplasms of the renal pelvis, and various sarcomas of renal origin.

With regard to pancreatic cancer, the disease is selected from the group including but not limited to primary and metastatic cancers of the exocrine pancreas, including adenocarcinoma, serous and mucinous cystadenocarcinomas, acinar cell carcinoma, undifferentiated carcinoma, pancreatoblastoma and neuroendocrine tumors such as insulinoma.

With regard to melanoma, the disease is selected from the group including but not limited to primary and metastatic malignant melanoma, including cutaneous melanoma such as superficial spreading melanoma, nodular melanoma, acral lentiginous melanoma and lentigo maligna melanoma, as well as mucosal melanoma, intraocular melanoma, desmoplastic/neurotropic melanoma and melanoma of soft parts (clear cell sarcoma).

With regard to prostate cancer, the disease is selected from the group including but not limited to primary and metastatic cancer of the prostate, including prostatic intraepithelial neoplasia, atypical adenomatous hyperplasia, prostate adenocarcinoma, mucinous or signet ring tumor, adenoid cystic carcinoma, prostatic duct carcinoma, carcinoid and small-cell undifferentiated cancer.

With regard to gastric cancer, the disease is selected from the group including but not limited to gastric carcinoma, gastric adenocarcinoma (Intestinal or Diffused), and wherein the cancer is non-metastatic, invasive or metastatic.

With regard to liver cancer, the disease is selected from the group including but not limited to Hepatocellular carcinoma (HCC), hepatocellular cancer, Intrahepatic cholangiocarcinomas (bile duct cancers), Angiosarcomas and hemangiosarcomas, and wherein the cancer is non-metastatic, invasive or metastatic.

With regard to head and neck cancer, the disease is selected from the group including but not limited to squamous cell carcinoma, Verrucous carcinoma, carcinoid of the head and neck, and wherein the cancer is non-metastatic, invasive or metastatic.

With regard to hematological malignancies, the disease is selected from the group including but not limited to acute lymphocytic leukemia, chronic lymphocytic leukemia, acute myelogenous leukemia, chronic myelogenous leukemia, multiple myeloma, Hodgkin's lymphoma and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy, and wherein the cancer is non-metastatic, invasive or metastatic.

The term “immune related conditions” as used herein will encompass any disease disorder or condition selected from inflammatory and/or autoimmune diseases, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

The term “lymphoproliferative disorder” as used herein will encompass any disease disorder or condition selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

The term “inflammation of the respiratory tract disorders” as used herein will encompass any disease disorder or condition selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

The term “ischemia-reperfusion injury disorders” as used herein will encompass any disease disorder or condition selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

Various aspects of the invention are described in further detail in the following subsections.

Nucleic Acids

A “nucleic acid fragment” or an “oligonucleotide” or a “polynucleotide” are used herein interchangeably to refer to a polymer of nucleic acid residues. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

Thus, the present invention encompasses nucleic acid sequences described hereinabove; fragments thereof, sequences hybridizable therewith, sequences homologous thereto [e.g., at least 90%, at least 95, 96, 97, 98 or 99% or more identical to the nucleic acid sequences set forth herein], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention), which include sequence regions unique to the polynucleotides of at least some embodiments of the present invention.

In cases where the polynucleotide sequences of the present invention encode previously unidentified polypeptides, the present invention also encompasses novel polypeptides or portions thereof in at least some embodiments, which are encoded by the isolated polynucleotide and respective nucleic acid fragments thereof described hereinabove.

Thus, the present invention, in at least some embodiments, also encompasses polypeptides encoded by the polynucleotide sequences of the present invention. The present invention also encompasses homologues of these polypeptides, such homologues can be at least 90%, at least 95, 96, 97, 98 or 99% or more homologous to the amino acid sequences set forth below, as can be determined using BlastP software of the National Center of Biotechnology Information (NCBI) using default parameters. Finally, the present invention also encompasses fragments of the above described polypeptides and polypeptides having mutations, such as deletions, insertions or substitutions of one or more amino acids, either naturally occurring or man induced, either randomly or in a targeted fashion.

As mentioned hereinabove, biomolecular sequences of the present invention can be efficiently utilized as tissue or pathological markers and as putative drugs or drug targets for treating or preventing a disease.

Oligonucleotides designed for carrying out the methods of the present invention for any of the sequences provided herein (designed as described above) can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases.

The oligonucleotides of the present invention may comprise heterocyclic nucleosides consisting of purines and the pyrimidines bases, bonded in a 3′ to 5′ phosphodiester linkage.

Preferable oligonucleotides are those modified in any of backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which optionally may be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications which can optionally be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

It will be appreciated that peptides identified according to the teachings of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted by synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclization does not severely interfere with peptide characteristics, cyclic forms of the peptide can also be utilized.

The peptides of the present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Expression Systems

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence optionally may be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining elements, or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptides of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors optionally may be used that are non-viral, such as cationic lipids, polylysine, and dendrimers.

Recombinant Expression Vectors and Host Cells

Another aspect of the invention pertains to vectors, preferably expression vectors, containing a nucleic acid encoding a protein of the invention, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” optionally may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequences in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for production of variant proteins in prokaryotic or eukaryotic cells. For example, proteins of the invention can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or C terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89)—not accurate, pET11a-d have N terminal T7 tag.

One strategy to maximize recombinant protein expression in E. coli is to express the protein in a host bacterium with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another strategy to solve codon bias is by using BL21-codon plus bacterial strains (Invitrogen) or Rosetta bacterial strain (Novagen), these strains contain extra copies of rare E. coli tRNA genes.

In another embodiment, the expression vector encoding for the protein of the invention is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, polypeptides of the present invention can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for protein of the invention. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, protein of the invention can be produced in bacterial cells such as E. coli, insect cells, yeast, plant or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding protein of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, optionally may be used to produce (i.e., express) protein of the invention. Accordingly, the invention further provides methods for producing proteins of the invention using the host cells of the invention. In one embodiment, the method comprises culturing the host cell of the present invention (into which a recombinant expression vector encoding protein of the invention has been introduced) in a suitable medium such that the protein of the invention is produced. In another embodiment, the method further comprises isolating protein of the invention from the medium or the host cell.

For efficient production of the protein, it is preferable to place the nucleotide sequences encoding the protein of the invention under the control of expression control sequences optimized for expression in a desired host. For example, the sequences may include optimized transcriptional and/or translational regulatory sequences (such as altered Kozak sequences).

Protein Modifications

Fusion Proteins

According to some embodiments of the present invention, a fusion protein may be prepared from a protein of the invention by fusion with a portion of an immunoglobulin comprising a constant region of an immunoglobulin. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG Fc fragment that comprises CH2 and CH3 domains. Although any IgG subtype may optionally be used, the IgG1subtype is preferred. The Fc chain may optionally be a known or “wild type” Fc chain, or alternatively may be mutated. Non-limiting, illustrative, exemplary types of mutations are described in US Patent Application No. 20060034852, published on Feb. 16, 2006, hereby incorporated by reference as if fully set forth herein. The term “Fc chain” also optionally comprises any type of Fc fragment.

Several of the specific amino acid residues that are important for antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in aglycosylation for example and/or other desired changes to the Fc chain. At least some changes may optionally be made to block a function of Fc which is considered to be undesirable, such as an undesirable immune system effect, as described in greater detail below.

Non-limiting, illustrative examples of mutations to Fc which may be made to modulate the activity of the fusion protein include the following changes (given with regard to the Fc sequence nomenclature as given by Kabat, from Kabat E A et al: Sequences of Proteins of Immunological Interest. US Department of Health and Human Services, NIH, 1991): 220C->S; 233-238 ELLGGP->EAEGAP; 265D->A, preferably in combination with 434N->A; 297N->A (for example to block N-glycosylation); 318-322 EYKCK->AYACA; 330-331AP->SS; or a combination thereof (see for example M. Clark, “Chemical Immunol and Antibody Engineering”, pp 1-31 for a description of these mutations and their effect). The construct for the Fc chain which features the above changes optionally and preferably comprises a combination of the hinge region with the CH2 and CH3 domains.

The above mutations may optionally be implemented to enhance desired properties or alternatively to block non-desired properties. For example, aglycosylation of antibodies was shown to maintain the desired binding functionality while blocking depletion of T-cells or triggering cytokine release, which may optionally be undesired functions (see M. Clark, “Chemical Immunol and Antibody Engineering”, pp 1-31). Substitution of 331 proline for serine may block the ability to activate complement, which may optionally be considered an undesired function (see M. Clark, “Chemical Immunol and Antibody Engineering”, pp 1-31). Changing 330alanine to serine in combination with this change may also enhance the desired effect of blocking the ability to activate complement.

Residues 235 and 237 were shown to be involved in antibody-dependent cell-mediated cytotoxicity (ADCC), such that changing the block of residues from 233-238 as described may also block such activity if ADCC is considered to be an undesirable function.

Residue 220 is normally a cysteine for Fc from IgG1, which is the site at which the heavy chain forms a covalent linkage with the light chain. Optionally, this residue may be changed to a serine, to avoid any type of covalent linkage (see M. Clark, “Chemical Immunol and Antibody Engineering”, pp 1-31).

The above changes to residues 265 and 434 may optionally be implemented to reduce or block binding to the Fc receptor, which may optionally block undesired functionality of Fc related to its immune system functions (see “Binding site on Human IgG1for Fc Receptors”, Shields et al, Vol 276, pp 6591-6604, 2001).

The above changes are intended as illustrations only of optional changes and are not meant to be limiting in any way. Furthermore, the above explanation is provided for descriptive purposes only, without wishing to be bound by a single hypothesis.

Addition of Groups

If a protein according to the present invention is a linear molecule, it is possible to place various functional groups at various points on the linear molecule which are susceptible to or suitable for chemical modification. Functional groups can be added to the termini of linear forms of the protein of the invention. In some embodiments, the functional groups improve the activity of the protein with regard to one or more characteristics, including but not limited to, improvement in stability, penetration (through cellular membranes and/or tissue barriers), tissue localization, efficacy, decreased clearance, decreased toxicity, improved selectivity, improved resistance to expulsion by cellular pumps, and the like. For convenience sake and without wishing to be limiting, the free N-terminus of one of the sequences contained in the compositions of the invention will be termed as the N-terminus of the composition, and the free C-terminal of the sequence will be considered as the C-terminus of the composition. Either the C-terminus or the N-terminus of the sequences, or both, can be linked to a carboxylic acid functional groups or an amine functional group, respectively.

Non-limiting examples of suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can optionally and preferably be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include but are not limited to acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—, Adamantan, naphtalen, myristoleyl, toluen, biphenyl, cinnamoyl, nitrobenzoy, toluoyl, furoyl, benzoyl, cyclohexane, norbornane, or Z-caproic. In order to facilitate the N-acylation, one to four glycine residues can be present in the N-terminus of the molecule.

The carboxyl group at the C-terminus of the compound can be protected, for example, by the group including but not limited to an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are optionally independently an aliphatic, substituted aliphatic, benzyl, substituted benzyl, aryl or a substituted aryl group. In addition, taken together with the nitrogen atom, R₂ and R₃ can optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include but are not limited to —NH₂, —NHCH₃, —N(CH₃)₂, —NH(ethyl), —N(ethyl)₂, —N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Substitution by Peptidomimetic Moieties

A “peptidomimetic organic moiety” can optionally be substituted for amino acid residues in the composition of this invention both as conservative and as non-conservative substitutions. These moieties are also termed “non-natural amino acids” and may optionally replace amino acid residues, amino acids or act as spacer groups within the peptides in lieu of deleted amino acids. The peptidomimetic organic moieties optionally and preferably have steric, electronic or configurational properties similar to the replaced amino acid and such peptidomimetics are used to replace amino acids in the essential positions, and are considered conservative substitutions. However such similarities are not necessarily required. According to preferred embodiments of the present invention, one or more peptidomimetics are selected such that the composition at least substantially retains its physiological activity as compared to the native protein according to the present invention.

Peptidomimetics may optionally be used to inhibit degradation of the peptides by enzymatic or other degradative processes. The peptidomimetics can optionally and preferably be produced by organic synthetic techniques. Non-limiting examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-prop enidone-6-carb oxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable but exemplary peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable exemplary peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R,3S)-methyl-phenylalanine and (2R,3R)-methyl-phenylalanine (Kazmierski and Hruby, Tetrahedron Lett. (1991)).

Exemplary, illustrative but non-limiting non-natural amino acids include beta-amino acids (beta3 and beta2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids or diamino acids. They are available from a variety of suppliers, such as Sigma-Aldrich (USA) for example.

Chemical Modifications

In the present invention any part of a protein of the invention may optionally be chemically modified, i.e. changed by addition of functional groups. For example the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide according to the present invention, refers to a protein or peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference as if fully set forth herein. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

Altered Glycosylation

Proteins of the present invention, according to at least some embodiments, may optionally be modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to proteins of the invention is conveniently accomplished by altering the amino acid sequence of the protein such that it contains one or more of the above-described tripeptide sequences (for N-linked glycosylation sites). The alteration may also be made by the addition of, or substitution by, one or more serine or threonine residues in the sequence of the original protein (for O-linked glycosylation sites). The protein's amino acid sequence may also be altered by introducing changes at the DNA level.

Another means of increasing the number of carbohydrate moieties on proteins is by chemical or enzymatic coupling of glycosides to the amino acid residues of the protein. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on proteins of the invention may be accomplished chemically or enzymatically. Chemical deglycosylation requires exposure of the protein to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on proteins can be achieved by the use of a variety of endo- and exo-glycosidases as described by Thotakura et al., Meth. Enzymol., 138: 350 (1987).

Methods of Treatment

As mentioned hereinabove the KIAA0746, CD20, CD55 proteins or KIAA0746, CD20, CD55 proteins and polypeptides of the present invention or nucleic acid sequence or fragments thereof, preferably the ectodomain or secreted forms of KIAA0746, CD20, CD55 proteins, as well as drugs which specifically bind to the KIAA0746, CD20, CD55 proteins and/or splice variants, and/or drugs which agonize or antagonize the binding of other moieties to the KIAA0746, CD20, CD55 proteins and/or splice variants, and/or drugs which modulate (agonize or antagonize) at least one KIAA0746, CD20, CD55 related biological activity (such drugs include by way of example antibodies, small molecules, peptides, ribozymes, antisense molecules, siRNA's and the like), optionally may be used to treat cancer.

The KIAA0746, CD20, CD55 proteins or KIAA0746, CD20, CD55 proteins and polypeptides according to at least some embodiments of the present invention or nucleic acid sequence or fragments thereof especially the ectodomain or secreted forms of KIAA0746, CD20, CD55 proteins, as well as drugs which specifically bind to the KIAA0746, CD20, CD55 proteins and/or splice variants, and/or drugs which agonize or antagonize the binding of other moieties to the KIAA0746, CD20, CD55 proteins and/or splice variants, and/or drugs which modulate (agonize or antagonize) at least one KIAA0746, CD20, CD55 related biological activity (such drugs include by way of example antibodies, small molecules, peptides, ribozymes, antisense molecules, siRNA's and the like), can be further used to treat non-malignant disorders such as immune related conditions and/or for blocking or promoting immune costimulation mediated by the KIAA0746, CD20, CD55 polypeptide.

CD55 proteins and polypeptides of the present invention or nucleic acid sequence or fragments thereof especially the ectodomain or secreted forms CD55 proteins, as well as drugs which specifically bind to the CD55 proteins and/or splice variants, and/or drugs which agonize or antagonize the binding of other moieties to the CD55 proteins and/or splice variants, and/or drugs which modulate (agonize or antagonize) at least one CD55 related biological activity (such drugs include by way of example antibodies, small molecules, peptides, ribozymes, antisense molecules, siRNA's and the like), can be further used to treat ischemia-reperfusion injury, inflammation of the respiratory tract disorder, transplant rejection, GVHD and rejection in xenotransplantation.

The KIAA0746 or CD20, proteins or polypeptides of the present invention or nucleic acid sequence or fragments thereof especially the ectodomain or secreted forms of KIAA0746 or CD20 proteins, as well as drugs which specifically bind to the KIAA0746 or CD20 proteins and/or splice variants, and/or drugs which agonize or antagonize the binding of other moieties to the KIAA0746 or CD20 proteins and/or splice variants, and/or drugs which modulate (agonize or antagonize) at least one KIAA0746 or CD20 related biological activity (such drugs include by way of example antibodies, small molecules, peptides, ribozymes, antisense molecules, siRNA's and the like), can be further used to treat lymphoproliferative disorder.

The subject according to the present invention is optionally and preferably a mammal, preferably a human which is diagnosed with one of the disease, disorder or conditions described hereinabove, or alternatively is predisposed to at least one of the diseases, disorders or conditions described hereinabove.

As used herein the term “treating” refers to preventing, curing, reversing, attenuating, alleviating, minimizing, suppressing or halting the deleterious effects of the above-described diseases, disorders or conditions.

Treating, according to the present invention, can be effected by specifically upregulating the expression of at least one of the polypeptides of the present invention in the subject.

Optionally, upregulation may be effected by administering to the subject at least one of the polypeptides of the present invention (e.g., recombinant or synthetic) or an active portion thereof, as described herein. However, since the bioavailability of large polypeptides may potentially be relatively small due to high degradation rate and low penetration rate, administration of polypeptides is preferably confined to small peptide fragments (e.g., about 100 amino acids). The polypeptide or peptide may optionally be administered in as part of a pharmaceutical composition, described in more detail below.

It will be appreciated that treatment of the above-described diseases according to the present invention may be combined with other treatment methods known in the art (i.e., combination therapy). Thus, treatment of malignancies using the agents of the present invention may be combined with, for example, radiation therapy, antibody therapy and/or chemotherapy.

In another specific example, the treatment of malignancies using CD20-related agents of the present invention may be combined with CVP chemotherapy (cyclophosphamide, vincristine and prednisolone), particularly when the malignancy is previously untreated follicular, CD20-positive, B-cell NHL. In another specific example, the treatment of malignancies using CD20-related agents of the present invention may be combined with CHOP (cyclophosphamide, doxorubicin, vincristine and prednisolone) or other anthracycline-based chemotherapy regimens, particularly when the malignancy is selected from previously untreated diffuse large B-cell, CD20-positive NHL, or previously untreated diffuse NHL mantle cell lymphoma.

Alternatively or additionally, an upregulating method may optionally be effected by specifically upregulating the amount (optionally expression) in the subject of at least one of the polypeptides of the present invention or active portions thereof.

As is mentioned hereinabove and in the Examples section which follows, the biomolecular sequences of this aspect of the present invention may be used as valuable therapeutic tools in the treatment of diseases, disorders or conditions in which altered activity or expression of the wild-type gene product (known protein) is known to contribute to disease, disorder or condition onset or progression. For example, in case a disease is caused by overexpression of a membrane bound-receptor, a soluble variant thereof may be used as an antagonist which competes with the receptor for binding the ligand, to thereby terminate signaling from the receptor.

Anti-KIAA0746, Anti-CD20, Anti-CD55 Antibodies

The antibodies of the invention including those having the particular germline sequences, homologous antibodies, antibodies with conservative modifications, engineered and modified antibodies are characterized by particular functional features or properties of the antibodies. For example, the antibodies bind specifically to human KIAA0746, CD20 or CD55. Preferably, an antibody of the invention binds to corresponding KIAA0746, CD20 or CD55 with high affinity, for example with a KD of 10−8 M or less or 10−9 M or less or even 10−10 M or less. The Anti-KIAA0746, Anti-CD20 or Anti-CD55 antibodies of the invention preferably exhibit one or more of the following characteristics:

(i) binds to corresponding human KIAA0746, CD20 or CD55 with a KD of 5.×10−8 M or less;

(ii) binds to KIAA0746, CD20 or CD55 antigen expressed by cancer cells, but does not substantially bind to normal cells. In addition, preferably these antibodies and conjugates thereof will be effective in eliciting selective killing of such cancer cells and for modulating immune responses involved in autoimmunity and cancer;

(iii) binds to KIAA0746 or CD20 antigen expressed by immune related condition cells, and/or by lymphoproliferative disorder cells, but does not substantially bind to normal cells;

(iv) binds to CD55 antigen expressed by inflammation of the respiratory tract disorder cells or ischemia-reperfusion disorder cells, but does not substantially bind to normal cells.

More preferably, the antibody binds to corresponding human KIAA0746, CD20 or CD55 antigen with a KD of 3×10−8 M or less, or with a KD of 1×10−9 M or less, or with a KD of 0.1.×10−9 M or less, or with a KD Of 0.05.×10−9 M or less or with a KD of between 1×10−9 and 1×10−11 M.

Standard assays to evaluate the binding ability of the antibodies toward KIAA0746, CD20 or CD55 are known in the art, including for example, ELISAs, Western blots and RIAs. Suitable assays are described in detail in the Examples. The binding kinetics (e.g., binding affinity) of the antibodies also can be assessed by standard assays known in the art, such as by Biacore analysis.

Upon production of Anti-KIAA0746, Anti-CD20, Anti-CD55 antibody sequences from antibodies can bind to KIAA0746, CD20 or CD55 the VH and VL sequences can be “mixed and matched” to create other anti-KIAA0746, CD20 or CD55 binding molecules of the invention. KIAA0746, CD20 or CD55 binding of such “mixed and matched” antibodies can be tested using the binding assays described above. e.g., ELISAs). Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence. For example, the VH and VL sequences of homologous antibodies are particularly amenable for mixing and matching.

Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

As used herein, a human antibody comprises heavy or light chain variable regions that is “the product of”0 or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95, 96, 97, 98 or 99%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Homologous Antibodies

In yet another embodiment, an antibody of the invention comprises heavy and light chain variable regions comprising amino acid sequences that are homologous to isolated Anti-KIAA0746, Anti-CD20, Anti-CD55 amino acid sequences of preferred Anti-KIAA0746, Anti-CD20, Anti-CD55 antibodies, respectively, wherein the antibodies retain the desired functional properties of the parent Anti-KIAA0746, Anti-CD20, Anti-CD55 antibodies.

As used herein, the percent homology between two amino acid sequences is equivalent to the percent identity between the two sequences. The percent identity between the two sequences is a function of the number of identical positions shared by the sequences (i.e., % homology=# of identical positions/total # of positions X 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) optionally may be used.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on preferred Anti-KIAA0746, Anti-CD20, Anti-CD55 antibodies isolated and produced using methods herein, or conservative modifications thereof, and wherein the antibodies retain the desired functional properties of the Anti-KIAA0746, Anti-CD20, Anti-CD55 antibodies of the invention, respectively.

In various embodiments, the Anti-KIAA0746, Anti-CD20, Anti-CD55 antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) through (j) above) using the functional assays described herein.

Antibodies that Bind to the Same Epitope as Anti-KIAA0746, Anti-CD20, Anti-CD55 Antibodies of the Invention

In another embodiment, the invention provides antibodies that bind to preferred epitopes on human KIAA0746, CD20, CD55 which possess desired functional properties. Other antibodies with desired epitope specificity may be selected and will have the ability to cross-compete for binding to KIAA0746, CD20 or CD55 antigen with the desired antibodies.

Engineered and Modified Antibodies

An antibody of the invention further can be prepared using an antibody having one or more of the VH and/or VL sequences derived from an Anti-KIAA0746, Anti-CD20, Anti-CD55 antibody starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant regions, for example to alter the effector functions of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Suitable framework sequences can be obtained from public DNA databases or published references that include germline antibody gene sequences. For example, germline DNA sequences for human heavy and light chain variable region genes can be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference.

Another type of variable region modification is to mutate amino acid residues within the VH and/or VL CDR 1, CDR2 and/or CDR3 regions to thereby improve one or more binding properties (e.g., affinity) of the antibody of interest. Site-directed mutagenesis or PCR-mediated mutagenesis can be performed to introduce the mutations and the effect on antibody binding, or other functional property of interest, can be evaluated in appropriate in vitro or in vivo assays. Preferably conservative modifications (as discussed above) are introduced. The mutations may be amino acid substitutions, additions or deletions, but are preferably substitutions. Moreover, typically no more than one, two, three, four or five residues within a CDR region are altered.

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcy receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1for Fc grammar, Fc gamma RII, Fc gammaRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcyRIII. Additionally, the following combination mutants are shown to improve Fcgamma.RIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and optionally may be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8.−/− cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

Methods of Engineering Antibodies

As discussed above, the Anti-KIAA0746, Anti-CD20, Anti-CD55 antibodies having VH and VK sequences disclosed herein optionally may be used to create new Anti-KIAA0746, Anti-CD20, Anti-CD55 antibodies, respectively, by modifying the VH and/or VL sequences, or the constant regions attached thereto. Thus, in another aspect of the invention, the structural features of an Anti-KIAA0746, Anti-CD20, Anti-CD55 antibody of the invention, are used to create structurally related Anti-KIAA0746, Anti-CD20, Anti-CD55 antibodies that retain at least one functional property of the antibodies of the invention, such as binding to human KIAA0746, CD20 or CD55, respectively. For example, one or more CDR regions of one KIAA0746, CD20 or CD55 antibody or mutations thereof, can be combined recombinantly with known framework regions and/or other CDRs to create additional, recombinantly-engineered, Anti-KIAA0746, Anti-CD20, Anti-CD55 antibodies of the invention, as discussed above. Other types of modifications include those described in the previous section. The starting material for the engineering method is one or more of the VH and/or VK sequences provided herein, or one or more CDR regions thereof. To create the engineered antibody, it is not necessary to actually prepare (i.e., express as a protein) an antibody having one or more of the VH and/or VK sequences provided herein, or one or more CDR regions thereof. Rather, the information contained in the sequences is used as the starting material to create a “second generation” sequence derived from the original sequences and then the “second generation” sequence is prepared and expressed as a protein.

Standard molecular biology techniques optionally may be used to prepare and express altered antibody sequence.

Preferably, the antibody encoded by the altered antibody sequences is one that retains one, some or all of the functional properties of the Anti-KIAA0746, Anti-CD20, Anti-CD55 antibodies, respectively, produced by methods and with sequences provided herein, which functional properties include binding to KIAA0746, CD20 or CD55 antigen with a specific KD level or less and/or selectively binding to desired target cells such as cancer cells, that express KIAA0746, CD20 or CD55 antigen.

The functional properties of the altered antibodies can be assessed using standard assays available in the art and/or described herein.

In certain embodiments of the methods of engineering antibodies of the invention, mutations can be introduced randomly or selectively along all or part of an Anti-KIAA0746, Anti-CD20, Anti-CD55 antibody coding sequence and the resulting modified Anti-KIAA0746, Anti-CD20, Anti-CD55 antibodies can be screened for binding activity and/or other desired functional properties.

Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker.

The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production Of Anti-KIAA0746, Anti-CD20, Anti-CD55 Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256:495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against KIAA0746, CD20 or CD55 can be generated using transgenic or transchromosomic mice carrying parts of the human immune system rather than the mouse system. These transgenic and transchromosomic mice include mice referred to herein as the HuMAb Mouse® and KM Mouse® respectively, and are collectively referred to herein as “human Ig mice.” The HuMAb Mouse™. (Medarex. Inc.) contains human immunoglobulin gene miniloci that encode unrearranged human heavy (.mu. and .gamma.) and .kappa. light chain immunoglobulin sequences, together with targeted mutations that inactivate the endogenous.mu. and .kappa. chain loci (see e.g., Lonberg, et al. (1994) Nature 368(6474): 856-859). Accordingly, the mice exhibit reduced expression of mouse IgM or .kappa., and in response to immunization, the introduced human heavy and light chain transgenes undergo class switching and somatic mutation to generate high affinity human IgGkappa. monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann N.Y. Acad. Sci. 764:536-546). The preparation and use of the HuMab Mouse®, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice®”, are described in detail in PCT Publication WO 02/43478 to Ishida et al.

Still further, alternative transgenic animal systems expressing human immunoglobulin genes are available in the art and optionally may be used to raise anti-KIAA0746, CD20 or CD55 antibodies of the invention. For example, an alternative transgenic system referred to as the Xenomouse (Abgenix, Inc.) optionally may be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and optionally may be used to raise Anti-KIAA0746, Anti-CD20, Anti-CD55 antibodies of the invention. For example, mice carrying both a human heavy chain transchromosome and a human light chain transchromosome, referred to as “TC mice” optionally may be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and optionally may be used to raise Anti-KIAA0746, Anti-CD20, Anti-CD55 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Immunization of Human Ig Mice

When human Ig mice are used to raise human antibodies of the invention, such mice can be immunized with a purified or enriched preparation of KIAA0746, CD20 or CD55 antigen and/or recombinant KIAA0746, CD20 or CD55, or an KIAA0746, CD20 or CD55 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50.mu.g) of KIAA0746, CD20 or CD55 antigen optionally may be used to immunize the human Ig mice intraperitoneally.

Prior experience with various antigens by others has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-KIAA0746, anti-CD20, anti-CD55 human immunoglobulin optionally may be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo 12). Alternatively or additionally, the KM Mouse® strain optionally may be used.

Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Cells are plated at approximately 2×10−5 in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-Sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein optionally may be used to create full-length antibody genes of any antibody isotype by inserting them into expression vectors already encoding heavy chain constant and light chain constant regions of the desired isotype such that the VH segment is operatively linked to the CH segments within the vector and the VK segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or beta-globin promoter. Still further, regulatory elements composed of sequences from different sources, such as the SR alpha promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vectors encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Characterization of Antibody Binding to Antigen

Antibodies of the invention can be tested for binding to KIAA0746, CD20 or CD55 by, for example, standard ELISA. Briefly, microtiter plates are coated with purified KIAA0746, CD20 or CD55 at 0.25.mu.g/ml in PBS, and then blocked with 5% bovine serum albumin in PBS. Dilutions of antibody (e.g., dilutions of plasma from KIAA0746, CD20 or CD55-immunized mice) are added to each well and incubated for 1-2 hours at 37 degrees C. The plates are washed with PBS/Tween and then incubated with secondary reagent (e.g., for human antibodies, a goat-anti-human IgG Fc-specific polyclonal reagent) conjugated to alkaline phosphatase for 1 hour at 37 degrees C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with KIAA0746, CD20 or CD55immunogen. Hybridomas that bind with high avidity to KIAA0746, CD20 or CD55 are subcloned and further characterized. One clone from each hybridoma, which retains the reactivity of the parent cells (by ELISA), can be chosen for making a 5-10 vial cell bank stored at −140 degrees C., and for antibody purification.

To purify anti-KIAA0746, anti-CD20 or anti-CD55 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

To determine if the selected anti-KIAA0746, anti-CD20 or anti-CD55 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using KIAA0746, CD20 or CD55 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1.mu.g/ml of anti-human immunoglobulin overnight at 4 degrees C. After blocking with 1% BSA, the plates are reacted with 1 mug/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-KIAA0746, anti-CD20 or anti-CD55 human IgGs can be further tested for reactivity with KIAA0746, CD20 or CD55 antigen, respectively, by Western blotting. Briefly, KIAA0746, CD20 or CD55 antigen can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Conjugates or Immunoconjugates

The present invention, according to at least some embodiments, encompasses conjugates for use in immune therapy comprising the KIAA0746, CD20 or CD55 antigen and soluble portions thereof including the ectodomain or portions or variants thereof. For example the invention encompasses conjugates wherein the ECD of the KIAA0746, CD20 or CD55 antigen is attached to an immunoglobulin or fragment thereof. The invention contemplates the use thereof for promoting or inhibiting KIAA0746, CD20 or CD55 antigen activities such as immune costimulation and the use thereof in treating transplant, autoimmune, and cancer indications described herein.

In another aspect, the present invention features immunoconjugates comprising an anti-KIAA0746, anti-CD20 or anti-CD55 antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg®; Wyeth).

Cytotoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine 131, indium 111, yttrium 90 and lutetium 177. Method for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods optionally may be used to prepare radioimmunoconjugates using the antibodies of the invention.

The antibody conjugates of the invention optionally may be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

In another aspect, the present invention features bispecific molecules comprising an anti-KIAA0746, anti-CD20 or anti-CD55 antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for KIAA0746, CD20 or CD55 and a second binding specificity for a second target epitope. In a particular embodiment of the invention, the second target epitope is an Fc receptor, e.g., human Fc gamma RI (CD64) or a human Fc alpha receptor (CD89). Therefore, the invention includes bispecific molecules capable of binding both to Fc gamma. R, Fc alpha R or Fc epsilon R expressing effector cells (e.g., monocytes, macrophages or polymorphonuclear cells (PMNs)), and to target cells expressing KIAA0746, CD20 or CD55, respectively. These bispecific molecules target KIAA0746, CD20 or CD55expressing cells to effector cell and trigger Fc receptor-mediated effector cell activities, such as phagocytosis of an KIAA0746, CD20 or CD55 expressing cells, antibody dependent cell-mediated cytotoxicity (ADCC), cytokine release, or generation of superoxide anion.

In an embodiment of the invention in which the bispecific molecule is multispecific, the molecule can further include a third binding specificity, in addition to an anti-Fc binding specificity and an anti-6f binding specificity. In one embodiment, the third binding specificity is an anti-enhancement factor (EF) portion, e.g., a molecule which binds to a surface protein involved in cytotoxic activity and thereby increases the immune response against the target cell.

The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′).sub.2, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which are expressly incorporated by reference as if fully incorporated herein, as for all references provided herein.

In one embodiment, the binding specificity for an Fcy receptor is provided by a monoclonal antibody, the binding of which is not blocked by human immunoglobulin G (IgG). As used herein, the term “IgG receptor” refers to any of the eight.gamma.-chain genes located on chromosome 1. These genes encode a total of twelve transmembrane or soluble receptor isoforms which are grouped into three Fc gamma receptor classes: Fc gamma R1 (CD64), Fc gamma RII (CD32), and Fc gamma.RIII (CD 16). In one preferred embodiment, the Fc gamma receptor a human high affinity Fc gamma RI. The human Fc gammaRI is a 72 kDa molecule, which shows high affinity for monomeric IgG (10 8-10−9 M.−1).

The production and characterization of certain preferred anti-Fc gamma monoclonal antibodies are described by Fanger et al. in PCT Publication WO 88/00052 and in U.S. Pat. No. 4,954,617, the teachings of which are fully incorporated by reference herein. These antibodies bind to an epitope of Fc.gamma.R1, FcyRII or FcyRIII at a site which is distinct from the Fc.gamma. binding site of the receptor and, thus, their binding is not blocked substantially by physiological levels of IgG. Specific anti-Fc.gamma.RI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcy receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line is deposited at the American Type Culture Collection under the designation HAO22CLI and has the accession no. CRL 11177.

In still other preferred embodiments, the binding specificity for an Fc receptor is provided by an antibody that binds to a human IgA receptor, e.g., an Fc-alpha receptor (Fc alpha RI (CD89)), the binding of which is preferably not blocked by human immunoglobulin A (IgA). The term “IgA receptor” is intended to include the gene product of one alpha-gene (Fc alpha.RI) located on chromosome 19. This gene is known to encode several alternatively spliced transmembrane isoforms of 55 to 10 kDa

Fc alpha RI (CD89) is constitutively expressed on monocytes/macrophages, eosinophilic and neutrophilic granulocytes, but not on non-effector cell populations. Fc alpha RI has medium affinity (Approximately 5×10−7 M−1) for both IgA1 and IgA2, which is increased upon exposure to cytokines such as G-CSF or GM-CSF (Morton, H. C. et al. (1996) Critical Reviews in Immunology 16:423-440). Four FcaRI-specific monoclonal antibodies, identified as A3, A59, A62 and A77, which bind Fc alpha RI outside the IgA ligand binding domain, have been described (Monteiro, R. C. et al. (1992) J. Immunol. 148:1764).

Fc alpha RI RI and Fc gamma RI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of the invention are murine, chimeric and humanized monoclonal antibodies.

The bispecific molecules of the present invention can be prepared by conjugating the constituent binding specificities, e.g., the anti-FcR and anti-KIAA0746, anti-CD20 or anti-CD55 binding specificities, using methods known in the art. For example, each binding specificity of the bispecific molecule can be generated separately and then conjugated to one another. When the binding specificities are proteins or peptides, a variety of coupling or cross-linking agents optionally may be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyld-ithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAbXmAb, mAbXFab, FabXF(ab′)2 or ligandXFab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma. counter or a scintillation counter or by autoradiography.

Pharmaceutical Compositions

In another aspect, the present invention provides a composition, e.g., a pharmaceutical composition, containing one or a combination of monoclonal antibodies, or antigen-binding portions thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

As discussed supra, KIAA0746, CD20 or CD55 as provided according to some embodiments of the present invention may optionally further other molecules such as small organic molecules, peptides, ribozymes, carbohydrates, glycoprotein, siRNAs, antisense RNAs and the like which specifically bind and/or modulate (enhance or inhibit) an activity elicited by the KIAA0746, CD20 or CD55 antigen, respectively. These molecules may be identified by known screening methods such as binding assays. Typically these assays will be high throughput and will screen a large library of synthesized or native compounds in order to identify putative drug candidates that bind and/or modulate KIAA0746, CD20 or CD55 related activities.

Specifically, the invention embraces the development of drugs containing the ectodomain of the KIAA0746, CD20 or CD55 antigen or a fragment or variant thereof or a corresponding nucleic acid sequence encoding. These conjugates may contain a targeting or other moiety such as an immunoglobulin domain. These conjugates may be expressed in known vector systems or cells or vectors containing the corresponding nucleic acid sequences may be used for cancer treatment and in immune therapy such as in the treatment of autoimmunity, transplant rejection, GVHD, cancer, and other immune disorders or conditions, as well as lymphoproliferative disorders, inflammation of the respiratory tract disorders, and/or ischemia-reperfusion injury related disorders.

Thus, the present invention features a pharmaceutical composition comprising a therapeutically effective amount of a therapeutic agent according to the present invention. According to the present invention the therapeutic agent could be any one of KIAA0746, CD20 or CD55 ectodomain, or a fragment or variant thereof, or a corresponding nucleic acid sequence encoding.

The pharmaceutical composition according to the present invention is further preferably used for the treatment of cancer including by way of example hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

The pharmaceutical composition according to the present invention is further used for the treatment of cancer, selected from colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

The pharmaceutical composition according to the present invention is further used for the treatment of cancer, selected from a hematological malignancy, preferably selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

The pharmaceutical composition according to the present invention is further used for the treatment of immune related conditions or disorders, wherein the immune related conditions or disorders are inflammatory and/or autoimmune diseasesand other immune related conditions such as transplant rejection, transplant rejection following allogenic transplantation or xenotransplantation, and graft versus host disease.

The pharmaceutical composition according to the present invention is further used for the treatment of immune related condition selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy, systemic lupus erythematosus (SLE), lupus nephtirits, inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and/or disease states in which complement activation and deposition is involved in pathogenesis.

The pharmaceutical composition according to the present invention is further used for the treatment of ischemia-reperfusion injury.

The pharmaceutical composition according to the present invention is further used for the treatment of inflammation of the respiratory tract disorder.

The pharmaceutical composition according to the present invention is further used for the treatment of lymphoproliferative disorder.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder (optionally as described herein non-mammals may be so treated, additionally or alternatively). “Mammal” for purposes of treatment refers to any animal classified as a mammal, including humans, domestic and farm animals, and zoo, sports, or pet animals, such as dogs, horses, cats, cows, etc. Preferably, the mammal is human.

The term “therapeutically effective amount” refers to an amount of agent according to the present invention that is effective to treat a disease or disorder in a mammal.

The therapeutic agents of the present invention can be provided to the subject alone, or as part of a pharmaceutical composition where they are mixed with a pharmaceutically acceptable carrier.

Pharmaceutical compositions of the invention also can be administered in combination therapy, i.e., combined with other agents. For example, the combination therapy can include an anti-KIAA0746, anti-CD20 or anti-CD55, or KIAA0746, CD20 or CD55 modulating agent according to the present invention such as a soluble polypeptide conjugate containing the ectodomain of the KIAA0746, CD20 or CD55 antigen or a small molecule such as a peptide, ribozyme, siRNA, or other drug that binds KIAA0746, CD20 or CD55 combined with at least one other therapeutic or immune modulatory agent. Examples of therapeutic agents that optionally may be used in combination therapy are described in greater detail below in the section on uses of the antibodies of the invention. In one specific example, for the treatment of malignancy, particularly wherein the malignancy is previously untreated follicular, CD20-positive, B-cell NHL, the combination therapy can include an anti-CD20, or CD20 modulating agent according to the present invention such as a soluble polypeptide conjugate containing the ectodomain of the CD20 antigen or a small molecule such as a peptide, ribozyme, siRNA, or other drug that binds CD20, combined with CVP chemotherapy (cyclophosphamide, vincristine and prednisolone).

In another specific example, for the treatment of malignancy, particularly wherein the malignancy is selected from previously untreated diffuse large B-cell, CD20-positive NHL, or previously untreated diffuse NHL mantle cell lymphoma, the combination therapy can include an anti-CD20, or CD20 modulating agent according to the present invention such as a soluble polypeptide conjugate containing the ectodomain of the CD20 antigen or a small molecule such as a peptide, ribozyme, siRNA, or other drug that binds CD20, combined with CHOP (cyclophosphamide, doxorubicin, vincristine and prednisolone) or other anthracycline-based chemotherapy regimens.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound. The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight, although optionally dosages may be in the microgram or nanogram, or even picogram, ranges for example. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-KIAA0746, anti-CD20 or anti-CD55 antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 micro-gram/ml and in some methods about 25-300 micro gram/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-KIAA0746, anti-CD20 or anti-CD55 antibody of the invention preferably results in a decrease in severity of disease symptoms, an increase in frequency and duration of disease symptom-free periods, an increase in lifepan, disease remission, or a prevention of impairment or disability due to the disease affliction. For example, for the treatment of KIAA0746 positive tumors, e.g., prostate tumors, pancreas tumors, ovary tumors, melanoma, lung tumors, liver tumors, kidney tumors, colon tumors, head and neck tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. For another example, for the treatment of CD20 positive tumors, e.g., hematological malignancies, primarily B-cell derived, such as acute lymphocytic leukemia, chronic lymphocytic leukemia, acute myelogenous leukemia, chronic myelogenous leukemia, multiple myeloma, and B-cell lymphoma, selected from the group consisting of, but not limited to non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, a “therapeutically effective dosage” preferably inhibits cell growth or tumor growth by at least about 20%, more preferably by at least about 40%, even more preferably by at least about 60%, and still more preferably by at least about 80% relative to untreated subjects. For another example, for the treatment of CD55 positive tumors, e.g., prostate tumors, pancreas tumors, ovary tumors, lung tumors, liver tumors, gastric tumors, colon tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody or other KIAA0746, CD20 or CD55 drug or molecule and their conjugates and combinations thereof that modulates a KIAA0746, CD20 or CD55 antigen activity according to the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers optionally may be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the antibodies or other KIAA0746, CD20 or CD55 related drugs of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Diagnostic uses of KIAA0746, CD20 or CD55 Antigen and Corresponding Polynucleotides

According to some embodiments, the sample taken from a subject (patient) to perform the diagnostic assay according to the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cells or tissues, wherein the cell or tissue can be obtained from an organ selected from, but not limited tolung, kidney, pancreas, ovary, prostate, liver, skin, bone marrow, lymph node, breast, and/or blood tissue; stool or a tissue sample, or any combination thereof. In some embodiments, the term encompasses samples of in vivo cell culture constituents. Prior to be subjected to the diagnostic assay, the sample can optionally be diluted with a suitable eluant.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the term “polypeptide” is to be understood to refer to a molecule comprising from at least 2 to several thousand or more amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to N terminus modification, C terminus modification, peptide bond modification, backbone modifications, residue modification, or others. Inclusion of such peptides within the polypeptides of this invention may produce a polypeptide sharing identity with the polypeptides described herein, for example, those provided in the sequence listing.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It will be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the phrase “qualitative” when in reference to differences in expression levels of a polynucleotide or polypeptide as described herein, refers to the presence versus absence of expression, or in some embodiments, the temporal regulation of expression, or in some embodiments, the timing of expression, or in some embodiments, any post-translational modifications to the expressed molecule, and others, as will be appreciated by one skilled in the art. In some embodiments, the phrase “quantitative” when in reference to differences in expression levels of a polynucleotide or polypeptide as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease. It will be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same marker in a similar sample obtained from a healthy individual (examples of biological samples are described herein).

Numerous well known tissue or fluid collection methods can be utilized to collect the biological sample from the subject in order to determine the level of DNA, RNA and/or polypeptide of the marker of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the marker can be determined and a diagnosis can thus be made.

Determining the level of the same marker in normal tissues of the same origin is preferably effected along-side to detect an elevated expression and/or amplification and/or a decreased expression, of the marker as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that optionally may be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that optionally may be used to determine specific immunoreactivity). Typically a specific or selective reaction will be at least twice background signal or noise and more typically more than 10 to 100 times background.

In another embodiment, this invention provides a method for detecting the polypeptides of this invention in a biological sample, comprising: contacting a biological sample with an antibody specifically recognizing a polypeptide according to the present invention and detecting said interaction; wherein the presence of an interaction correlates with the presence of a polypeptide in the biological sample.

In some embodiments of the present invention, the polypeptides described herein are non-limiting examples of markers for diagnosing a disease and/or an indicative condition. Each marker of the present invention optionally may be used alone or in combination, for various uses, including but not limited to, prognosis, prediction, screening, early diagnosis, determination of progression, therapy selection and treatment monitoring of a disease and/or an indicative condition.

In a related embodiment the detected diseases will include cancer, selected from the group including but not limited to hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic.

In a related embodiment the detected diseases will include cancer, selected from the group consisting of colorectal cancer, lung cancer, prostate cancer, pancreas cancer, ovarian cancer, gastric cancer, liver cancer, melanoma, kidney cancer, head and neck cancer, and wherein the cancer is non-metastatic, invasive or metastatic.

In a related embodiment the detected diseases will include cancer, selected from the group consisting of hematological malignancy, selected from the group consisting of acute lymphocytic leukemia, chronic lymphocytic leukemia, acute myelogenous leukemia, chronic myelogenous leukemia, multiple myeloma, and B-cell lymphoma, selected from the group consisting of non-Hodgkin's lymphoma (NHL), low grade/follicular non-Hodgkin's lymphoma (NHL), small lymphocytic (SL) NHL, small cell NHL, grade I small cell follicular NHL, grade II mixed small and large cell follicular NHL, grade III large cell follicular NHL, large cell NHL, Diffuse Large B-Cell NHL, intermediate grade diffuse NHL, chronic lymphocytic leukemia (CLL), high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinernia, and wherein the hematological malignancy non-metastatic, invasive or metastatic.

In another related embodiment the detected diseases will include immune related conditions or disorders, wherein the immune related conditions or disorders are inflammatory and autoimmune diseases, selected from the group including but not limited to multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease.

In a related embodiment the detected diseases will include immune related conditions or disorders, selected from the group consisting of rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders, pemphigus, pemphigoid, atopic eczema, type 1 diabetes mellitus, Sjogren's syndrome, Devic's disease and systemic lupus erythematosus, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, Graves' Disease and Graves' Ophthalmopathy, systemic lupus erythematosus (SLE), lupus nephtirits, inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

In a related embodiment the detected diseases will include ischemia-reperfusion injury, selected from the group including but not limited to ischemia-reperfusion injury related disorder associated with ischemic and post-ischemic events in organs and tissues, and is selected from the group consisting of thrombotic stroke, myocardial infarction, angina pectoris, embolic vascular occlusions, peripheral vascular insufficiency, splanchnic artery occlusion, arterial occlusion by thrombi or embolisms, arterial occlusion by non-occlusive processes such as following low mesenteric flow or sepsis, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, or disorders resulting from procedures such as angiography, cardiopulmonary and cerebral resuscitation, cardiac surgery, organ surgery, organ transplantation, systemic and intragraft inflammatory responses that occur after cold ischemia-reperfusion in the setting of organ transplantation.

In a related embodiment the detected diseases will include inflammation of the respiratory tract disorder, selected from the group including but not limited to chronic obstructive pulmonary disease (COPD), acute respiratory distress syndrome (ARDS), severe acute respiratory syndrome (SARS), asthma, allergy, bronchial disease, pulmonary emphysema, pulmonary inflammation, environmental airway disease, airway hyper-responsiveness, chronic bronchitis, acute lung injury, bronchial disease, lung diseases, and cystic fibrosis.

In a related embodiment the detected diseases will include lymphoproliferative disorder, selected from the group including but not limited to EBV-related lymphoproliferative disorders, posttransplant lymphoproliferative disorders, Waldenstrom's macroglobulinemia, mixed cryoglobulinemia, immune-complex mediated vasculitis, cryoglobulinemic vasculitis, immunocytoma, monoclonal gammopathy of undetermined significance (MGUS).

Each polypeptide/polynucleotide of the present invention optionally may be used alone or in combination, for various uses, including but not limited to, prognosis, prediction, screening, early diagnosis, determination of progression, therapy selection and treatment monitoring of disease and/or an indicative condition, as detailed above.

Such a combination may optionally comprise any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker.

According to further embodiments of the present invention markers of the present invention might optionally be used alone or in combination one or more other compounds described herein, and/or in combination with known markers for lung cancer, including but not limited to CEA, CA15-3, Beta-2-microglobulin, CA19-9, TPA, and/or in combination with the known proteins for the variant marker as described herein.

According to further embodiments of the present invention markers of the present invention might optionally be used alone or in combination with one or more other compounds described herein, and/or in combination known markers for ovarian cancer, including but not limited to CEA, CA125 (Mucin 16), CA72-4TAG, CA-50, CA 54-61, CA-195 and CA 19-9 in combination with CA-125, and/or in combination with the known proteins for the variant marker as described herein.

According to further embodiments of the present invention markers of the present invention might optionally be used alone or in combination with one or more other compounds described herein, and/or in combination with known markers for breast cancer, including but not limited to Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), and/or in combination with the known proteins for the variant marker as described herein.

According to further embodiments of the present invention markers of the present invention might optionally be used alone or in combination with one or more other compounds described herein, and/or in combination with known markers for renal cancer, including but not limited to urinary protein, creatinine or creatinine clearance, and/or markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase and/or in combination with the known proteins for the variant marker as described herein.

According to further embodiments of the present invention markers of the present invention might optionally be used alone or in combination with one or more other compounds described herein, and/or in combination with known markers for liver cancer, including but not limited to Alpha fetoprotein (AFP), des-gamma-carboxyprothrombin (DCP), Squamous cell carcinoma antigen (SCCA)-immunoglobulin M (IgM), AFP (L3), or fucosylated AFP, GP73 (a golgi protein marker) and its fucosylated form, (TGF)-beta1, HS-GGT, free insulin-like growth factor (IGF)-II.

According to further embodiments of the present invention markers of the present invention might optionally be used alone or in combination with one or more other compounds described herein, and/or in combination with known markers for melanoma cancer, including but not limited to S100-beta, melanoma inhibitory activity (MIA), lactate dehydrogenase (LDH), tyrosinase, 5-S-Cysteinyldopa, L-Dopa/L-tyrosine, VEGF, bFGF, IL-8, ICAM-1, MMPs, IL-6, IL-10, sIL-2R (soluble interleukin-2-receptor), sHLA-DR (soluble HLA-DR), sHLA-class-I (soluble HLA-class I), TuM2-PK, Fas/CD95, sHLA-class-I (soluble HLA-class I), Albumin, TuM2-PK (Tumor pyruvate kinase type M2), sFas/CD95, YKL-40, CYT-MAA (cytoplasmic melanoma-associated antigen), HMW-MAA (high-molecular-weight melanoma-associated antigen), STAT3, STAT1, gp100/HMB45, p16 INK4A, PTEN, pRb (retinoblastoma protein), EGFR, p-Akt, c-Kit, c-myc, AP-2, HDM2, bcl-6, Ki67 (detected by Mib1), Cyclin A, B, D, E, p21CIP1, Geminin, PCNA (proliferating cell nuclear antigen), bcl-2, bax, bak, APAF-1, LYVE-1 (lymphatic vascular endothelial hyaluronan receptor-1), PTN, P-Cadherin, E-Cadherin, Beta-catenin, Integrins beta1 and beta3, MMPs (matrix metalloproteinases), Dysadherin, CEACAM1 (carcinoembryonic-antigen-related cell-adhesion molecule 1), Osteonectin, TA, Melastatin, ALCAM/CD166 (Activated leukocyte cell adhesion molecule), CXCR4, Metallothionein.

According to further embodiments of the present invention markers of the present invention might optionally be used alone or in combination with one or more other compounds described herein, and/or in combination with known markers for prostate cancer, including but not limited to PSA, PAP (prostatic acid phosphatase), CPK-BB, PSMA, PCA3, DD3, and/or in combination with the known protein(s) for the variant marker as described herein.

According to further embodiments of the present invention markers of the present invention might optionally be used alone or in combination with one or more other compounds described herein, and/or in combination with known markers for pancreatic cancer, including but not limited to CA 19-9, and/or in combination with the known protein(s) for the variant marker as described herein.

According to further embodiments of the present invention markers of the present invention might optionally be used alone or in combination with one or more other compounds described herein, and/or in combination with known markers for hematological cancer, including but not limited to soluble forms of tumor markers like P-Selectin, CD-22, interleukins, cytokines, and/or in combination with the known protein(s) for the variant marker as described herein.

According to further embodiments of the present invention markers of the present invention might optionally be used alone or in combination with one or more other compounds described herein, and/or in combination with known markers for colon cancer, including but not limited to CEA, CA19-9, CA50, and/or in combination with the known protein(s) for the variant marker as described herein.

According to further embodiments of the present invention markers of the present invention might optionally be used alone or in combination with one or more other compounds described herein, and/or in combination with known markers for gastric cancer, including but not limited to carbohydrate antigen (CA) 19-9, Carcinoembryonic antigen (CEA), Alpha-Fetoprotein and/or in combination with the known protein(s) for the variant marker as described herein.

According to further embodiments of the present invention markers of the present invention might optionally be used alone or in combination with one or more other compounds described herein, and/or in combination with known markers for head and neck cancer, including but not limited to carcinoembryonic antigen (CEA), carbohydrate antigen (CA) 19-9, squamous cell carcinoma antigen (SCC), thymidine kinase (TK), and deoxythymidine-5′-triphosphatase (dTTPase) and/or in combination with the known protein(s) for the variant marker as described herein.

According to further embodiments of the present invention markers of the present invention might optionally be used alone or in combination with one or more other compounds described herein, and/or in combination with known markers for immune related conditions, including but not limited to anti-Ro/SSA and anti-La/SSB antibodies for Sjogren's Syndrome; anti-dsDNA, Anti-RNP, Anti-Sm, ribosomal-P antibodies for systemic lupus erythematosus (SLE); anti-Scl-70/Topoisomerase antibodies for diffuse scleroderma; proMMP-3, proMMP-8 and proMMP-9, MMP/α2-macroglobulin (α2M) complexes for rheumatoid arthritis (RA); and/or in combination with the known protein(s) for the variant marker as described herein.

In some embodiments of the present invention, there are provided of methods, uses, devices and assays for the diagnosis of a disease or condition. Optionally a plurality of markers may be used with the present invention. The plurality of markers may optionally include a markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purposes.

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicators. In other embodiments, threshold levels of a diagnostic or prognostic indicators can be established, and the level of the indicators in a patient sample can simply be compared to the threshold levels. The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment.

According to embodiments of the present invention, KIAA0746, CD20 or CD55 protein, polynucleotide or a fragment thereof, may be featured as a biomarker for detecting disease and/or an indicative condition, as detailed above.

According to still other embodiments, the present invention optionally and preferably encompasses any amino acid sequence or fragment thereof encoded by a nucleic acid sequence corresponding to KIAA0746, CD20 or CD55 as described herein. Any oligopeptide or peptide relating to such an amino acid sequence or fragment thereof may optionally also (additionally or alternatively) be used as a biomarker.

In still other embodiments, the present invention provides a method for detecting a polynucleotide of this invention in a biological sample, using NAT based assays, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of the polynucleotide in the biological sample. Non-limiting examples of methods or assays are described below.

The present invention also relates to kits based upon such diagnostic methods or assays.

Nucleic Acid Technology (NAT) Based Assays:

Detection of a nucleic acid of interest in a biological sample may also optionally be effected by NAT-based assays, which involve nucleic acid amplification technology, such as PCR for example (or variations thereof such as real-time PCR for example). As used herein, a “primer” defines an oligonucleotide which is capable of annealing to (hybridizing with) a target sequence, thereby creating a double stranded region which can serve as an initiation point for DNA synthesis under suitable conditions. Amplification of a selected, or target, nucleic acid sequence may be carried out by a number of suitable methods known in the art. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Non-limiting examples of Nucleic Acid Technology-based assay is selected from the group consisting of a PCR, Real-Time PCR, LCR, Self-Sustained Synthetic Reaction, Q-Beta Replicase, Cycling probe reaction, Branched DNA, RFLP analysis, DGGE/TGGE, Single-Strand Conformation Polymorphism, Dideoxy fingerprinting, microarrays, Fluorescense In Situ Hybridization and Comparative Genomic Hybridization. The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. As commonly known in the art, the oligos are designed to bind to a complementary sequence under selected conditions. In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences. The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

Immunoassays

In another embodiment of the present invention, an immunoassay optionally may be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers optionally may be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay optionally may be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Radio-immunoassay (RIA): In one version, this method involves precipitation of the desired substrate and in the methods detailed herein below, with a specific antibody and radiolabeled antibody binding protein (e.g., protein A labeled with 1125) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Radio-Imaging Methods

These methods include but are not limited to, positron emission tomography (PET) single photon emission computed tomography (SPECT). Both of these techniques are non-invasive, and optionally may be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that optionally may be used. For example, U.S. Pat. No. 6,696,686 describes the use of SPECT for detection of breast cancer, and is hereby incorporated by reference as if fully set forth herein.

Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests optionally may be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker will be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

Uses and Methods of the Invention

The KIAA0746, CD20 or CD55 drugs according to the invention, especially antibodies, particularly the human antibodies, antibody compositions, and soluble conjugates containing the ectodomain of the KIAA0746, CD20 or CD55 antigen or a fragment or variant thereof, or a corresponding nucleic acid sequence or vector or cell expressing same and methods of the present invention have numerous in vitro and in vivo diagnostic and therapeutic utilities involving the diagnosis and treatment of KIAA0746, CD20 or CD55 antigen related disorders. As noted these conditions include in cancer that differentially express the KIAA0746, CD20 or CD55 antigen, including invasive and metastatic forms thereof. The subject anti-KIAA0746, anti-CD20 or anti-CD55 antibodies may prevent T cell activity against cancer cells and/or prevent positive stimulation of T cell activity. Such antibodies may be used in the treatment of conditions including cancer; as well as non-malignant disorders such as immune related conditions; diseases in which complement activation and deposition is involved in pathogenesis, inflammation of the respiratory tract disorder; ischemia reperfusion injury related disorder, or lymphoproliferative disorder

For example, these molecules can be administered to cells in culture, in vitro or ex vivo, or to human subjects, e.g., in vivo, to treat, prevent and to diagnose a variety of disorders. Preferred subjects include human patients having disorders mediated by cells expressing the KIAA0746, CD20 or CD55 antigen and cells that posses KIAA0746, CD20 or CD55 activity. The methods are particularly suitable for treating human patients having a disorder associated with aberrant KIAA0746, CD20 or CD55 antigen expression using antibodies that specifically bind Z43375_{—}1_P4 (SEQ ID NO:18), Z43375_{—}1_P8 (SEQ ID NO:19), Z43375_{—}1_P40 (SEQ ID NO:20), Z43375_{—}1_P46 (SEQ ID NO:21), Z43375_{—}1_P47 (SEQ ID NO:22), Z43375_{—}1_P50 (SEQ ID NO:23), Z43375_{—}1_P51 (SEQ ID NO:24), Z43375_{—}1_P52 (SEQ ID NO:25), Z43375_{—}1_P53 (SEQ ID NO:26), Z43375_{—}1_P54 (SEQ ID NO:27), Z43375_{—}1_P55 (SEQ ID NO:28), Z43375_{—}1_P56 (SEQ ID NO:29), Z43375_{—}1_P60 (SEQ ID NO:30), HSCD20B_{—}1_P5 (SEQ ID NO:33), HUMDAF_P14 (SEQ ID NO:51), HUMDAF_P15 (SEQ ID NO:52), HUMDAF_P20 (SEQ ID NO:53), HUMDAF_P26 (SEQ ID NO:54), HUMDAF_P29 (SEQ ID NO:55), HUMDAF_P30 (SEQ ID NO:56), HUMDAF_P31 (SEQ ID NO:57).

KIAA0746, CD20 or CD55 drugs according to the invention, are administered together with another agent, the two can be administered in either order or simultaneously.

Given the specific binding of the antibodies of the invention for KIAA0746, CD20 or CD55 the antibodies of the invention optionally may be used to specifically detect KIAA0746, CD20 or CD55 expression on the surface of cells and, moreover, optionally may be used to purify KIAA0746, CD20 or CD55 antigen via immunoaffinity purification.

Furthermore, given the expression of KIAA0746, CD20 or CD55 on various tumor cells, the human antibodies, antibody compositions and methods of the present invention optionally may be used to treat a subject with a tumorigenic disorder, e.g., a disorder characterized by the presence of tumor cells expressing KIAA0746, CD20 or CD55 antigen, as mentioned.

In one embodiment, the antibodies (e.g., human monoclonal antibodies, multispecific and bispecific molecules and compositions) of the invention optionally may be used to detect levels of KIAA0746, CD20 or CD55 or levels of cells which contain KIAA0746, CD20 or CD55, respectively, on their membrane surface, which levels can then be linked to certain disease symptoms. Alternatively, the antibodies optionally may be used to inhibit or block KIAA0746, CD20 or CD55 function which, in turn, can be linked to the prevention or amelioration of certain disease symptoms, thereby implicating KIAA0746, CD20 or CD55, respectively, as a mediator of the disease. This can be achieved by contacting a sample and a control sample with the anti-KIAA0746, anti-CD20 or anti-CD55 antibody under conditions that allow for the formation of a complex between the corresponding antibody and KIAA0746, CD20 or CD55, respectively. Any complexes formed between the antibody and KIAA0746, CD20 or CD55 are detected and compared in the sample and the control.

In another embodiment, the antibodies (e.g., human antibodies, multispecific and bispecific molecules and compositions) of the invention can be initially tested for binding activity associated with therapeutic or diagnostic use in vitro. For example, compositions of the invention can be tested using low cytometric assays.

The antibodies (e.g., human antibodies, multispecific and bispecific molecules, immunoconjugates and compositions) of the invention have additional utility in therapy and diagnosis of KIAA0746, CD20 or CD55-related diseases. For example, the human monoclonal antibodies, the multispecific or bispecific molecules and the immunoconjugates optionally may be used to elicit in vivo or in vitro one or more of the following biological activities: to inhibit the growth of and/or kill a cell expressing KIAA0746, CD20 or CD55; to mediate phagocytosis or ADCC of a cell expressing KIAA0746, CD20 or CD55 in the presence of human effector cells, or to block KIAA0746, CD20 or CD55 ligand binding to KIAA0746, CD20 or CD55, respectively.

In a particular embodiment, the antibodies (e.g., human antibodies, multispecific and bispecific molecules and compositions) are used in vivo to treat, prevent or diagnose a variety of KIAA0746, CD20 or CD55-related diseases. Examples of KIAA0746, CD20 or CD55-related diseases include, among others, cancer, such as hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic. Additional examples of KIAA0746, CD20 or CD55-related diseases include, among others, non-malignant disorders such as immune related conditions or disorders including but not limited to inflammatory or autoimmune diseases, ischemia-reperfusion injury, respiratory tract disorder, transplant rejection, transplant rejection following allogenic transplantation or xenotransplantation, and graft versus host disease. Such disorders include by way of example multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease. Additional examples of CD55-related diseases include, among others, diseases in which complement activation and deposition is involved in pathogenesis, inflammation of the respiratory tract disorders, ischemia reperfusion injury related disorders, immune related conditions related to transplantation, such as acute and chronic rejection of organ transplantation and of allogeneic stem cell transplantation, autologous stem cell transplantation, bone marrow transplantation, treatment of Graft Versus Host Disease (GVHD), rejection in xenotransplantation, and use of CD55 variant-transgenic animals for xenotransplantation. Additional examples of KIAA0746 or CD20-related diseases include, among others, lymphoproliferative disorders.

Suitable routes of administering the antibody compositions (e.g., human monoclonal antibodies, multispecific and bispecific molecules and immunoconjugates) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-KIAA0746, anti-CD20, anti-CD55, antibodies of the invention can be co-administered with one or other more therapeutic agents, e.g., an cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-KIAA0746, anti-CD20, anti-CD55 antibodies, or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10−8 to 10−9 but will vary depending on the therapeutic purpose. In general, the amount will be sufficient to obtain localization at the target cell, e.g., a tumor cell expressing KIAA0746, CD20 or CD55 and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions optionally may be used in conjunction with chemotherapy. Additionally, combination immunotherapy may be used to direct two distinct cytotoxic effector populations toward tumor cell rejection. For example, anti-KIAA0746, anti-CD20 or anti-CD55 antibodies linked to anti-Fc-gamma R1 or anti-CD3 may be used in conjunction with IgG- or IgA-receptor specific binding agents.

Bispecific and multispecific molecules of the invention can also be used to modulate FcgammaR or FcgammaR levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of the invention which have complement binding sites, such as portions from IgG1, -2, or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions (e.g., human antibodies, multispecific and bispecific molecules and immunoconjugates) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Also within the scope of the present invention are kits comprising the KIAA0746, CD20 or CD55 antigen or KIAA0746, CD20 or CD55 conjugates or antibody compositions of the invention (e.g., human antibodies, bispecific or multispecific molecules, or immunoconjugates) and instructions for use. The kit can further contain one or more additional reagents, such as an immunosuppressive reagent, a cytotoxic agent or a radiotoxic agent, or one or more additional human antibodies of the invention (e.g., a human antibody having a complementary activity which binds to an epitope in the KIAA0746, CD20 or CD55 antigen distinct from the first human antibody).

Accordingly, patients treated with antibody compositions of the invention can be additionally administered (prior to, simultaneously with, or following administration of a human antibody of the invention) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcy or Fcy receptors by, for example, treating the subject with a cytokine Preferred cytokines for administration during treatment with the multispecific molecule include of granulocyte colony-stimulating factor (G-CSF), granulocyte-macrophage colony-stimulating factor (GM-CSF), interferon-.gamma. (IFN-.gamma.), and tumor necrosis factor (TNF).

The compositions (e.g., human antibodies, multispecific and bispecific molecules) of the invention can also be used to target cells expressing Fc gamma R or KIAA0746, CD20 or CD55, for example for labeling such cells. For such use, the binding agent can be linked to a molecule that can be detected. Thus, the invention provides methods for localizing ex vivo or in vitro cells expressing Fc receptors, such as FcgammaR, or KIAA0746, CD20 or CD55 antigen. The detectable label can be, e.g., a radioisotope, a fluorescent compound, an enzyme, or an enzyme co-factor.

In a particular embodiment, the invention provides methods for detecting the presence of KIAA0746, CD20 or CD55 antigen in a sample, or measuring the amount of KIAA0746, CD20 or CD55 antigen, respectively, comprising contacting the sample, and a control sample, with a human monoclonal antibody, or an antigen binding portion thereof, which specifically binds to KIAA0746, CD20 or CD55, respectively, under conditions that allow for formation of a complex between the antibody or portion thereof and KIAA0746, CD20 or CD55. The formation of a complex is then detected, wherein a difference complex formation between the sample compared to the control sample is indicative the presence of KIAA0746, CD20 or CD55 antigen in the sample. As noted the invention in particular embraces assays for detecting KIAA0746, CD20 or CD55 antigen in vitro and in vivo such as immunoassays, radioimmunoassays, radioassays, radioimaging assays, ELISAs, Western blot, FACS, slot blot, immunohistochemical assays, and other assays well known to those skilled in the art.

In other embodiments, the invention provides methods for treating an KIAA0746, CD20 or CD55 mediated disorder in a subject, e.g., cancer, selected from the group consisting of hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and non-solid or solid tumors of breast, prostate, lung, colon, ovary, spleen, kidney, bladder, head and neck, uterus, testicles, stomach, cervix, liver, bone, skin, pancreas, brain and wherein the cancer is non-metastatic, invasive or metastatic; as well as non-malignant disorders such as immune related conditions or disorders, inflammatory and autoimmune diseases, selected from the group consisting of multiple sclerosis; psoriasis; rheumatoid arthritis; psoriatic arthritis, systemic lupus erythematosus; ulcerative colitis; Crohn's disease; immune disorders associated with graft transplantation rejection; benign lymphocytic angiitis, thrombocytopenic purpura, idiopathic thrombocytopenia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, ANCA-associated vasculitis, Wegener's granulomatosis, microscopic polyangiitis, cryoglobulinemic vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, pernicious anaemia, pemphigus, pemphigus vulgaris, primary biliary cirrhosis, dermatomyositis, polymyositis, fibromyositis, myogelosis, celiac disease, immunoglobulin A nephropathy, Henoch-Schonlein purpura, atopic dermatitis, atopic eczema, chronic urticaria, psoriasis, psoriasis arthropathica, Graves' disease, Graves' ophthalmopathy, scleroderma, systemic scleroderma, asthma, allergy, primary biliary cirrhosis, Hashimoto's thyroiditis, primary myxedema, sympathetic ophthalmia, autoimmune uveitis, chronic action hepatitis, collagen diseases, ankylosing spondylitis, periarthritis humeroscapularis, panarteritis nodosa, chondrocalcinosis and other immune related conditions such as transplant rejection, transplant rejection following allogenic transplantation or xenotransplantation, and graft versus host disease, diseases in which complement activation and deposition is involved in pathogenesis, ischemia-reperfusion injury, respiratory tract disorder, lymphoproliferative disorder, and methods of treating any condition wherein modulation of immune costimulation that involves KIAA0746, CD20 or CD55 is therapeutically desirable using anti-KIAA0746, anti-CD20 or anti-CD55 antibodies or soluble KIAA0746, CD20 or CD55 antigen conjugates or other drugs that target and modulate (promote or inhibit) one or more KIAA0746, CD20 or CD55 biological activities.

By administering the anti-KIAA0746, anti-CD20 or anti-CD55 antibody, soluble KIAA0746, CD20 or CD55 antigen conjugate or other drug that targets the KIAA0746, CD20 or CD55 antigen or a portion thereof to a subject, the ability of KIAA0746, CD20 or CD55 antigen to induce such activities is inhibited or promoted and, thus, the associated disorder is treated. The soluble KIAA0746, CD20 or CD55 antigen or antigen conjugate or anti-KIAA0746, anti-CD20 or anti-CD55 antibody or fragment containing composition or other drug that targets and modulates KIAA0746, CD20 or CD55, can be administered alone or along with another therapeutic agent, such as a cytotoxic or a radiotoxic agent which acts in conjunction with or synergistically with the antibody composition to treat or prevent the KIAA0746, CD20 or CD55 antigen mediated disease.

In yet another embodiment, immunoconjugates of the invention optionally may be used to target compounds (e.g., therapeutic agents, labels, cytotoxins, radiotoxins immunosuppressants, etc.) to cells which have KIAA0746, CD20 or CD55 cell surface receptors by linking such compounds to the antibody. Thus, the invention also provides methods for localizing ex vivo or in vivo cells expressing KIAA0746, CD20 or CD55 (e.g., with a detectable label, such as a radioisotope, a fluorescent compound, an enzyme, or an enzyme co-factor). Alternatively, the immunoconjugates optionally may be used to kill cells which have KIAA0746, CD20 or CD55 cell surface receptors by targeting cytotoxins or radiotoxins to KIAA0746, CD20 or CD55 antigen.

The present invention is further illustrated by the following sequence characterization of a DNA transcript encoding the KIAA0746, CD20 or CD55 antigen, its domains and expression data in normal and cancerous tissues as well as examples describing the manufacture of fully human antibodies thereto. This information and examples is illustrative and will not be construed as further limiting. The contents of all figures and all references, patents and published patent applications cited throughout this application are expressly incorporated herein by reference.

### EXAMPLES

### Example 1

### Methods Used to Analyze the Expression of the RNA Encoding the Proteins of the Invention

The targets of the present invention were tested with regard to their expression in various cancerous and non-cancerous tissue samples and/or with regard to its expression in a wide panel of human samples which contains various types of immune cells, and hematological malignancies samples and cell lines, as well as several samples of normal tissues. A description of the samples used in a wide panel of various cancer types and normal tissues, as well as various cell lines, is provided in Table 1 below. In Table 1, samples A1-A15 are serial dilutions of known amounts of the amplicon, which were used to quantitate the mRNA copies per 5 ng of cDNA. Table 1 also shows the Ct and the corresponding quantity of the amplicon calculated for each samples, as explained below. A description of the samples used in a blood-specific panel which contains various types of immune cells, and hematological malignancies samples and cell lines, as well as several samples of normal tissues, is provided in Table 2 below. A description of the samples used in the normal tissue panels is provided in Table 3. A description of the samples used in the ovary cancer testing panel is provided in Table 4 below. The key for the table 4 is given in table 4_{—}1. Table 5 provides a list of tissue samples in a combined panel, which includes samples from blood specific panel and from normal panel. The details of the blood specific samples listed in Table 5 are provided in Table 2 (samples 3-47) and the details of the normal samples listed in Table 3 (samples 1-69). A description of the samples used in a colon cancer tissue panel is provided in Table 6 below. The key for Table 6 is presented in Table 6_{—}1 below. Tests were then performed as described in the “Materials and Experimental Procedures” section below.

TABLE 1

| Well | Sample Name | Ct | Quantity |
|---|---|---|---|
| 1 | A1 | 33.500107 | 100 |
| 2 | A2 | 29.234596 | 1000 |
| 3 | A3 | 25.594917 | 10000 |
| 4 | A4 | 21.399303 | 100000 |
| 5 | A5 | 18.545624 | 1000000 |
| 6 | A6 | 32.06496 | 100 |
| 7 | A7 | 28.484388 | 1000 |
| 8 | A8 | 25.041174 | 10000 |
| 9 | A9 | 21.563948 | 100000 |
| 10 | A10 | 18.278769 | 1000000 |
| 11 | A11 | 32.41434 | 100 |
| 12 | A12 | 27.97173 | 1000 |
| 13 | A13 | 24.636381 | 10000 |
| 14 | A14 | 21.145983 | 100000 |
| 15 | A15 | 17.963104 | 1000000 |
| 17 | Water | Undetermined | 0 |
| 25 | N. Adipose (#301) | 32.954025 | 69.57206 |
| 26 | N. Artery (#303) | 32.850517 | 74.3355 |
| 27 | N. Bladder (#257) | 33.722763 | 42.54298 |
| 28 | N. Bone Marrow Stromal | 32.816658 | 75.96344 |
| | Cells (#394) | | |
| 29 | N. Brain (#258) | 31.321669 | 197.7028 |
| 30 | N. Brain: Cerebellum (#123) | 30.761787 | 282.8691 |
| 31 | N. Brain: Cerebellum (#130) | 29.24173 | 748.0998 |
| 32 | N. Brain: Cerebral Cortex | 33.451355 | 50.61081 |
| | (#304) | | |
| 33 | N. Brain: Hippocampus | 31.652502 | 159.9872 |
| | (#121) | | |
| 34 | N. Brain: Hippocampus | 30.501091 | 334.2139 |
| | (#128) | | |
| 35 | N. Brain: Pituitary Gland | 34.92184 | 19.75349 |
| | (#452) | | |
| 36 | N. Brain: Thalamus (#131) | 36.58653 | 6.808976 |
| 37 | N. Breast (#259) | 33.39013 | 52.63273 |
| 38 | N. Cecum (#305) | 29.745876 | 541.8434 |
| 39 | N. Cervix (#260) | 35.12822 | 17.31003 |
| 40 | N. Colon (#261) | 30.089859 | 434.8028 |
| 41 | N. Dendritic Cells (#439) | 30.961166 | 248.9916 |
| 42 | N. Diaphragm (#316) | 35.96481 | 10.13531 |
| 43 | N. Duodenum (#306) | 28.779663 | 1005.431 |
| 44 | N. Esophagus (#262) | 33.51607 | 48.55799 |
| 45 | N. Esophagus (#307) | 32.6811 | 82.84609 |
| 46 | N. Heart (#118) | 35.36428 | 14.88347 |
| 47 | N. Heart (#125) | 34.846024 | 20.73533 |
| 48 | N. Heart (#263) | 39.431232 | 1.103144 |
| 49 | N. Heart: Atrium (#309) | 37.2544 | 4.441226 |
| 50 | N. Heart: Ventricle (#310) | 35.99771 | 9.924184 |
| 51 | N. HUVEC (#254) | 32.787766 | 77.38075 |
| 52 | N. HUVEC (#361) | 31.025959 | 238.8807 |
| 53 | N. Kidney (#264) | 36.12211 | 9.164925 |
| 54 | N. Kidney (#265) | 31.673235 | 157.8789 |
| 55 | N. Kidney (#311) | 32.763958 | 78.56847 |
| 56 | N. Liver (#266) | Undetermined | 0 |
| 57 | N. Liver (#267) | 37.629097 | 3.494511 |
| 58 | N. Liver (#312) | 34.39538 | 27.66498 |
| 59 | N. Liver (#333) | 32.70491 | 81.59352 |
| 60 | N. Liver (#334) | 32.98493 | 68.20999 |
| 61 | N. Liver (#335) | 32.779373 | 77.79736 |
| 62 | N. Liver (#336) | 32.540375 | 90.65144 |
| 63 | N. Liver (#337) | 31.32468 | 197.3222 |
| 64 | N. Liver (#338) | Undetermined | 0 |
| 65 | N. Liver (#340) | Undetermined | 0 |
| 66 | N. Liver (Fetal; #341) | Undetermined | 0 |
| 67 | N. Lung (#268) | 33.178585 | 60.2611 |
| 68 | N. Lung (#313) | 30.769547 | 281.4683 |
| 69 | N. Lung (NAT, #38) | 30.911793 | 256.9828 |
| 70 | N. Lymph Node (#269) | 31.70463 | 154.7393 |
| 71 | N. Lymph Node (#315) | 29.920544 | 484.551 |
| 72 | N. Monocytes (#438) | 34.805653 | 21.27789 |
| 73 | N. Ovary (#270) | 34.897247 | 20.06677 |
| 74 | N. Pancreas (#271) | 31.925358 | 134.3592 |
| 75 | N. Pancreas (#317) | 33.002888 | 67.43068 |
| 76 | N. Peripheral Blood | 30.26283 | 389.2504 |
| | Leukocytes (#302) | | |
| 77 | N. Prostate (#272) | 35.382385 | 14.71206 |
| 78 | N. Retina (#256) | 29.037664 | 852.4372 |
| 79 | N. Skeletal Muscle (#119) | 35.067318 | 17.99784 |
| 80 | N. Skeletal Muscle (#126) | 38.998566 | 1.454977 |
| 81 | N. Skin (#273) | Undetermined | 0 |
| 82 | N. Skin (#319) | 32.862514 | 73.7671 |
| 83 | N. Small Intestine (#320) | 28.056322 | 1597.149 |
| 84 | N. Small Intestine: Jejunum | 29.956089 | 473.6554 |
| | (#321) | | |
| 85 | N. Spinal Cord (#122) | 33.68272 | 43.64702 |
| 86 | N. Spinal Cord (#129) | 31.524664 | 173.623 |
| 87 | N. Spleen (#274) | Undetermined | 0 |
| 88 | N. Spleen (#322) | 29.351997 | 697.1395 |
| 89 | N. Stomach (#275) | 39.04655 | 1.410986 |
| 90 | N. Stomach (#323) | 27.659506 | 2058.768 |
| 91 | N. Testis (#276) | 35.438683 | 14.19157 |
| 92 | N. Tongue (#324) | 30.848854 | 267.5423 |
| 93 | N. Tonsil (#325) | 32.875484 | 73.15749 |
| 94 | N. Trachea (#314) | 31.725376 | 152.6989 |
| 95 | Brain T. (Glioblastoma, #192) | 31.579073 | 167.6829 |
| 96 | Breast T. (#176) | 31.411497 | 186.6606 |
| 97 | Breast T. (#177) | 30.182444 | 409.7944 |
| 98 | Breast T. (#178) | 31.37305 | 191.3091 |
| 99 | Breast T. (#179) | 33.378407 | 53.02898 |
| 100 | Breast T. (#180) | 31.597816 | 165.684 |
| 101 | Colon T. (#181) | 27.673208 | 2040.798 |
| 102 | Colon T. (#182) | 28.554306 | 1161.374 |
| 103 | Colon T. (#183) | 27.027317 | 3085.119 |
| 104 | Colon T. (#184) | 27.242493 | 2688.323 |
| 105 | Colon T. (#185) | 27.351913 | 2506.555 |
| 106 | Head/Neck T. (Larynx, #402) | 29.831688 | 512.8964 |
| 107 | Head/Neck T. (Pharynx, | 27.577345 | 2169.888 |
| | #403) | | |
| 108 | Head/Neck T. (Tongue, | 30.616032 | 310.5176 |
| | #404) | | |
| 109 | Head/Neck T. (Tonsil, #405) | 28.76841 | 1012.697 |
| 110 | Kidney T. (#167) | 26.694164 | 3818.07 |
| 111 | Kidney T. (#168) | 29.429527 | 663.4018 |
| 112 | Kidney T. (#169) | 31.18958 | 215.1373 |
| 113 | Kidney T. (#170) | 26.71874 | 3758.504 |
| 114 | Kidney T. (#171) | 28.957127 | 897.5142 |
| 115 | Liver T. (#326) | 30.07056 | 440.2049 |
| 116 | Liver T. (#327) | 28.975212 | 887.1885 |
| 117 | Liver T. (#328) | 29.460033 | 650.5792 |
| 118 | Liver T. (#329) | 30.290613 | 382.3924 |
| 119 | Liver T. (#330) | 32.162376 | 115.4537 |
| 120 | Liver T. (#331) | 34.410873 | 27.39206 |
| 121 | Liver T. (#332) | 35.637173 | 12.49904 |
| 122 | Liver T. (#339) | 28.150501 | 1503.751 |
| 123 | Lung T. (NSC, #157) | 26.755575 | 3670.963 |
| 124 | Lung T. (NSC, #158) | 29.013521 | 865.7072 |
| 125 | Lung T. (NSC, #159) | 27.643871 | 2079.465 |
| 126 | Lung T. (NSC, #160) | 29.6046 | 593.1021 |
| 127 | Lung T. (NSC, #161) | 28.192722 | 1463.673 |
| 128 | Lung T. (NSC, #35) | 28.015759 | 1639.142 |
| 129 | Lung T. (NSC, #36) | 29.7458 | 541.8696 |
| 130 | Lung T. (NSC, #37) | 32.259342 | 108.5086 |
| 131 | Lung T. (SC, #34) | 37.795494 | 3.141595 |
| 132 | Lung T. (SC, #39) | 27.320545 | 2557.368 |
| 133 | Lung T. (SC, #40) | 28.720863 | 1043.977 |
| 134 | Lymphoma (#287) | 25.64843 | 7454.51 |
| 135 | Lymphoma (#288) | 27.76279 | 1927.116 |
| 136 | Lymphoma (#290) | 26.22954 | 5139.818 |
| 137 | Lymphoma (#289) | 27.019264 | 3101.055 |
| 138 | Lymphoma (#291) | 27.507656 | 2268.827 |
| 139 | Lymphoma (#292) | 27.970276 | 1687.542 |
| 140 | Lymphoma (#293) | 26.63365 | 3968.798 |
| 141 | Lymphoma (#294) | 27.818335 | 1859.833 |
| 142 | Lymphoma (#295) | 29.355621 | 695.5251 |
| 143 | Lymphoma (#296) | 29.52 | 626.0908 |
| 144 | Lymphoma (#297) | 27.164728 | 2825.465 |
| 145 | Lymphoma (#298) | 28.1519 | 1502.406 |
| 146 | Lymphoma (#299) | 29.073183 | 833.284 |
| 147 | Lymphoma (#300) | 28.995129 | 875.9549 |
| 148 | Melanoma (#162) | 33.16848 | 60.65197 |
| 149 | Melanoma (#163) | 29.423386 | 666.0138 |
| 150 | Melanoma (#166) | 35.558308 | 13.14591 |
| 151 | Melanoma (#165) | 27.99184 | 1664.418 |
| 152 | Melanoma (#164) | 29.217522 | 759.7769 |
| 153 | Ovary T. (#187) | 28.231396 | 1427.9 |
| 154 | Ovary T. (#188) | 27.92376 | 1738.521 |
| 155 | Ovary T. (#189) | 34.033978 | 34.86195 |
| 156 | Ovary T. (#190) | 30.116852 | 427.3581 |
| 157 | Ovary T. (#191) | 27.00813 | 3123.226 |
| 158 | Pancreas T. (#172) | 30.090097 | 434.7364 |
| 159 | Pancreas T. (#173) | 26.466 | 4418.18 |
| 160 | Pancreas T. (#174) | 30.753923 | 284.2959 |
| 161 | Pancreas T. (#175) | 32.415817 | 98.17143 |
| 162 | Pancreas T. (#186) | 32.11713 | 118.8448 |
| 163 | Prostate T. (#378) | 28.772379 | 1010.129 |
| 164 | Prostate T. (#379) | 28.68595 | 1067.561 |
| 165 | Prostate T. (#380) | 29.480915 | 641.9449 |
| 166 | Prostate T. (#381) | 27.953196 | 1706.085 |
| 167 | Prostate T. (#382) | 28.379261 | 1299.006 |
| 168 | Prostate T. (#383) | 30.391417 | 358.5085 |
| 169 | SW780 (#440) | 33.27011 | 56.83357 |
| 170 | MCF-7 (#390) | 31.551472 | 170.6704 |
| 171 | MCF-7/Adr (#375) | 33.331547 | 54.64294 |
| 172 | MD-MB-361 (#279) | 30.262936 | 389.2243 |
| 173 | T47D (#395) | 31.259138 | 205.7729 |
| 174 | A431 (#386) | 30.936993 | 252.8725 |
| 175 | HT-1080 (#393) | 28.37019 | 1306.567 |
| 176 | 293F (#362) | 32.41837 | 98.01127 |
| 177 | 786-0 (#344) | 30.283682 | 384.092 |
| 178 | 786-0 (#392) | 34.459076 | 26.56017 |
| 179 | 786-0 (#450) | 30.802727 | 275.556 |
| 183 | NCl-H226* (#347) | 29.57432 | 604.7053 |
| 184 | SHP-77 (#346) | Undetermined | 0 |
| 185 | SHP-77 (#444) | 33.134804 | 61.97299 |
| 186 | NCl-H1688 (#445) | 25.00451 | 11254.94 |
| 187 | H16AR (#446) | 27.081669 | 2979.678 |
| 188 | NCl-H69 (#447) | Undetermined | 0 |
| 189 | NCl-H446 (#448) | 32.87046 | 73.39301 |
| 190 | NCl-H82 (#449) | 31.627363 | 162.5813 |
| 191 | DMS-79 (#451) | 27.93514 | 1725.908 |
| 192 | HL-60 (#373) | 36.750946 | 6.129085 |
| 193 | Raji (#376) | 30.963068 | 248.6889 |
| 194 | Ramos (3377) | 33.32551 | 54.85447 |
| 195 | L540 (#391) | 29.111725 | 812.9869 |
| 196 | SU-DHL-6 (#372) | 27.750486 | 1942.347 |
| 197 | Jurkat (#278) | 29.63703 | 580.9232 |
| 198 | KARPAS-299 (#255) | 27.747408 | 1946.176 |
| 199 | U-937 (#281) | 32.83805 | 74.93078 |
| 200 | THP-1 (#277) | Undetermined | 0 |
| 201 | ES-2 (#280) | Undetermined | 0 |
| 202 | OVCAR-3 (#284) | 29.619244 | 587.5717 |
| 203 | OVCAR-3 (#399) | 29.963314 | 471.471 |
| 204 | PA-1 (#286) | 28.80499 | 989.27 |
| 205 | PA-1 (#400) | 26.97693 | 3186.196 |
| 206 | SKOV3 (#343) | 30.626667 | 308.4118 |
| 207 | SW-626 (#396) | 28.588648 | 1136.134 |
| 208 | OV-90 (#397) | 28.603535 | 1125.364 |
| 209 | TOV-21G (#398) | 33.755844 | 41.652 |
| 210 | CAOV1 (#401) | 29.268953 | 735.1823 |
| 211 | HPAC (#374) | 29.337992 | 703.4146 |
| 212 | HPAC (#385) | 27.625755 | 2103.708 |
| 213 | DU145 (#360) | 29.67416 | 567.285 |
| 214 | DU145 (#387) | 28.723919 | 1041.939 |
| 215 | LNCaP (#359) | 25.980776 | 6026.582 |
| 216 | PC3 (#384) | 29.793344 | 525.6345 |
| 217 | SK-MEL-28 (#345) | 37.69728 | 3.345341 |
| 218 | 4T1 (#389) | Undetermined | 0 |
| 219 | CHO-S (#388) | Undetermined | 0 |

Slope −3.5988433

TABLE 2

| | | Organ/Cell | |
|---|---|---|---|
| Blood panel sample | Description | type | Tumor Type |
| 1_PBMC2 | PBMCs | blood-derived | |
| | | cells | |
| 2_PBMC3 | PBMCs | blood-derived | |
| | | cells | |
| 3_Bcell1 | B cells | blood-derived | |
| | | cells | |
| 4_Bcell2 | B cells | blood-derived | |
| | | cells | |
| 5_J_Bcells | B cells | blood-derived | |
| | | cells | |
| 6_K_Bcells_act | Bcells activated | blood-derived | |
| | | cells | |
| 7_Tcell1 | T cells | blood-derived | |
| | | cells | |
| 8_Tcell2 | T cells | blood-derived | |
| | | cells | |
| 9_M_CD8 | CD4+ T cells | blood-derived | |
| | | cells | |
| 10_G_CD4_unt | CD8+ T cells | blood-derived | |
| | | cells | |
| 11_H_CD4_Beads | CD4+ w Activation | blood-derived | |
| | beads | cells | |
| 12_I_CD4_Beads_IL12 | CD4 w act. | blood-derived | |
| | Beads + IL12 | cells | |
| 13_95_CD4+CD25− | CD4+CD25− | blood-derived | |
| | | cells | |
| 15_NK | NK cells | blood-derived | |
| | | cells | |
| 16_CD34+_1548 | CD34+(PCBM1548) | blood-derived | |
| | | cells | |
| 17_CD34+_1028 | CD34+(PCBM1028) | blood-derived | |
| | | cells | |
| 18_PMN | PMNs | blood-derived | |
| | | cells | |
| 19_A_Mono | Monocytes | blood-derived | |
| | | cells | |
| 20_B_Macro_imma | Macrophages | blood-derived | |
| | immature | cells | |
| 21_C_Macro_mat | Macrophages | blood-derived | |
| | mature | cells | |
| 22_D_DCs_immat | DCs immature | blood-derived | |
| | | cells | |
| 23_E_DCs_mat_LPS | DCs mature LPS | blood-derived | |
| | | cells | |
| 24_F_DCs_mat_CK | DCs mature CK | blood-derived | |
| | | cells | |
| 25_L_DCs + T | DCs + T cells | blood-derived | |
| | | cells | |
| 26_Lym1 | 13987A1 | Lymph Node | Lymphoma |
| 27_Lym2 | 43594B1 | Muscle | lymphoma |
| 28_Lym3 | 65493A1 | Testis | Lymphoma |
| 29_MalLym3 | 75894A1 | Brain | Lymphoma |
| 30_NonHod_SCLym | 83325A1 | Lymph Node | NHL Small |
| | | | Cell |
| 31_NonHod_FolLym | 76943A1(5 tubes) | Lymph Node | NHL Follicular |
| 32_Lym_Fol_GI | CN_4_ASRBNA35 | | NHL Follicular |
| | | | Grade I (Small |
| | | | Cell) |
| 33_Lym_Fol_GII | CN_1_113GHA8J | | NHL Follicular |
| | | | Grade II |
| | | | (mixed Small & |
| | | | Large Cell) |
| 34_Lym_Fol_GIII | CN_8_VXML6AXI | | NHL Follicular |
| | | | Grade III |
| | | | (Large Cell) |
| 35_MalLym1 | 76218B1 | Testis | NHL Large |
| | | | Cell |
| 36_MalLym2 | 76102A1 | Lymph Node | NHL Large |
| | | | Cell |
| 37_Lym_DifBCell1 | CN_2_4HDLNA2R | | NHL Diffuse |
| | | | Large B-Cell |
| 38_Lym_DifBCell2 | CN_3_4M4S7AAM | | NHL Diffuse |
| | | | Large B-Cell |
| 39_Lym_DifBCell3 | CN_5_HEODOAR2 | | NHL Diffuse |
| | | | Large B-Cell |
| 40_NonHod_Lym1 | 77332A1(5 tubes) | Colon | NHL Diffuse |
| | | | Large B-Cell |
| 41_MalLym4 | 76161A1 | Spleen | NHL Diffuse |
| | | | Large B-Cell |
| 42_Lym_MantleCell1 | CN_6_MAE47AOY | | NHL Mantle |
| | | | Cell |
| 43_Lym_MantleCell2 | CN_7_VJU9OAO9 | | NHL Mantle |
| | | | Cell |
| 44_NonHod_Lym2 | 95377A1(5 tubes) | Spleen | NHL |
| 45_THP_1 | THP-1 | monocytes | AML cell line |
| 46_KG_1 | KG-1 | myeloblast | AML cell line |
| 47_BDCM | BDCM | B and DC like | AML cell line |
| 48_CESS | CESS | lymphoblasts | AML cell line |
| 49_HL60 | HL60 | myeloblast | AML cell line |
| 50_K562 | K562 | lymphoblasts | CML cell line |
| 51_Jurkat | Jurkat | T lymphoblasts | T ALL cell line |
| 52_GA10 | GA10 | B lymphoblasts | Burkitts |
| | | | lymphoma cell |
| | | | line |
| 53_RAMOS | RAMOS | B lymphoblasts | Burkitts |
| | | | lymphoma cell |
| | | | line |
| 54_RAJI | RAJI | B lymphoblasts | Burkitts |
| | | | lymphoma cell |
| | | | line |
| 55_Daudi | Daudi | B lymphoblasts | Burkitts |
| | | | lymphoma cell |
| | | | line |
| 56_NL564 | NL553 | B lymphoblasts | EBV |
| | | | transformed |
| | | | cell line |
| 57_NL553 | NL564 | B lymphoblasts | EBV |
| | | | transformed |
| | | | cell line |
| 58_SKW6.4 | SKW6.4 | B cells | EBV |
| | | lymphoblasts | transformed |
| | | | cell line |
| 59_NCI_H929 | NCI-H929 | B lymphoblasts | Multiple |
| | | | Myeloma cell |
| | | | line |
| 60_MC/CAR | MC/CAR | B lymphoblasts | Multiple |
| | | | Myeloma cell |
| | | | line |
| 61_U266 | U266 | B lymphoblasts | Multiple |
| | | | Myeloma cell |
| | | | line |
| 62_RPMI8226 | RPMI8226 | B lymphoblasts | Multiple |
| | | | Myeloma cell |
| | | | line |
| 63_IM_9 | IM-9 | B lymphoblasts | Multiple |
| | | | Myeloma cell |
| | | | line |
| 64_cereN | cerebellum normal | cerebellum | |
| | | normal | |
| 65_kidneyN1 | kidney normal | kidney normal | |
| 66_kidneyN2 | kidney normal | kidney normal | |
| 67_KidneyN3 | kidney normal | kidney normal | |
| 68_colonN1 | colon normal | colon normal | |
| 69_colonN2 | colon normal | colon normal | |
| 70_stomN | stomach normal | stomach normal | |
| 71_liverN | liver normal | liver normal | |
| 72_lungN1 | lung normal | lung normal | |
| 73_lungN2 | lung normal | lung normal | |
| 74_smallbowlN | small bowel normal | small bowel | |
| | | normal | |
| 75_brainN | brain normal mix | brain normal | |
| | | mix | |
| 76_heartN | heart normal mix | heart normal | |
| | | mix | |

TABLE 3
Tissue samples in normal panel:

| | | Sample | | |
|---|---|---|---|---|
| | | id (GCI)/case id | Tissue id | Sample id |
| | | (Asterand) Lot | (GCI)/Specimen | (Asterand)/RNA |
| sample name | Source | no. | id (Asternd) | id (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowel | GCI | V9L7D | V9L7DN6Z | |
| 7-GC-Small bowel | GCI | M3GVT | M3GVTN5R | |
| 8-GC-Small bowel | GCI | 196S2 | 196S2AJN | |
| 9-(9)-Am-Stomach | Ambion | 110P04A | | |
| 10-(10)-Bc-Stomach | Biochain | A501159 | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 16-As-Liver | Asterand | 7916 | 7203 | 7203B1 |
| 17-(28)-Am-Bladder | Ambion | 071P02C | | |
| 18-(29)-Bc-Bladder | Biochain | A504088 | | |
| 19-(64)-Am-Kidney | Ambion | 111P0101B | | |
| 20-(65)-Cl-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 25-(43)-Bc-Adrenal | Biochain | A610374 | | |
| 26-(16)-Am-Lung | Ambion | 111P0103A | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 36-GC-cervix | GCI | E2P2N | E2P2NAP4 | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am-Prostate | Ambion | 25955 | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 49-GC-Artery | GCI | YGTVY | YGTVYAIN | |
| 50-TH-Blood-PBMC | Tel- | 52497 | | |
| | Hashomer | | | |
| 51-TH-Blood-PBMC | Tel- | 31055 | | |
| | Hashomer | | | |
| 52-TH-Blood-PBMC | Tel- | 31058 | | |
| | Hashomer | | | |
| 53-(54)-Ic-Spleen | Ichilov | CG-267 | | |
| 54-(55)-Am-Spleen | Ambion | 111P0106B | 54-(55)-Am- | Ambion |
| | | | Spleen | |
| 56-(58)-Am-Thymus | Ambion | 101P0101A | | |
| 57-(60)-Bc-Thyroid | Biochain | A610287 | | |
| 58-(62)-Ic-Thyroid | Ichilov | CG-119-2 | | |
| 59-Gc-Sali gland | GCI | NNSMV | NNSMVNJC | |
| 60-(67)-Ic-Cerebellum | Ichilov | CG-183-5 | | |
| 61-(68)-Ic-Cerebellum | Ichilov | CG-212-5 | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

TABLE 4
Tissue samples in ovary panel

| | | | sample_id | | | | | | | |
|---|---|---|---|---|---|---|---|---|---|---|
| | | | (GCI)/ | | | | | | | |
| | | | case | | | | | | | |
| | | | id | RNA | | | | | | |
| | | | (Asterand)/ | ID | | | | | | |
| | | | lot | (GCI)/ | | | | | | |
| | | | no. | Sample | | | | | | |
| | Source/ | sample | (old | ID | | C | | | Ethnic | CA125 |
| Tissue | Delivery | name | samples) | (Asterand) | Diag | Stage | Tumor % | age | BG | PRE |
| OVC | Asterand | 1- | 23074 | 71900A2 | SA | I | 80 | 49 | CAU | |
| | | As- | | | | | | | | |
| | | SerSI | | | | | | | | |
| OVC | Asterand | 3- | 18700 | 40771B1 | SA | IB | 100 | 62 | WCAU | |
| | | As- | | | | | | | | |
| | | SerSIB | | | | | | | | |
| OVC | Asterand | 4- | 17646 | 32667B1 | SA | IB | 100 | 68 | W | |
| | | As- | | | | | | | | |
| | | SerSIB | | | | | | | | |
| OVC | Asterand | 5- | 15644 | 22996A1 | SA | IC | 100 | 48 | CAU | |
| | | As- | | | | | | | | |
| | | SerSIC | | | | | | | | |
| OVC | Asterand | 6- | 18701 | 40773C1 | SA | IIA | 100 | 59 | CAU | |
| | | As- | | | | | | | | |
| | | SerSIIA | | | | | | | | |
| OVC | GCI | 7- | 2O37O | | SA | IIB | 75 | 43 | WCAU | . |
| | | GC- | | | | | | | | |
| | | SerSIIB | | | | | | | | |
| OVC | GCI | 8- | 7B3DP | | SA | IIB | 70 | 70 | WCAU | . |
| | | GC- | | | | | | | | |
| | | SerSIIB | | | | | | | | |
| OVC | Asterand | 9- | 13268 | 19832A1 | SA | IIIC | 90 | 48 | C | |
| | | As- | | | | | | | | |
| | | SerSIIIC | | | | | | | | |
| OVC | GCI | 10- | 3NTIS | | SA | IIIC | 70 | 53 | WCAU | 70 |
| | | GC- | | | | | | | | |
| | | SerSIIIC | | | | | | | | |
| OVC | GCI | 11- | CEJUS | | SA | IIIC | 70 | 53 | WCAU | 4814 |
| | | GC- | | | | | | | | |
| | | SerSIIIC | | | | | | | | |
| OVC | GCI | 12- | 5NCLK | | SA | IIIC | 70 | 54 | WCAU | 209 |
| | | GC- | | | | | | | | |
| | | SerSIIIC | | | | | | | | |
| OVC | GCI | 13- | 1HI5H | | SA | IIIC | 90 | 61 | WCAU | 34 |
| | | GC- | | | | | | | | |
| | | SerSIIIC | | | | | | | | |
| OVC | GCI | 14- | 7RMHZ | | SA | IIIC | 80 | 63 | WCAU | . |
| | | GC- | | | | | | | | |
| | | SerSIIIC | | | | | | | | |
| OVC | GCI | 15- | 4WAAB | | SA | IIIC | 90 | 63 | WCAU | . |
| | | GC- | | | | | | | | |
| | | SerSIIIC | | | | | | | | |
| OVC | GCI | 16- | 79Z67 | | SA | IIIC | 85 | 67 | WCAU | . |
| | | GC- | | | | | | | | |
| | | SerSIIIC | | | | | | | | |
| OVC | GCI | 17- | DDSNL | | SA | IIIC | 70 | 68 | WCAU | . |
| | | GC- | | | | | | | | |
| | | SerSIIIC | | | | | | | | |
| OVC | GCI | 18- | DH8PH | | SA | IV | 95 | 70 | WCAU | . |
| | | GC- | | | | | | | | |
| | | SerSIV | | | | | | | | |
| OVC | GCI | 19- | E2WKF | | EA | IA | 70 | 30 | WCAU | . |
| | | GC- | | | | | | | | |
| | | EndoSIA | | | | | | | | |
| OVC | GCI | 20- | 5895C | | EA | IA | 95 | 39 | WCAU | . |
| | | GC- | | | | | | | | |
| | | EndoSIA | | | | | | | | |
| OVC | GCI | 21- | 533DX | | EA | IA | 95 | 50 | WCAU | 190 |
| | | GC- | | | | | | | | |
| | | EndoSIA | | | | | | | | |
| OVC | GCI | 22- | HZ2EY | | EA | IA | 90 | 55 | WCAU | 1078 |
| | | GC- | | | | | | | | |
| | | EndoSIA | | | | | | | | |
| OVC | GCI | 23- | RWOIV | | EA | IA | 65 | 47 | WCAU | 1695 |
| | | GC- | | | | | | | | |
| | | EndoSIA | | | | | | | | |
| OVC | GCI | 24- | 1U52X | | EA | IIA | 95 | 61 | WCAU | 275 |
| | | GC- | | | | | | | | |
| | | EndoSIIA | | | | | | | | |
| OVC | GCI | 25- | A17WS | | EA | IIB | 70 | 67 | WCAU | 78 |
| | | GC- | | | | | | | | |
| | | EndoSIIB | | | | | | | | |
| OVC | GCI | 26- | 1VT3I | | EA | IIIC | 90 | 50 | WCAU | . |
| | | GC- | | | | | | | | |
| | | EndoSIIIC | | | | | | | | |
| OVC | GCI | 27- | PZQXH | | EA | IIIC | 80 | 52 | WCAU | . |
| | | GC- | | | | | | | | |
| | | EndoSIIIC | | | | | | | | |
| OVC | GCI | 28- | I8VHZ | | EA | IV | 90 | 68 | WCAU | . |
| | | GC- | | | | | | | | |
| | | EndoSIV | | | | | | | | |
| OVC | GOG | 29- | 95- | | MC | IA | | 44 | | |
| | | (21)- | 10- | | | | | | | |
| | | GO- | G020 | | | | | | | |
| | | MucSIA | | | | | | | | |
| OVC | GCI | 30- | IMDA1 | | MA | IC | 70 | 41 | WCAU | 50 |
| | | GC- | | | | | | | | |
| | | MucSIC | | | | | | | | |
| OVC | Asterand | 31- | 12742 | 18920A1 | MA | IC | 70 | 61 | C | |
| | | As- | | | | | | | | |
| | | MucSIC | | | | | | | | |
| OVC | ABS | 32- | A0139 | | MC | IC | | 72 | Asian | |
| | | (22)- | | | | | | | | |
| | | AB- | | | | | | | | |
| | | MucSIC | | | | | | | | |
| OVC | GCI | 33- | NJM4U | | MA | IIA | 80 | 51 | WCAU | |
| | | GC- | | | | | | | | |
| | | MucSIIA | | | | | | | | |
| OVC | ABS | 34- | USA- | | PMC | IIIA | | 45 | C | |
| | | (20)- | 00273 | | | | | | | |
| | | AB- | | | | | | | | |
| | | MucSIIIA | | | | | | | | |
| OVC | GCI | 35- | RAFCW | | MA | IIIA | 75 | 55 | WCAU | 95 |
| | | GC- | | | | | | | | |
| | | MucSIIIA | | | | | | | | |
| OVC | Asterand | 36- | 23177 | 72888A1 | MA | IIIC | 60 | 52 | C | |
| | | As- | | | | | | | | |
| | | MucSIIIC | | | | | | | | |
| OVC | Asterand | 37- | 16103 | 29374B1 | MA | IIIC | 100 | 62 | W | |
| | | As- | | | | | | | | |
| | | MucSIIIC | | | | | | | | |
| OVC_BT | GCI | 38- | SC656 | | MBT | IA | 75 | 40 | WCAU | 138 |
| | | GC- | | | | | | | | |
| | | MucBorderSIA | | | | | | | | |
| OVC_BT | GCI | 39- | 3D5FO | | MBT | IA | 85 | 51 | WCAU | 19 |
| | | GC- | | | | | | | | |
| | | MucBorderSIA | | | | | | | | |
| OVC_BT | GCI | 40- | 7JP3F | | MBT | IA | 75 | 56 | WCAU | 125 |
| | | GC- | | | | | | | | |
| | | MucBorderSIA | | | | | | | | |
| OVC_B | GOG | 41- | 99- | | BMC | | | 32 | | 6 |
| | | (62)- | 10- | | | | | | | |
| | | Go- | G442 | | | | | | | |
| | | BenMuc | | | | | | | | |
| OVC_B | GCI | 43- | QLIKY | | BMC | | 100 | 42 | WCAU | |
| | | GC- | | | | | | | | |
| | | BenMuc | | | | | | | | |
| OVC_B | Asterand | 44- | 16870 | 30534A1 | BMC | | 100 | 45 | W | |
| | | As- | | | | | | | | |
| | | BenMuc | | | | | | | | |
| OVC_B | GOG | 45- | 99- | | BMC | | | 46 | | |
| | | (56)- | 01- | | | | | | | |
| | | GO- | G407 | | | | | | | |
| | | BenMuc | | | | | | | | |
| OVC_B | GCI | 47- | JO8W7 | | BMC | | 50 | 56 | WCAU | |
| | | GC- | | | | | | | | |
| | | BenMuc | | | | | | | | |
| OVC_B | Asterand | 48- | 17016 | 30645B1 | BSC | IA | 100 | 38 | C | |
| | | As- | | | | | | | | |
| | | BenSer | | | | | | | | |
| OVC_B | GOG | 49- | 99- | | BSC | | | 57 | | |
| | | (64)- | 06- | | | | | | | |
| | | GO- | G039 | | | | | | | |
| | | BenSer | | | | | | | | |
| OVC_B | GCI | 50- | DQQ2F | | BSCF | | 95 | 68 | WCAU | |
| | | GC- | | | | | | | | |
| | | BenSer | | | | | | | | |
| OVC_B | Asterand | 51- | 8786 | 8275A1 | BSC | | 100 | 80 | CAU | |
| | | As- | | | | | | | | |
| | | BenSer | | | | | | | | |
| OVC_NBM | Asterand | 52- | 15690 | 23054A1 | NO- | | | 52 | CAU | |
| | | As- | | | BM | | | | | |
| | | NBM | | | | | | | | |
| OVC_NBM | Asterand | 53- | 16843 | 30488A1 | NO- | | | 57 | W | |
| | | As- | | | BM | | | | | |
| | | NBM | | | | | | | | |
| OVC_NBM | Asterand | 54- | 16850 | 30496B1 | NO- | | | 65 | W | |
| | | As- | | | BM | | | | | |
| | | NBM | | | | | | | | |
| OVC_NBM | Asterand | 55- | 16848 | 30499C1 | NO- | | | 66 | CAU | |
| | | As- | | | BM | | | | | |
| | | NBM | | | | | | | | |
| OVC_N | GCI | 56- | WPU1U | | NO- | | 0 | 32 | WC | |
| | | GC- | | | PS | | | | | |
| | | NPS | | | | | | | | |
| OVC_N | GCI | 57- | Y9VHI | | NO- | | 0 | 35 | WCAU | |
| | | GC- | | | PS | | | | | |
| | | NPS | | | | | | | | |
| OVC_N | GCI | 58- | 76VM9 | | NO- | | 0 | 41 | WCAU | |
| | | GC- | | | PS | | | | | |
| | | NPS | | | | | | | | |
| OVC_N | GCI | 59- | DWHTZ | | NO- | | 0 | 42 | WCAU | |
| | | GC- | | | PS | | | | | |
| | | NPS | | | | | | | | |
| OVC_N | GCI | 60- | SJ2R2 | | NO- | | 0 | 43 | WCAU | |
| | | GC- | | | PS | | | | | |
| | | NPS | | | | | | | | |
| OVC_N | GCI | 61- | 9RQMN | | NO- | | 0 | 45 | WCAU | |
| | | GC-NPS | | | PS | | | | | |
| OVC_N | GCI | 62- | TOAE5 | | NO- | | 0 | 45 | WCAU | |
| | | GC-NPS | | | PS | | | | | |
| OVC_N | GCI | 63- | TW9PM | | NO- | | 0 | 46 | WCAU | |
| | | GC-NPS | | | PS | | | | | |
| OVC_N | GCI | 64- | 2VND2 | | NO- | | 0 | 46 | WCAU | |
| | | GC-NPS | | | PS | | | | | |
| OVC_N | GCI | 65- | L629F | | NO- | | 0 | 47 | WCAU | |
| | | GC-NPS | | | PS | | | | | |
| OVC_N | GCI | 66- | XLB23 | | NO- | | 0 | 47 | WCAU | |
| | | GC-NPS | | | PS | | | | | |
| OVC_N | GCI | 67- | IDUVY | | NO- | | 0 | 47 | WCAU | |
| | | GC-NPS | | | PS | | | | | |
| OVC_N | GCI | 68- | ZCXAD | | NO- | | 0 | 48 | WCAU | |
| | | GC-NPS | | | PS | | | | | |
| OVC_N | GCI | 69- | PEQ6C | | NO- | | 0 | 49 | WCAU | |
| | | GC-NPS | | | PS | | | | | |
| OVC_N | GCI | 70- | DD73B | | NO- | | 0 | 49 | WCAU | |
| | | GC-NPS | | | PS | | | | | |
| OVC_N | GCI | 71- | E2UF7 | | NO- | | 0 | 53 | WCAU | |
| | | GC-NPS | | | PS | | | | | |
| OVC_N | GCI | 72- | GWXUZ | | NO- | | 0 | 53 | WCAU | |
| | | GC-NPS | | | PS | | | | | |
| OVC_N | GCI | 73- | 4YG5P | | NO- | | 0 | 55 | WCAU | |
| | | GC-NPS | | | PS | | | | | |
| OVC_N | GCI | 74- | FDPL9 | | NO- | | 0 | 56 | WCAU | |
| | | GC-NPS | | | PS | | | | | |
| OVC_N | BioChain | 75- | A503274 | | NO- | | | 41 | Asian | |
| | | (45)- | | | PM | | | | | |
| | | Bc-NPM | | | | | | | | |
| OVC_N | BioChain | 76- | A504086 | | NO- | | | 41 | Asian | |
| | | (46)- | | | PM | | | | | |
| | | Bc-NPM | | | | | | | | |
| OVC_N | Ichilov | 77- | CG- | | NO- | | | 49 | | |
| | | (71)- | 188-7 | | PM | | | | | |
| | | Ic-NPM | | | | | | | | |
| OVC_N | BioChain | 78- | A504087 | | NO- | | | 51 | Asian | |
| | | (48)- | | | PM | | | | | |
| | | Bc-NPM | | | | | | | | |

| | | | | | Age | | | | | |
|---|---|---|---|---|---|---|---|---|---|---|
| | | | | | at | | Oral | Oral | | |
| | Menopausal | Mens | Preg | Preg | first | | Con | Con | Tubal | Recovery |
| Tissue | Status | Age | Times | Toterm | child | OC | Length | Unit | ligation | Type |
| OVC | Pre-M | | 2 | 1 | | | | | | Surg |
| OVC | Post-M | | 3 | 3 | | | | | | Surg |
| OVC | Post-M | | 9 | 2 | | | | | | Surg |
| OVC | M | | 4 | 2 | | | | | | Surg |
| OVC | Post-M | | 1 | 1 | | | | | | Surg |
| OVC | Pre-M | 12 | 0 | 0 | 0 | NO | . | | NO | Surg |
| OVC | Post-M | 14 | 5 | 3 | 20 | YES | 6 | months | NO | Surg |
| OVC | Post-M | | | | | | | | | Surg |
| OVC | Post-M | 12 | 1 | 1 | 26 | YES | 3 | months | NO | Surg |
| OVC | Pre-M | . | 2 | 2 | 30 | NO | . | | NO | Surg |
| OVC | Post-M | 13 | 2 | 2 | 21 | YES | 1 | years | NO | Surg |
| OVC | Post-M | 12 | 6 | 3 | 22 | NO | . | | NO | Surg |
| OVC | Post-M | 12 | 2 | 2 | 20 | YES | 10 | years | NO | Surg |
| OVC | Post-M | 11 | 2 | 1 | 29 | YES | 4 | years | NO | Surg |
| OVC | Post-M | 12 | 6 | 5 | 24 | YES | 2 | years | YES | Surg |
| OVC | Post-M | 11 | 4 | 4 | 19 | NO | . | | NO | Surg |
| OVC | Post-M | 13 | 4 | 3 | 20 | NO | . | | NO | Surg |
| OVC | Pre-M | 12 | 6 | 5 | 17 | YES | 6 | years | NO | Surg |
| OVC | Pre-M | 14 | 2 | 2 | 20 | NO | . | | NO | Surg |
| OVC | Pre-M | 11 | 0 | . | . | YES | 2 | years | NO | Surg |
| OVC | Pre-M | 13 | 0 | . | . | NO | . | | NO | Surg |
| OVC | Pre-M | 14 | 0 | . | . | NO | . | | NO | Surg |
| OVC | | . | . | . | . | | . | | | Surg |
| OVC | Post-M | 14 | 0 | . | . | NO | . | | NO | Surg |
| OVC | Pre-M | 12 | 2 | 2 | 24 | YES | 1 | years | NO | Surg |
| OVC | Pre-M | 11 | 0 | . | . | YES | 5 | years | NO | Surg |
| OVC | Post-M | . | 2 | 2 | 27 | NO | . | | NO | Surg |
| OVC | >100 | | | | | | | | | |
| OVC | Pre-M | 12 | 2 | 1 | 24 | NO | . | | | Surg |
| OVC | Post-M | | 3 | 3 | | | | | | Surg |
| OVC | | | | | | | | | | |
| OVC | | | | | | | | | | Surg |
| OVC | | | | | | | | | | |
| OVC | Post-M | 13 | 4 | 3 | 22 | NO | . | | NO | Surg |
| OVC | Pre-M | | | | | | | | | Surg |
| OVC | Post-M | | 1 | 1 | | | | | | Surg |
| OVC_BT | Pre-M | 13 | 2 | 2 | 23 | NO | . | | YES | Surg |
| OVC_BT | ? | 15 | 0 | . | . | NO | . | | NO | Surg |
| OVC_BT | Post-M | 14 | 3 | 3 | 19 | YES | 5 | years | NO | Surg |
| OVC_B | | | | | | | | | | |
| OVC_B | | | | | | | | | | Surg |
| OVC_B | Pre-M | | 2 | 2 | | | | | | Surg |
| OVC_B | | | | | | | | | | |
| OVC_B | | | | | | | | | | Surg |
| OVC_B | Pre-M | | 2 | 2 | | | | | | Surg |
| OVC_B | | | | | | | | | | |
| OVC_B | | | | | | | | | | Surg |
| OVC_B | Post-M | | 10 | 9 | | | | | | Surg |
| OVC_NBM | Pre-M | | 10 | 3 | | | | | | Surg |
| OVC_NBM | Post-M | | 4 | 2 | | | | | | Surg |
| OVC_NBM | Post-M | | 2 | 2 | | | | | | Surg |
| OVC_NBM | Post-M | | 9 | 2 | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | Surg |
| OVC_N | | | | | | | | | | |
| OVC_N | | | | | | | | | | |
| OVC_N | | | | | | | | | | |
| OVC_N | | | | | | | | | | |

TABLE 4_1

| Key | Full Name |
|---|---|
| A | Adenocarcinoma |
| APP | Adenocarcinoma from primary peritoneal |
| BMC | BENIGN MUCINOUS CYSTADENOMA |
| BSC | BENIGN SEROUS CYSTADENOMA |
| BSCF | BENIGN SEROUS CYSTADENOFIBROMA |
| C | Carcinoma |
| C Stage | Cancer stage |
| CAU | Caucasian |
| CCA | Clear cell adenocarcinoma |
| CNOS | Carcinoma NOS |
| EA | ENDOMETROID ADENOCARCINOMA |
| EA of BM | Endometroid adenocarcinoma of borderline malignancy |
| M | Menopausal |
| MA | MUCINOUS ADENOCARCINOMA |
| MBT | MUCINOUS BORDERLINE TUMOR |
| MC | Mucinous cystadenocarcinoma |
| MC Low M | Mucinous cystadenocarcinoma with low malignant |
| Mens. Age | Menstrual Age |
| Mixed . . . | Mixed epithelial cystadenocarcinoma with mucinous, |
| | endometrioid, squamous and papillary serous |
| MS & EA | Mixed serous and endometrioid adenocarcinoma |
| MS & EAM | Mixed serous and endometrioid adenocarcinoma of |
| | mullerian |
| NO-BM | NORMAL OVARY-BM |
| NO-PM | NORMAL OVARY-PM |
| NO-PS | NORMAL OVARY-PS |
| OC | Oral Contraceptive |
| OVC | Ovary Cancer |
| OVC_B | Ovary Benign |
| OVC_BT | Ovary Borderline Tumor |
| OVC_N | Ovary Normal |
| OVC_NBM | Ovary normal-benign matched |
| PA | Papillary adenocarcinoma |
| PC | Papillary cystadenocarcinoma |
| PEA | Papillary endometrioid adenocarcinoma |
| PMC | Papillary mucinous cystadenocarcinoma |
| Post-M | Post-menopausal |
| Pre-M | Pre-menopausal |
| PS & EC | Papillary serous and endometrioid cystadenocarcinoma |
| PSA | Papillary serous adenocarcinoma |
| PSC | Papillary serous carcinoma |
| SA | SEROUS ADENOCARCINOMA |
| SPC | Serous papillary cystadenocarcinoma |
| W | White |
| WCAU | WHITE/CAUCASIAN |

TABLE 5

| | |
|---|---|
| 3-Bcell1 | Details on Table 2 |
| 4-Bcell2 | Details on Table 2 |
| 5-J_Bcells | Details on Table 2 |
| 6-K_Bcells_act | Details on Table 2 |
| 26-Lym1 | Details on Table 2 |
| 27-Lym2 | Details on Table 2 |
| 30- | Details on Table 2 |
| NonHod_SCLym | |
| 31- | Details on Table 2 |
| NonHod_FolLym | |
| 32-Lym_Fol_GI | Details on Table 2 |
| 33-Lym_Fol_GII | Details on Table 2 |
| 34-Lym_Fol_GIII | Details on Table 2 |
| 35-MalLym1 | Details on Table 2 |
| 37-Lym_DifBCell1 | Details on Table 2 |
| 38-Lym_DifBCell2 | Details on Table 2 |
| 39-Lym_DifBCell3 | Details on Table 2 |
| 40- | Details on Table 2 |
| NonHod_Lym1 | |
| 41-MalLym4 | Details on Table 2 |
| 42- | Details on Table 2 |
| Lym_MantleCell1 | |
| 43- | Details on Table 2 |
| Lym_MantleCell2 | |
| 44- | Details on Table 2 |
| NonHod_Lym2 | |
| 47-BDCM | Details on Table 2 |
| 1-(7)-Bc-rectum | Details on Table 3 |
| 2-(8)-Bc-rectum | Details on Table 3 |
| 3-GC-colon | Details on Table 3 |
| 4-As-colon | Details on Table 3 |
| 5-As-colon | Details on Table 3 |
| 6-GC-small bowel | Details on Table 3 |
| 7-GC-small bowel | Details on Table 3 |
| 8-GC-small bowel | Details on Table 3 |
| 9-(9)-Am-stomach | Details on Table 3 |
| 11-(11)-Bc-esoph | Details on Table 3 |
| 12-(12)-Bc-esoph | Details on Table 3 |
| 13-As-panc | Details on Table 3 |
| 14-As-panc | Details on Table 3 |
| 16-As-liver | Details on Table 3 |
| 17-(28)-Am- | Details on Table 3 |
| bladder | |
| 18-(29)-Bc- | Details on Table 3 |
| bladder | |
| 22-GC-kidney | Details on Table 3 |
| 23-GC-kidney | Details on Table 3 |
| 25-(43)-Bc- | Details on Table 3 |
| adrenal | |
| 26-(16)-Am-lung | Details on Table 3 |
| 29-As-lung | Details on Table 3 |
| 30-As-lung | Details on Table 3 |
| 31-(75)-GC-ovary | Details on Table 3 |
| 32-(76)-GC-ovary | Details on Table 3 |
| 36-GC-cervix | Details on Table 3 |
| 38-(26)-Bc-uterus | Details on Table 3 |
| 39-(30)-Am- | Details on Table 3 |
| placen | |
| 40-(32)-Bc-placen | Details on Table 3 |
| 41-GC-breast | Details on Table 3 |
| 42-GC-breast | Details on Table 3 |
| 43-GC-breast | Details on Table 3 |
| 44-(38)-Am- | Details on Table 3 |
| prostate | |
| 45-Bc-prostate | Details on Table 3 |
| 46-As-testis | Details on Table 3 |
| 47-As-testis | Details on Table 3 |
| 49-GC-artery | Details on Table 3 |
| 50-TH-blood- | Details on Table 3 |
| PBMC | |
| 51-TH-blood- | Details on Table 3 |
| PBMC | |
| 52-TH-blood- | Details on Table 3 |
| PBMC | |
| 53-(54)-Ic-spleen | Details on Table 3 |
| 54-(55)-Am- | Details on Table 3 |
| spleen | |
| 56-(58)-Am- | Details on Table 3 |
| thymus | |
| 58-(62)-Ic-thyroid | Details on Table 3 |
| 59-Gc-sali gland | Details on Table 3 |
| 64-(72)-Ic-brain | Details on Table 3 |
| 65-(44)-Bc-heart | Details on Table 3 |
| 66-(46)-Ic-heart | Details on Table 3 |
| 69-GC-skel Mus | Details on Table 3 |

TABLE 6
Colon cancer testing panel

| | | | sample_id | | | | | | | | |
|---|---|---|---|---|---|---|---|---|---|---|---|
| | | | (GCI)/ | TISSUE | | | | | | | |
| | | | case id | ID | | | | | | | |
| | | | (Asterand)/ | (GCI)/ | | | | | | | |
| | | | lot no. | specimen | Sample | | | | | | |
| | Source/ | sample | (old | ID | ID | | Diag | Specimen | | | |
| Tissue | Delivery | name | samples | (Asterand) | (Asterand) | Diag | remarks | location | Gr | TNM | CS |
| CC | Asterand | 1-As- | 18036 | 31312 | 31312B1 | Aden | | Cec | 3 | TXN0M0 | 0 |
| | | AdenS0 | | | | | | | | | |
| CC | GCI | 2-GC- | 4QDH8 | 4QDH8ADT | | Aden | | DisC | | | I |
| | | AdenoSI | | | | | | | | | |
| CC | Ichilov | 3-(7)- | CG-235 | | | AI | | Rectum | UN | | I |
| | | Ic- | | | | | | | | | |
| | | AdenoSI | | | | | | | | | |
| CC | GCI | 4-GC- | NTAI8 | NTAI8AOU | | Aden | | Cec | | | I |
| | | AdenoSI | | | | | | | | | |
| CC | GCI | 5-GC- | ARA7P | ARA7PAQA | | Aden | | Ret, | | | I |
| | | AdenoSI | | | | | | LowAnt | | | |
| CC | Ichilov | 6- | CG-249 | | | UA | | | 3 | | IIA |
| | | (20)- | | | | | | | | | |
| | | Ic- | | | | | | | | | |
| | | AdenoSIIA | | | | | | | | | |
| CC | GCI | 7-GC- | AFTS6 | AFTS6AP6 | | Aden | | | | | IIA |
| | | AdenoSIIA | | | | | | | | | |
| CC | GCI | 8-GC- | 5CYDK | 5CYDKACS | | Aden | | | | | IIA |
| | | AdenoSIIA | | | | | | | | | |
| CC | GCI | 9-GC- | XKSLS | XKSLSAF7 | | Aden | | | | | IIA |
| | | AdenoSIIA | | | | | | | | | |
| CC | GCI | 10- | B4RU8 | B4RU8A8Q | | Aden | | | | | IIA |
| | | GC- | | | | | | | | | |
| | | AdenoSIIA | | | | | | | | | |
| CC | GCI | 11- | HB8EY | HB8EYA8I | | Aden | | | | | IIA |
| | | GC- | | | | | | | | | |
| | | AdenoSIIA | | | | | | | | | |
| CC | Ichilov | 12- | CG-229C | | | Aden | | | 2 | | II |
| | | (22)- | | | | | | | | | |
| | | AdenoSII | | | | | | | | | |
| CC | GCI | 13- | X8C7X | X8C7XATL | | Aden | | | | | IIA |
| | | GC- | | | | | | | | | |
| | | AdenoSIIA | | | | | | | | | |
| CC | GCI | 14- | HCP6K | HCP6KA8Z | | Aden | | | | | IIA |
| | | GC- | | | | | | | | | |
| | | AdenoSIIA | | | | | | | | | |
| CC | GCI | 15- | ZX4X7 | ZX4X7AXA | | Aden | | | | | IIA |
| | | GC- | | | | | | | | | |
| | | AdenoSIIA | | | | | | | | | |
| CC | Asterand | 16- | 17915 | 31176 | 31176A1 | Aden | | | 2-3 | T3N0M0 | IIA |
| | | As- | | | | | | | | | |
| | | AdenoSIIA | | | | | | | | | |
| CC | Ichilov | 17- | CG-335 | | | Aden | | Cec | 2 | | IIA |
| | | (1)- | | | | | | | | | |
| | | Ic- | | | | | | | | | |
| | | AdenoSIIA | | | | | | | | | |
| CC | Asterand | 19- | 12772 | 18885 | 18885A1 | Aden | | rectum | 2 | T3NXM0 | IIA |
| | | As- | | | | | | | | | |
| | | AdenoSIIA | | | | | | | | | |
| CC | GCI | 20- | JFYXP | JFYXPAMP | | Aden | | | | | IIA |
| | | GC- | | | | | | | | | |
| | | AdenoSIIA | | | | | | | | | |
| CC | GCI | 21- | OJXW9 | OJXW9ASR | | Aden | | | | | IIA |
| | | GC- | | | | | | | | | |
| | | AdenoSIIA | | | | | | | | | |
| CC | Ichilov | 22- | CG-284 | | | Aden | | sigma | 2 | | IIA |
| | | (28)- | | | | | | | | | |
| | | Ic- | | | | | | | | | |
| | | AdenoSIIA | | | | | | | | | |
| CC | Ichilov | 23- | CG-311 | | | Aden | | SigCol | 1-2 | | eIIA |
| | | (10)- | | | | | | | | | |
| | | Ic- | | | | | | | | | |
| | | AdenoSIIA | | | | | | | | | |
| CC | Ichilov | 24- | CG-222 | | | WPAden | | Rectum | | | III |
| | | (14)- | (2) | | | | | | | | |
| | | Ic- | | | | | | | | | |
| | | AdenoSIII | | | | | | | | | |
| CC | Ichilov | 25- | CG-282 | | | MA | | sigma | UN | | III |
| | | (23)- | | | | | | | | | |
| | | Ic- | | | | | | | | | |
| | | AdenoSIII | | | | | | | | | |
| CC | GCI | 26- | OTPI7 | OTPI7AWY | | Aden | | | | | III |
| | | GC- | | | | | | | | | |
| | | AdenoSIII | | | | | | | | | |
| CC | GCI | 27- | IG9NK | IG9NKAD3 | | MA | | | | | III |
| | | GC- | | | | | | | | | |
| | | AdenoSIII | | | | | | | | | |
| CC | GCI | 28- | 53OM7 | 53OM7AGL | | Aden | | | | | III |
| | | GC- | | | | | | | | | |
| | | AdenoSIII | | | | | | | | | |
| CC | GCI | 29- | BLUW6 | BLUW6A6Y | | Aden | | | | | III |
| | | GC- | | | | | | | | | |
| | | AdenoSIII | | | | | | | | | |
| CC | GCI | 30- | VZ6QA | VZ6QAAFA | | Aden | | RECTUM | | | III |
| | | GC- | | | | | | | | | |
| | | AdenoSIII | | | | | | | | | |
| CC | Ichilov | 31- | CG-303(3) | | | Aden | | | 1-2 | | III |
| | | (6)- | | | | | | | | | |
| | | Ic- | | | | | | | | | |
| | | AdenoSIII | | | | | | | | | |
| CC | Ichilov | 32- | CG-307 | | | Aden | | Cecum | 2 | | III |
| | | (2)- | | | | | | | | | |
| | | Ic- | | | | | | | | | |
| | | AdenoSIII | | | | | | | | | |
| CC | Ichilov | 33- | CG-337 | | | Aden | | | 1-2 | | III |
| | | (11)- | | | | | | | | | |
| | | Ic- | | | | | | | | | |
| | | AdenoSIII | | | | | | | | | |
| CC | Asterand | 34- | 18462 | 40971 | 40971A1 | TA | | SigCol | 2 | TXN2M0 | IIIC |
| | | As- | | | | | | | | | |
| | | AdenoSIIIC | | | | | | | | | |
| CC | Ichilov | 35- | CG-290 | | | Aden | | RectCol | 2 | | IV |
| | | (13)- | | | | | | | | | |
| | | Ic- | | | | | | | | | |
| | | AdenoSIV | | | | | | | | | |
| CC | GCI | 36- | 7D7QV | 7D7QVAE6 | | Aden | | | | | IV |
| | | GC- | | | | | | | | | |
| | | AdenoSIV | | | | | | | | | |
| CC | GCI | 37- | 38U4V | 38U4VAA4 | | Aden | | | | | IV |
| | | GC- | | | | | | | | | |
| | | AdenoSIV | | | | | | | | | |
| CC | Ichilov | 38- | CG-297 | | | Aden | | Rectum | 2 | | IV |
| | | (9)- | | | | | | | | | |
| | | Ic- | | | | | | | | | |
| | | AdenoSIV | | | | | | | | | |
| CB | GCI | 40- | IG3OY | IG3OYN7S | | TSAden | | RTCol | | | |
| | | GC- | | | | | | | | | |
| | | Ben | | | | | | | | | |
| CB | GCI | 41- | GKIEY | GKIEYAV4 | | TSAdenHGD | | ProxTCol | | | |
| | | GC- | | | | | | | | | |
| | | Ben | | | | | | | | | |
| CN | GCI | 42- | AGVTC | AGVTCNK7 | | NC | DIV | | | | |
| | | GC-NPS | | | | | | | | | |
| C | Asterand | 43- | 8956 | 9153 | 9153B1 | NC | | | | | |
| | | As-NPS | | | | | | | | | |
| CN | GCI | 44- | IG3OY | IG3OYN7S | | NC | | RTCol | | | |
| | | GC-NPS | | | | | | | | | |
| CN | GCI | 45- | K9OYX | K9OYXN4F | | NC | Divsw/FDIV | LTCol | | | |
| | | GC-NPS | | | | | | | | | |
| CN | Asterand | 46- | 23024 | 74445 | 74445B1 | NC | ChrDivs | | | | |
| | | As-NPS | | | | | | | | | |
| CN | Asterand | 47- | 23049 | 71410 | 71410B2 | NC | ChrDivs | | | | |
| | | As-NPS | | | | | | | | | |
| CN | GCI | 48- | G7JJX | G7JJXAX7 | | NC | Divsw/DIV . . . | SigCol | | | |
| | | GC-NPS | | | | | | | | | |
| CN | Asterand | 49- | 22900 | 74446 | 74446B1 | NC | ADw/AF | | | | |
| | | As-NPS | | | | | | | | | |
| CN | GCI | 50- | XVPZ2 | XVPZ2NDD | | NC | Div | | | | |
| | | GC-NPS | | | | | | | | | |
| CN | GCI | 51- | CDSUV | CDSUVNR3 | | NC | CU | | | | |
| | | GC-NPS | | | | | | | | | |
| CN | GCI | 52- | GP5KH | GP5KHAOC | | NC | Div | | | | |
| | | GC-NPS | | | | | | | | | |
| CN | GCI | 53- | YUZNR | YUZNRNDN | | NC | Divs | SigCol | | | |
| | | GC-NPS | | | | | | | | | |
| CN | GCI | 54- | 28QN6 | 28QN6NI1 | | NC | TSAden | RTCol | | | |
| | | GC-NPS | | | | | | | | | |
| CN | GCI | 55- | GV6N8 | GV6N8NG9 | | NC | Divs, | | | | |
| | | GC-NPS | | | | | PA | | | | |
| CN | GCI | 56- | ZJ17R | ZJ17RNIH | | NC | TubAden | RTCol | | | |
| | | GC-NPS | | | | | | | | | |
| CN | GCI | 57- | 2EEBJ | 2EEBJN2Q | | NC | Div/ | | | | |
| | | GC-NPS | | | | | ChrInfl | | | | |
| CN | GCI | 58- | 68IX5 | 68IX5N1H | | NC | ChrDiv | LTCol | | | |
| | | GC-NPS | | | | | | | | | |
| CN | GCI | 59- | 9GEGL | 9GEGLN1V | | NC | ExtDivs | SigCol | | | |
| | | GC-NPS | | | | | | | | | |
| CN | GCI | 60- | PKU8O | PKU8OAJ3 | | NC | Divs, | SigCol | | | |
| | | GC-NPS | | | | | ChrDiv . . . | | | | |
| CN | Asterand | 61- | 22903 | 74452 | 74452B1 | NC | MUw/MI | | | | |
| | | As-NPS | | | | | | | | | |
| CN | Asterand | 62- | 16364 | 31802 | 31802B1 | NC | UC | | | | |
| | | As-NPS | | | | | | | | | |
| CN | biochain | 63- | A607115 | | | N-PM | PM | | | | |
| | | (65)- | | | | | | | | | |
| | | Bc-NPM | | | | | | | | | |
| CN | Ambion | 64- | 071P10B | | | N-PM | PM | | | | |
| | | (71)- | | | | | | | | | |
| | | Am-NPM | | | | | | | | | |
| CN | biochain | 65- | A609262 | | | N-PM | PM | | | | |
| | | (66)- | | | | | | | | | |
| | | Bc-NPM | | | | | | | | | |
| CN | biochain | 66- | A609260 | | | N-PM | PM | | | | |
| | | (63)- | | | | | | | | | |
| | | Bc-NPM | | | | | | | | | |
| CN | biochain | 67- | A608273 | | | N-PM | PM | | | | |
| | | (62)- | | | | | | | | | |
| | | Bc-NPM | | | | | | | | | |
| CN | biochain | 68- | A609261 | | | N-PM | PM | | | | |
| | | (64)- | | | | | | | | | |
| | | Bc-NPM | | | | | | | | | |
| CN | biochain | 69- | A501156 | | | N-PM | PM | | | | |
| | | (41)- | | | | | | | | | |
| | | Bc-NPM | | | | | | | | | |
| CN | biochain | 70- | A406029 + | | | N-PMP10 | PM | | | | |
| | | (67)- | A411078 | | | | | | | | |
| | | Bc-NPM | | | | | | | | | |

| | | | | | | | Dr. | | | |
|---|---|---|---|---|---|---|---|---|---|---|
| | | | | | | Alcohol | per | Alc. | Recovery | Exc. |
| Tissue | CS2 | Tumor % | Gender | age | Ethnic B | Status | day | Dur. | Type | Y. |
| CC | | 80 | F | 43 | CAU | NU | | | Auto | 2004 |
| CC | Duke A | 85 | F | 44 | WCAU | Y | 4 | | Surg | |
| CC | Duke A | | F | 66 | | | | | | |
| CC | Duke B1 | 80 | M | 53 | WCAU | Y | . | | Surg | |
| CC | DukeB1 | 70 | F | 70 | WCAU | Y | 0 | | Surg | |
| CC | DukeB2 | | M | 36 | | | | | | |
| CC | DukeB2 | 75 | M | 39 | WCAU | N | 0 | | Surg | |
| CC | DukeB2 | 65 | M | 44 | WCAU | N | . | | Surg | |
| CC | DukeB2 | 65 | M | 48 | WCAU | Y | 10 | | Surg | |
| CC | DukeB2 | 65 | F | 50 | WCAU | N | . | | Surg | |
| CC | DukeB2 | 65 | M | 53 | WCAU | N | . | | Surg | |
| CC | DukeB | | F | 55 | | | | | | |
| CC | DukeB2 | 90 | M | 56 | WCAU | N | . | | Surg | |
| CC | DukeB2. | 80 | M | 58 | WCAU | Y | 4 | | Surg | |
| CC | DukeB2 | 90 | M | 60 | WCAU | Y | 5 | | Surg | |
| CC | DukeB2 | 60 | F | 64 | CAU | occ | 1 | 21-30 | Auto | 2004 |
| | | | | | | | drink/ | years | | |
| | | | | | | | week | | | |
| CC | DukeB2 | | F | 66 | | | | | | |
| CC | DukeB2 | 60 | F | 67 | CAU | NU | | | Surg | 2004 |
| CC | DukeB2 | 60 | F | 68 | WCAU | Y | . | | Surg | |
| CC | DukeB2 | 90 | F | 69 | WCAU | N | . | | Surg | |
| CC | DukeB2 | | F | 72 | | | | | | |
| CC | DukeB2 | | M | 88 | | | | | | |
| CC | DukeC | | F | 49 | | | | | | |
| CC | DukeC | | M | 51 | | | | | | |
| CC | DukeC2 | 70 | F | 54 | WCAU | N | . | | Surg | |
| CC | DukeC2 | 90 | F | 54 | WCAU | N | . | | Surg | |
| CC | DukeC2 | 75 | F | 61 | WCAU | N | . | | Surg | |
| CC | DukeC2 | 85 | F | 64 | WCAU | N | . | | Surg | |
| CC | DukeC2 | 60 | M | 67 | WCAU | Y | 14 | | Surg | |
| CC | DukeC2. | | F | 77 | | | | | | |
| CC | DukeC2. | | F | 89 | | | | | | |
| CC | DukeC2. | | NA | NA | | | | | | |
| CC | | 76 | F | 68 | CAU | NU | | | Surg | 2005 |
| CC | DukeD. | | M | 47 | | | | | | |
| CC | DukeD | 80 | F | 52 | WCAU | Y | 3 | | Surg | |
| CC | DukeD | 85 | F | 53 | WCAU | | . | | Surg | |
| CC | DukeD. | | M | 62 | | | | | | |
| CB | | | F | 48 | WCAU | Y | 1 | | Surg | |
| CB | | | F | 75 | WCAU | N | . | | Surg | |
| CN | | 0 | M | 45 | WCAU | N | . | | Surg | |
| C | | 0 | F | 46 | CAU | NU | | | Surg | 2002 |
| CN | | 0 | F | 48 | WCAU | Y | 1 | | Surg | |
| CN | | 0 | F | 50 | WCAU | N | . | | Surg | |
| CN | | 0 | F | 52 | CAU | Occ | | | Surg | 2005 |
| CN | | 0 | F | 52 | CAU | occ | | | Surg | 2005 |
| CN | | 0 | M | 52 | WCAU | N | . | | Surg | |
| CN | | 0 | M | 54 | CAU | CurU | | | Surg | 2005 |
| CN | | 0 | F | 55 | WCAU | N | . | | Surg | |
| CN | | 0 | M | 55 | WCAU | N | . | | Surg | |
| CN | | 0 | F | 57 | WCAU | Y | 6 | | Surg | |
| CN | | 0 | F | 57 | WCAU | Y | 1 | | Surg | |
| CN | | 0 | M | 59 | WCAU | Y | 42 | | Surg | |
| CN | | 0 | F | 61 | WCAU | Y | 3 | | Surg | |
| CN | | 0 | M | 61 | WCAU | Y | . | | Surg | |
| CN | | 0 | F | 66 | WCAU | Y | 4 | | Surg | |
| CN | | 0 | F | 66 | WCAU | N | . | | Surg | |
| CN | | 0 | M | 68 | WCAU | N | . | | Surg | |
| CN | | 0 | F | 69 | WCAU | N | . | | Surg | |
| CN | | 0 | M | 71 | CAU | Occ | | | Surg | 2005 |
| CN | | 0 | F | 74 | WCAU | Occ | | | Surg | 2004 |
| CN | | | M | 24 | | | | | | |
| CN | | | F | 34 | | | | | | |
| CN | | | M | 58 | | | | | | |
| CN | | | M | 61 | | | | | | |
| CN | | | M | 66 | | | | | | |
| CN | | | F | 68 | | | | | | |
| CN | | | M | 78 | | | | | | |
| CN | | | F&M | M(26-78) & F(53-77). | | | | | | |

TABLE 6

| Key | Full Name |
|---|---|
| CC | Colon Cancer |
| CB | Colon Benign |
| CN | Colon Normal |
| WT | Weight |
| HT | Height |
| Aden | Adenocarcinoma |
| AI | Adenocarcinoma intramucosal |
| UA | Ulcerated adenocarcinoma |
| WP Aden | Well polypoid adeocarcinoma |
| MA | Mucinus adenocarcinoma |
| TA | Tubular adenocarcinoma |
| Carc | Carcinoma |
| TS Aden | TUBULOVILLOUS ADENOMA |
| TS Aden HGD | TUBULOVILLOUS ADENOMA with HIGH GRADE |
| | DYSPLASIA |
| NC | Normal Colon |
| N-PM | Normal PM |
| N-PM P10 | Normal PM (Pool 10) |
| Diag | Diagnosis |
| Div | DIVERTICULITIS |
| Divs w/F DIV | Diverticulosis with Focal DIVERTICULITIS |
| Chr Divs | Chronic diverticulosis |
| Divs w/DIV . . . | DIVERTICULOSIS WITH DIVERTICULITIS AND |
| | FOCAL ABSCESS FORMATION; NO |
| | MALIGNANCY |
| AD w/AF | Acute diverticulitis with abscess formation |
| CU | CECAL ULCERATION |
| Divs, PA | DIVERTICULOSIS AND PERICOLIC ABSCESS |
| Tub Aden | TUBULAR ADENOMA |
| Div/Chr Infl | DIVERTICULOSIS/CHRONIC INFLAMMATION |
| Chr Div | CHRONIC DIVERTICULITIS |
| Ext Divs | EXTENSIVE DIVERTICULOSIS |
| Divs, Chr Div . . . | DIVERTICULOSIS AND CHRONIC |
| | DIVERTICULITIS, SEROSAL FIBROSIS AND |
| | CHRONIC SEROSITIS |
| MU w/MI | Mucosal ulceration with mural inflammation |
| UC | Ulcerative colitis |
| Cec | cecum |
| Dis C | DISTAL COLON |
| Ret, Low Ant | RETROSIGMOID, LOW ANTERIOR |
| Rect Col | Rectosigmoidal colon |
| Sig col | Sigmod colon |
| Col Sig | Colon Sigma |
| RT Col | RIGHT COLON |
| Prox T Col | PROXIMAL TRANSVERSE COLON |
| LT Col | Left Colon |
| Gr | Grade |
| CS | Cancer Stage |
| Ethnic B | Ethnic background |
| NU | Never Used |
| Occ | Occasion |
| Cur U | Current use |
| Dr. per day | Drinks per day |
| Alc. Dur. | Alcohol Duration |
| Auto. | Autopsy |
| Surg. | Surgical |
| Exc. Y. | Excision Year |

Materials and Experimental Procedures Used to Obtain Expression Data

RNA Preparation—

RNA was obtained from ABS (Wilmington, Del. 19801, USA, http://www.absbioreagents.com), BioChain Inst. Inc. (Hayward, Calif. 94545 USA www.biochain.com), GOG for ovary samples—Pediatric Cooperative Human Tissue Network, Gynecologic Oncology Group Tissue Bank, Children Hospital of Columbus (Columbus Ohio 43205 USA), Clontech (Franklin Lakes, N.J. USA 07417, www.clontech.com), Ambion (Austin, Tex. 78744 USA, http://www.ambion.com), Asterand (Detroit, Mich. 48202-3420, USA, www.asterand.com), AllCells, LLC. (Emeryville, Calif. 94608 USA, www.allcells.com), and from Genomics Collaborative Inc. a Division of Seracare (Cambridge, Mass. 02139, USA, www.genomicsinc.com). Alternatively, RNA was generated from blood cells, cell lines or tissue samples using TRI-Reagent (Molecular Research Center), according to Manufacturer's instructions. Tissue and RNA samples were obtained from patients or from postmortem. Total RNA of most samples were treated with DNaseI (Ambion).

RT PCR—Purified RNA (2-10 μg) was mixed with 300-1500 ng Random Hexamer primers (Invitrogen) and 500 μM dNTP in a total volume of 31.2 to 156 μl. The mixture was incubated for 5 min at 65° C. and then quickly chilled on ice. Thereafter, 10-50 μl of 5× SuperscriptII first strand buffer (Invitrogen), 4.8 to 24 μl 0.1M DTT and 80-400 units RNasin (Promega) were added, and the mixture was incubated for 10 min at 25° C., followed by further incubation at 42° C. for 2 min. Then, 2-10 μl (400-2000 units) of SuperscriptII (Invitrogen) was added and the reaction (final volume of 50-2500 was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis carried out as described below—cDNA (5 μl), prepared as described above, was used as a template in Real-Time PCR reactions (final volume of 20).11) using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min, following by dissociation step. Detection was performed by using the PE Applied Biosystem SDS 7000. The cycle in which the reactions achieved a threshold level of fluorescence (Ct=Threshold Cycle, described in detail below) was registered and was used to calculate the relative transcript quantity in the RT reactions. The relative quantity was calculated using the equation Q=efficiencŷ-Ct. The efficiency of the PCR reaction was calculated from a standard curve, created by using different dilutions of several reverse transcription (RT) reactions. To minimize inherent differences in the RT reaction, the resulting relative quantities were normalized using a normalization factor calculated in the following way:

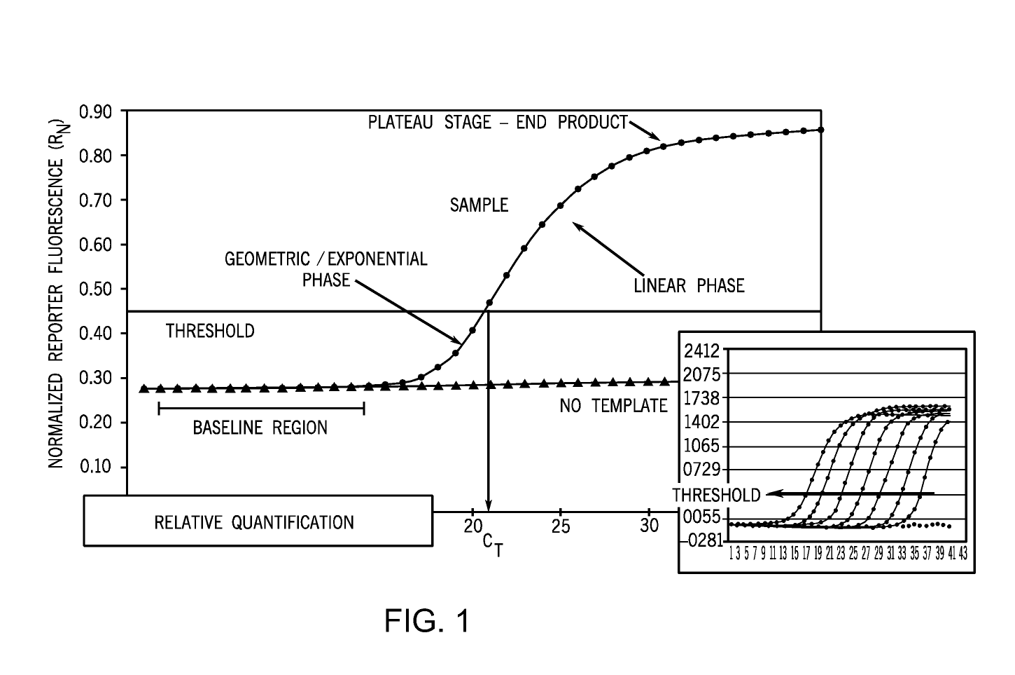

The expression of several housekeeping (HSKP) genes was checked on every panel. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 1. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It will be noted that this type of analysis provides relative quantification.

For each RT sample, the expression of the specific amplicon was normalized to the normalization factor calculated from the expression of different house keeping genes as described in section above. These house keeping genes are different for each panel. For ovary panel—SDHA (GenBank Accession No. NM_{—}004168 (SEQ ID NO:148); amplicon—SDHA-amplicon (SEQ ID NO:151)), HPRT1 (GenBank Accession No. NM_{—}000194 (SEQ ID NO:152) (amplicon—HPRT1-amplicon (SEQ ID NO:155)) and G6PD (GenBank Accession No. NM_{—}000402 (SEQ ID No:156); amplicon—G6PD amplicon (SEQ ID NO: 159)).). For normal panel—SDHA (GenBank Accession No. NM_{—}004168 (SEQ ID NO:148); amplicon—SDHA-amplicon (SEQ ID NO:151)), Ubiquitin (GenBank Accession No. BC000449 (SEQ ID No:164); amplicon—Ubiquitin-amplicon (SEQ ID NO:167)), and TATA box (GenBank Accession No. NM_{—}003194 (SEQ ID NO:160); TATA amplicon (SEQ ID NO:163)). For blood panel—HSB1L_HUMAN (Accession No. Q9Y450 (SEQ ID NO:132)), DHSA HUMAN (Accession No P31040 (SEQ ID NO:136)), SLC25A3 (Accession No Q7Z7N7 (SEQ ID NO:144)) and SFRS4_HUMSRP75A (Accession NO Q08170 (SEQ ID NO:140)). For colon panel—G6PD (GenBank Accession No. NM_{—}000402 (SEQ ID NO:156); G6PD amplicon (SEQ ID NO:159)). HPRT1 (GenBank Accession No. NM_{—}000194 (SEQ ID NO:152); amplicon—HPRT1-amplicon (SEQ ID NO:155) and PBGD (GenBank Accession No. BC019323 (SEQ ID NO:168); amplicon—PBGD-amplicon (SEQ ID NO:171). For blood and normal combined panel—HSB1L_HUMAN (Accession No. Q9Y450 (SEQ ID NO:132), DHSA HUMAN (Accession No P31040 (SEQ ID NO:136)), SLC25A3 (Accession No Q7Z7N7 (SEQ ID NO:144)), SFRS4_HUMSRP75A (Accession NO Q08170 (SEQ ID NO:140)) and TBP-TATA Box binding protein (Accession NO P20226 (SEQ ID NO:172)).

The sequences for primers and amplicons of the housekeeping genes measured in all samples detailed in Table 2 were as follows:

HSB1L_HUMAN (Accession No. Q9Y450 (SEQ ID NO: 132))
T05337_seg30-34F1-Forward primer (SEQ ID NO: 133):
GCTCCAGGCCATAAGGACTTC
T05337_seg30-34R1-Reverse primer (SEQ ID NO: 134):
CAGCTTCAAACTCTCCCCTGC
Amplicon (SEQ ID NO: 135):
GCTCCAGGCCATAAGGACTTCATTCCAAATATGATTACAGGAGCAGCCCAGGCGGATGTAG
CTGTTTTAGTTGTAGATGCCAGCAGGGGAGAGTTTGAAGCTG
DHSA_HUMAN (Accession No P31040 (SEQ ID NO: 136))
M78124_seg45-48F1-Forward primer (SEQ ID NO: 137):
TTCCTTGCCAGGACCTAGAG
M78124_seg45-48R1-Reverse primer (SEQ ID NO: 138):
CATAAACCTTTCGCCTTGAC
Amplicon (SEQ ID NO: 139):
TTCCTTGCCAGGACCTAGAGTTTGTTCAGTTCCACCCCACAGGCATATATGGTGCTGGTTG
TCTCATTACGGAAGGATGTCGTGGAGAGGGAGGCATTCTCATTAACAGTCAAGGCGAAAGG
TTTATG
SFRS4_HUMAN (Accession No Q08170 (SEQ ID NO: 140))
HUMSRP75Aseg30-33F1-Forward primer (SEQ ID NO: 141):
AATTTGTCAAGTCGGTGCAGC
HUMSRP75Aseg30-33R1-Reverse primer (SEQ ID NO: 142):
TCACCCCTTCATTTTTGCGT
Amplicon (SEQ ID NO: 143):
AATTTGTCAAGTCGGTGCAGCTGGCAAGACCTAAAGGATTATATGCGTCAGGCAGGAGAA
GTGACTTATGCAGATGCTCACAAGGGACGCAAAAATGAAGGGGTGA
SLC25A3 (Accession No Q7Z7N7 (SEQ ID NO: 144))
SSMPCPseg24-25-29F1-Forward primer (SEQ ID NO: 145):
CAGCCAGGTTATGCCAACAC
SSMPCPseg24-25-29R1-Reverse primer (SEQ ID NO: 146):
TCAAAGCAGGCGAACTTCATC
Amplicon (SEQ ID NO: 147):
CAGCCAGGTTATGCCAACACTTTGAGGGATGCAGCTCCCAAAATGTATAAGGAAGAAGGC
CTAAAAGCATTCTACAAGGGGGTTGCTCCTCTCTGGATGAGACAGATACCATACACCATG
ATGAAGTTCGCCTGCTTTGA

The sequences of the housekeeping genes measured in all the examples on ovary cancer tissue testing panel were as follows: SDHA (GenBank Accession No. NM_{—}004168 (SEQ ID NO:148):

SDHA Forward primer (SEQ ID NO: 149): TGGGAACAAGAGGGCATCTG
SDHA Reverse primer (SEQ ID NO: 150): CCACCACTGCATCAAATTCATG
SDHA-amplicon (SEQ ID NO: 151): TGGGAACAAGAGGGCATCTGCTAAAGTT
TCAGATTCCATTTCTGCTCAGTATCCAGTAGTGGATCATGAATTTGATGCAGTGGTGG
HPRT1 (GenBank Accession No. NM_000194 (SEQ ID NO: 152)),
HPRT1 Forward primer (SEQ ID NO: 153): TGACACTGGCAAAACAATGCA
HPRT1 Reverse primer (SEQ ID NO: 154): GGTCCTTTTCACCAGCAAGCT
HPRT1-amplicon (SEQ ID NO: 155): TGACACTGGCAAAACAATGCAGACTTT
GCTTTCCTTGGTCAGGCAGTATAATCCAAAGATGGTCAAGGTCGCAAGCTTGCTGGTG
AAAAGGACC
G6PD (GenBank Accession No. NM_000402) (SEQ ID NO: 156)
G6PD Forward primer (SEQ ID NO: 157): gaggccgtcaccaagaacat
G6PD Reverse primer (SEQ ID NO: 158): ggacagccggtcagagctc
G6PD-amplicon (SEQ ID NO: 159): gaggccgtcaccaagaacattcacgag
tcctgcatgagccagataggctggaaccgcatcatcgtggagaagccatcgggaggga
cctgcagagctctgaccggctgtcc

The sequences of the housekeeping genes measured in all the examples on normal tissue samples panel were as follows:

TATA box (GenBank Accession No. NM_003194) (SEQ ID NO: 160),
TATA box Forward primer (SEQ ID NO: 161): CGGTTTGCTGCGGTAATCAT
TATA box Reverse primer (SEQ ID NO: 162): TTTCTTGCTGCCAGTCTGGAC
TATAbox-amplicon (SEQ ID NO: 163): CGGTTTGCTGCGGTAATCATGAGGATAAGA
GAGCCACGAACCACGGCACTGATTTTCAGTTCTGGGAAAATGGTGTGCACAGGAGCCAA
GAGTGAAGAACAGTCCAGACTGGCAGCAAGAAA
Ubiquitin (GenBank Accession No. BC000449) (SEQ ID NO: 164)
Ubiquitin Forward primer (SEQ ID NO: 165): ATTTGGGTCGCGGTTCTTG
Ubiquitin Reverse primer (SEQ ID NO: 166): TGCCTTGACATTCTCGATGGT
Ubiquitin-amplicon (SEQ ID NO: 167) ATTTGGGTCGCGGTTCTTGTTTGTGGATC
GCTGTGATCGTCACTTGACAATGCAGATCTTCGTGAAGACTCTGACTGGTAAGACCATCACCC
TCGAGGTTGAGCCCAGTGACACCATCGAGAATGTCAAGGCA
SDHA (GenBank Accession No. NM_004168 (SEQ ID NO: 148))
SDHA Forward primer (SEQ ID NO: 149): TGGGAACAAGAGGGCATCTG
SDHA Reverse primer (SEQ ID NO: 150): CCACCACTGCATCAAATTCATG
SDHA-amplicon (SEQ ID NO: 151): TGGGAACAAGAGGGCATCTGCTAAAGTTTCAG
ATTCCATTTCTGCTCAGTATCCAGTAGTGGATCATGAATTTGATGCAGTGGTGG

The sequences of the housekeeping genes measured in all the examples on colon cancer tissue testing panel were as follows:

PBGD (GenBank Accession No. BC019323 (SEQ ID NO: 168)),
PBGD Forward primer (SEQ ID NO: 169): TGAGAGTGATTCGCGTGGG
PBGD Reverse primer (SEQ ID NO: 170): CCAGGGTACGAGGCTTTCAAT
PBGD-amplicon (SEQ ID NO: 171): TGAGAGTGATTCGCGTGGGTACCCG
CAAGAGCCAGCTTGCTCGCATACAGACGGA
CAGTGTGGTGGCAACATTGAAAGCCTCGTACCCTGG
HPRT1 (GenBank Accession No. NM_000194 (SEQ ID NO: 152)),
HPRT1 Forward primer (SEQ ID NO: 153): TGACACTGGCAAAACAATGCA
HPRT1 Reverse primer (SEQ ID NO: 154): GGTCCTTTTCACCAGCAAGCT
HPRT1-amplicon (SEQ ID NO: 155): TGACACTGGCAAAACAATGCAGACTT
TGCTTTCCTTGGTCAGGCAGTATAATCCA
AAGATGGTCAAGGTCGCAAGCTTGCTGGTGAAAAGGACC
G6PD (GenBank Accession No. NM_000402) (SEQ ID NO: 156)
G6PD Forward primer (SEQ ID NO: 157): gaggccgtcaccaagaacat
G6PD Reverse primer (SEQ ID NO: 158): ggacagccggtcagagctc
G6PD-amplicon (SEQ ID NO: 159): gaggccgtcaccaagaacattcacgag
tcctgcatgagccagataggctggaaccgcatcatcgtggagaagccatcgggaggga
cctgcagagctctgaccggctgtcc

The sequences of the housekeeping genes measured in all the examples of combined panel presented in Table 5 were the four genes used for the blood panel (Table 2) and an additional housekeeping gene:

TBP (TATA Box binding protein) (Accession No P20226 (SEQ ID NO: 172))
HSTFIIDX seg7-9F1-Forward primer (SEQ ID NO: 173):
AACATCATGGATCAGAACAACAGC
HSTFIIDX seg7-9R1-Reverse primer (SEQ ID NO: 174):
ATCATTGGACTAAAGATAGGGATTCC
Amplicon (SEQ ID NO: 174):
AACATCATGGATCAGAACAACAGCCTGCCACCTTACGCTCAGGGCTTGGCCTCCC
CTCAGGGTGCCATGACTCCCGGAATCCCTATCTTTAGTCCAATGAT

Another methodology used to predict the expression pattern of the proteins of the invention was MED discovery engine:

MED is a platform for collection of public gene-expression data, normalization, annotation and performance of various queries. Expression data from the most widely used Affymetrix microarrays is downloaded from the Gene Expression Omnibus (GEO—www.ncbi.nlm.nih.gov/GEO). Data is multiplicatively normalized by setting the 95 percentile to a constant value (normalized expression=1200), and noise is filtered by setting the lower 30% to 0. Experiments are annotated, first automatically, and then manually, to identify tissue and condition, and chips are grouped according to this annotation, and cross verification of this grouping by comparing the overall expression pattern of the genes of each chip to the overall average expression pattern of the genes in this group. Each probeset in each group is assigned an expression value which is the median of the expressions of that probeset in all chips included in the group. The vector of expression of all probesets within a certain group is the virtual chip of that group, and the collection of all such virtual chips is a virtual panel. The panel (or sub-panels) can be queried to identify probesets with a required behavior (e.g. specific expression in a sub-set of tissues, or differential expression between disease and healthy tissues). These probesets are linked to LEADS contigs and to RefSeqs (http://www.ncbi.nlm.nih.gov/RefSeq/) by probe-level mapping, for further analysis.

The Affymetrix platforms that are downloaded are HG-U95A and the HG-U133 family (A, B, A2.0 and PLUS 2.0). Than three virtual panels were created: U95 and U133 Plus 2.0, based on the corresponding platforms, and U133 which uses the set of common probesets for HG-U133A, HG-U133A2.0 and HG-U133 PLUS 2.0+.

The results of the MED discovery engine are presented in scatter plots. The scatter plot is a compact representation of a given panel (collection of groups). The y-axis is the (normalized) expression and the x-axis describes the groups in the panel. For each group, the median expression is represented by a solid marker, and the expression values of the different chips in the group are represented by small dashes (“-”). The groups are ordered and marked as follows—“Other” groups (e.g. benign, non-cancer diseases, etc.) with a triangle, Treated cells with a square, Normal with a circle, Matched with a cross, and Cancer with a diamond. The number of chips in each group is also written adjacent to it's name.

### Example 2

### KIAA0746 Polypeptides and Polynucleotides, and Uses Thereof as a Drug Target for Producing Drugs and Biologics

### Example 2_{—}1

### Description for Cluster Z43375

As noted supra, the present invention relates to KIAA0746 polypeptides, novel splice variants and diagnostics and therapeutics based thereon, especially but not exclusively antibody-based diagnostics and therapeutics. With respect thereto, a known wild type KIAA0746 nucleic acid sequence has been reported in various patent references. For example, the sequence of the known KIAA0746 is disclosed in US20070020666. This US application alleges that the known KIAA0746 is differentially expressed in large granular lymphocyte leukemias (LGL).

In addition, WO06034573, WO05080601 and WO03083140 list the known KIAA0746 transcript among many other genes. WO06034573 mentions the use of the disclosed genes in screens for identifying agonists or antagonists thereof that may be used in treatment of hematological malignancies, however, antibodies are not enumerated. WO05080601 is predominantly focused on diagnostic applications of the disclosed genes specific for acute myeloid leukemia (AML). WO03083140, is focused on differentially expressed genes specific for AML and the diagnostic applications thereof in disease detection and prognosis, also seems to suggest the screening of drug candidates including antibodies for selection of potential therapeutic agonist or antagonists for treatment of AML. However, there is no explicit mention of the use of antibodies against the known KIAA0746 for treatment of B cell malignancies.

KIAA0746 was previously identified as a “hypothetical” protein (hypothetical protein LOC23231 (SEQ ID NO:14)) that was discovered by The National Institutes of Health Mammalian Gene Collection (MGC) Program, a multiinstitutional effort to identify and sequence a cDNA clone containing a complete ORF for each human and mouse gene (Strausberg R L et al. Proc Natl Acad Sci USA. 2002 Dec. 24; 99(26):16899-903) and in the sequences of 100 cDNA clones from a set of size-fractionated human brain cDNA libraries (Nagase T et al. DNA Res. 1998 Oct. 30; 5(5):277-86). The hypothetical protein is annotated in NCBI as having a TPR repeat and belonging to SELL subfamily. A recent article showed the association of where KIAA0746 resides with bipolar disorder and schizophrenia (Christoforou A et al. Mol Psychiatry. 2007 November; 12(11):1011-25). According to the present invention, KIAA0746 was predicted to be a type I membrane protein. According to the present invention, KIAA0746 was shown to be overexpressed in B-cells and Dendritic cells, and in several types of lymphomas, as well as a variety of solid tumors, including ovary, pancreas, prostate, liver, kidney, colon, head & neck, lung and melanoma.

Cluster Z43375 (internal ID 76553061) features 13 transcripts of interest, the names for which are given in Table 7. The selected protein variants are given in table 8.

TABLE 7
Transcripts of interest
Transcript Name

Z43375_1_T0 (SEQ ID NO: 1)
Z43375_1_T3 (SEQ ID NO: 2)
Z43375_1_T6 (SEQ ID NO: 3)
Z43375_1_T7 (SEQ ID NO: 4)
Z43375_1_T14 (SEQ ID NO: 5)
Z43375_1_T16 (SEQ ID NO: 6)
Z43375_1_T20 (SEQ ID NO: 7)
Z43375_1_T22 (SEQ ID NO: 8)
Z43375_1_T23 (SEQ ID NO: 9)
Z43375_1_T28 (SEQ ID NO: 10)
Z43375_1_T30 (SEQ ID NO: 11)
Z43375_1_T31 (SEQ ID NO: 12)
Z43375_1_T33 (SEQ ID NO: 13)

TABLE 8
Proteins of interest

| Protein Name | | Corresponding Transcript(s) | |
|---|---|---|---|
| Z43375_1_P4 | (SEQ ID NO: 18) | Z43375_1_T0 | (SEQ ID NO: 1) |
| Z43375_1_P8 | (SEQ ID NO: 19) | Z43375_1_T3 | (SEQ ID NO: 2) |
| Z43375_1_P40 | (SEQ ID NO: 20) | Z43375_1_T6 | (SEQ ID NO: 3) |
| Z43375_1_P46 | (SEQ ID NO: 21) | Z43375_1_T14 | (SEQ ID NO: 5) |
| Z43375_1_P47 | (SEQ ID NO: 22) | Z43375_1_T16 | (SEQ ID NO: 6) |
| Z43375_1_P50 | (SEQ ID NO: 23) | Z43375_1_T20 | (SEQ ID NO: 7) |
| Z43375_1_P51 | (SEQ ID NO: 24) | Z43375_1_T22 | (SEQ ID NO: 8) |
| Z43375_1_P52 | (SEQ ID NO: 25) | Z43375_1_T23 | (SEQ ID NO: 9) |
| Z43375_1_P53 | (SEQ ID NO: 26) | Z43375_1_T28 | (SEQ ID NO: 10) |
| Z43375_1_P54 | (SEQ ID NO: 27) | Z43375_1_T30 | (SEQ ID NO: 11) |
| Z43375_1_P55 | (SEQ ID NO: 28) | Z43375_1_T31 | (SEQ ID NO: 12) |
| Z43375_1_P56 | (SEQ ID NO: 29) | Z43375_1_T33 | (SEQ ID NO: 13) |
| Z43375_1_P60 | (SEQ ID NO: 30) | Z43375_1_T7 | (SEQ ID NO: 4) |

These sequences are variants of the known protein hypothetical protein LOC23231 (SwissProt accession identifier NP_{—}056002; KIAA0746 (SEQ ID NO:14)), referred to herein as the previously known protein.

As noted above, cluster Z43375 features 13 transcript(s), which were listed in Table 7 above. These transcripts encode for proteins which are variants of protein hypothetical protein LOC23231 (SEQ ID NO:14). A description of each variant protein according to the present invention is now provided.

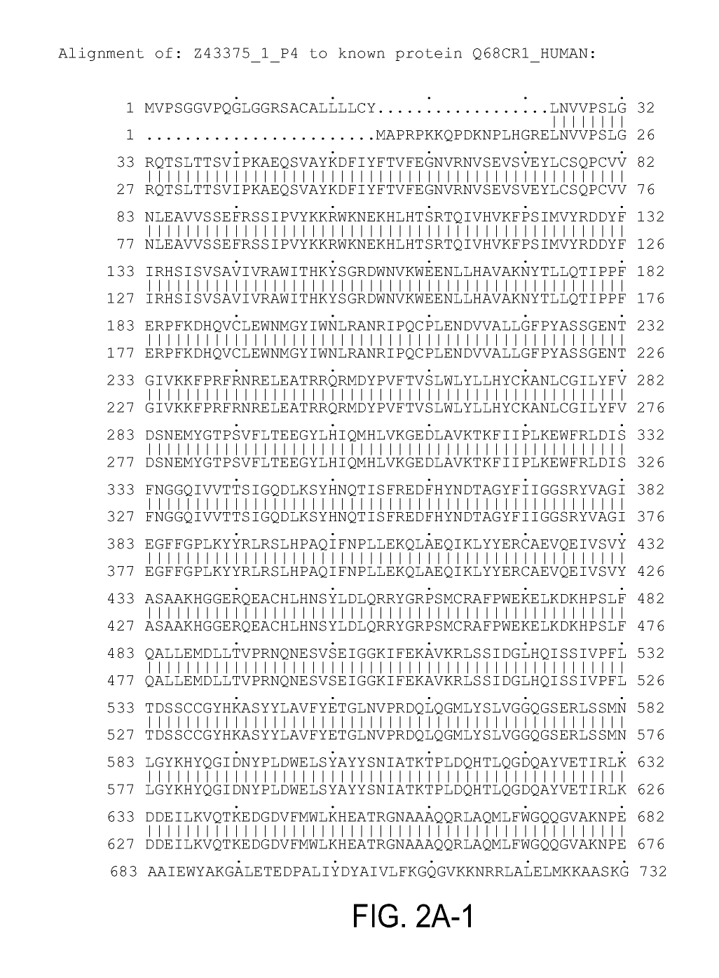

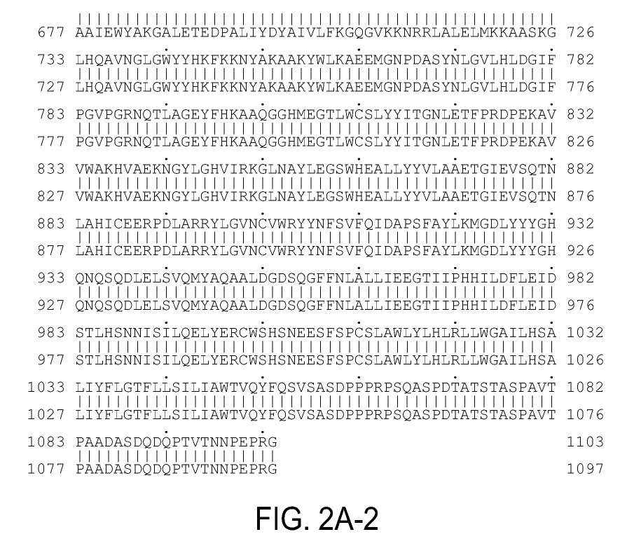

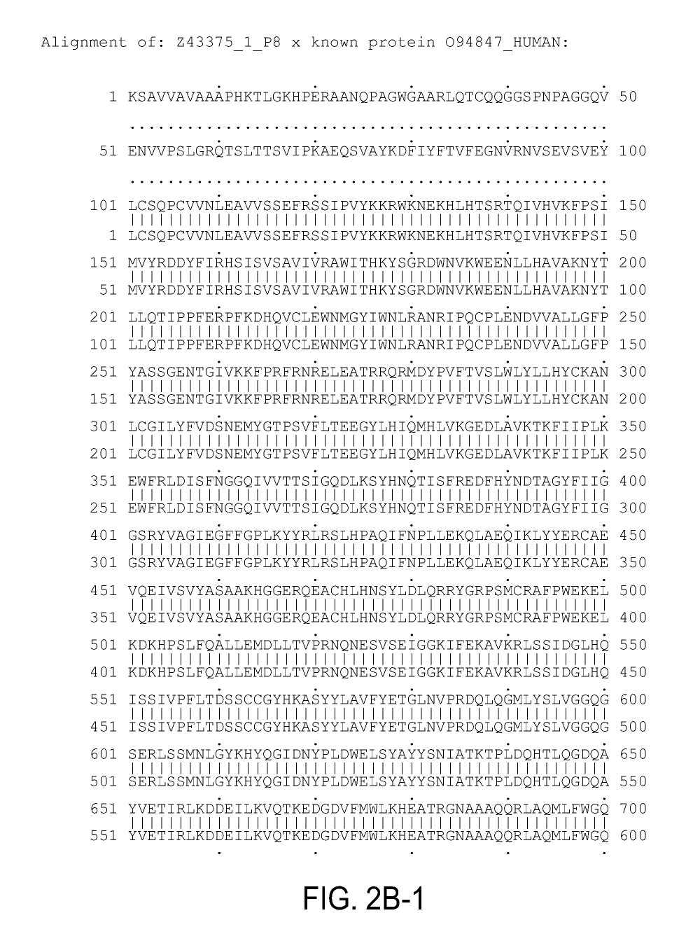

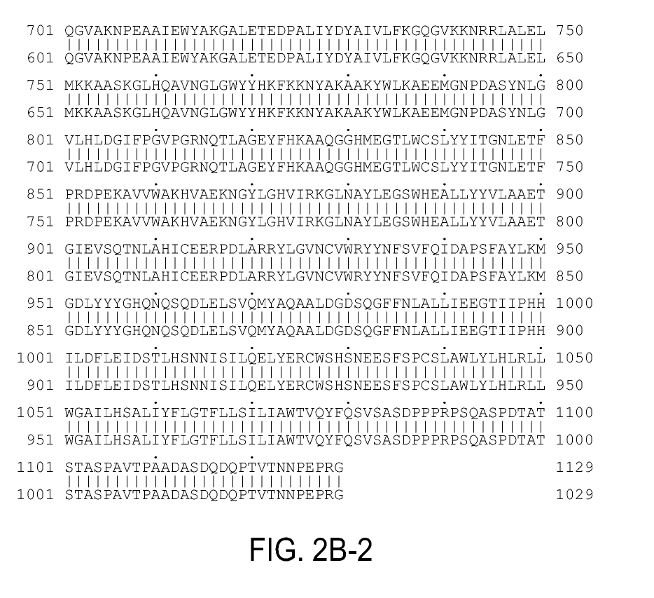

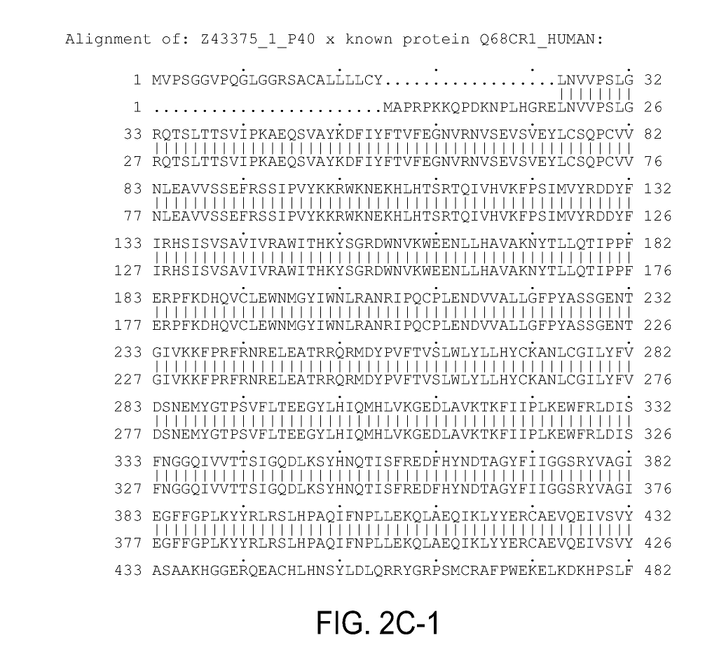

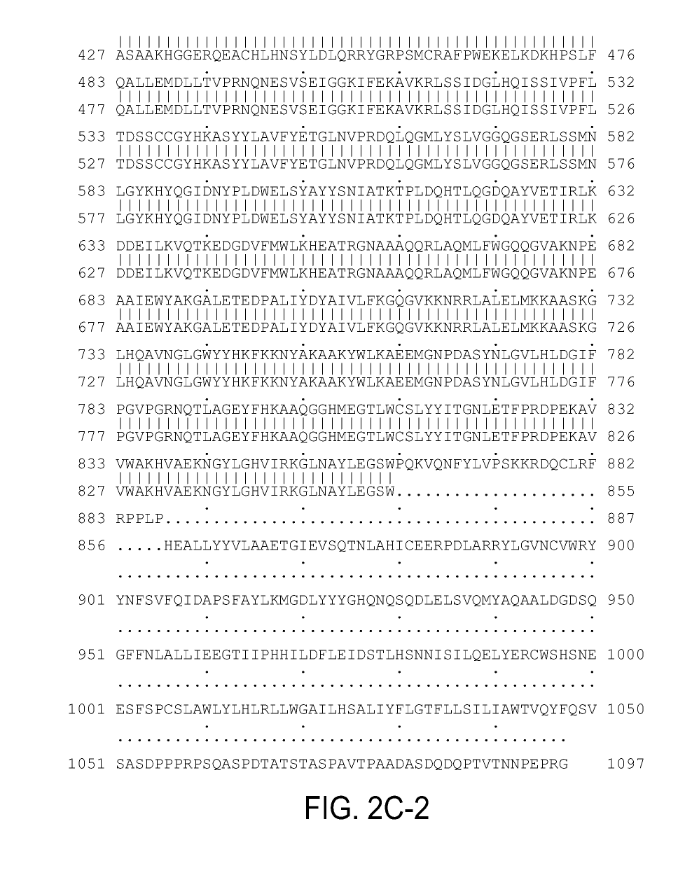

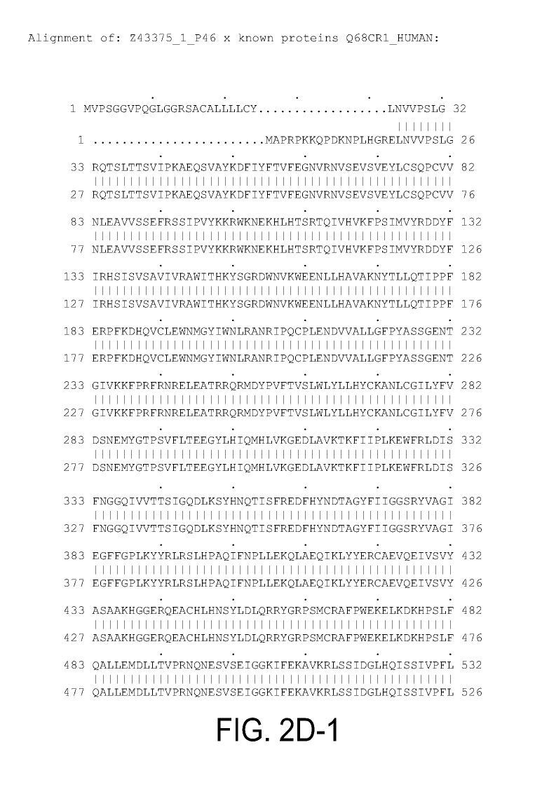

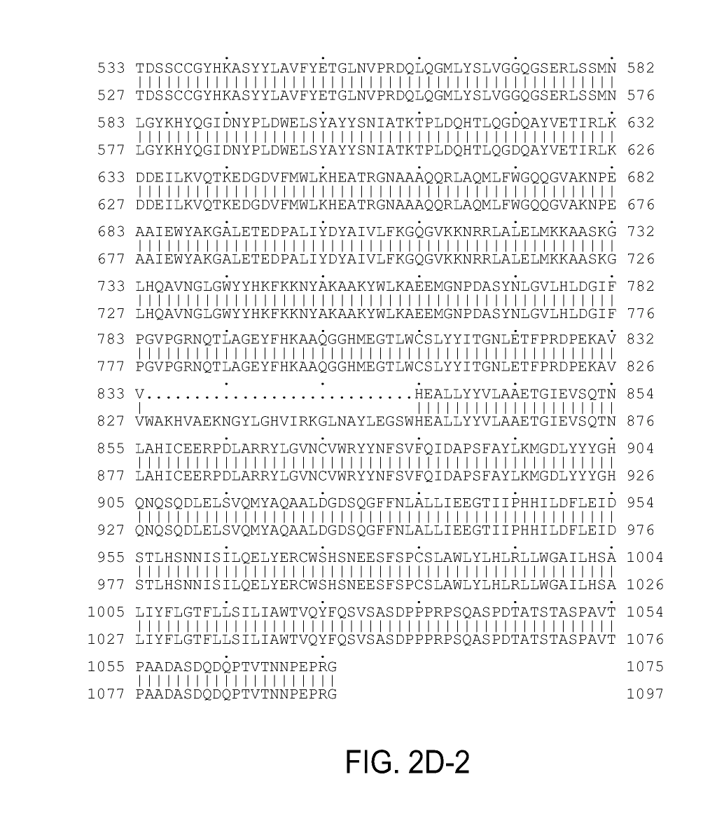

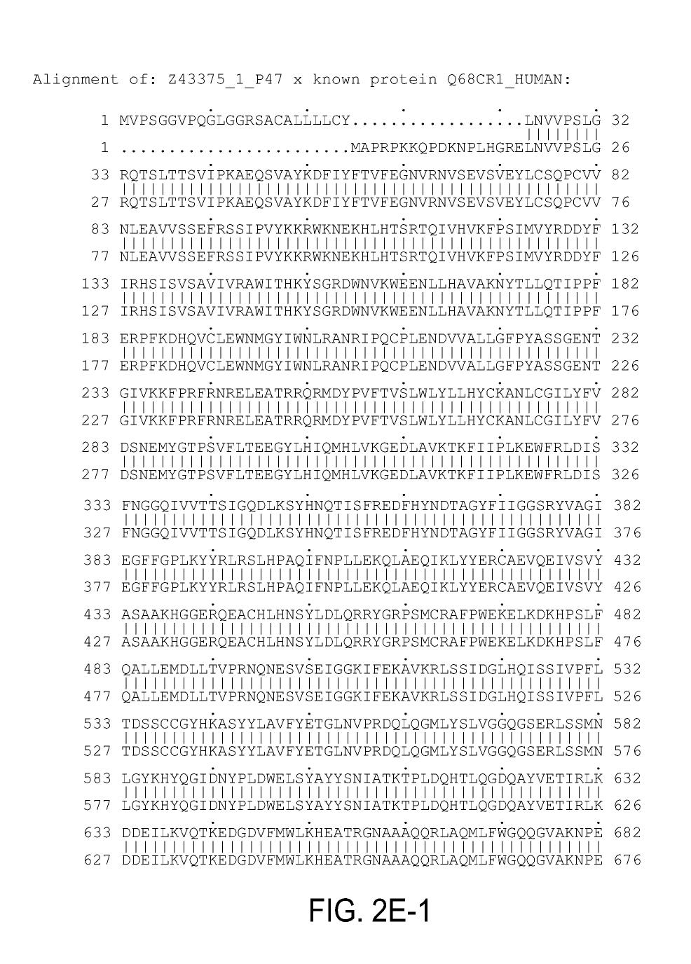

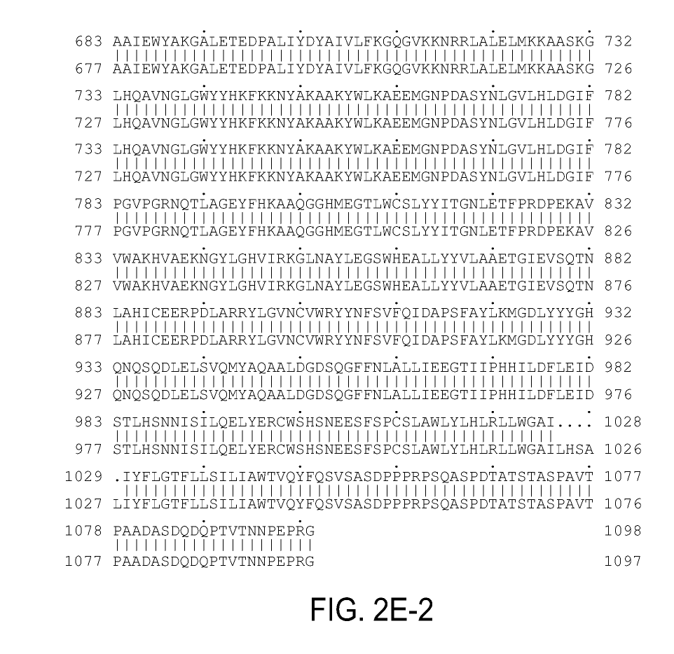

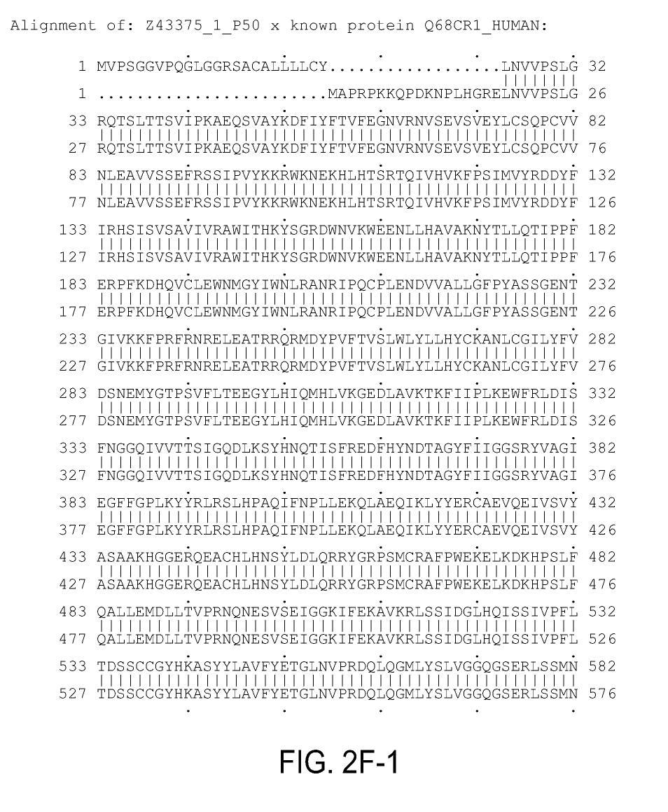

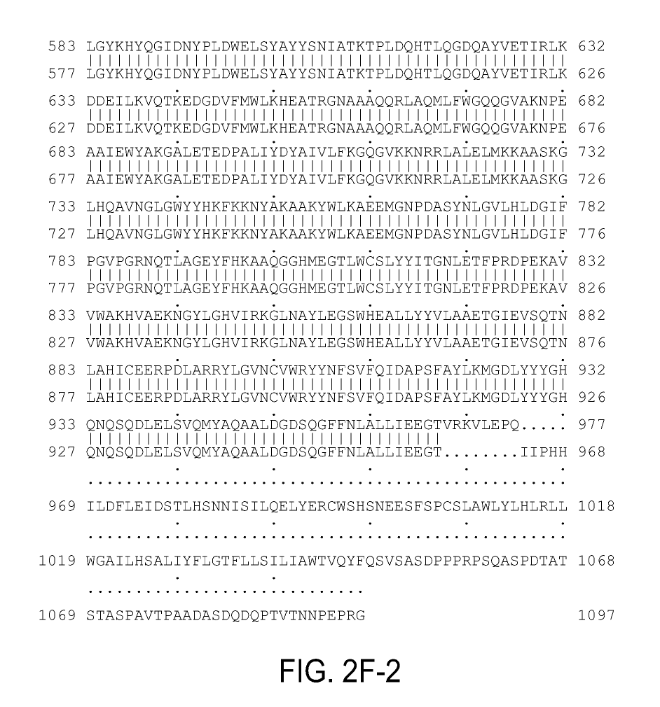

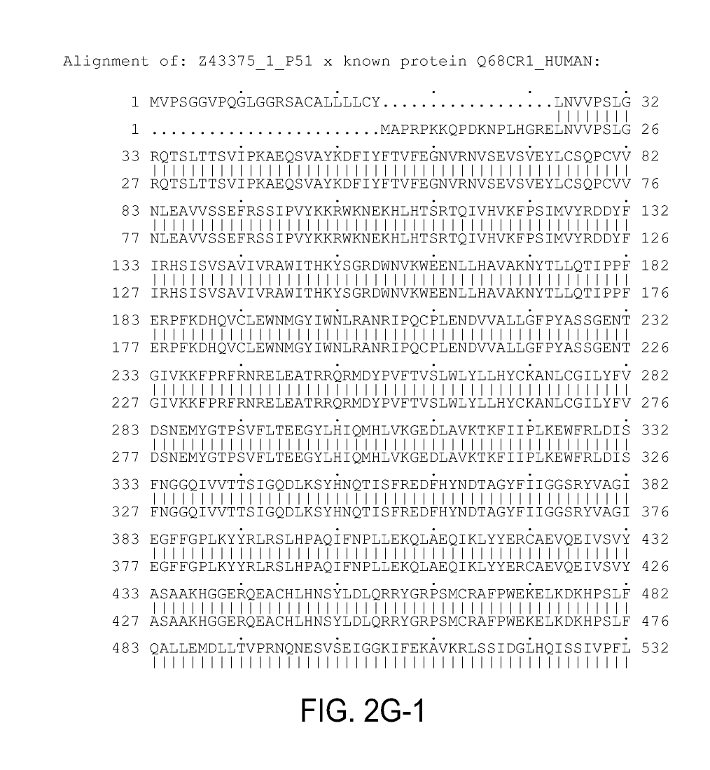

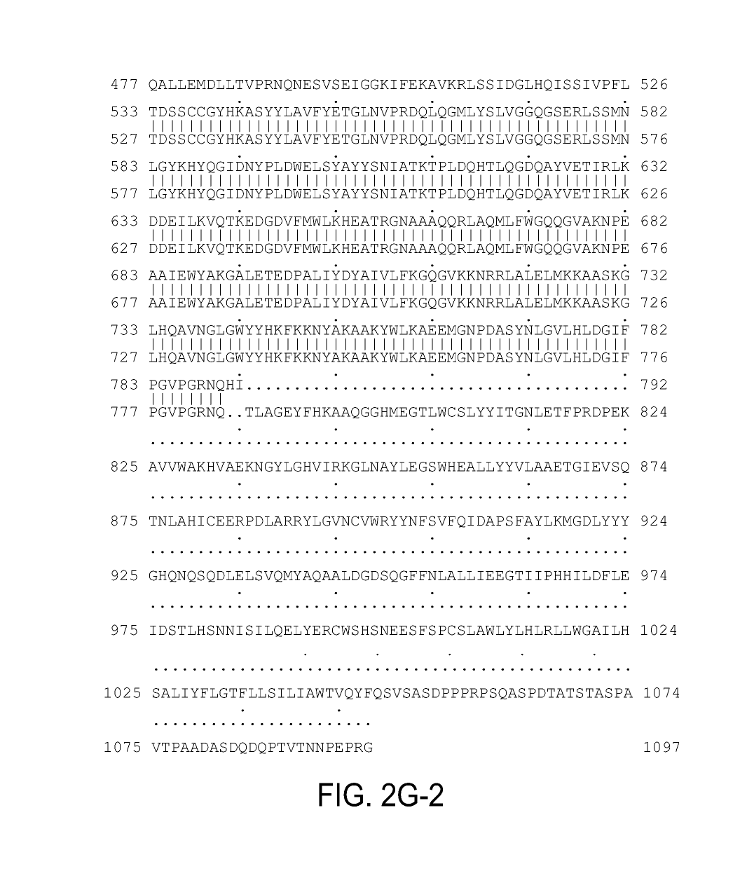

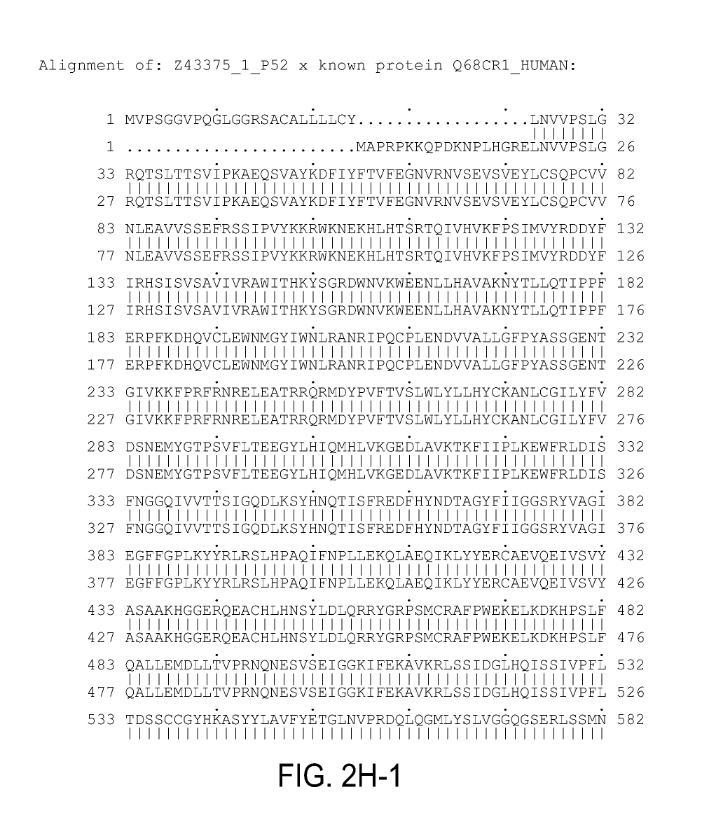

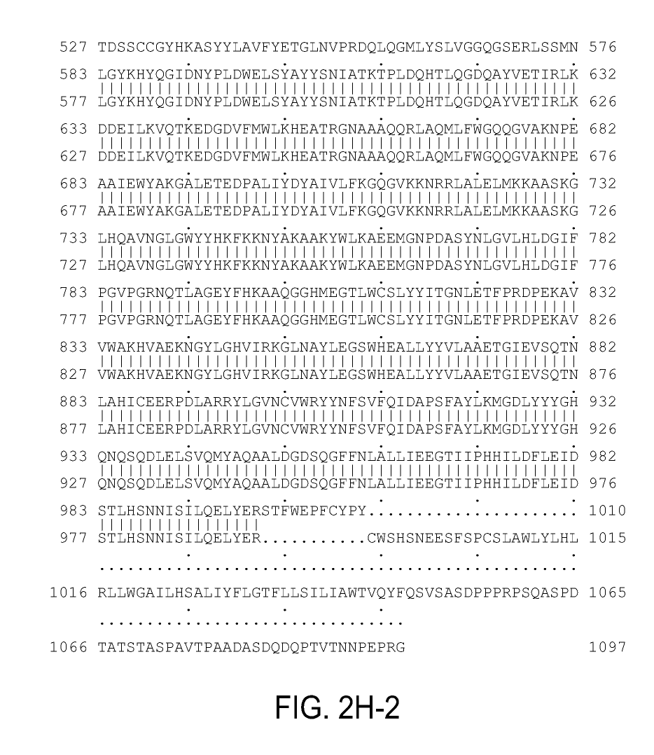

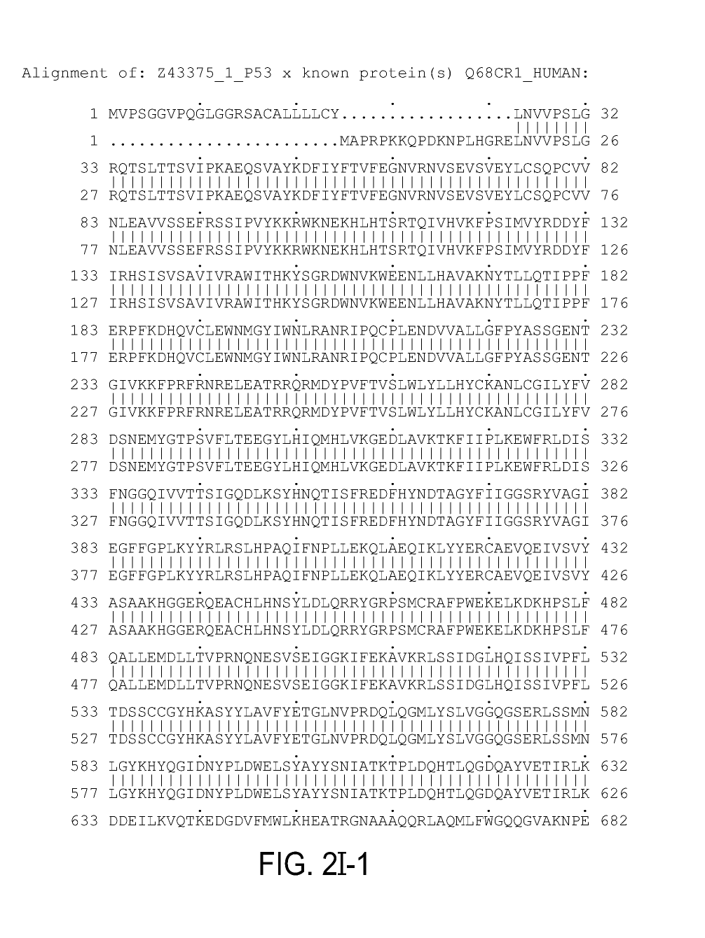

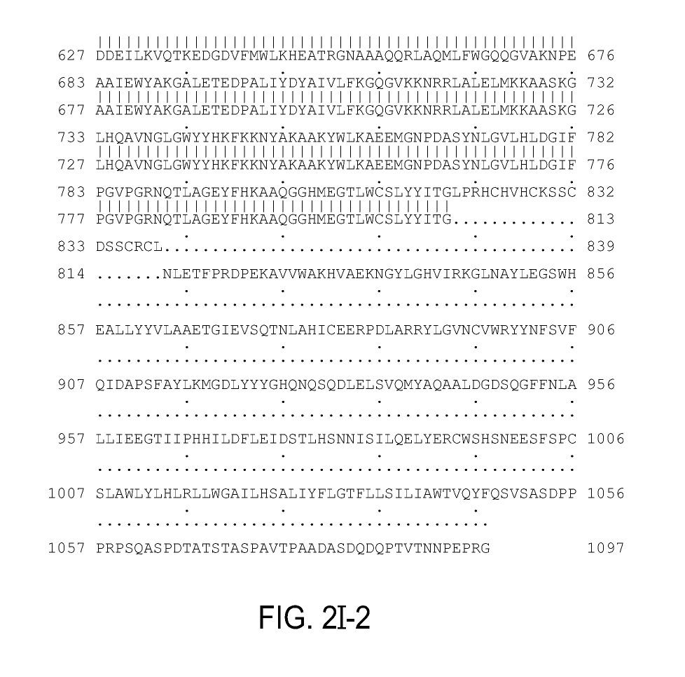

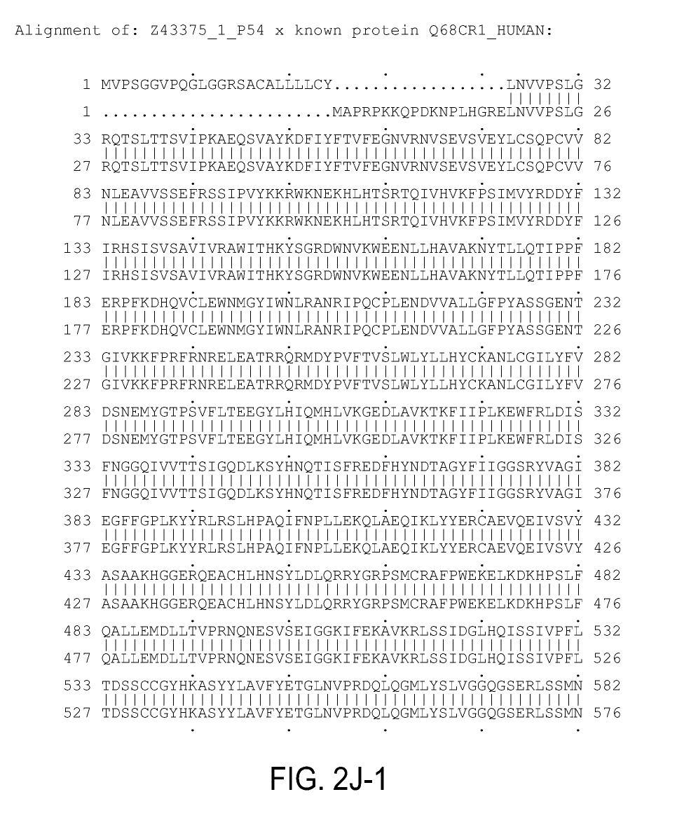

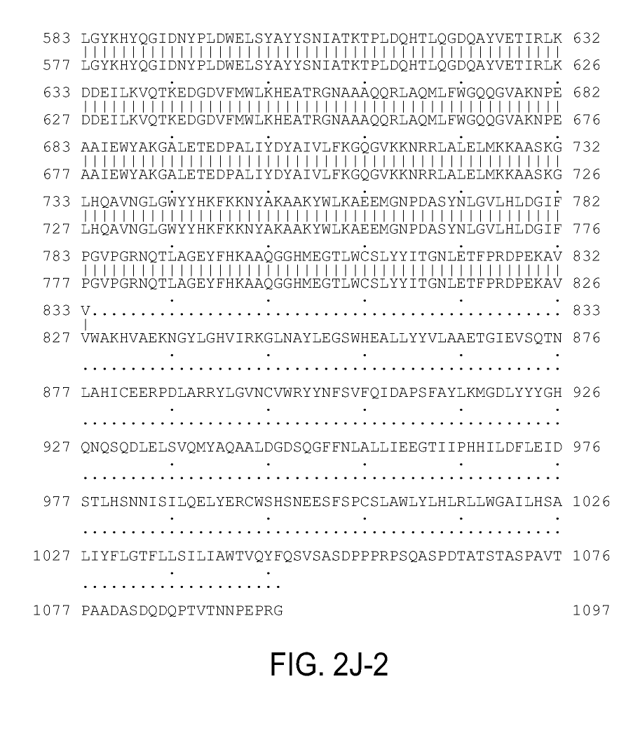

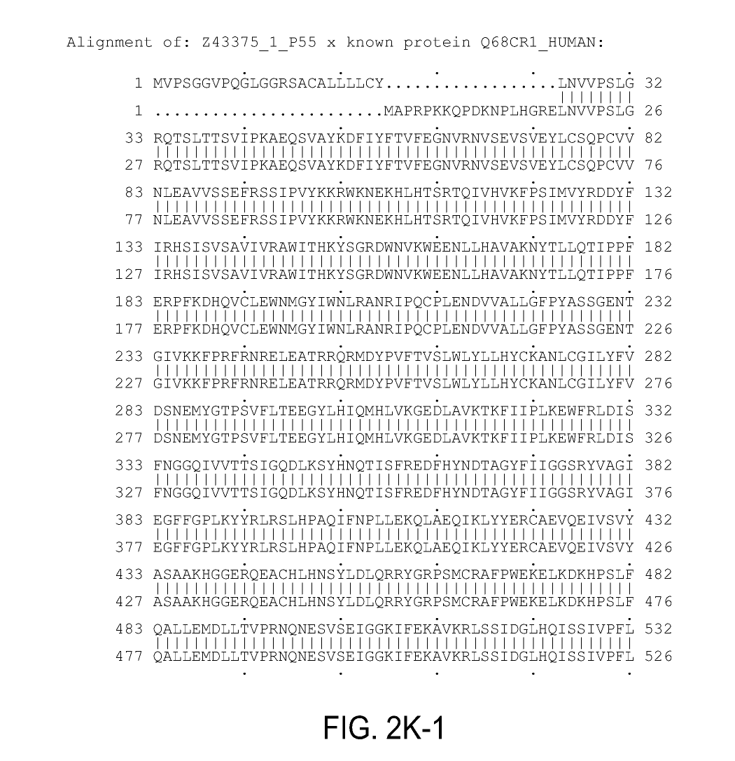

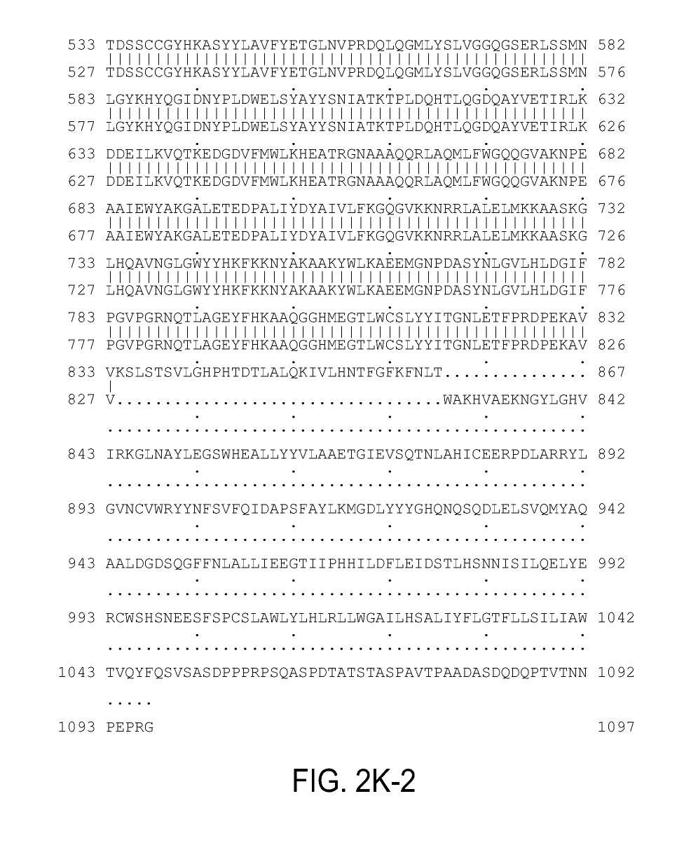

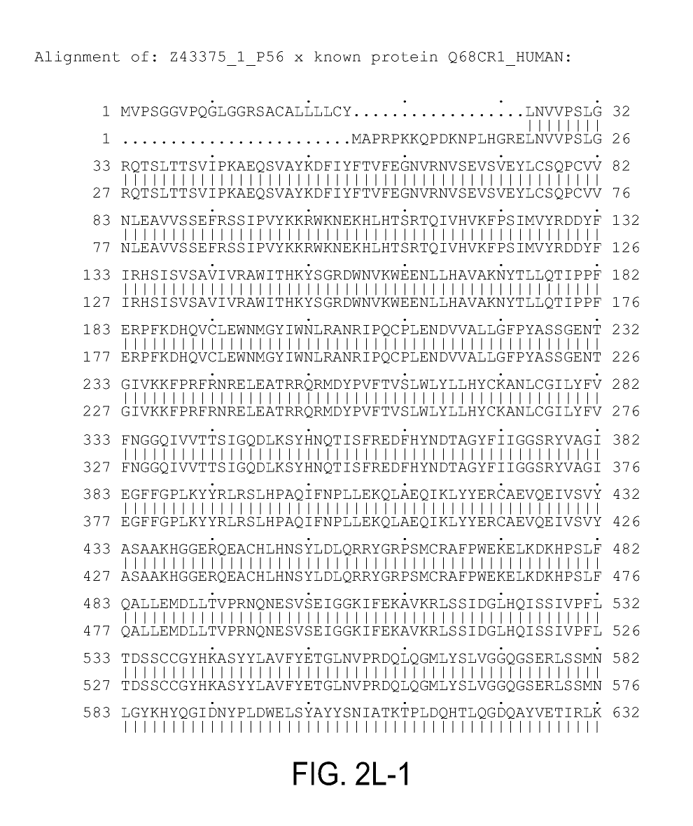

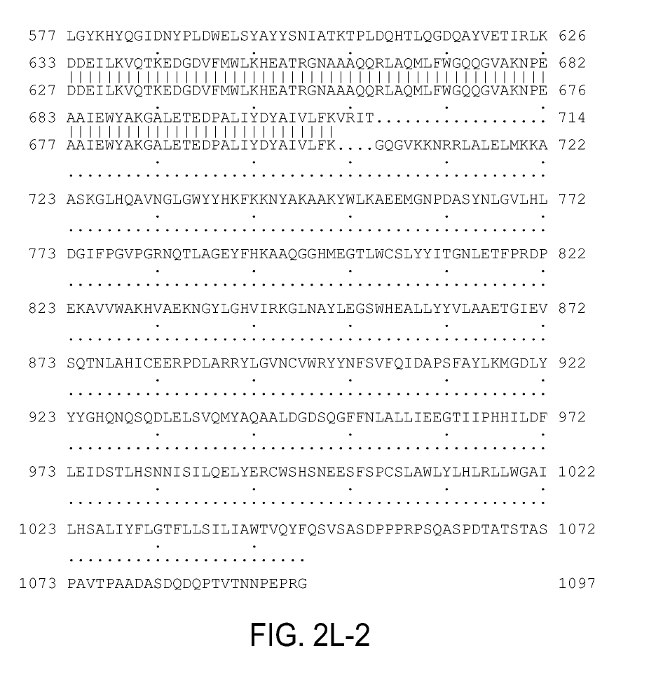

Variant protein Z43375_{—}1_P4 (SEQ ID NO:18) according to the present invention is encoded by transcript(s) Z43375_{—}1_T0 (SEQ ID NO:1). One or more alignments to one or more previously published protein sequences are given in FIG. 2A. A brief description of the relationship of the variant protein according to the present invention to each such aligned protein is as follows:

1. Comparison report between Z43375_{—}1_P4 (SEQ ID NO:18) and known protein Q68CR1_HUMAN (SEQ ID NO:16) (FIG. 2A):

A. An isolated chimeric polypeptide encoding for Z43375_{—}1_P4 (SEQ ID NO:18), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P4 (SEQ ID NO:18), and a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVVWAKHVAEKNGYLGHVIRKGLNAYLEGSWHEALLYYVLAAETGIE VSQTNLAHICEERPDLARRYLGVNCVWRYYNFSVFQIDAPSFAYLKMGDLYYYGHQ NQSQDLELSVQMYAQAALDGDSQGFFNLALLIEEGTIIPHHILDFLEIDSTLHSNNISIL QELYERCWSHSNEESFSPCSLAWLYLHLRLLWGAILHSALIYFLGTFLLSILIAWTVQY FQSVSASDPPPRPSQASPDTATSTASPAVTPAADASDQDQPTVTNNPEPRG corresponding to amino acids 19-1097 of known protein(s) Q68CR1_HUMAN (SEQ ID NO:16), which also corresponds to amino acids 25-1103 of Z43375_{—}1_P4 (SEQ ID NO:18), wherein said first amino acid sequence and second amino acid sequence are contiguous and in a sequential order.

B. An isolated polypeptide encoding for a head of Z43375_{—}1_P4 (SEQ ID NO:18), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P4 (SEQ ID NO:18).

The localization of the variant protein was determined according to results from a number of different software programs and analyses, including analyses from SignalP and other specialized programs. The variant protein is believed to be located as follows with regard to the cell: membrane.

Variant protein Z43375_{—}1_P4 (SEQ ID NO:18) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein Z43375_{—}1_P4 (SEQ ID NO:18) sequence provides support for the deduced sequence of this variant protein according to the present invention).

TABLE 9
Amino acid mutations

| SNP position(s) on | | |
|---|---|---|
| amino acid sequence | Alternative amino acid(s) | Previously known SNP? |
| 144 | V −> G | No |
| 1075 | S −> A | Yes |
| 1075 | S −> T | Yes |
| 1082 | T −> N | Yes |
| 1082 | T −> S | Yes |
| 1093 | P −> L | Yes |

The variant protein has the following domains, as determined by using InterPro. The domains are described in Table 10:

TABLE 10
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 806-843, |
| | | 918-954 |

Variant protein Z43375_{—}1_P4 (SEQ ID NO:18) is encoded by transcript Z43375_{—}1_T0 (SEQ ID NO:1), for which the coding portion starts at position 240 and ends at position 3548. The transcript also has the following SNPs as listed in Table 11 (given according to their position on the nucleotide sequence, with the alternative nucleic acid listed).

TABLE 11
Nucleic acid SNPs

| Polymorphism | SNP position(s) on nucleotide sequence |
|---|---|
| T −> G | 670, 3462, 3977, 4180, 4295 |
| T −> A | 3462, 3849, 3977, 4180 |
| C −> A | 3484, 4291 |
| C −> G | 3484, 4291 |
| C −> T | 3517 |
| A −> C | 3709, 3939 |
| A −> G | 3709, 3939, 4402 |
| T −> C | 3849 |
| G −> T | 4296 |
| G −> A | 4401, 4403 |

Variant protein Z43375_{—}1_P8 (SEQ ID NO:19) according to the present invention is encoded by transcript Z43375_{—}1_T3 (SEQ ID NO:2). One or more alignments to one or more previously published protein sequences are given in FIG. 2B. A brief description of the relationship of the variant protein according to the present invention to each such aligned protein is as follows:

Comparison report between Z43375_{—}1_P8 (SEQ ID NO:19) and known protein O94847_HUMAN (SEQ ID NO:17) (FIG. 2B):

A. An isolated chimeric polypeptide encoding for Z43375_{—}1_P8 (SEQ ID NO:19), comprising a first amino acid sequence being at least 70%, optionally at least 80%, preferably at least 85%, more preferably at least 90% and most preferably at least 95%, homologous to a polypeptide having the sequence KSAVVAVAAAPHKTLGKHPERAANQPAGWGAARLQTCQQGGSPNPAGGQVENVVP SLGRQTSLTTSVIPKAEQSVAYKDFIYFTVFEGNVRNVSEVSVEY corresponding to amino acids 1-100 of Z43375_{—}1_P8 (SEQ ID NO:19), and a second amino acid sequence being at least 90% homologous to LCSQPCVVNLEAVVSSEFRSSIPVYKKRWKNEKHLHTSRTQIVHVKFPSIMVYRDDYF IRHSISVSAVIVRAWITHKYSGRDWNVKWEENLLHAVAKNYTLLQTIPPFERPFKDHQ VCLEWNMGYIWNLRANRIPQCPLENDVVALLGFPYASSGENTGIVKKFPRFRNRELE ATRRQRMDYPVFTVSLWLYLLHYCKANLCGILYFVDSNEMYGTPSVFLTEEGYLHIQ MHLVKGEDLAVKTKFIIPLKEWFRLDISFNGGQIVVTTSIGQDLKSYHNQTISFREDFH YNDTAGYFIIGGSRYVAGIEGFFGPLKYYRLRSLHPAQIFNPLLEKQLAEQIKLYYERC AEVQEIVSVYASAAKHGGERQEACHLHNSYLDLQRRYGRPSMCRAFPWEKELKDKH PSLFQALLEMDLLTVPRNQNESVSEIGGKIFEKAVKRLSSIDGLHQISSIVPFLTDSSCC GYHKASYYLAVFYETGLNVPRDQLQGMLYSLVGGQGSERLSSMNLGYKHYQGIDN YPLDWELSYAYYSNIATKTPLDQHTLQGDQAYVETIRLKDDEILKVQTKEDGDVFM WLKHEATRGNAAAQQRLAQMLFWGQQGVAKNPEAAIEWYAKGALETEDPALIYDY AIVLFKGQGVKKNRRLALELMKKAASKGLHQAVNGLGWYYHKFKKNYAKAAKYW LKAEEMGNPDASYNLGVLHLDGIFPGVPGRNQTLAGEYFHKAAQGGHMEGTLWCS LYYITGNLETFPRDPEKAVVWAKHVAEKNGYLGHVIRKGLNAYLEGSWHEALLYYV LAAETGIEVSQTNLAHICEERPDLARRYLGVNCVWRYYNFSVFQIDAPSFAYLKMGD LYYYGHQNQSQDLELSVQMYAQAALDGDSQGFFNLALLIEEGTIIPHHILDFLEIDSTL HSNNISILQELYERCWSHSNEESFSPCSLAWLYLHLRLLWGAILHSALIYFLGTFLLSIL IAWTVQYFQSVSASDPPPRPSQASPDTATSTASPAVTPAADASDQDQPTVTNNPEPRG corresponding to amino acids 1-1029 of known protein(s) O94847_HUMAN (SEQ ID NO:17), which also corresponds to amino acids 101-1129 of Z43375_{—}1_P8 (SEQ ID NO:19), wherein said first amino acid sequence and second amino acid sequence are contiguous and in a sequential order.

B. An isolated polypeptide encoding for a head of Z43375_{—}1_P8 (SEQ ID NO:19), comprising a polypeptide being at least 70%, optionally at least about 80%, preferably at least about 85%, more preferably at least about 90% and most preferably at least about 95% homologous to the sequence KSAVVAVAAAPHKTLGKHPERAANQPAGWGAARLQTCQQGGSPNPAGGQVENVVP SLGRQTSLTTSVIPKAEQSVAYKDFIYFTVFEGNVRNVSEVSVEY of Z43375_{—}1_P8 (SEQ ID NO:19).

The localization of the variant protein was determined according to results from a number of different software programs and analyses, including analyses from SignalP and other specialized programs. The variant protein is believed to be located as follows with regard to the cell: membrane.

Variant protein Z43375_{—}1_P8 (SEQ ID NO:19) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein Z43375_{—}1_P8 (SEQ ID NO:19) sequence provides support for the deduced sequence of this variant protein according to the present invention).

TABLE 12
Amino acid mutations

| SNP position(s) on | | |
|---|---|---|
| amino acid sequence | Alternative amino acid(s) | Previously known SNP? |
| 170 | V −> G | No |
| 1101 | S −> A | Yes |
| 1101 | S −> T | Yes |
| 1108 | T −> N | Yes |
| 1108 | T −> S | Yes |
| 1119 | P −> L | Yes |

The variant protein has the following domains, as determined by using InterPro. The domains are described in Table 13:

TABLE 13
InterPro domain(s)

| Domain description | Analysis type | Position(s) on protein |
|---|---|---|
| Sel1-like repeat | HMMSmart | 567-601, 603-639, 686-722, 724- |
| | | 759, 760-792, 793-831, 832-869, |
| | | 944-980 |

Variant protein Z43375_{—}1_P8 (SEQ ID NO:19) is encoded by the following transcript: Z43375_{—}1_T3 (SEQ ID NO:2), for which the coding portion starts at position 2 and ends at position 3388. The transcript also has the following SNPs as listed in Table 14 (given according to their position on the nucleotide sequence, with the alternative nucleic acid listed).

TABLE 14
Nucleic acid SNPs

| Polymorphism | SNP position(s) on nucleotide sequence |
|---|---|
| T −> G | 510, 3302, 3817, 4020, 4135 |
| T −> A | 3302, 3689, 3817, 4020 |
| C −> A | 3324, 4131 |
| C −> G | 3324, 4131 |
| C −> T | 3357 |
| A −> C | 3549, 3779 |
| A −> G | 3549, 3779, 4242 |
| T −> C | 3689 |
| G −> T | 4136 |
| G −> A | 4241, 4243 |

Variant protein Z43375_{—}1_P40 (SEQ ID NO:20) according to the present invention is encoded by transcript Z43375_{—}1_T6 (SEQ ID NO:3). One or more alignments to one or more previously published protein sequences are given in FIG. 2C. A brief description of the relationship of the variant protein according to the present invention to each such aligned protein is as follows:

1. Comparison report between Z43375_{—}1_P40 (SEQ ID NO:20) and known protein Q68CR1_HUMAN (SEQ ID NO:16) (FIG. 2C):

A. An isolated chimeric polypeptide encoding for Z43375_{—}1_P40 (SEQ ID NO:20), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P40 (SEQ ID NO:20), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVVWAKHVAEKNGYLGHVIRKGLNAYLEGSW corresponding to amino acids 19-855 of known protein(s) Q68CR1_HUMAN (SEQ ID NO:16), which also corresponds to amino acids 25-861 of Z43375_{—}1_P40 (SEQ ID NO:20), and a third amino acid sequence being at least 70%, optionally at least 80%, preferably at least 85%, more preferably at least 90% and most preferably at least 95% homologous to a polypeptide having the sequence PQKVQNFYLVPSKKRDQCLRFRPPLP corresponding to amino acids 862-887 of Z43375_{—}1_P40 (SEQ ID NO:20), wherein said first amino acid sequence, second amino acid sequence and third amino acid sequence are contiguous and in a sequential order.

B. An isolated polypeptide encoding for a head of Z43375_{—}1_P40 (SEQ ID NO:20), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P40 (SEQ ID NO:20).

C. An isolated polypeptide encoding for an edge portion of Z43375_{—}1_P40 (SEQ ID NO:20), comprising an amino acid sequence being at least 70%, optionally at least about 80%, preferably at least about 85%, more preferably at least about 90% and most preferably at least about 95% homologous to the sequence PQKVQNFYLVPSKKRDQCLRFRPPLP of Z43375_{—}1_P40 (SEQ ID NO:20).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P40 (SEQ ID NO:20) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein Z43375_{—}1_P40 (SEQ ID NO:20) sequence provides support for the deduced sequence of this variant protein according to the present invention).

TABLE 15
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 16:

TABLE 16
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 806-843 |

Variant protein Z43375_{—}1_P40 (SEQ ID NO:20) is encoded by the following transcript: Z43375_{—}1_T6 (SEQ ID NO:3), for which the coding portion starts at position 240 and ends at position 2900. The transcript also has the following SNPs as listed in Table 17 (given according to their position on the nucleotide sequence, with the alternative nucleic acid listed).

TABLE 17
Nucleic acid SNPs

| Polymorphism | SNP position(s) on nucleotide sequence |
|---|---|
| T −> G | 670, 3724, 4239, 4442, 4557 |
| T −> A | 3724, 4111, 4239, 4442 |
| C −> A | 3746, 4553 |
| C −> G | 3746, 4553 |
| C −> T | 3779 |
| A −> C | 3971, 4201 |
| A −> G | 3971, 4201, 4664 |
| T −> C | 4111 |
| G −> T | 4558 |
| G −> A | 4663, 4665 |

Variant protein Z43375_{—}1_P46 (SEQ ID NO:21) according to the present invention is encoded by transcript Z43375_{—}1_T14 (SEQ ID NO:5). One or more alignments to one or more previously published protein sequences are given in FIG. 2D. A brief description of the relationship of the variant protein according to the present invention to each such aligned protein is as follows:

1. Comparison report between Z43375_{—}1_P46 (SEQ ID NO:21) and known protein Q68CR1_HUMAN (SEQ ID NO:16) (FIG. 2D):

A. An isolated chimeric polypeptide encoding for Z43375_{—}1_P46 (SEQ ID NO:21), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P46 (SEQ ID NO:21), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVV corresponding to amino acids 19-827 of known protein(s) Q68CR1_HUMAN (SEQ ID NO:16), which also corresponds to amino acids 25-833 of Z43375_{—}1_P46 (SEQ ID NO:21), and a third amino acid sequence being at least 90% homologous to HEALLYYVLAAETGIEVSQTNLAHICEERPDLARRYLGVNCVWRYYNFSVFQIDAPSF AYLKMGDLYYYGHQNQSQDLELSVQMYAQAALDGDSQGFFNLALLIEEGTIIPHHIL DFLEIDSTLHSNNISILQELYERCWSHSNEESFSPCSLAWLYLHLRLLWGAILHSALIYF LGTFLLSILIAWTVQYFQSVSASDPPPRPSQASPDTATSTASPAVTPAADASDQDQPTV TNNPEPRG corresponding to amino acids 856-1097 of known protein(s) Q68CR1_HUMAN (SEQ ID NO:16), which also corresponds to amino acids 834-1075 of Z43375_{—}1_P46 (SEQ ID NO:21), wherein said first amino acid sequence, second amino acid sequence and third amino acid sequence are contiguous and in a sequential order.

B. An isolated polypeptide encoding for a head of Z43375_{—}1_P46 (SEQ ID NO:21), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P46 (SEQ ID NO:21).

C. An isolated chimeric polypeptide encoding for an edge portion of Z43375_{—}1_P46 (SEQ ID NO:21), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VH, having a structure as follows: a sequence starting from any of amino acid numbers 833−x to 833; and ending at any of amino acid numbers 834+((n−2)−x), in which x varies from 0 to n−2.

The localization of the variant protein was determined according to results from a number of different software programs and analyses, including analyses from SignalP and other specialized programs. The variant protein is believed to be located as follows with regard to the cell: membrane.

Variant protein Z43375_{—}1_P46 (SEQ ID NO:21) also has the following non-silent SNPs (Single Nucleotide Polymorphisms) as listed in Table 18, (given according to their position(s) on the amino acid sequence, with the alternative amino acid(s) listed; the last column indicates whether the SNP is known or not; the presence of known SNPs in variant protein Z43375_{—}1_P46 (SEQ ID NO:21) sequence provides support for the deduced sequence of this variant protein according to the present invention).

TABLE 18
Amino acid mutations

| SNP position(s) on | | |
|---|---|---|
| amino acid sequence | Alternative amino acid(s) | Previously known SNP? |
| 144 | V −> G | No |
| 1047 | S −> A | Yes |
| 1047 | S −> T | Yes |
| 1054 | T −> N | Yes |
| 1054 | T −> S | Yes |
| 1065 | P −> L | Yes |

The variant protein has the following domains, as determined by using InterPro. The domains are described in Table 19:

TABLE 19
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 890-926 |

Variant protein Z43375_{—}1_P46 (SEQ ID NO:21) is encoded by transcript Z43375_{—}1_T14 (SEQ ID NO:5), for which the coding portion starts at position 240 and ends at position 3464. The transcript also has the following SNPs as listed in Table 20 (given according to their position on the nucleotide sequence, with the alternative nucleic acid listed).

TABLE 20
Nucleic acid SNPs

| Polymorphism | SNP position(s) on nucleotide sequence |
|---|---|
| T −> G | 670, 3378, 3893, 4096, 4211 |
| T −> A | 3378, 3765, 3893, 4096 |
| C −> A | 3400, 4207 |
| C −> G | 3400, 4207 |
| C −> T | 3433 |
| A −> C | 3625, 3855 |
| A −> G | 3625, 3855, 4318 |
| T −> C | 3765 |
| G −> T | 4212 |
| G −> A | 4317, 4319 |

Variant protein Z43375_{—}1_P47 (SEQ ID NO:22) according to the present invention is encoded by transcript Z43375_{—}1_T16 (SEQ ID NO:6). One or more alignments to one or more previously published protein sequences are given in FIG. 2E. A brief description of the relationship of the variant protein according to the present invention to each such aligned protein is as follows:

1. Comparison report between Z43375_{—}1_P47 (SEQ ID NO:22) and known protein Q68CR1_HUMAN (SEQ ID NO:16) (FIG. 2E):

A. An isolated chimeric polypeptide encoding for Z43375_{—}1_P47 (SEQ ID NO:22), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P47 (SEQ ID NO:22), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVVWAKHVAEKNGYLGHVIRKGLNAYLEGSWHEALLYYVLAAETGIE VSQTNLAHICEERPDLARRYLGVNCVWRYYNFSVFQIDAPSFAYLKMGDLYYYGHQ NQSQDLELSVQMYAQAALDGDSQGFFNLALLIEEGTIIPHHILDFLEIDSTLHSNNISIL QELYERCWSHSNEESFSPCSLAWLYLHLRLLWGAI corresponding to amino acids 19-1022 of known protein(s) Q68CR1_HUMAN (SEQ ID NO:16), which also corresponds to amino acids 25-1028 of Z43375_{—}1_P47 (SEQ ID NO:22), and a third amino acid sequence being at least 90% homologous to IYFLGTFLLSILIAWTVQYFQSVSASDPPPRPSQASPDTATSTASPAVTPAADASDQDQ PTVTNNPEPRG corresponding to amino acids 1028-1097 of known protein(s) Q68CR1_HUMAN (SEQ ID NO:16), which also corresponds to amino acids 1029-1098 of Z43375_{—}1_P47 (SEQ ID NO:22), wherein said first amino acid sequence, second amino acid sequence and third amino acid sequence are contiguous and in a sequential order.

B. An isolated polypeptide encoding for a head of Z43375_{—}1_P47 (SEQ ID NO:22), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P47 (SEQ ID NO:22).

C. An isolated chimeric polypeptide encoding for an edge portion of Z43375_{—}1_P47 (SEQ ID NO:22), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise II, having a structure as follows: a sequence starting from any of amino acid numbers 1028−x to 1028; and ending at any of amino acid numbers 1029+((n−2)−x), in which x varies from 0 to n−2.

The localization of the variant protein was determined according to results from a number of different software programs and analyses, including analyses from SignalP and other specialized programs. The variant protein is believed to be located as follows with regard to the cell: membrane.

Variant protein Z43375_{—}1_P47 (SEQ ID NO:22) also has the following non-silent SNPs (Single Nucleotide Polymorphisms) as listed in Table 21, (given according to their position(s) on the amino acid sequence, with the alternative amino acid(s) listed; the last column indicates whether the SNP is known or not; the presence of known SNPs in variant protein Z43375_{—}1_P47 (SEQ ID NO:22) sequence provides support for the deduced sequence of this variant protein according to the present invention).

TABLE 21
Amino acid mutations

| SNP position(s) on | | |
|---|---|---|
| amino acid sequence | Alternative amino acid(s) | Previously known SNP? |
| 144 | V −> G | No |
| 1070 | S −> A | Yes |
| 1070 | S −> T | Yes |
| 1077 | T −> N | Yes |
| 1077 | T −> S | Yes |
| 1088 | P −> L | Yes |

The variant protein has the following domains, as determined by using InterPro. The domains are described in Table 22:

TABLE 22
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 806-843, |
| | | 918-954 |

Variant protein Z43375_{—}1_P47 (SEQ ID NO:22) is encoded by the transcript Z43375_{—}1_T16 (SEQ ID NO:6), for which the coding portion starts at position 240 and ends at position 3533. The transcript also has the following SNPs as listed in Table 23 (given according to their position on the nucleotide sequence, with the alternative nucleic acid listed).

TABLE 23
Nucleic acid SNPs

| Polymorphism | SNP position(s) on nucleotide sequence |
|---|---|
| T −> G | 670, 3447 |
| T −> A | 3447 |
| C −> A | 3469 |
| C −> G | 3469 |
| C −> T | 3502 |

Variant protein Z43375_{—}1_P50 (SEQ ID NO:23) according to the present is encoded by transcript Z43375_{—}1_T20 (SEQ ID NO:7). One or more alignments to one or more previously published protein sequences are given in FIG. 2F. A brief description of the relationship of the variant protein according to the present invention to each such aligned protein is as follows:

1. Comparison report between Z43375_{—}1_P50 (SEQ ID NO:23) and known protein(s) Q68CR1_HUMAN (SEQ ID NO:16) (FIG. 2F):

A. An isolated chimeric polypeptide encoding for Z43375_{—}1_P50 (SEQ ID NO:23), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P50 (SEQ ID NO:23), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVVWAKHVAEKNGYLGHVIRKGLNAYLEGSWHEALLYYVLAAETGIE VSQTNLAHICEERPDLARRYLGVNCVWRYYNFSVFQIDAPSFAYLKMGDLYYYGHQ NQSQDLELSVQMYAQAALDGDSQGFFNLALLIEEGT corresponding to amino acids 19-963 of known protein(s) Q68CR1_HUMAN (SEQ ID NO:16), which also corresponds to amino acids 25-969 of Z43375_{—}1_P50 (SEQ ID NO:23), and a third amino acid sequence being at least 70%, optionally at least 80%, preferably at least 85%, more preferably at least 90% and most preferably at least 95% homologous to a polypeptide having the sequence VRKVLEPQ corresponding to amino acids 970-977 of Z43375_{—}1_P50 (SEQ ID NO:23), wherein said first amino acid sequence, second amino acid sequence and third amino acid sequence are contiguous and in a sequential order.

B. An isolated polypeptide encoding for a head of Z43375_{—}1_P50 (SEQ ID NO:23), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P50 (SEQ ID NO:23).

C. An isolated polypeptide encoding for an edge portion of Z43375_{—}1_P50 (SEQ ID NO:23), comprising an amino acid sequence being at least 70%, optionally at least about 80%, preferably at least about 85%, more preferably at least about 90% and most preferably at least about 95% homologous to the sequence VRKVLEPQ of Z43375_{—}1_P50 (SEQ ID NO:23).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P50 (SEQ ID NO:23) also has the following non-silent SNPs (Single Nucleotide Polymorphisms) as listed in Table 24, (given according to their position(s) on the amino acid sequence, with the alternative amino acid(s) listed; the last column indicates whether the SNP is known or not; the presence of known SNPs in variant protein Z43375_{—}1_P50 (SEQ ID NO:23) sequence provides support for the deduced sequence of this variant protein according to the present invention).

TABLE 24
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 25:

TABLE 25
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 806-843, |
| | | 918-954 |

The coding portion of transcript Z43375_{—}1_T20 (SEQ ID NO:7) starts at position 240 and ends at position 3170. The transcript also has the following SNPs as listed in Table 26 (given according to their position on the nucleotide sequence, with the alternative nucleic acid listed).

TABLE 26
Nucleic acid SNPs

| Polymorphism | SNP position(s) on nucleotide sequence |
|---|---|
| T −> G | 670 |

Variant protein Z43375_{—}1_P51 (SEQ ID NO:24) according to the present invention has an amino acid sequence encoded by transcript Z43375_{—}1_T22 (SEQ ID NO:8). One or more alignments to one or more previously published protein sequences are given in FIG. 2G. A brief description of the relationship of the variant protein according to the present invention to each such aligned protein is as follows:

1. Comparison report between Z43375_{—}1_P51 (SEQ ID NO:24) and known protein(s) Q68CR1_HUMAN (SEQ ID NO:16) (FIG. 2G):

A. An isolated chimeric polypeptide encoding for Z43375_{—}1_P51 (SEQ ID NO:24), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P51 (SEQ ID NO:24), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQ corresponding to amino acids 19-784 of known protein(s) Q68CR1_HUMAN (SEQ ID NO:16), which also corresponds to amino acids 25-790 of Z43375_{—}1_P51 (SEQ ID NO:24), and a third amino acid sequence being at least 70%, optionally at least 80%, preferably at least 85%, more preferably at least 90% and most preferably at least 95% homologous to a polypeptide having the sequence HI corresponding to amino acids 791-792 of Z43375_{—}1_P51 (SEQ ID NO:24), wherein said first amino acid sequence, second amino acid sequence and third amino acid sequence are contiguous and in a sequential order.

B. An isolated polypeptide encoding for a head of Z43375_{—}1_P51 (SEQ ID NO:24), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P51 (SEQ ID NO:24).

C. An isolated polypeptide encoding for an edge portion of Z43375_{—}1_P51 (SEQ ID NO:24), comprising an amino acid sequence being at least 70%, optionally at least about 80%, preferably at least about 85%, more preferably at least about 90% and most preferably at least about 95% homologous to the sequence HI of Z43375_{—}1_P51 (SEQ ID NO:24).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P51 (SEQ ID NO:24) also has the following non-silent SNPs (Single Nucleotide Polymorphisms) as listed in Table 27, (given according to their position(s) on the amino acid sequence, with the alternative amino acid(s) listed; the last column indicates whether the SNP is known or not; the presence of known SNPs in variant protein Z43375_{—}1_P51 (SEQ ID NO:24) sequence provides support for the deduced sequence of this variant protein according to the present invention).

TABLE 27
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 28:

TABLE 28
InterPro domain(s)

| Domain description | Analysis type | Position(s) on protein |
|---|---|---|
| Sel1-like repeat | HMMSmart | 541-575, 577-613, 660-696, 698- |
| | | 733, 734-766 |

Variant protein Z43375_{—}1_P51 (SEQ ID NO:24) is encoded by the transcript Z43375_{—}1_T22 (SEQ ID NO:8), for which the coding portion starts at position 240 and ends at position 2615. The transcript also has the following SNPs as listed in Table 29 (given according to their position on the nucleotide sequence, with the alternative nucleic acid listed).

TABLE 29
Nucleic acid SNPs

| Polymorphism | SNP position(s) on nucleotide sequence |
|---|---|
| T −> G | 670, 3074, 3589, 3792, 3907 |
| T −> A | 3074, 3461, 3589, 3792 |
| C −> A | 3096, 3903 |
| C −> G | 3096, 3903 |
| C −> T | 3129 |
| A −> C | 3321, 3551 |
| A −> G | 3321, 3551, 4014 |
| T −> C | 3461 |
| G −> T | 3908 |
| G −> A | 4013, 4015 |

Variant protein Z43375_{—}1_P52 (SEQ ID NO:25) according to the present invention has an amino acid sequence encoded by transcript Z43375_{—}1_T23 (SEQ ID NO:9). One or more alignments to one or more previously published protein sequences are given in FIG. 2H. A brief description of the relationship of the variant protein according to the present invention to each such aligned protein is as follows:

1. Comparison report between Z43375_{—}1_P52 (SEQ ID NO:25) and known protein(s) Q68CR1_HUMAN (SEQ ID NO:16) (FIG. 2H):

A. An isolated chimeric polypeptide encoding for Z43375_{—}1_P52 (SEQ ID NO:25), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P52 (SEQ ID NO:25), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVVWAKHVAEKNGYLGHVIRKGLNAYLEGSWHEALLYYVLAAETGIE VSQTNLAHICEERPDLARRYLGVNCVWRYYNFSVFQIDAPSFAYLKMGDLYYYGHQ NQSQDLELSVQMYAQAALDGDSQGFFNLALLIEEGTIIPHHILDFLEIDSTLHSNNISIL QELYER corresponding to amino acids 19-993 of known protein(s) Q68CR1_HUMAN (SEQ ID NO:16), which also corresponds to amino acids 25-999 of Z43375_{—}1_P52 (SEQ ID NO:25), and a third amino acid sequence being at least 70%, optionally at least 80%, preferably at least 85%, more preferably at least 90% and most preferably at least 95% homologous to a polypeptide having the sequence STFWEPFCYPY corresponding to amino acids 1000-1010 of Z43375_{—}1_P52 (SEQ ID NO:25), wherein said first amino acid sequence, second amino acid sequence and third amino acid sequence are contiguous and in a sequential order.

B. An isolated polypeptide encoding for a head of Z43375_{—}1_P52 (SEQ ID NO:25), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P52 (SEQ ID NO:25).

C. An isolated polypeptide encoding for an edge portion of Z43375_{—}1_P52 (SEQ ID NO:25), comprising an amino acid sequence being at least 70%, optionally at least about 80%, preferably at least about 85%, more preferably at least about 90% and most preferably at least about 95% homologous to the sequence STFWEPFCYPY of Z43375_{—}1_P52 (SEQ ID NO:25).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P52 (SEQ ID NO:25) also has the following non-silent SNPs (Single Nucleotide Polymorphisms) as listed in Table 30, (given according to their position(s) on the amino acid sequence, with the alternative amino acid(s) listed; the last column indicates whether the SNP is known or not; the presence of known SNPs in variant protein Z43375_{—}1_P52 (SEQ ID NO:25) sequence provides support for the deduced sequence of this variant protein according to the present invention).

TABLE 30
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 31:

TABLE 31
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805, 806-843, |
| | | 918-954 |

Variant protein Z43375_{—}1_P52 (SEQ ID NO:25) is encoded by the transcript Z43375_{—}1_T23 (SEQ ID NO:9), for which the coding portion starts at position 240 and ends at position 3269. The transcript also has the following SNPs as listed in Table 32 (given according to their position on the nucleotide sequence, with the alternative nucleic acid listed).

TABLE 32
Nucleic acid SNPs

| Polymorphism | SNP position(s) on nucleotide sequence |
|---|---|
| T −> G | 670 |
| G −> C | 3378 |

Variant protein Z43375_{—}1_P53 (SEQ ID NO:26) according to the present invention has an amino acid sequence encoded by transcript Z43375_{—}1_T28 (SEQ ID NO:10). One or more alignments to one or more previously published protein sequences are given in FIG. 21. A brief description of the relationship of the variant protein according to the present invention to each such aligned protein is as follows:

1. Comparison report between Z43375_{—}1_P53 (SEQ ID NO:26) and known protein(s) Q68CR1_HUMAN (SEQ ID NO:16) (FIG. 21):

A. An isolated chimeric polypeptide encoding for Z43375_{—}1_P53 (SEQ ID NO:26), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P53 (SEQ ID NO:26), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITG corresponding to amino acids 19-813 of known protein(s) Q68CR1_HUMAN (SEQ ID NO:16), which also corresponds to amino acids 25-819 of Z43375_{—}1_P53 (SEQ ID NO:26), and a third amino acid sequence being at least 70%, optionally at least 80%, preferably at least 85%, more preferably at least 90% and most preferably at least 95% homologous to a polypeptide having the sequence LPRHCHVHCKSSCDSSCRCL corresponding to amino acids 820-839 of Z43375_{—}1_P53 (SEQ ID NO:26), wherein said first amino acid sequence, second amino acid sequence and third amino acid sequence are contiguous and in a sequential order.

B. An isolated polypeptide encoding for a head of Z43375_{—}1_P53 (SEQ ID NO:26), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P53 (SEQ ID NO:26).

C. An isolated polypeptide encoding for an edge portion of Z43375_{—}1_P53 (SEQ ID NO:26), comprising an amino acid sequence being at least 70%, optionally at least about 80%, preferably at least about 85%, more preferably at least about 90% and most preferably at least about 95% homologous to the sequence LPRHCHVHCKSSCDSSCRCL of Z43375_{—}1_P53 (SEQ ID NO:26).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P53 (SEQ ID NO:26) also has the following non-silent SNPs (Single Nucleotide Polymorphisms) as listed in Table 33, (given according to their position(s) on the amino acid sequence, with the alternative amino acid(s) listed; the last column indicates whether the SNP is known or not; the presence of known SNPs in variant protein Z43375_{—}1_P53 (SEQ ID NO:26) sequence provides support for the deduced sequence of this variant protein according to the present invention).

TABLE 33
Amino acid mutations

| SNP position(s) on | | |
|---|---|---|
| amino acid sequence | Alternative amino acid(s) | Previously known SNP? |
| 144 | V −> G | No |
| 826 | V −> D | Yes |
| 826 | V −> G | Yes |
| 833 | D −> E | Yes |

The variant protein has the following domains, as determined by using InterPro. The domains are described in Table 34:

TABLE 34
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805 |

Variant protein Z43375_{—}1_P53 (SEQ ID NO:26) is encoded by the transcript Z43375_{—}1_T28 (SEQ ID NO:10), for which the coding portion starts at position 240 and ends at position 2756. The transcript also has the following SNPs as listed in Table 35 (given according to their position on the nucleotide sequence, with the alternative nucleic acid listed).

TABLE 35
Nucleic acid SNPs

| Polymorphism | SNP position(s) on nucleotide sequence |
|---|---|
| T −> G | 670, 2716, 3231, 3434, 3549 |
| T −> A | 2716, 3103, 3231, 3434 |
| C −> A | 2738, 3545 |
| C −> G | 2738, 3545 |
| C −> T | 2771 |
| A −> C | 2963, 3193 |
| A −> G | 2963, 3193, 3656 |
| T −> C | 3103 |
| G −> T | 3550 |
| G −> A | 3655, 3657 |

Variant protein Z43375_{—}1_P54 (SEQ ID NO:27) according to the present invention has an amino acid sequence encoded by transcript Z43375_{—}1_T30 (SEQ ID NO:11). One or more alignments to one or more previously published protein sequences are given FIG. 2J. A brief description of the relationship of the variant protein according to the present invention to each such aligned protein is as follows:

1. Comparison report between Z43375_{—}1_P54 (SEQ ID NO:27) and known protein Q68CR1_HUMAN (SEQ ID NO:16) (FIG. 2J):

A. An isolated chimeric polypeptide encoding for Z43375_{—}1_P54 (SEQ ID NO:27), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P54 (SEQ ID NO:27), and a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVV corresponding to amino acids 19-827 of known protein(s) Q68CR1_HUMAN (SEQ ID NO:16), which also corresponds to amino acids 25-833 of Z43375_{—}1_P54 (SEQ ID NO:27), wherein said first amino acid sequence and second amino acid sequence are contiguous and in a sequential order.

B. An isolated polypeptide encoding for a head of Z43375_{—}1_P54 (SEQ ID NO:27), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P54 (SEQ ID NO:27).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P54 (SEQ ID NO:27) also has the following non-silent SNPs (Single Nucleotide Polymorphisms) as listed in Table 36, (given according to their position(s) on the amino acid sequence, with the alternative amino acid(s) listed; the last column indicates whether the SNP is known or not; the presence of known SNPs in variant protein Z43375_{—}1_P54 (SEQ ID NO:27) sequence provides support for the deduced sequence of this variant protein according to the present invention).

TABLE 36
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 37:

TABLE 37
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805 |

Variant protein Z43375_{—}1_P54 (SEQ ID NO:27) is encoded by the transcript Z43375_{—}1_T30 (SEQ ID NO:11), for which the coding portion of transcript Z43375_{—}1_T30 (SEQ ID NO:11) is shown in bold; this coding portion starts at position 240 and ends at position 2738. The transcript also has the following SNPs as listed in Table 38 (given according to their position on the nucleotide sequence, with the alternative nucleic acid listed).

TABLE 38
Nucleic acid SNPs

| Polymorphism | SNP position(s) on nucleotide sequence |
|---|---|
| T −> G | 670 |
| T −> C | 3450 |

Variant protein Z43375_{—}1_P55 (SEQ ID NO:28) according to the present invention has an amino acid sequence encoded by transcript Z43375_{—}1_T31 (SEQ ID NO:12). One or more alignments to one or more previously published protein sequences are given in FIG. 2K. A brief description of the relationship of the variant protein according to the present invention to each such aligned protein is as follows:

1. Comparison report between Z43375_{—}1_P55 (SEQ ID NO:28) and known protein Q68CR1_HUMAN (SEQ ID NO:16) (FIG. 2K):

A. An isolated chimeric polypeptide encoding for Z43375_{—}1_P55 (SEQ ID NO:28), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P55 (SEQ ID NO:28), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFKGQ GVKKNRRLALELMKKAASKGLHQAVNGLGWYYHKFKKNYAKAAKYWLKAEEMG NPDASYNLGVLHLDGIFPGVPGRNQTLAGEYFHKAAQGGHMEGTLWCSLYYITGNL ETFPRDPEKAVV corresponding to amino acids 19-827 of known protein(s) Q68CR1_HUMAN (SEQ ID NO:16), which also corresponds to amino acids 25-833 of Z43375_{—}1_P55 (SEQ ID NO:28), and a third amino acid sequence being at least 70%, optionally at least 80%, preferably at least 85%, more preferably at least 90% and most preferably at least 95% homologous to a polypeptide having the sequence KSLSTSVLGHPHTDTLALQKIVLHNTFGFKFNLT corresponding to amino acids 834-867 of Z43375_{—}1_P55 (SEQ ID NO:28), wherein said first amino acid sequence, second amino acid sequence and third amino acid sequence are contiguous and in a sequential order.

B. An isolated polypeptide encoding for a head of Z43375_{—}1_P55 (SEQ ID NO:28), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P55 (SEQ ID NO:28).

C. An isolated polypeptide encoding for an edge portion of Z43375_{—}1_P55 (SEQ ID NO:28), comprising an amino acid sequence being at least 70%, optionally at least about 80%, preferably at least about 85%, more preferably at least about 90% and most preferably at least about 95% homologous to the sequence KSLSTSVLGHPHTDTLALQKIVLHNTFGFKFNLT of Z43375_{—}1_P55 (SEQ ID NO:28).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P55 (SEQ ID NO:28) also has the following non-silent SNPs (Single Nucleotide Polymorphisms) as listed in Table 39, (given according to their position(s) on the amino acid sequence, with the alternative amino acid(s) listed; the last column indicates whether the SNP is known or not; the presence of known SNPs in variant protein Z43375_{—}1_P55 (SEQ ID NO:28) sequence provides support for the deduced sequence of this variant protein according to the present invention).

TABLE 39
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 40:

TABLE 40
InterPro domain(s)

| Domain description | Analysis type | Position(s) on protein |
|---|---|---|
| Sel1-like repeat | HMMSmart | 541-575, 577-613, 660-696, 698- |
| | | 733, 734-766, 767-805 |

Variant protein Z43375_{—}1_P55 (SEQ ID NO:28) is encoded by the transcript Z43375_{—}1_T31 (SEQ ID NO:12), for which coding portion starts at position 240 and ends at position 2840. The transcript also has the following SNPs as listed in Table 41 (given according to their position on the nucleotide sequence, with the alternative nucleic acid listed).

TABLE 41
Nucleic acid SNPs

| Polymorphism | SNP position(s) on nucleotide sequence |
|---|---|
| T −> G | 670 |

Variant protein Z43375_{—}1_P56 (SEQ ID NO:29) according to the present invention has an amino acid sequence encoded by transcript(s) Z43375_{—}1_T33 (SEQ ID NO:13). One or more alignments to one or more previously published protein sequences are given in FIG. 2L. A brief description of the relationship of the variant protein according to the present invention to each such aligned protein is as follows:

1. Comparison report between Z43375_{—}1_P56 (SEQ ID NO:29) and known protein Q68CR1_HUMAN (SEQ ID NO:16) (FIG. 2L):

A. An isolated chimeric polypeptide encoding for Z43375_{—}1_P56 (SEQ ID NO:29), comprising a first amino acid sequence being at least 70%, optionally at least 80%, preferably at least 85%, more preferably at least 90% and most preferably at least 95%, homologous to a polypeptide having the sequence MVPSGGVPQGLGGRSACALLLLCY corresponding to amino acids 1-24 of Z43375_{—}1_P56 (SEQ ID NO:29), a second amino acid sequence being at least 90% homologous to LNVVPSLGRQTSLTTSVIPKAEQSVAYKDFIYFTVFEGNVRNVSEVSVEYLCSQPCVV NLEAVVSSEFRSSIPVYKKRWKNEKHLHTSRTQIVHVKFPSIMVYRDDYFIRHSISVSA VIVRAWITHKYSGRDWNVKWEENLLHAVAKNYTLLQTIPPFERPFKDHQVCLEWNM GYIWNLRANRIPQCPLENDVVALLGFPYASSGENTGIVKKFPRFRNRELEATRRQRM DYPVFTVSLWLYLLHYCKANLCGILYFVDSNEMYGTPSVFLTEEGYLHIQMHLVKGE DLAVKTKFIIPLKEWFRLDISFNGGQIVVTTSIGQDLKSYHNQTISFREDFHYNDTAGY FIIGGSRYVAGIEGFFGPLKYYRLRSLHPAQIFNPLLEKQLAEQIKLYYERCAEVQEIVS VYASAAKHGGERQEACHLHNSYLDLQRRYGRPSMCRAFPWEKELKDKHPSLFQALL EMDLLTVPRNQNESVSEIGGKIFEKAVKRLSSIDGLHQISSIVPFLTDSSCCGYHKASY YLAVFYETGLNVPRDQLQGMLYSLVGGQGSERLSSMNLGYKHYQGIDNYPLDWELS YAYYSNIATKTPLDQHTLQGDQAYVETIRLKDDEILKVQTKEDGDVFMWLKHEATR GNAAAQQRLAQMLFWGQQGVAKNPEAAIEWYAKGALETEDPALIYDYAIVLFK corresponding to amino acids 19-704 of known protein(s) Q68CR1_HUMAN (SEQ ID NO:16), which also corresponds to amino acids 25-710 of Z43375_{—}1_P56 (SEQ ID NO:29), and a third amino acid sequence being at least 70%, optionally at least 80%, preferably at least 85%, more preferably at least 90% and most preferably at least 95% homologous to a polypeptide having the sequence VRIT corresponding to amino acids 711-714 of Z43375_{—}1_P56 (SEQ ID NO:29), wherein said first amino acid sequence, second amino acid sequence and third amino acid sequence are contiguous and in a sequential order.

B. An isolated polypeptide encoding for a head of Z43375_{—}1_P56 (SEQ ID NO:29), comprising a polypeptide being at least 70%, optionally at least about 80%, preferably at least about 85%, more preferably at least about 90% and most preferably at least about 95% homologous to the sequence MVPSGGVPQGLGGRSACALLLLCY of Z43375_{—}1_P56 (SEQ ID NO:29).

C. An isolated polypeptide encoding for an edge portion of Z43375_{—}1_P56 (SEQ ID NO:29), comprising an amino acid sequence being at least 70%, optionally at least about 80%, preferably at least about 85%, more preferably at least about 90% and most preferably at least about 95% homologous to the sequence VRIT of Z43375_{—}1_P56 (SEQ ID NO:29).

The localization of the variant protein was determined according to results from a number of different software programs and analyses, including analyses from SignalP and other specialized programs. The variant protein is believed to be located as follows with regard to the cell: secreted.

Variant protein Z43375_{—}1_P56 (SEQ ID NO:29) also has the following non-silent SNPs (Single Nucleotide Polymorphisms) as listed in Table 42, (given according to their position(s) on the amino acid sequence, with the alternative amino acid(s) listed; the last column indicates whether the SNP is known or not; the presence of known SNPs in variant protein Z43375_{—}1_P56 (SEQ ID NO:29) sequence provides support for the deduced sequence of this variant protein according to the present invention).

TABLE 42
Amino acid mutations

| SNP position(s) on | | |
|---|---|---|
| amino acid sequence | Alternative amino acid(s) | Previously known SNP? |
| 144 | V −> G | No |

The variant protein has the following domains, as determined by using InterPro. The domains are described in Table 43:

TABLE 43
InterPro domain(s)

| Domain description | Analysis type | Position(s) on protein |
|---|---|---|
| Sel1-like repeat | HMMSmart | 541-575, 577-613, 660-696 |

Variant protein Z43375_{—}1_P56 (SEQ ID NO:29) is encoded by the transcript Z43375_{—}1_T33 (SEQ ID NO:13), for which the coding portion starts at position 240 and ends at position 2381. The transcript also has the following SNPs as listed in Table 44 (given according to their position on the nucleotide sequence, with the alternative nucleic acid listed).

TABLE 44
Nucleic acid SNPs

| Polymorphism | SNP position(s) on nucleotide sequence |
|---|---|
| T −> G | 670 |

Variant protein Z43375_{—}1_P60 (SEQ ID NO:30) according to the present invention has an amino acid sequence encoded by transcript Z43375_{—}1_T7 (SEQ ID NO:4).

The localization of the variant protein was determined according to results from a number of different software programs and analyses, including analyses from SignalP and other specialized programs. The variant protein is believed to be located as follows with regard to the cell: membrane.

Variant protein Z43375_{—}1_P60 (SEQ ID NO:30) also has the following non-silent SNPs (Single Nucleotide Polymorphisms) as listed in Table 45, (given according to their position(s) on the amino acid sequence, with the alternative amino acid(s) listed; the last column indicates whether the SNP is known or not; the presence of known SNPs in variant protein Z43375_{—}1_P60 (SEQ ID NO:30) sequence provides support for the deduced sequence of this variant protein according to the present invention).

TABLE 45
Amino acid mutations

| SNP position(s) on | | |
|---|---|---|
| amino acid sequence | Alternative amino acid(s) | Previously known SNP? |
| 822 | S −> A | Yes |
| 822 | S −> T | Yes |
| 829 | T −> N | Yes |
| 829 | T −> S | Yes |
| 840 | P −> L | Yes |

The variant protein has the following domains, as determined by using InterPro. The domains are described in Table 46:

TABLE 46
InterPro domain(s)

| Domain description | Analysis type | Position(s) on protein |
|---|---|---|
| Sel1-like repeat | HMMSmart | 288-322, 324-360, 407-443, 445- |
| | | 480, 481-513, 514-552, 553-590, |
| | | 665-701 |

Variant protein Z43375_{—}1_P60 (SEQ ID NO:30) is encoded by the transcript Z43375_{—}1_T7 (SEQ ID NO:4), for which the coding portion starts at position 428 and ends at position 2977. The transcript also has the following SNPs as listed in Table 47 (given according to their position on the nucleotide sequence, with the alternative nucleic acid listed).

TABLE 47
Nucleic acid SNPs

| Polymorphism | SNP position(s) on nucleotide sequence |
|---|---|
| T −> A | 2891, 3278, 3406, 3609 |
| T −> G | 2891, 3406, 3609, 3724 |
| C −> A | 2913, 3720 |
| C −> G | 2913, 3720 |
| C −> T | 2946 |
| A −> C | 3138, 3368 |
| A −> G | 3138, 3368, 3831 |
| T −> C | 3278 |
| G −> T | 3725 |
| G −> A | 3830, 3832 |

### Example 2_{—}2

### Analysis of the Expression of KIAA0746 Transcripts Expression of KIAA0746 Cluster Using MED Discovery Engine

MED discovery engine described in Example 1 herein was used to assess the expression of KIAA0746 transcripts. Expression data for Affymetrix probe 235353 at representing KIAA0746 family data is shown in FIG. 3. As is evident from the scatter plot, presented in FIG. 3, the expression of KIAA0746 transcripts detectable with the above probe set was higher in specific samples, including normal bone marrow CD138+ cells, peripheral blood CD20+ B cells, splenocytes, stomach and in disease samples including follicular lymphoma, diffuse large B cell lymphoma, multiple myeloma, and monoclonal gammopathy of undetermined significance, stomach pylorus or fundus, ulcerative colitis, and peripheral blood CD20+ B cells of rheumatoid arthritis or systemic lupus erythematosus. For each group, the median expression is represented by a marker, and the expression values of the different chips in the group are represented by small dashes (“-”). The groups are ordered and marked as follows—“Other” groups (e.g. benign, non-cancer diseases, etc.) with an “x”, Treated cells with a square, Normal with a circle, Matched with a “+”, and Cancer with a diamond.

Expression of KIAA0746 Transcripts which are Detectable by Amplicon as Depicted in Sequence Name CGEN-790_seg33-34-36-1 (SEQ ID NO:81) in Normal and Cancerous Tissues

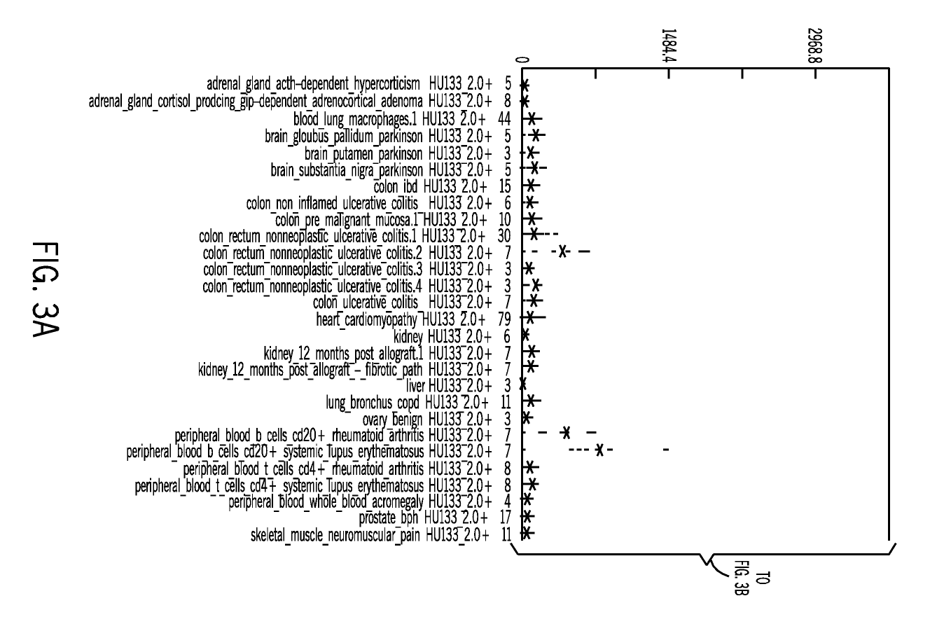

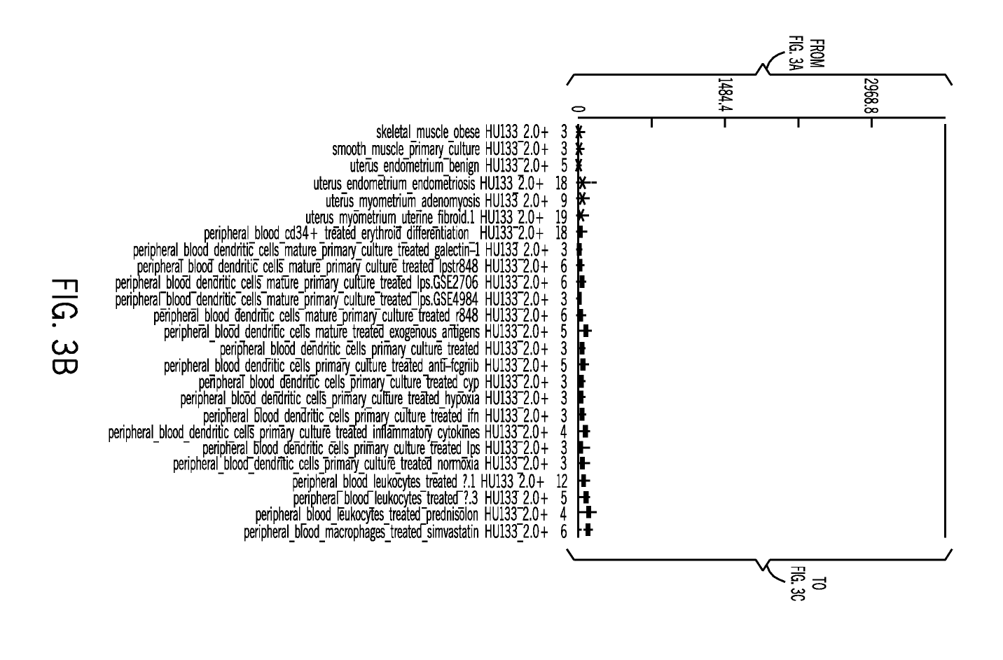

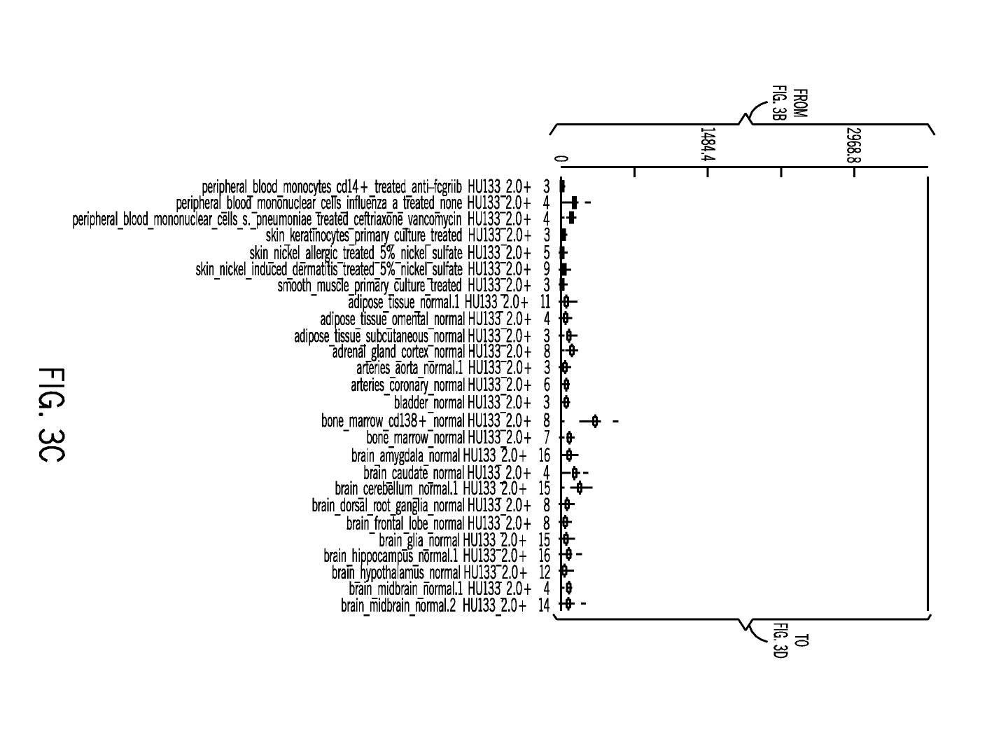

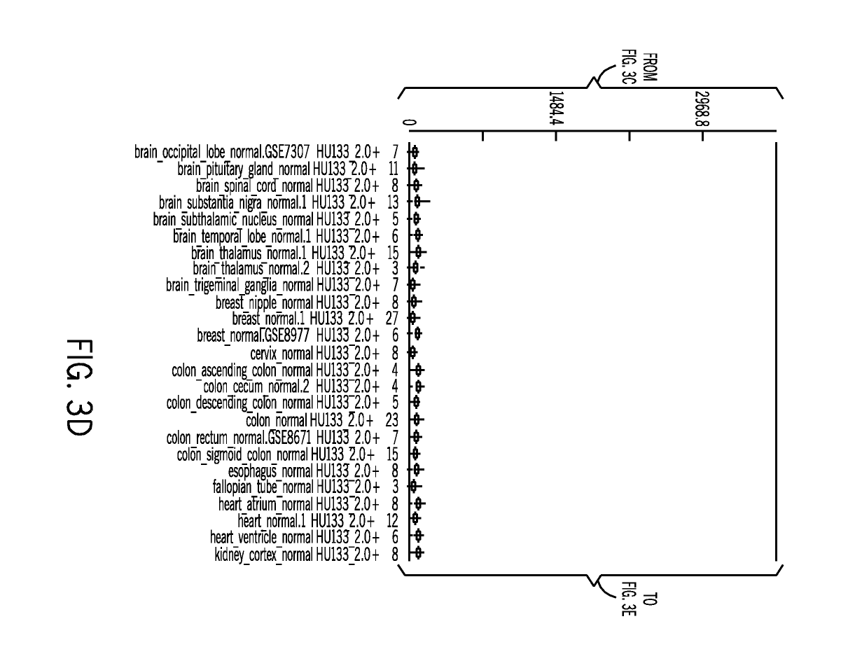

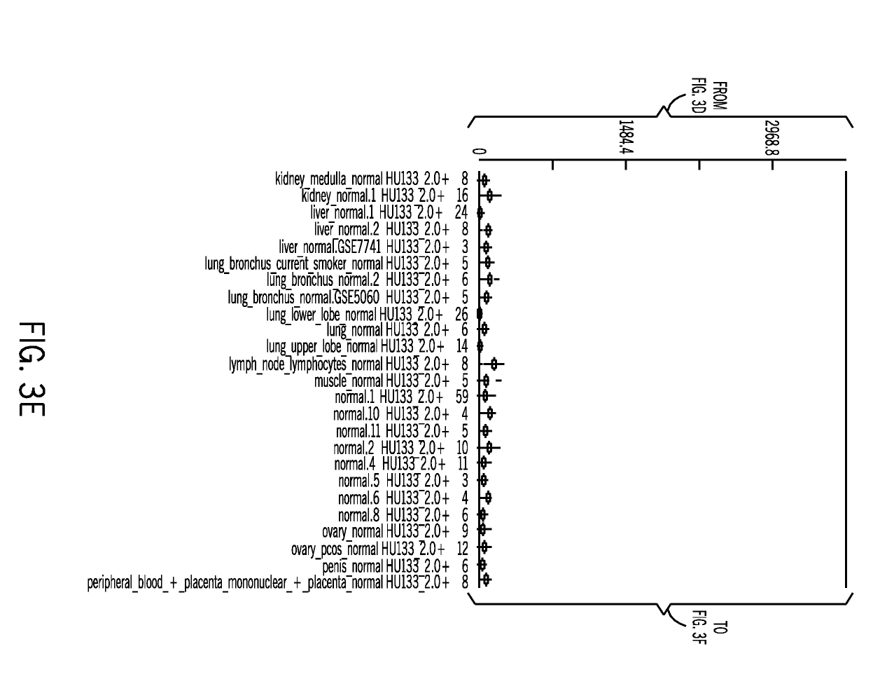

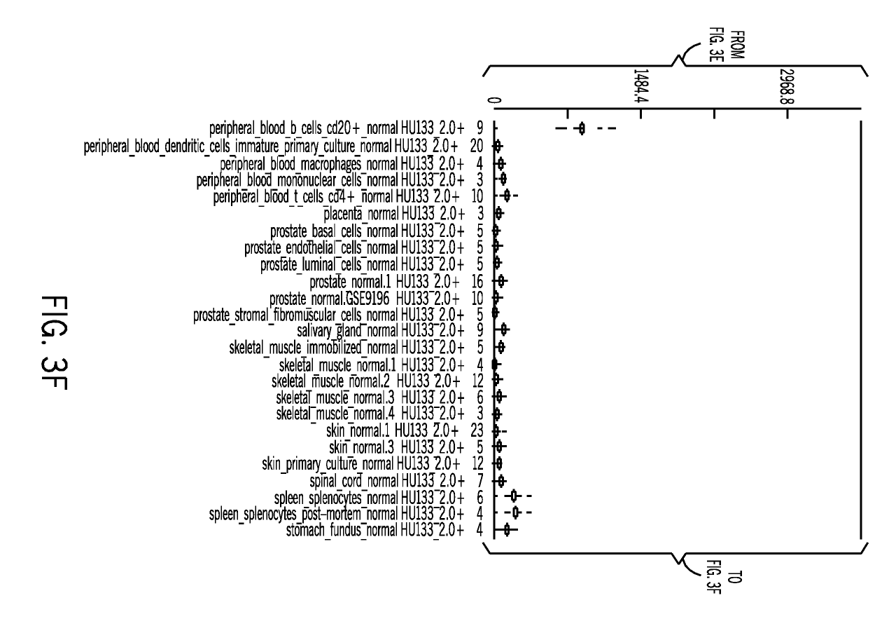

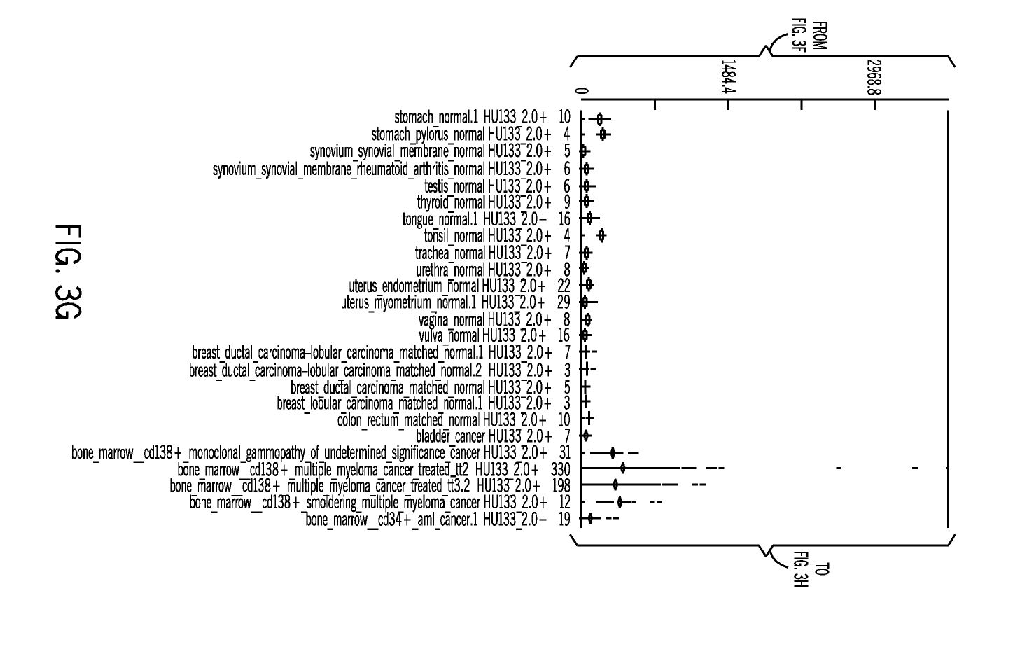

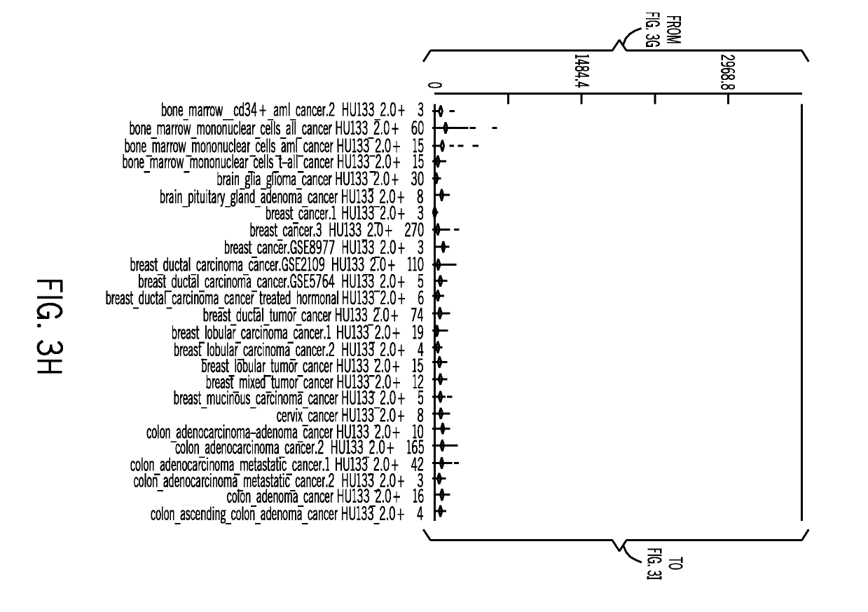

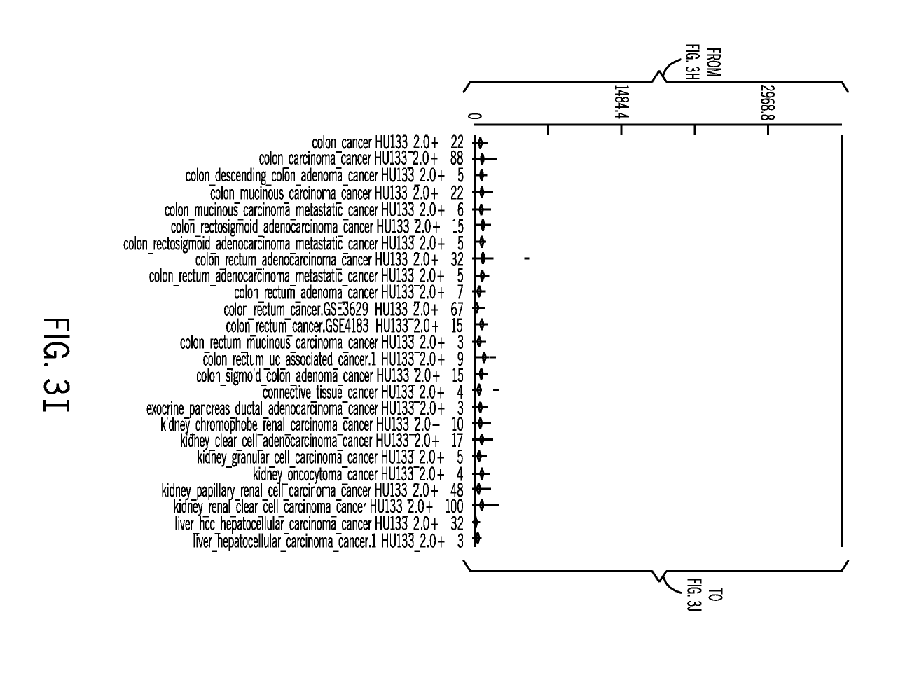

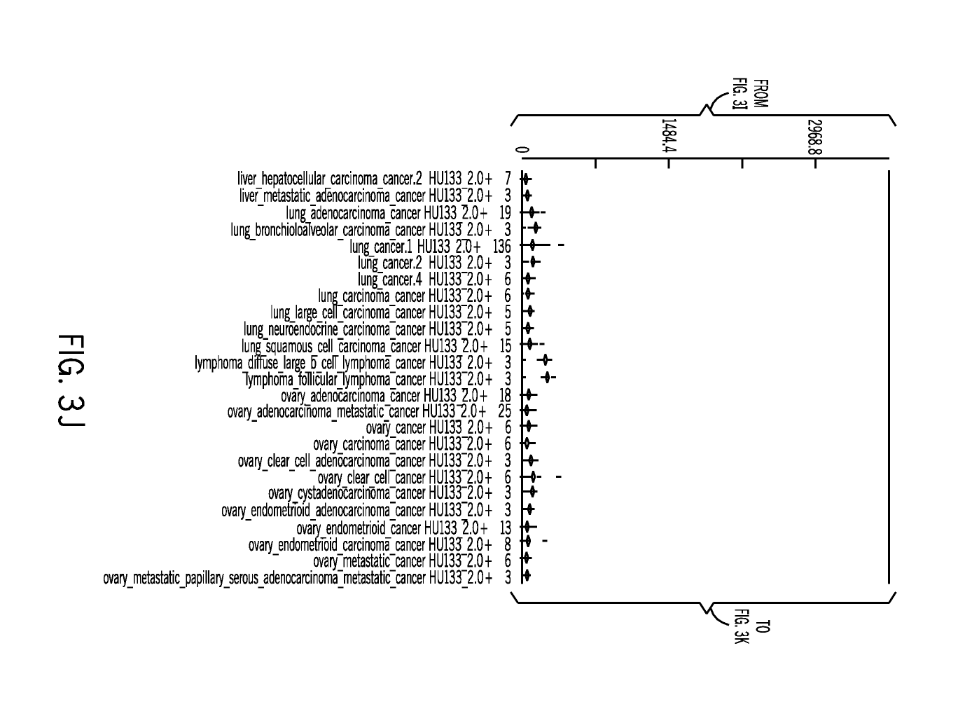

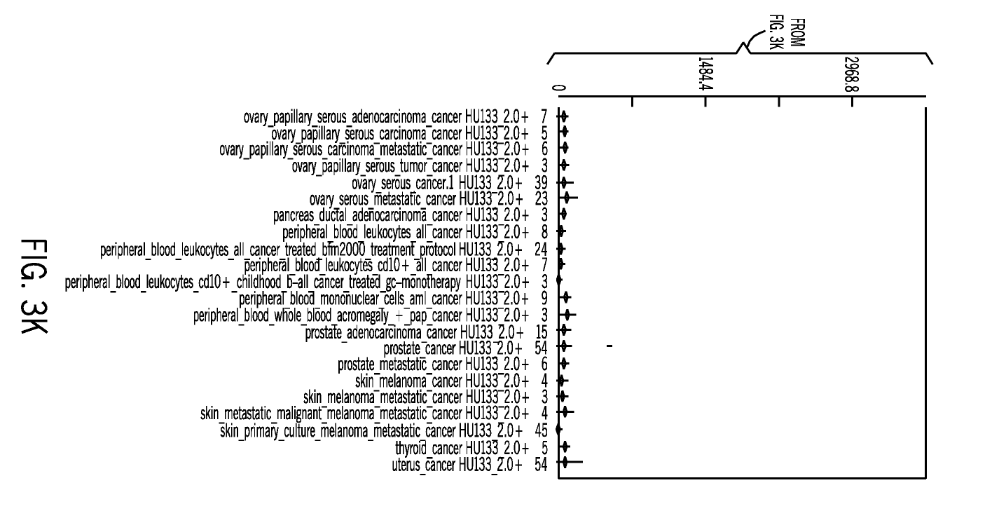

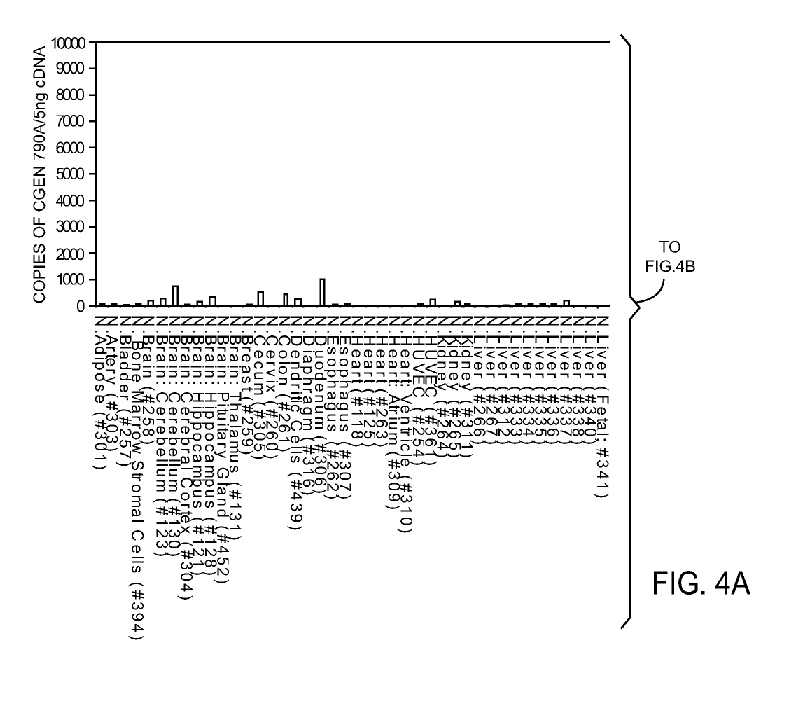

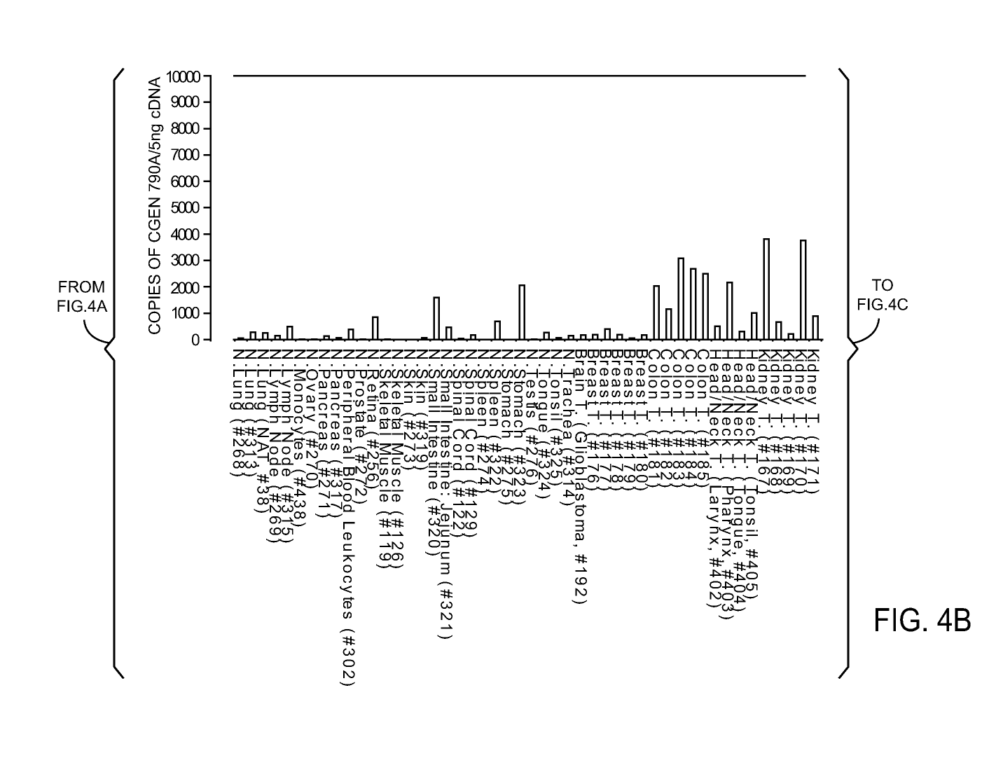

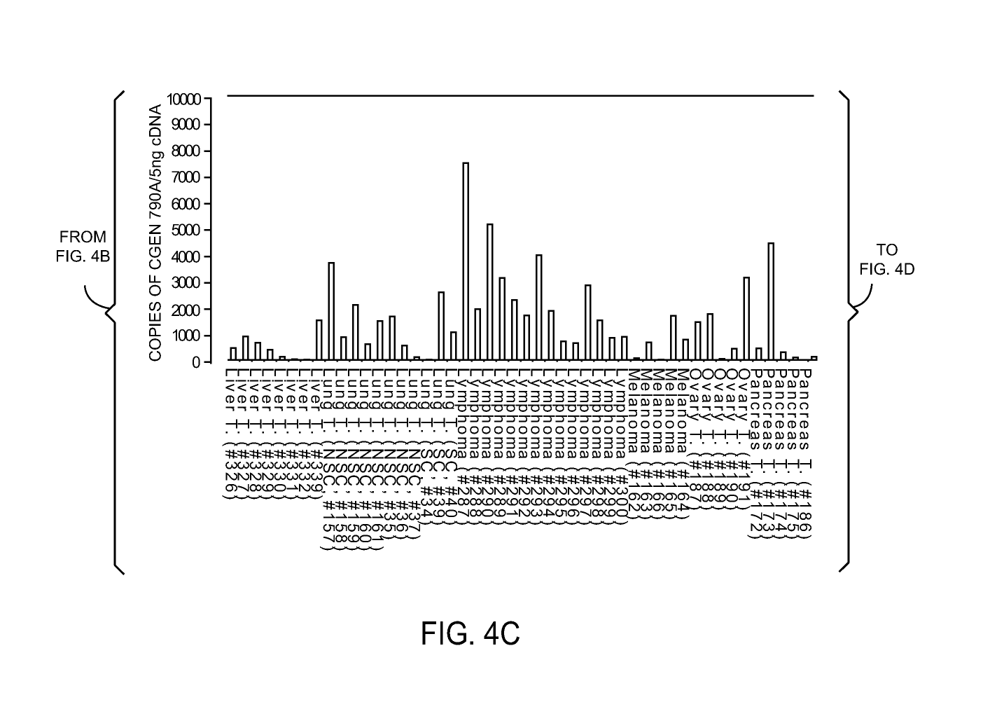

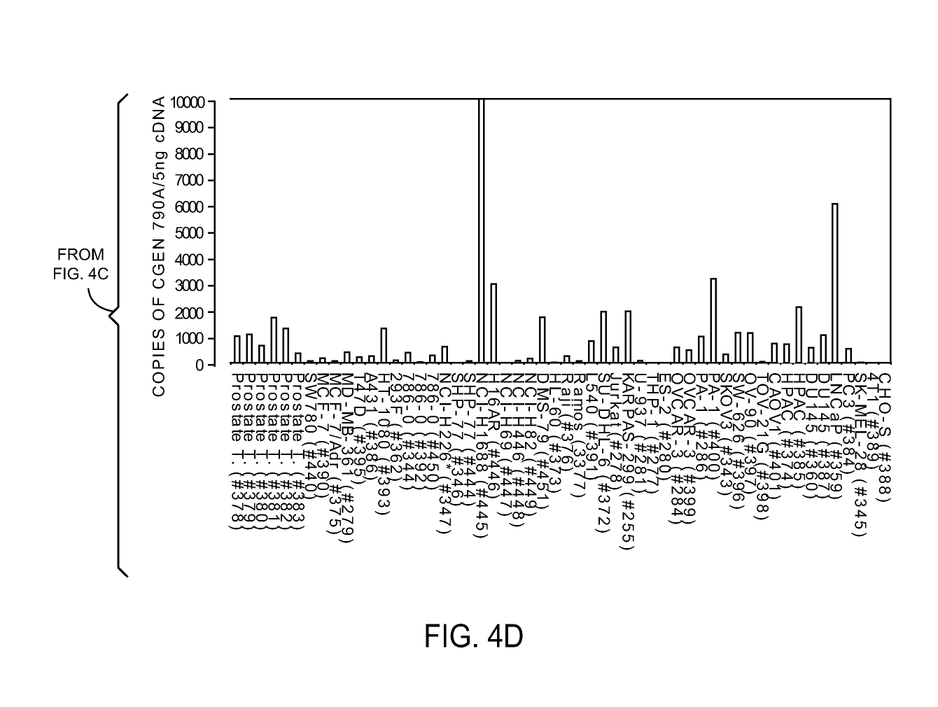

Expression of KIAA0746 detectable by or according to CGEN-790_seg33-34-36-1 amplicon (SEQ ID NO:81) and primers CGEN-790_seg33-34-36F1 (SEQ ID NO:79) and CGEN-790_seg33-34-36R1 (SEQ ID NO:80) was further measured by real time PCR. The samples used are detailed in Table 1 above. For each RT sample, the copy number of the amplicon, reflecting the expression of the KIAA0746 mRNA, was calculated from the corresponding Ct (see Table 1). The results of this analysis are depicted in the histogram in FIG. 4. High expression of the above-indicated KIAA0746 transcript in clearly seen in all lymphoma samples, and in several samples of other types of tumors: pancreas, prostate, ovary, melanoma, lung, liver, kidney, head & neck, and colon. Certain cell lines also show high expression of this gene.

Primer pairs are also optionally and preferably encompassed within the present invention; for example, for the above experiment, the following primer pair was used as a non-limiting illustrative example only of a suitable primer pair: CGEN-790_seg33-34-36F1 forward primer (SEQ ID NO:79); and CGEN-790_seg33-34-36R1 reverse primer (SEQ ID NO:80).

The present invention also preferably encompasses any amplicon obtained through the use of any suitable primer pair; for example, for the above experiment, the following amplicon was obtained as a non-limiting illustrative example only of a suitable amplicon:

### CGEN-790_seg33-34-36-1 (SEQ ID NO :81).

Forward primer: >CGEN-790_seg33-34-36F1

(SEQ ID NO: 79)

CCTTTCTGACGGATTCCAGC
Reverse primer: >CGEN-790_seg33-34-36R1

(SEQ ID NO: 80)

TCCAACCAAACTATACAACATGCC
Amplicon: CGEN-790_seg33-34-36-1

(SEQ ID NO: 81)

CCTTTCTGACGGATTCCAGCTGCTGTGGATACCATAAAGCATCCTACTAC
CTTGCAGTCTTTTATGAGACTGGATTAAATGTTCCTCGGGATCAGCTGCA
GGGCATGTTGTATAGTTTGGTTGGA

Expression of KIAA0746 Transcripts which are Detectable by Amplicon as Depicted in Sequence Name CGEN-790_seg33-34-36-2 (SEQ ID NO:84) in Normal and Cancerous Tissues

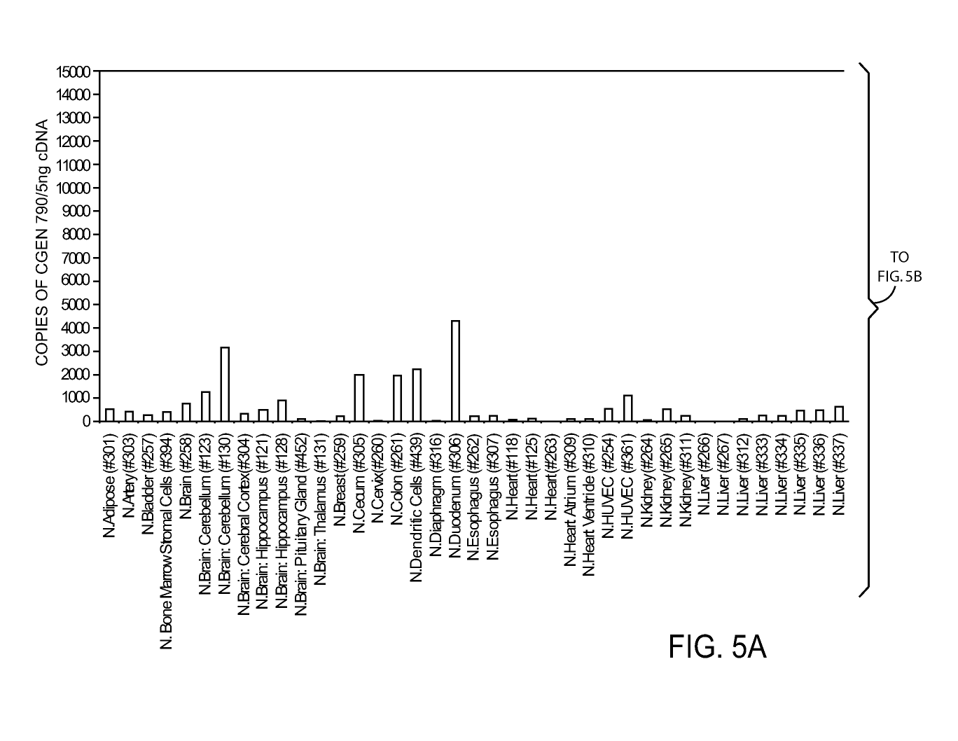

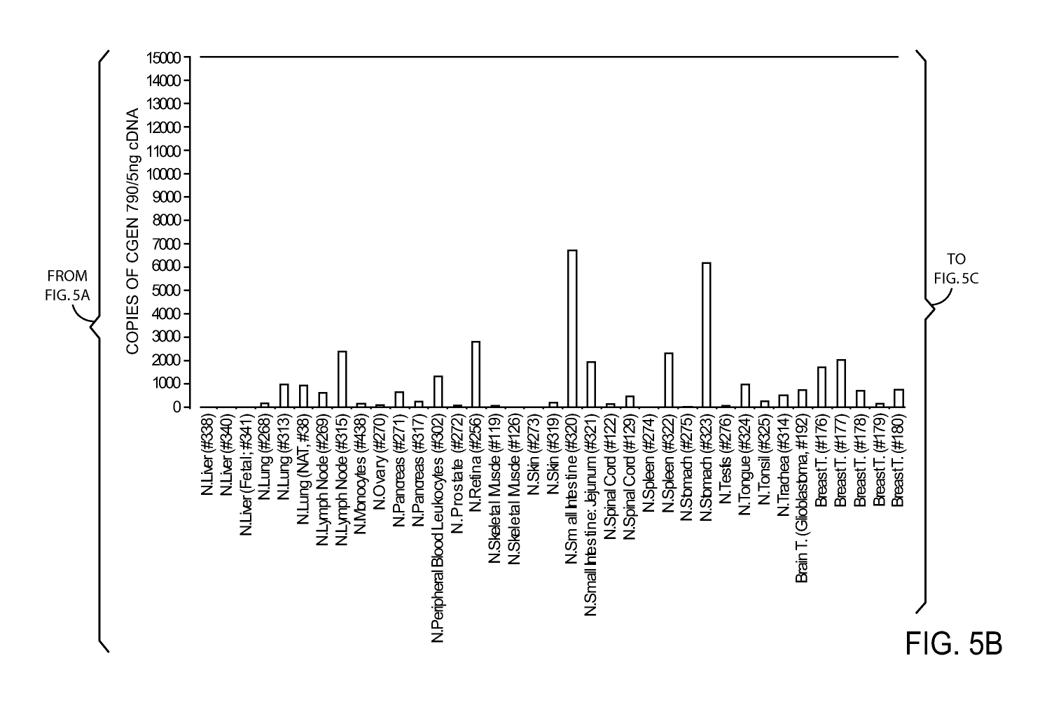

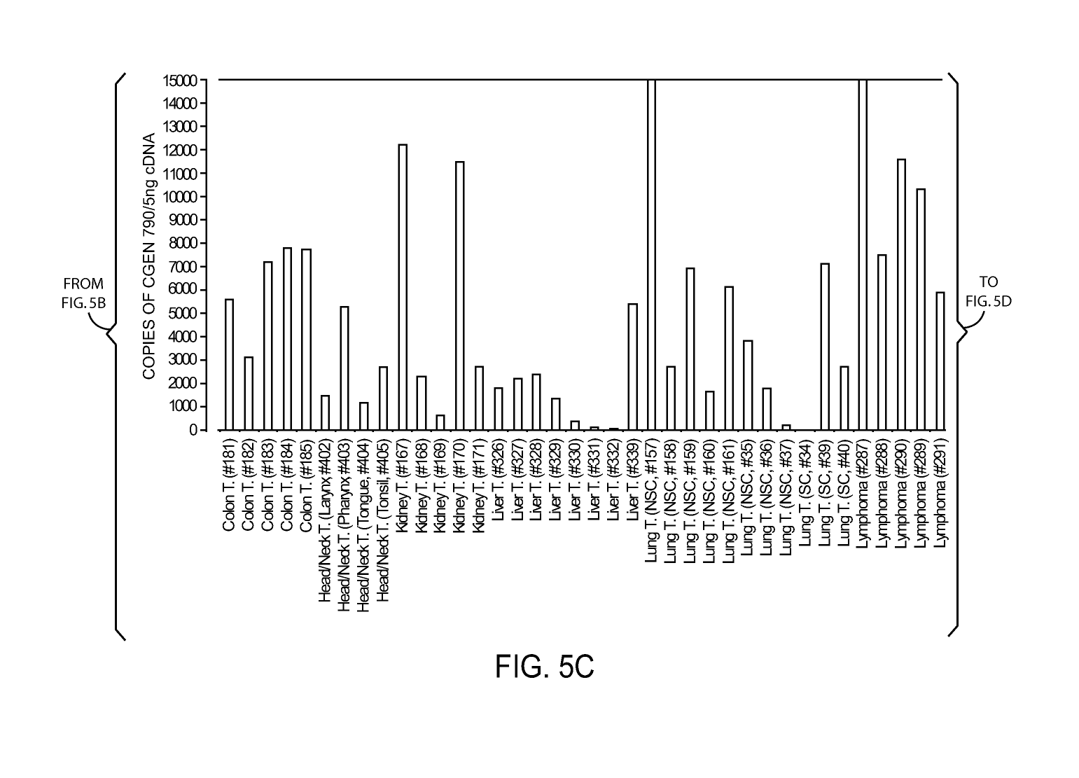

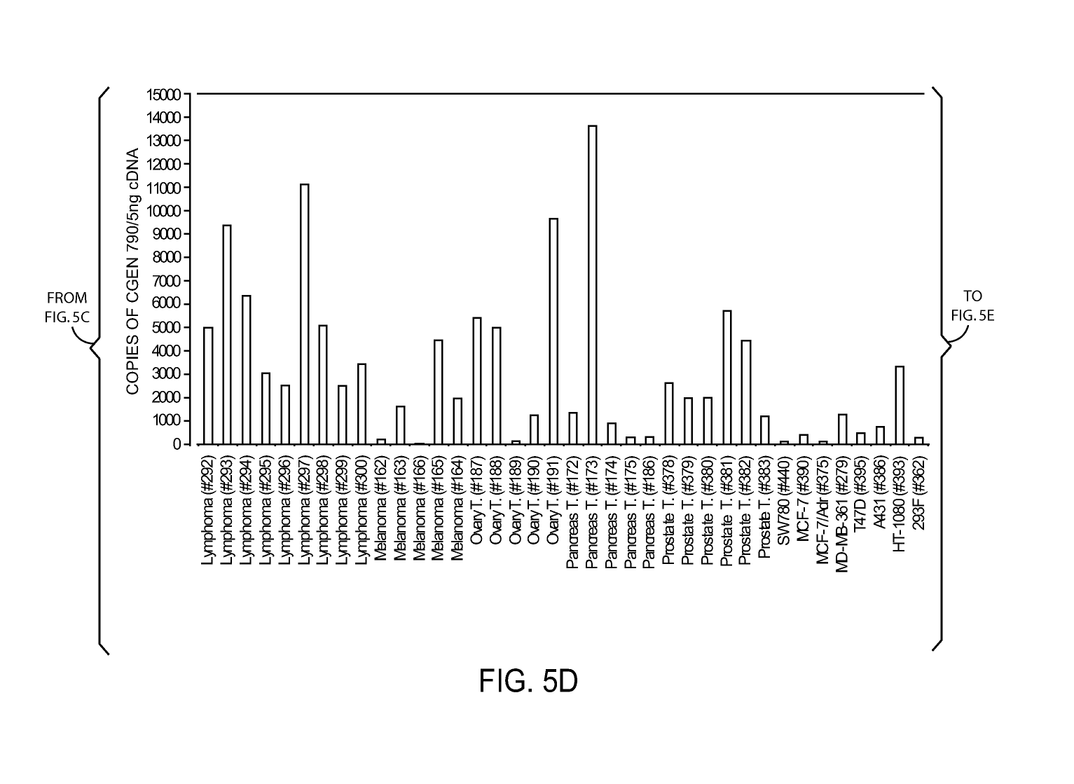

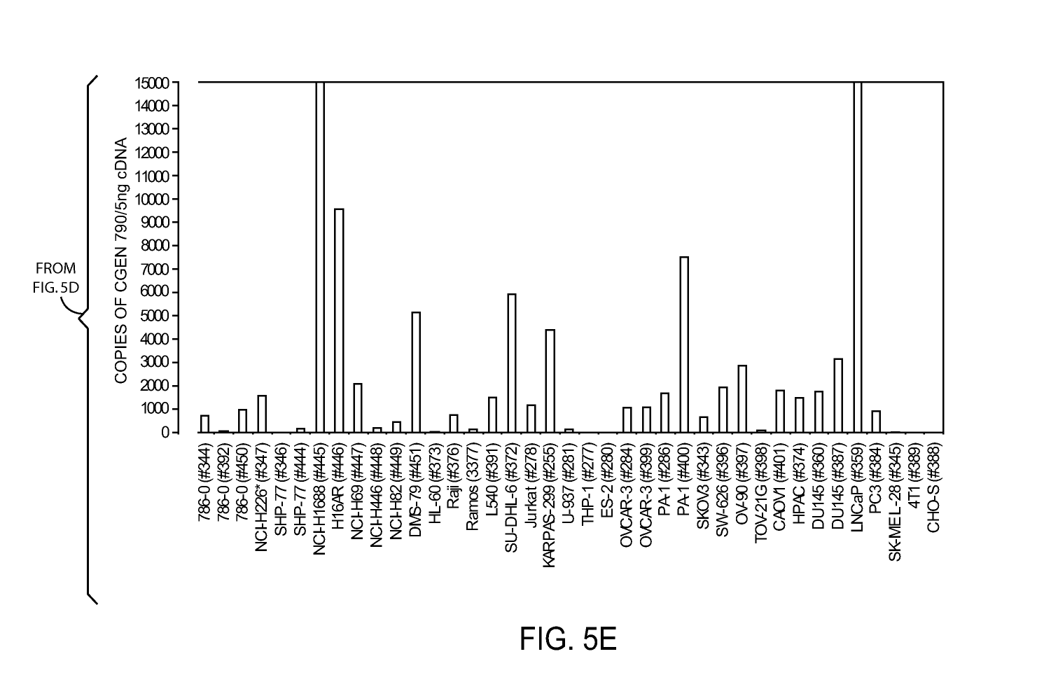

Expression of KIAA0746 detectable by or according to CGEN-790_seg33-34-36-2 amplicon (SEQ ID NO:84) and primers CGEN-790_seg33-34-36F2 (SEQ ID NO:82) and CGEN-790_seg33-34-36R2 (SEQ ID NO:83) was measured by real time PCR. The samples used are detailed in Table 1 above. For each RT sample, the copy number of the amplicon, reflecting the expression of the KIAA0746 mRNA, was calculated from the corresponding Ct, as described above (Table 1). The results of this analysis are depicted in the histogram in FIG. 5. High expression of the above-indicated KIAA0746 transcript in clearly seen in all lymphoma samples, and in several samples of other types of tumors: pancreas, prostate, ovary, melanoma, lung, liver, kidney, head & neck, and colon. Certain cell lines also show high expression of this gene. These results are similar to those obtained with the previous amplicon, and shown in FIG. 4. However, the overall levels of expression detected with this amplicon is higher.

Primer pairs are also optionally and preferably encompassed within the present invention; for example, for the above experiment, the following primer pair was used as a non-limiting illustrative example only of a suitable primer pair: CGEN-790_seg33-34-36F2 forward primer (SEQ ID NO:82); and CGEN-790_seg33-34-36R2 reverse primer (SEQ ID NO:83).

The present invention also preferably encompasses any amplicon obtained through the use of any suitable primer pair; for example, for the above experiment, the following amplicon was obtained as a non-limiting illustrative example only of a suitable amplicon:

### CGEN-790_seg33-34-36-2 (SEQ ID NO :84).

Forward primer: CGEN-790_seg33-34-36F2

(SEQ ID NO: 82)

TGACGGATTCCAGCTGCTG
Reverse primer: >CGEN-790_seg33-34-36R2

(SEQ ID NO: 83)

CCTGGCCTCCAACCAAACT
Amplicon: CGEN-790_seg33-34-36-2

(SEQ ID NO: 84)

TGACGGATTCCAGCTGCTGTGGATACCATAAAGCATCCTACTACCTTGCA
GTCTTTTATGAGACTGGATTAAATGTTCCTCGGGATCAGCTGCAGGGCAT
GTTGTATAGTTTGGTTGGAGGCCAGG

Expression of KIAA0746 Z43375 Transcripts which are Detectable by Amplicon as Depicted in Sequence Name Z43375_seg33-34-36F1R1 (SEQ ID NO:81) in the Blood-Specific Panel and in Normal and Cancerous Ovary Tissues.

Expression of KIAA0746 detectable by or according to Z43375_seg33-34-36F1R1 (SEQ ID NO:81) amplicon and primers Z43375_seg33-34-36F1 (SEQ ID NO:79) and Z43375_seg33-34-36R1 (SEQ ID NO:80) was measured by real time PCR on both blood and ovary panels. The samples used are detailed in Table 2 and Table 4 respectively above. For each RT sample, the expression of the above amplicon was normalized to the normalization factor calculated from the expression of several house keeping genes as described in section “Materials and Experimental Procedures” herein.

For blood panel—The normalized quantity of each RT sample was then divided by the median of the quantities of the kidney normal samples (sample numbers 65-67, Table 2 above), to obtain a value of relative expression of each sample relative to median of the kidney normal samples.

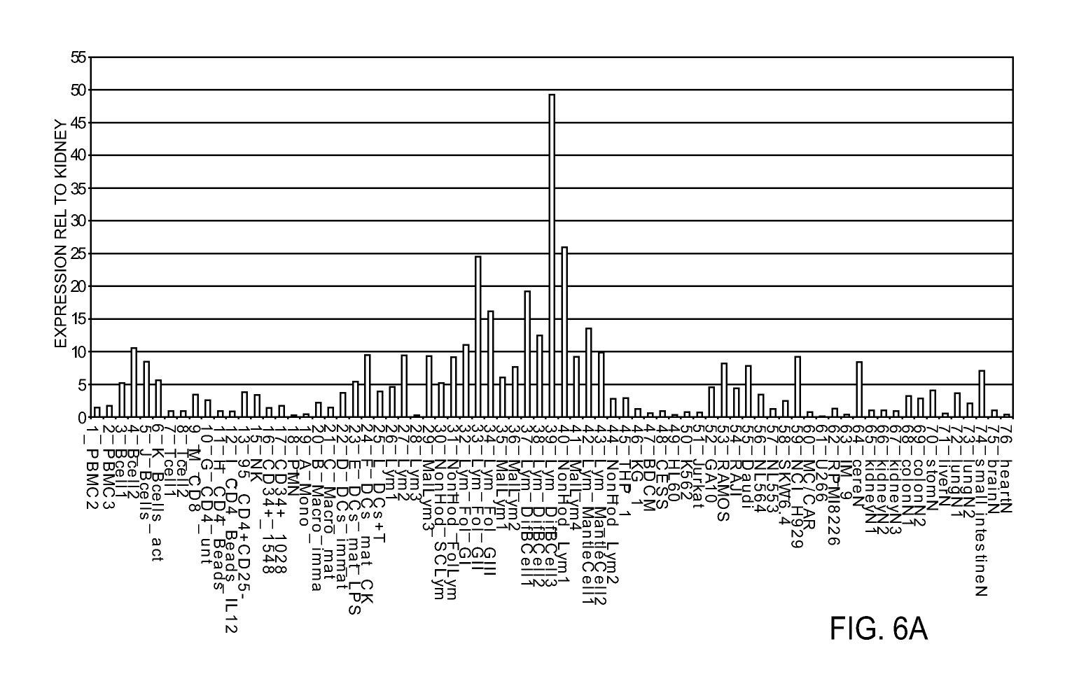

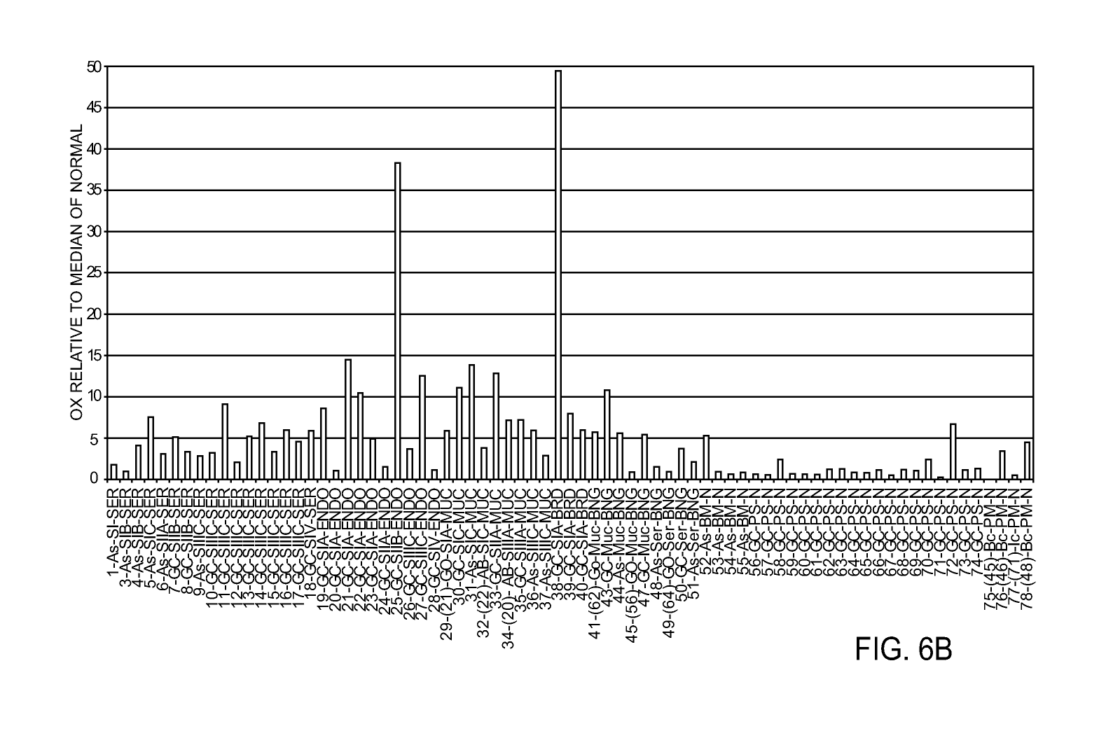

The results of this analysis are depicted in the histogram in FIG. 6A. High expression of the above-indicated KIAA0746 transcript is clearly seen in B-cells and in DCs, as well as all lymphoma samples and some of the cell lines.

Ovary panel—The normalized quantity of each RT sample was then divided by the median of the quantities of the normal samples (sample numbers 52-65, Table 4), to obtain a value of fold up-regulation for each sample relative to median of the normal samples.

FIG. 6B is a histogram showing over expression of the above-indicated KIAA0746 transcripts in cancerous ovary samples relative to the normal samples.

As is evident from FIG. 6B, the expression of KIAA0746 transcripts detectable by the above amplicon in serous carcinoma, mucinous carcinoma and adenocarcinoma samples was significantly higher than in the non-cancerous samples (sample numbers 52-65, Table 4). Notably an over-expression of at least 5 fold was found in 7 out of 17 serous carcinoma samples, in 7 out of 9 mucinous carcinoma samples, in 5 out of 10 endometroid samples and 19 out of 36 adenocarcinoma samples.

Statistical analysis was applied to verify the significance of these results, as described below.

The P value for the difference in the expression levels of KIAA0746 transcripts detectable by the above amplicon in ovary serous carcinoma samples, mucinous carcinoma samples, endometroid samples and adenocarcinoma samples and versus the normal tissue samples was determined by T test as 4.56e-005, 5.63e-004, 2.41e-002 and 2.67e-005, respectively.

Threshold of 5 fold over expression was found to differentiate between serous carcinoma, mucinous carcinoma, endometroid and adenocarcinoma samples and normal samples with P value of 1.23e-002, 1.67e-004, 1.03e-002 and 1.62e-004, respectively, as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: Z43375_seg33-34-36F1 forward primer (SEQ ID NO:79); and Z43375_seg33-34-36R1 reverse primer (SEQ ID NO:80).

The present invention also preferably encompasses any amplicon obtained through the use of any suitable primer pair; for example, for the above experiment, the following amplicon was obtained as a non-limiting illustrative example only of a suitable amplicon:

### Z43375_seg33-34-36-F1R1 (SEQ ID NO:81).

Forward primer: >CGEN-790_Z43375_seg33-34-36F1

(SEQ ID NO: 79)

CCTTTCTGACGGATTCCAGC
Reverse primer: >CGEN-790 Z43375_seg33-34-36R1

(SEQ ID NO: 80)

TCCAACCAAACTATACAACATGCC
Amplicon: CGEN-790 Z43375_seg33-34-36-1_F1R1

(SEQ ID NO: 81)

CCTTTCTGACGGATTCCAGCTGCTGTGGATACCATAAAGCATCCTACTAC
CTTGCAGTCTTTTATGAGACTGGATTAAATGTTCCTCGGGATCAGCTGCA
GGGCATGTTGTATAGTTTGGTTGGA

In one experiment, carried out using primers Z43375_seg33-34-36F1 (SEQ ID NO:79) and Z43375_seg33-34-36R1 (SEQ ID NO:80), no differential expression was observed in breast cancerous samples, lung cancerous samples and colon cancerous samples, relative to the corresponding normal samples.

### Example 2_{—}3

### Cloning and Expression of KIAA0746 Extra Cellular Domain (ECD) fused to mouse Fc

In order to produce antibodies against the extra cellular domain (ECD) of KIAA0746_T0_P4 (SEQ ID NO:93), KIAA0746 ECD fragments fused to mouse Fc IgG2a (GenBank Accession-CAA49868, amino acid residues 237-469) were expressed in HEK-293T ((ATCC-CRL-11268).

KIAA0746 ECD was divided into four domains, as follows: amino acids residues at positions 34-305; amino acids residues at positions 306-508; amino acids residues at positions 509-765; and amino acids residues at positions 766-1023 of KIAA0746_T0_P4 (SEQ ID NO: 18) KIAA0746_T0_P4 ECD sequence corresponding to amino acids residues at positions 34-1023 (SEQ ID 130) of the KIAA0746 P4 protein (SEQ ID NO: 18), followed by IL6 signal peptide, was codon optimized to boost protein expression in mammalian system. DNA was synthesized by GeneArt (Germany). The DNA was then subcloned in frame to mFc pIRESpuro3 (SEQ ID 219) and used as a temple for PCR amplification of the four ECDs fragments described above.

PCR was done using Platinum PFX™ (Invitrogen., Carlsbad, Calif., USA, catalog number: 1178-021) under the following conditions: 5 μl Platinum PFX 10× buffer; 1 μl (20 ng)—DNA from Gene Art; 1 μl—10 mM dNTPs (2.5 mM of each nucleotide); 1 μl—Platinum PFX enzyme; 34.5 μl—H2O; and 1 μl—of each primer (10 μM) in a total reaction volume of 50 μl; with a reaction program of 3 minutes in 94° C.; 30 cycles of: 30 seconds at 94° C., 30 seconds at 57° C., 60 seconds at 68° C.; then 10 minutes at 68° C. Primers (SEQ ID NOs: 115-116; 117-118; 117-119; 120-121) which were used include gene specific sequences corresponding to the desired coordinates of the proteins described above.

50 μl of PCR products were loaded onto a 1.3% agarose gel stained with ethidium bromide, electrophoresed in 1×TAE solution at 100V, and visualized with UV light. After verification of expected band size, the PCR product was excised and extracted from the gel using QiaQuick™ Gel Extraction kit (Qiagen, catalog number: 28707). The extracted PCR products were digested with the appropriate restriction enzymes (New England Biolabs, Beverly, Mass., U.S.A.). After digestion, DNAs were loaded onto a 1% agarose gel as described above. The expected bands size were excised and extracted from the gel using QiaQuick™ Gel Extraction kit (Qiagen, catalog number: 28707).

The digested DNAs were ligated to mFc_pIRESpuro3 vector, or IL6-mFc_pIRESpuro vector previously digested with the same enzymes, using the LigaFast™ Rapid DNA Ligation System (Promega, catalog number: M8221). The resulting DNAs were transformed into competent E. coli bacteria DH5α (RBC Bioscience, Taipei, Taiwan, catalog number: RH816) according to manufacturer's instructions, then plated on LB-ampicillin agar plates for selection of recombinant plasmids, and incubated overnight at 37° C. The following day, a number of colonies from each transformation that grew on the selective plates were taken for further analysis by streak-plating on another selective plate and by PCR using GoTaq ReadyMix (Promega, catalog number: M7122). Screening of positive clones was performed by PCR using pIRESpuro3 vector specific primer and gene specific primer (data not shown). After completion of all PCR cycles, half of the reaction was analyzed using 1% agarose gel as described above. After verification of expected band size, two positive colonies from each ligation reactions were grown in 5 ml Terrific Broth supplemented with 100 μg/ml ampicillin, with shaking overnight at 37° C. Plasmid DNA was isolated from bacterial cultures using Qiaprep™ Spin Miniprep Kit (Qiagen, catalog number: 27106). Accurate cloning was verified by sequencing the inserts (Weizmann Institute, Rehovot, Israel). Upon verification of an error-free colony (i.e. no mutations within the ORF), recombinant plasmids were processed for further analyses.

Cloning details of each of the four constructs are presented in Table 48, below:

TABLE 48

| | | | Restriction | |
|---|---|---|---|---|
| Construct | Forward primer | Reverse primer | Enzymes | Ligated to |
| KIAA0746_(aa 34-305) | 100-808 (SEQ | 100-913 (SEQ | NheI- | mFc |
| ECD-_mFc_pIRESpuro | ID NO: 115) | ID NO: 116) | BamHI | pIRESpuro3 |
| KIAA0746_(aa 306-508) | 100-914 (SEQ | 100-915 (SEQ | BstBI- | IL6- |
| ECD-_mFc_pIRESpuro | ID NO: 117) | ID NO: 118) | BamHI | mFc_pIRESpuro3 |
| KIAA0746_(aa 509-765) | 100-914 (SEQ | 100-917 (SEQ | BstBI- | IL6- |
| ECD-mFc_pIRESpuro | ID NO: 117) | ID NO: 119) | BamHI | mFc_pIRESpuro3 |
| KIAA0746_(aa 766-1023) | 100-918 (SEQ | 100-856 (SEQ | BstBI- | IL6- |
| ECD-_mFc_pIRESpuro | ID NO: 120) | ID NO: 121) | BamHI | mFc_pIRESpuro3 |

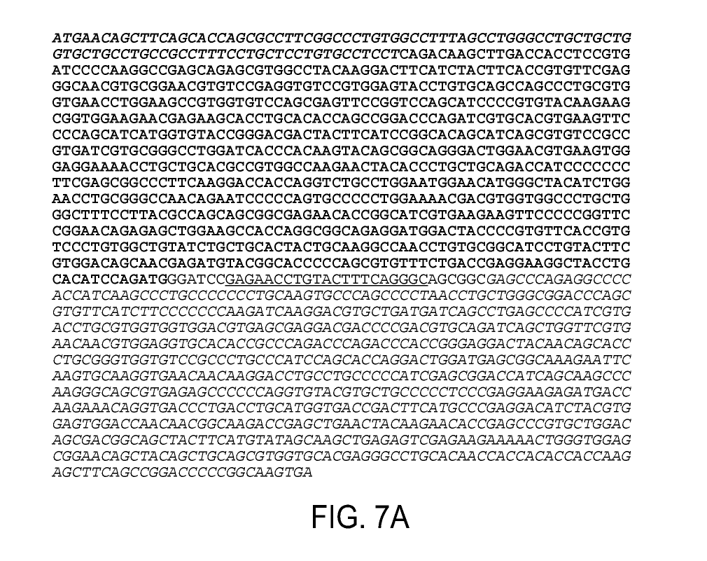

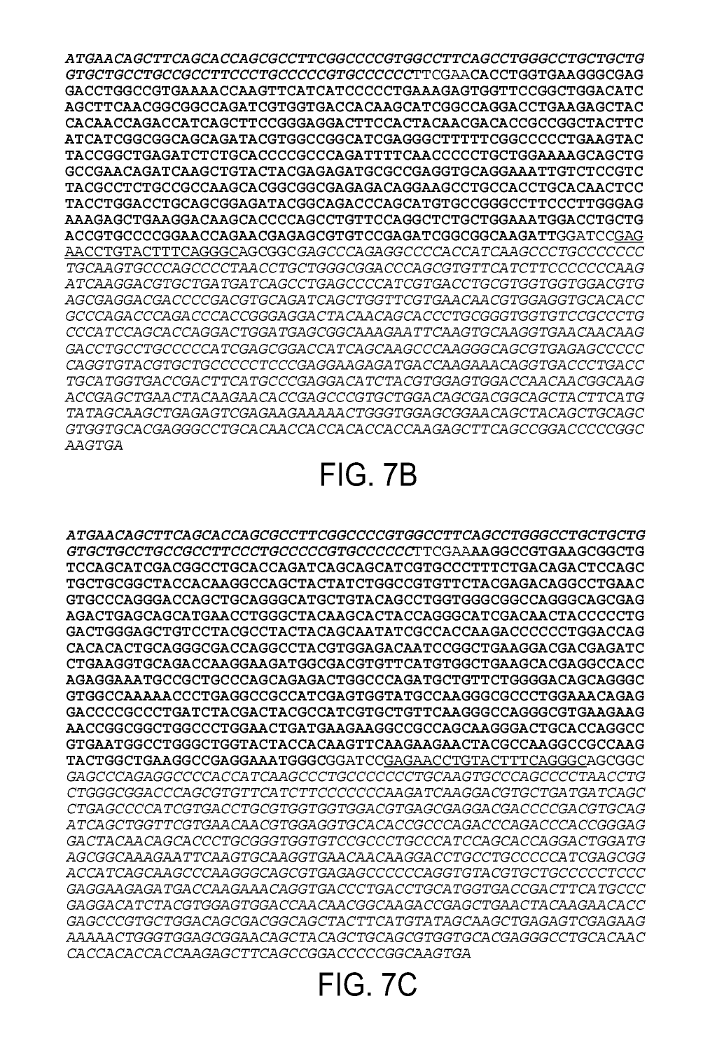

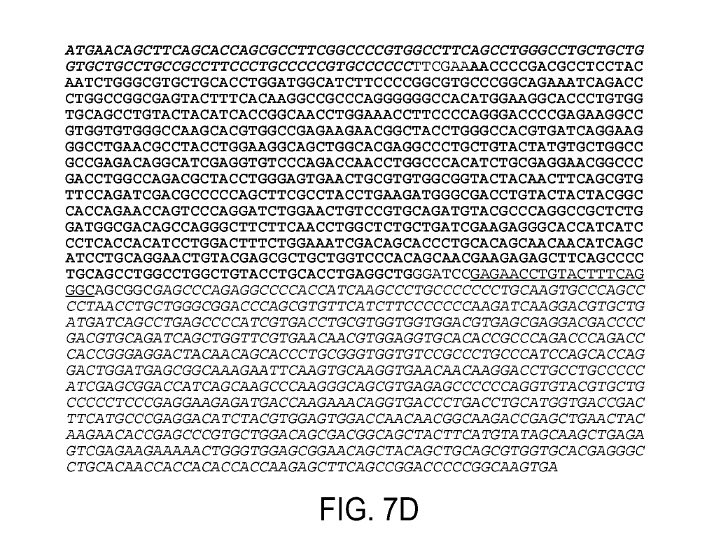

The nucleotide sequences of the resulting KIAA0746_T0_P4 ECD_mFc ORFs are shown in FIG. 7A-D: gene specific sequence correspond to the ECD sequence is marked in bold faced, TEV cleavage site sequence is underlined, mFc sequence is Italic and IL6 signal peptide sequence is bold Italic. FIG. 7A shows the KIAA0746_(aa 34-305) ECD_mFc DNA sequence (1647 bp) (SEQ ID NO:122); FIG. 7B shows the KIAA0746_(a.a 306-508) ECD-mFc DNA sequence (1446 bp) (SEQ ID NO:123), FIG. 7C shows the KIAA0746_(a.a 509-765) ECD_mFc DNA sequence (1602 bp) (SEQ ID NO:124); FIG. 7D shows the KIAA0746_(a.a 766-1023) ECD_mFc DNA sequence (1611 bp) (SEQ ID NO:125).

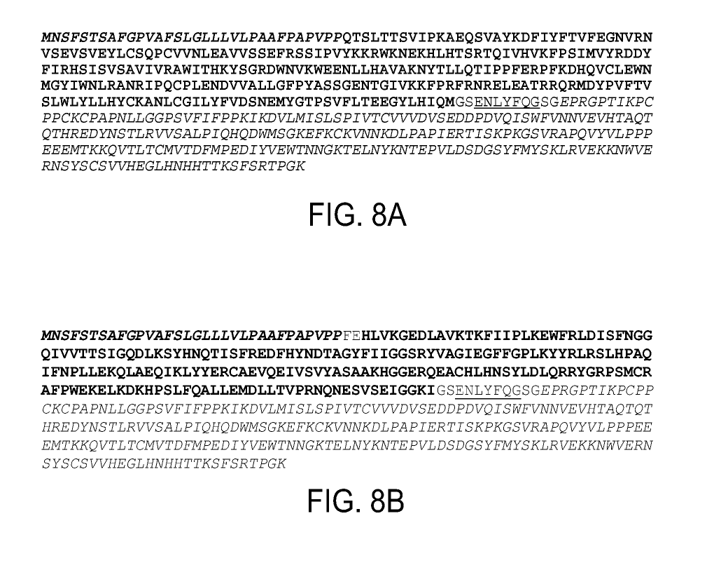

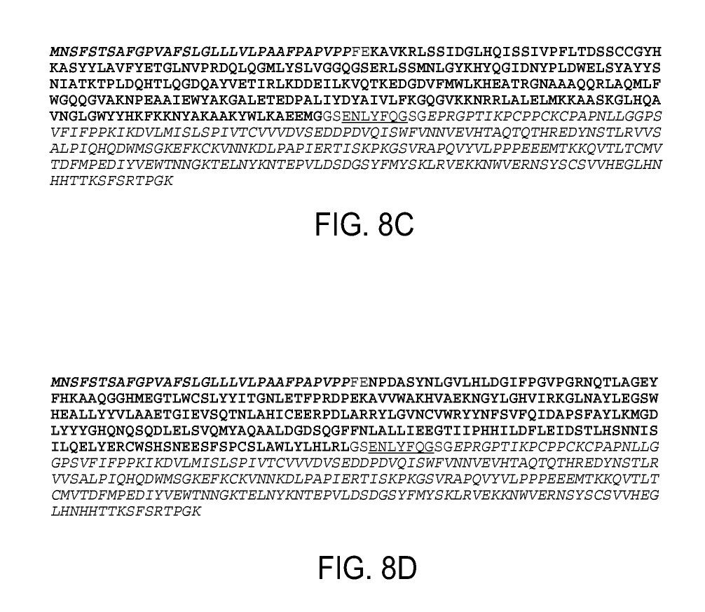

The sequence of the resulting ECD_mFc fusion proteins are shown in FIG. 8A-D; gene specific sequence correspond to the ECD sequence is marked in bold faced, TEV cleavage site sequence is underlined, mFc sequence is Italic and IL6 signal peptide sequence is bold Italic. FIG. 8A shows the KIAA0746_(a.a 34-305) ECD_mFc amino acid sequence (SEQ ID NO:126); FIG. 8B shows the KIAA0746_(a.a 306-508) ECD_mFc amino acid sequence (SEQ ID NO:127), FIG. 8C shows the KIAA0746_(a.a 509-765) ECD_mFc amino acid sequence (SEQ ID NO:128); FIG. 8D shows the KIAA0746_(a.a 766-1023) ECD_mFc amino acid sequence (SEQ ID NO:129).

To generate cells that stably express ECD-mFc, HEK-293T cells were transfected with the above described constructs corresponding to KIAA0746 extra cellular domain fused to mouse Fc, or pIRES puro3 empty vector, as follows:

HEK-293T (ATCC, CRL-11268) cells were plated in a sterile 6 well plate suitable for tissue culture, using 2 ml pre-warmed of complete media, DMEM [Dulbecco's modified Eagle's Media, Biological Industries (Beit Ha'Emek, Israel), catalog number: 01-055-1A]+10% FBS [Fetal Bovine Serum, Biological Industries (Beit Ha'Emek, Israel), catalog number: 04-001-1A]+4 mM L-Glutamine [Biological Industries (Beit Ha'Emek, Israel), catalog number: 03-020-1A]. 500,000 cells per well were transfected with 2 μg of DNA construct using 6 μl FuGENE 6 reagent (Roche, catalog number: 11-814-443-001) diluted into 94 μl DMEM. The mixture was incubated at room temperature for 15 minutes. The complex mixture was added dropwise to the cells and swirled. Cells were placed in incubator maintained at 37° C. with 5% CO₂ content. 48 hours following transfection, the transfected cells were transferred to a 75 cm² tissue culture flask containing 15 ml of selection media: complete media supplemented with 5 μg\ml puromycin (Sigma, catalog number P8833). Cells were placed in incubator, and media was changed every 3-4 days, until clone formation observed. To verify the identity of cells, genomic PCR was performed, indicating the correct sequences integrated into the cell genome (data not shown).

In order to verify the expression of KIAA0746 ECD mFc proteins, cell-deprived medium was collected and purified by Protein A-Sepharose beads as follows: 1 ml of cell-deprived medium was incubated with 60 μl Protein A sepharose beads (Amersham catalog number 17-5280-04) for 45 minutes at room temperature. At the end of incubation time proteins were eluted from the beads pellet with 50 ml sample buffer containing 100 mM Citrate Phosphate pH 3.5 and 100 mM DTT. The samples were boiled for 3 minutes and 30 μl were loaded on 4-12% NuPAGE Bis Tris gel (Invitrogen, catalog number NPO322). The proteins were transferred to a nitrocellulose membrane and blocked with 10% low fat milk in PBST (PBS supplemented with 0.05% tween-20). The membrane was then blotted for over night at 4° C. with Goat anti mouse IgG2a Fc fragment HRP (Jackson, catalog number 115-035-206) diluted 1:20,000 in blocking solution. Following incubation with ECL solution (Amersham Biosciences, Catalog No. RPN2209), the membrane was exposed to film.

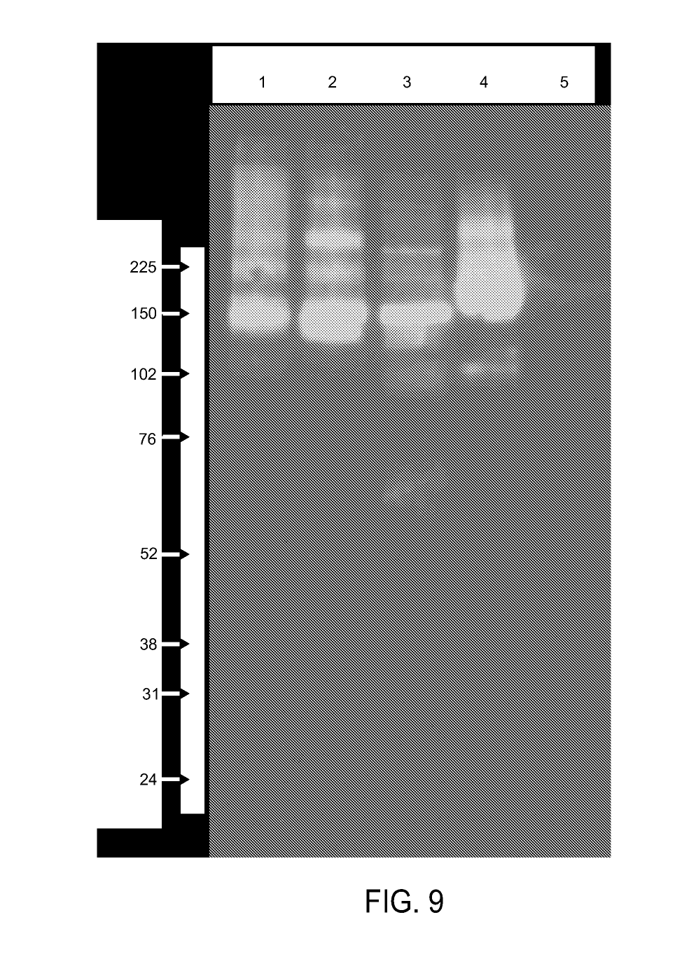

FIG. 9 shows the results of a Western blot analysis of KIAA0746_(aa 34-305) ECD_mFc (SEQ ID NO:126), KIAA0746_(aa 306-508) ECD_mFc (SEQ ID NO:127), KIAA0746 (aa 509-765) ECD_mFc (SEQ ID NO:128) and KIAA0746_(aa 766-1023) ECD_mFc (SEQ ID NO:129) constructs in the medium of HEK-293T stably transfected cells.

The lanes are as follows: Molecular weight marker (Amersham, full range rainbow, catalog number RPN800) are marked; 1—KIAA0746_(aa 34-305) ECD_mFc (SEQ ID NO: 126); 2—KIAA0746_(aa 306-508) ECD_mFc (SEQ ID NO: 127); 3—KIAA0746_(aa 509-765) ECD-_mFc (SEQ ID NO: 128); 4—KIAA0746_(aa 766-1023) ECD_mFc (SEQ ID NO: 129); 5-pIRES puro3 empty vector.

### Example 3

### CD20 Polypeptides and Polynucleotides, and Uses Thereof as a Drug Target for Producing Drugs and Biologics

### Example 3_{—}1

### Description for Cluster HSCD20B

CD20 is encoded by a member of the Membrane-spanning 4A gene family. The CD20 protein is an integral membrane protein that crosses the cell membrane four times. It plays a role in the development and differentiation of B-cells. It has no known natural ligand, and it functions as a calcium ion channel. CD20 is expressed on the surface of pre-B and mature B lymphocytes, but not on stem cells. Plasma blasts and stimulated plasma cells may also express CD20. This antigen is expressed on the vast majority of B-cell leukemias and lymphomas. Only a smaller fraction of plasma cell neoplasms (i.e. multiple myeloma) and myeloid leukemias are CD20 positive.

B-cells play an important role in the pathogenesis of various immune related conditions, such as autoimmune diseases and transplant rejection (Jeffrey Browning, 2006, Nature Reviews Drug Discovery, 5:564-576). In addition, several hematopoietic malignancies derive from pre-B or B-lymphocytes. Antagonistic antibodies targeting these immune cells are gaining increasing role in the management of such diseases (Fanale and Younes, 2007, Drugs 67: 333-350; Martin and Leonard, Li and Zhu, 2007, Expert. Opin. Biol. Ther. 7: 319-330).

The first antibody target in this regard was CD20, which is the target of rituximab, an antibody which is successfully used in the clinic for various B-cell malignancies and autoimmune diseases (Pescovitz, 2006, Am. J. Transplant. 6: 859-866). CD20 is the target of other antibodies, such as ibritumomab tiuxetan and toxitumomab, which are radioconjugates and are also used in the treatment of B cell lymphomas and leukemias. Rituximab causes B-cell depletion, thus eliminating B-cell derived malignant cells in various hematopoietic malignancies, such as leukemias, lymphomas and multiple myeloma (Coiffier, 2007, Oncogene 26: 3603-3613; Bosly et al, 2002 Anticancer Drugs Suppl 2:S25-33). In addition, B-lymphocyte depletion has proven efficacious in various autoimmune diseases where auto-antibodies play a role in the clinical pathology or where removal of B-cells might starve T-cells of autoantigen presenting cells (Goldblatt and Isenberg, 2008, Handb Exp Pharmacol. 181: 163-181; Dass et al 2006, Expert. Opin. Pharmacother. 7: 2559-2570; Prajapati and Mydlarski, 2007, Skin Therapy Lett. 12: 6-9; Cianchini et al, 2007, Arch Dermatol. 143: 1033-1038). Recently, Rituximab has been used to treat autoimmune diseases, especially those associated with a prominent humoral component and with potentially pathogenic autoantibodies. Autoimmune diseases that have shown benefit from targeting CD20 include rheumatoid arthritis (RA), psoriatic arthritis, thrombocytopenic purpura, primary Sjogren's syndrome, systemic lupus erythematosus (SLE), rheumatoid arthritis (RA), psoriatic arthritis, Myasthenia Gravis, idiopathic autoimmune hemolytic anemia, pure red cell aplasia, thrombocytopenic purpura, Evans syndrome, vasculitis, cryoglobulinemic vasculitis, ANCA-associated vasculitis, Wegener's granulomatosis, microscopic polyangiitis, primary biliary cirrhosis, chronic urticaria, dermatomyositis, polymyositis, multiple sclerosis, bullous skin disorders (such as pemphigus), atopic eczema, type 1 diabetes mellitus, Devic's disease, pure red cell aplasia, Evan's syndrome, vasculitis, multiple sclerosis, bullous skin disorders (for example pemphigus, pemphigoid), type 1 diabetes mellitus.

Furthermore, immunomodulation of B-cells has therapeutic value in graft rejection following organ transplantation, since B-cells and the alloantibodies made by them, are pathogenic in both acute and chronic graft rejection (Venetz and Pascual, 2007, Expert Opin. Invest. Drugs 16: 625-633; Kaczmarek et al 2007, J. Heart Lung Transplant. 26: 511-515). Rituximab is now being used in the management of renal transplant recipients to diminish levels of alloreactive antibodies in highly sensitized patients, to manage ABO-incompatible transplants, and to treat rejection associated with activation of B cells and development of anti-donor antibodies. In addition, Rituximab is being evaluated in patients undergoing stem cell transplantation and in patients who have developed GVHD (Graft Versus Host Disease) following allogeneic stem cell transplantation.

In addition, B-lymphocyte depletion has also proven efficacious in various lymphoproliferative diseases, such as PTLD (posttransplant lymphoproliferative disorder), Waldenstrom's macroglobulinemia, cryoglobulinemia, etc. (Frey and Tsai, 2007, Med. Oncol. 24: 125-136; Vijay and Gertz, 2007, blood 109: 5096-5103; Tedeschi et al 2007, Blood Rev. 21: 183-200).

Harnessing the immune system to treat chronic diseases is a major goal of immunotherapy. Active and passive immunotherapies are proving themselves as effective therapeutic strategies. Passive immunotherapy, using monoclonal antibodies or receptor Fc-fusion proteins, has come of age and has shown great clinical success. A growing number of such therapeutic agents have been approved or are in clinical trials to prevent allograft rejection or to treat autoimmune diseases and cancer. Active immunotherapy (i.e. vaccines) has been effective against agents that normally cause acute self-limiting infectious diseases followed by immunity and has been at the forefront of efforts to prevent the infectious diseases that plague humankind. However, active immunotherapy has been much less effective against cancer or chronic infectious diseases primarily because these have developed strategies to escape normal immune responses.

Passive tumor immunotherapy uses the exquisite specificity and lytic capability of the immune system to target tumor specific antigens and treat malignant disease with a minimum of damage to normal tissue. Several approaches have been used to identify tumor-associated antigens as target candidates for immunotherapy. The identification of novel tumor specific antigens expands the spectrum of tumor antigen targets available for immune recognition and provides new target molecules for the development of therapeutic agents for passive immunotherapy, including monoclonal antibodies, whether unmodified or armed. Such novel antigens may also point the way to more effective therapeutic vaccines for active or adoptive immunotherapy. Three different mechanisms have been proposed for the elimination of B cells by rituximab, including complement-dependent cytotoxicity (CDC), antibody-dependent cellular cytotoxicity (ADCC), and stimulation of the apoptotic pathway.

Cluster HSCD20B (internal ID 76553270) features 1 transcript HSCD20B_{—}1_T12 (SEQ ID NO:31) of interest, encoding protein variant HSCD20B_{—}1_P5 (SEQ ID NO:33). These sequences are variants of the known protein B-lymphocyte antigen CD20 (SEQ ID NO:32) (SwissProt accession identifier CD20 HUMAN (SEQ ID NO:32); known also according to the synonyms B-lymphocyte surface antigen B1; Leu-16; Bp35), referred to herein as the previously known protein. Known polymorphisms for this sequence are as shown in Table 49.

TABLE 49
Amino acid mutations for Known Protein

| SNP position(s) on | |
|---|---|
| amino acid sequence | Comment |
| 13 | P −> L |
| 71 | M −> I |

Protein B-lymphocyte antigen CD20 (SEQ ID NO:32) localization is believed to be Membrane; multi-pass membrane protein.

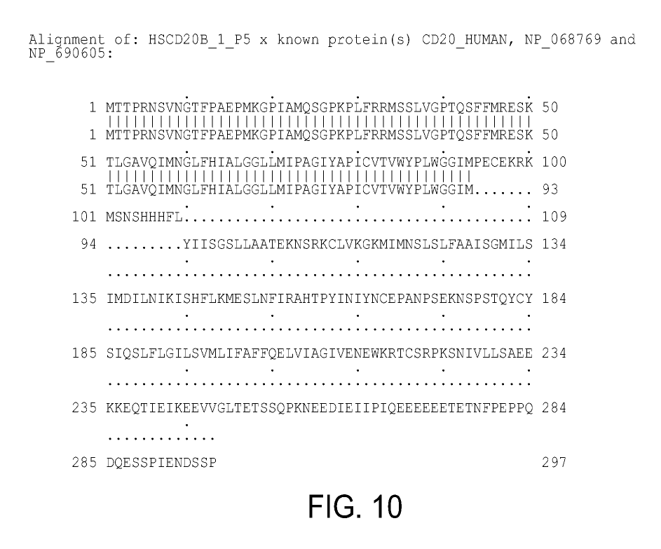

The splice variant of CD20, HSCD20B_{—}1_P5 (SEQ ID NO:33), contains the first two coding exons of the wild type CD20, followed by a novel exon of 501 bp, encoding a unique coding region of 16 amino acids. The variant maintains the first transmembrane region of the wild type CD20 but doesn't have the following three transmembrane regions. Therefore the HSCD20B_{—}1_P5 (SEQ ID NO:33) variant is predicted to expose a different epitope of CD20 upon the cell membrane as compared with the wild type CD20. This unique region will not be recognized with the currently available anti-CD20 antibodies, directed to the known CD20. A description of HSCD20B_{—}1_P5 (SEQ ID NO:33) variant protein according to the present invention is now provided. Variant protein HSCD20B_{—}1_P5 (SEQ ID NO:33) according to the present invention has an amino acid sequence encoded by transcript HSCD20B_{—}1_T12 (SEQ ID NO:31). One or more alignments to one or more previously published protein sequences are given in FIG. 10. A brief description of the relationship of the variant protein according to the present invention to each such aligned protein is as follows:

Comparison report between HSCD20B_{—}1_P5 (SEQ ID NO:33) and known protein CD20 HUMAN (SEQ ID NO:32) (FIG. 10):

A. An isolated chimeric polypeptide encoding for HSCD20B_{—}1_P5 (SEQ ID NO:33), comprising a first amino acid sequence being at least 90% homologous to MTTPRNSVNGTFPAEPMKGPIAMQSGPKPLFRRMSSLVGPTQSFFMRESKTLGAVQI MNGLFHIALGGLLMIPAGIYAPICVTVWYPLWGGIM corresponding to amino acids 1-93 of known protein CD20 HUMAN (SEQ ID NO:32), which also corresponds to amino acids 1-93 of HSCD20B_{—}1_P5 (SEQ ID NO:33), and a second amino acid sequence being at least 70%, optionally at least 80%, preferably at least 85%, more preferably at least 90% and most preferably at least 95% homologous to a polypeptide having the sequence PECEKRKMSNSHHHFL corresponding to amino acids 94-109 of HSCD20B_{—}1_P5 (SEQ ID NO:33), wherein said first amino acid sequence and second amino acid sequence are contiguous and in a sequential order.

B. An isolated polypeptide encoding for an edge portion of HSCD20B_{—}1_P5 (SEQ ID NO:33), comprising an amino acid sequence being at least 70%, optionally at least about 80%, preferably at least about 85%, more preferably at least about 90% and most preferably at least about 95% homologous to the sequence PECEKRKMSNSHHHFL of HSCD20B_{—}1_P5 (SEQ ID NO:33).

The localization of the variant protein was determined according to results from a number of different software programs and analyses, including analyses from SignalP and other specialized programs. The variant protein is believed to be located as follows with regard to the cell: membrane.

Variant protein HSCD20B_{—}1_P5 (SEQ ID NO:33) also has the following non-silent SNPs (Single Nucleotide Polymorphisms) as listed in Table 50, (given according to their position(s) on the amino acid sequence, with the alternative amino acid(s) listed; the last column indicates whether the SNP is known or not; the presence of known SNPs in variant protein HSCD20B_{—}1_P5 (SEQ ID NO:33) sequence provides support for the deduced sequence of this variant protein according to the present invention).

TABLE 50
Amino acid mutations

| SNP position(s) on | | |
|---|---|---|
| amino acid sequence | Alternative amino acid(s) | Previously known SNP? |
| 13 | P −> Q | Yes |
| 13 | P −> R | Yes |
| 72 | I −> T | Yes |

The coding portion of transcript HSCD20B_{—}1_T12 (SEQ ID NO:31) starts at position 484 and ends at position 810. The transcript also has the following SNPs as listed in Table 51 (given according to their position on the nucleotide sequence, with the alternative nucleic acid listed).

TABLE 51
Nucleic acid SNPs

| Polymorphism | SNP position(s) on nucleotide sequence |
|---|---|
| G −> T | 312 |
| G −> C | 312 |
| C −> A | 521 |
| C −> G | 521 |
| T −> C | 698 |

### Example 3_{—}2

### Analysis of the Expression of CD20 Transcripts Expression of CD20-Variant Transcripts which are Detectable by Amplicon as Depicted in Sequence Name seg10-12F2R2 (SEQ ID NO:87) on The Blood-Specific Panel, on Different Normal Tissues and on Combined Panel

Expression of CD20-variant transcripts detectable by or according to seg10-12F2R2 amplicon (SEQ ID NO:87) and primers seg10-12F2 (SEQ ID NO:85) and seg10-12R2 (SEQ ID NO:86) was measured by real time PCR on blood panel, normal panel and combined panel. The samples used are detailed in Table 2, Table 3 and Table 5, respectively. For each RT sample, the expression of the above amplicon was normalized to the normalization factor calculated from the expression of several house keeping genes as described in section “Materials and Experimental Procedures” above.

Blood Panel—

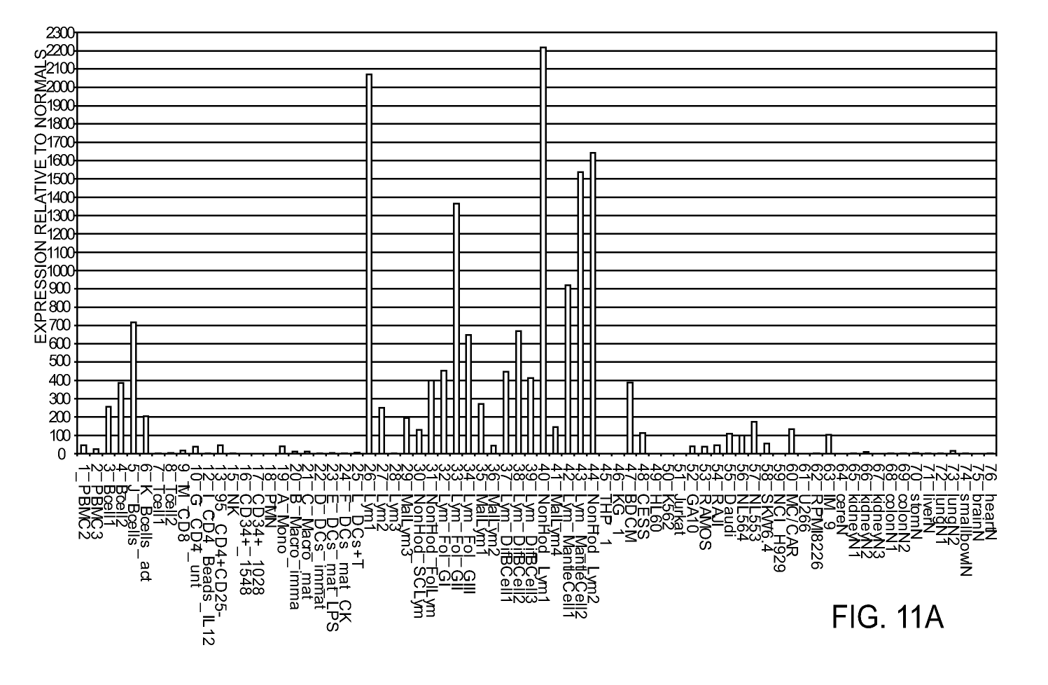

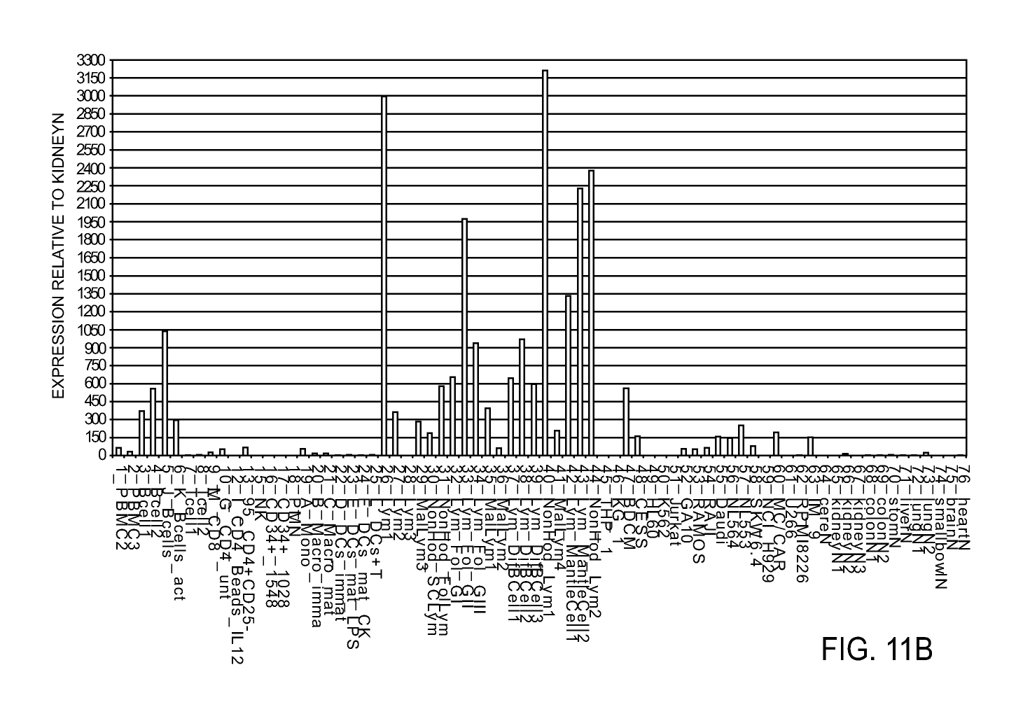

Non-detected samples (samples no. 16, 18, 45, 49-51, 59, 61, 64 and 75, Table 2) were assigned Ct value of 41 and were calculated accordingly. The normalized quantity of each RT sample was then divided by the median of the quantities of the normal samples (sample numbers 64-76, Table 2 above), to obtain a value of relative expression of each sample relative to median of the normal samples, as shown in FIG. 11A. The normalized quantity of each RT sample was also divided by the median of the quantities of the kidney normal samples (sample numbers 65-67, Table 2 above), to obtain a value of relative expression of each sample relative to median of the kidney normal samples, as shown in FIG. 11B.

The results of this analysis are depicted in the histogram in FIG. 11A. High expression of the above-indicated CD20-variant transcript is clearly seen in a wide range of lymphomas (100-1350 fold increase over median normal tissue expression). Expression is also high in B cells (130-430 fold increase over median normal tissue expression) and in B cell derived cell lines (BDCM and CESS). High overexpression is also seen NL553, NL564, Daudi, and MC/CAR (lymphoblast derived cell line). Similar expression pattern is seen the FIG. 11B.

Normal Panel—

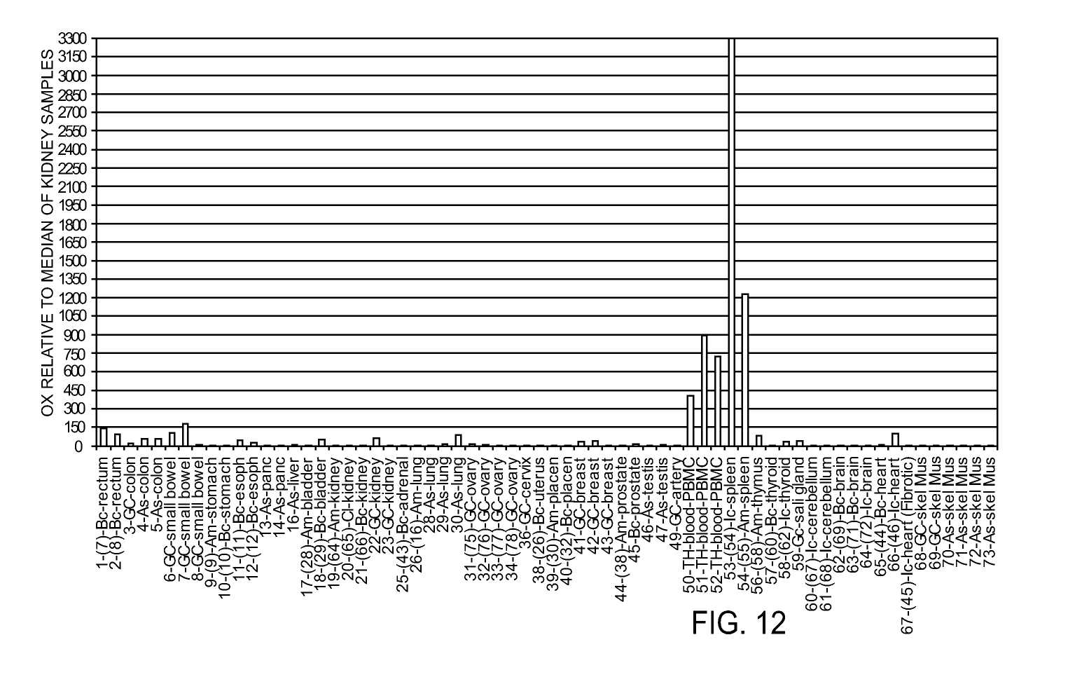

Non-detected samples (samples no. 9, 10, 13, 14, 17, 19-21, 25, 26, 28, 33, 34, 36, 38-40, 43, 44, 49, 57, 60-64 and 67-73, Table 3) were assigned Ct value of 41 and were calculated accordingly. The normalized quantity of each RT sample was then divided by the median of the quantities of the kidney normal samples (sample numbers 19-23, Table 3), to obtain a value of relative expression of each sample relative to median of the kidney normal samples, as shown in FIG. 12.

FIG. 12 is a histogram showing expression of CD20-variant transcripts which are detectable by amplicon as depicted in sequence name seg10-12F2R2 (SEQ ID NO:87) in normal panel. High overexpression is seen in blood-PBMC and spleen samples.

In order to compare the overexpression in both blood specific and normal panels, a combined panel containing samples from blood and from normal panels was used (Table 5).

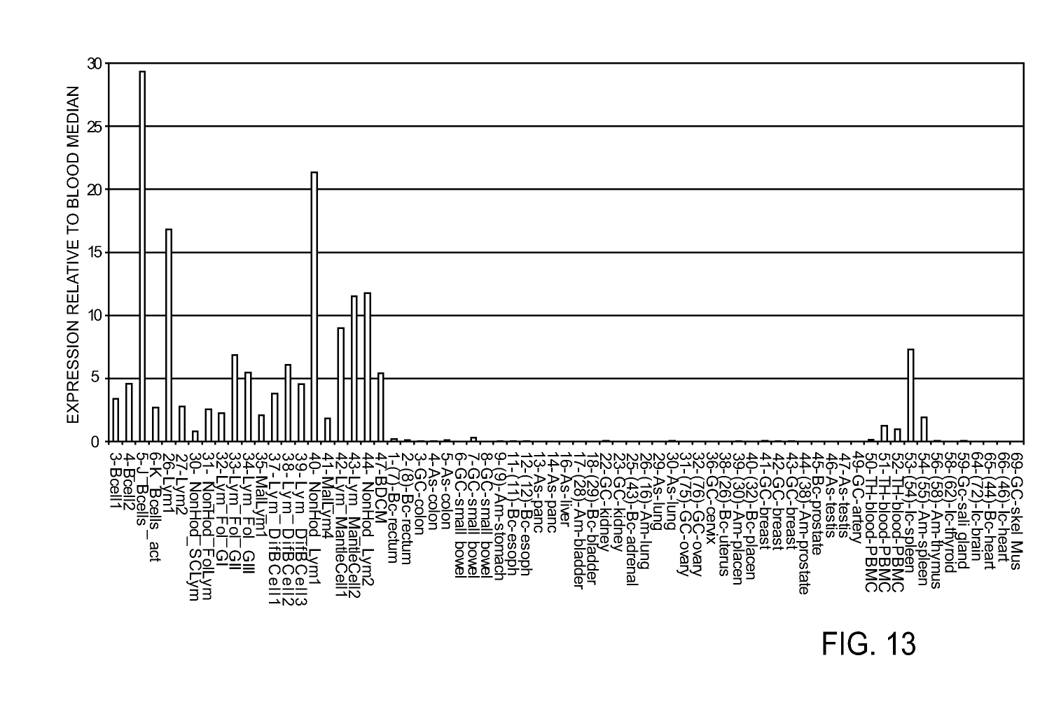

Non-detected samples (samples no. 13, 14, 31, 36, 38, 40, 45, 65 and 69, Table 5) were assigned Ct value of 41 and were calculated accordingly. The normalized quantity of each RT sample was then divided by the median of the quantities of the blood-PBMCs samples (sample numbers 50-52, Table 5), to obtain a value of relative expression of each sample relative to median of the blood-PBMCs samples, as shown in FIG. 13.

The results of this analysis are depicted in the histogram in FIG. 13. The overexpression of the above-indicated CD20-variant transcript seen in the blood sampels is higher than the overexpression seen in different the normal samples.

Primer pairs are also optionally and preferably encompassed within the present invention; for example, for the above experiment, the following primer pair was used as a non-limiting illustrative example only of a suitable primer pair: seg10-12F2 forward primer (SEQ ID NO:85); and seg10-12R2 reverse primer (SEQ ID NO:86).

The present invention also preferably encompasses any amplicon obtained through the use of any suitable primer pair; for example, for the above experiment, the following amplicon was obtained as a non-limiting illustrative example only of a suitable amplicon:

seg10-12F2R2 (SEQ ID NO:87).

Forward primer: >seg10-12F2

(SEQ ID NO: 85)

CCCATCTGTGTGACTGTGTGGTAC
Reverse primer: >seg10-12R2

(SEQ ID NO: 86)

TCTGATGCCCTCTGAAGAGTGAACTG
Amplicon: seg10-12F2R2

(SEQ ID NO: 87)

CCCATCTGTGTGACTGTGTGGTACCCTCTCTGGGGAGGCATTATGCCTGA
ATGTGAGAAAAGGAAGATGAGCAATAGTCATCATCACTTCCTGTAACAGC
CAATGTTTTCATGGAGTGCCTGTGCCATTCAGGTCAAGTATTTCCTTCTG
CATCAGTTCACTCTTCAGAGGGCATCAGA

### Example 3_{—}3

### Cloning of CD20 Variants

### Example 3_{—}3_{—}1

### Cloning of HSCD20B_{—}1_P5 (SEQ ID NO:33) ORF Fused to Flag Tag

Cloning of HSCD20B_{—}1_P5 (SEQ ID NO:33) (also referred herein as CD20_T12 P5) open reading frame (ORF) fused to FLAG was carried out by RT PCR as described below.

A reverse transcription reaction was carried out as follows: 10 μg of purified Lymph Node Lymphoma RNA or NHL Diffuse Large B-Cell Lymphoma RNA were mixed with 150 ng Random Hexamer primers (Invitrogen, Carlsbad, Calif., USA, catalog number: 48190-011) and 500 μM dNTPs in a total volume of 1564 The mixture was incubated for 5 min at 65° C. and then quickly chilled on ice. Thereafter, 50 ml of 5× SuperscriptII first strand buffer (Invitrogen, catalog number: 18064-014, part number: Y00146), 24 μl 0.1M DTT and 400 units RNasin (Promega, Milwaukee, Wis., U.S.A., catalog number: N2511) were added, and the mixture was incubated for 10 min at 25° C., followed by further incubation at 42° C. for 2 min. Then, 10 μl (2000 units) of SuperscriptII (Invitrogen, catalog number: 18064-014) was added and the reaction (final volume of 2500 was incubated for 50 min at 42° C. and then inactivated at 70° C. for 15 min. The resulting cDNA was diluted 1:20 in TE buffer (10 mM Tris, 1 mM EDTA pH 8) and served as a template for PCR.

PCR was done using GoTaq ReadyMix (Promega, catalog number M122) under the following conditions: 5 μl Platinum PFX 10× buffer; 1.5 μl MgSO₄ (50 mM); 5 μl—cDNA; 2 μl—10 mM dNTPs (2.5 mM of each nucleotide); 1 μl —Platinum PFX enzyme; 36 μl—H2O; and 1.5 μl (10 μM)—of each primer #100-871 (SEQ ID NO:113) and #100-875 (SEQ ID NO: 114) in a total reaction volume of 50 μl; with a reaction program of 2 minutes in 94° C.; 35 cycles of: 30 seconds at 94° C., 30 seconds at 51° C., 1 minute at 68° C.; then 10 minutes at 68° C. Primers which were used include gene specific sequences; restriction enzyme sites; Kozak sequence and FLAG tag.

50 μl of PCR product were loaded onto a 1% agarose gel stained with ethidium bromide, electrophoresed in 1×TAE solution at 100V, and visualized with UV light. After verification of expected band size, PCR product was excised and extracted from the gel using QiaQuick™ Gel Extraction kit (Qiagen, catalog number: 28707). The extracted PCR product was digested with NheI and AgeI restriction enzymes (New England Biolabs, Beverly, Mass., U.S.A.). After digestion, DNA was loaded onto a 1% agarose gel as described above. The expected band size was excised and extracted from the gel using QiaQuick™ Gel Extraction kit (Qiagen, catalog number: 28707). The digested DNA was then ligated into pIRESpuro3 vector, previously digested with the above restriction enzymes, using LigaFast™ Rapid DNA Ligation System (Promega, catalog number: M8221). The resulting DNA was transformed into competent E. coli bacteria DH5α (RBC Bioscience, Taipei, Taiwan, catalog number: RH816) according to manufacturer's instructions, then plated on LB-ampicillin agar plates for selection of recombinant plasmids, and incubated overnight at 37° C. The following day, a number of colonies that grew on the selective plates, were taken for further analysis by streak-plating on another selective plate. Screening of positive clones was performed by PCR using pIRESpuro3 vector specific primer and gene specific primer (data not shown). After completion of all PCR cycles, half of the reaction was analyzed using 1% agarose gel as described above. After verification of expected band size, two positive colonies were grown in 5 ml Terrific Broth supplemented with 100 μg/ml ampicillin, with shaking overnight at 37° C. Plasmid DNA was isolated from bacterial cultures using Qiaprep™ Spin Miniprep Kit (Qiagen, catalog number: 27106). Accurate cloning was verified by sequencing the inserts (Weizmann Institute, Rehovot, Israel). Upon verification of an error-free colony (i.e. no mutations within the ORF), recombinant plasmids were processed for further analyses.

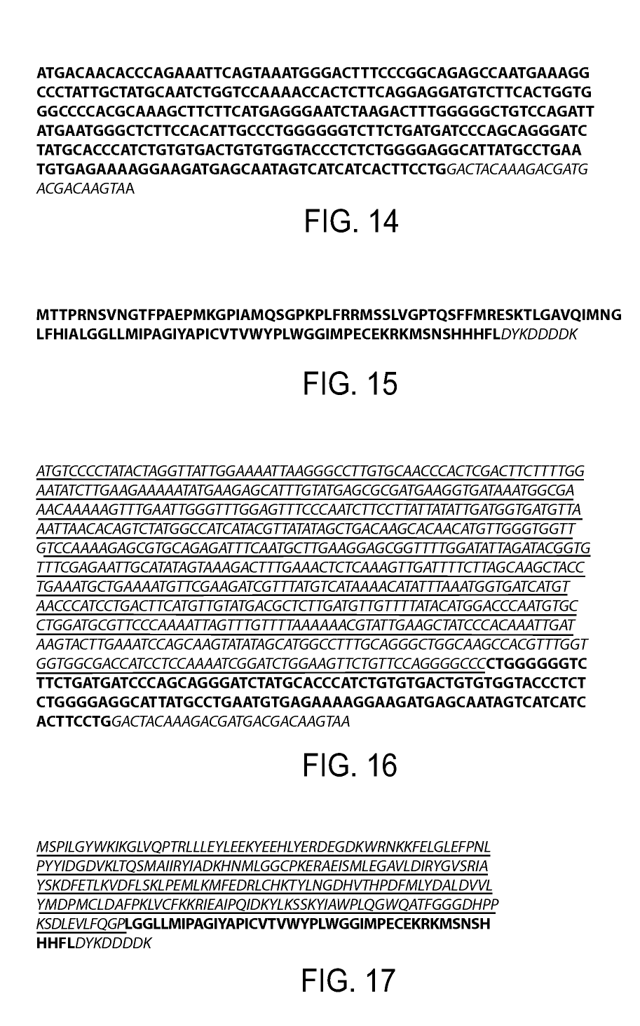

The DNA sequence of the resulting CD20_T12_FLAG (SEQ ID NO:73) is shown in FIG. 14; gene specific sequence corresponding to CD20_T12 ORF sequence is marked in bold faced, FLAG sequence is in italics.

The amino acid sequence of CD20_P5_FLAG (SEQ ID NO:74) is shown in FIG. 15; amino acid sequence corresponding to CD20_P5 ORF is marked in bold faced, FLAG sequence is in italics.

### Example 3_{—}3_{—}2

### Cloning of Cd20_T12 P5 (Amino Acids 66-109) Fused to FLAG TAG and to GST

CD20_T12 (amino acids 66-109)_FLAG (SEQ ID NO: 75) was cloned in frame to Glutathione S-Transferase (GST) as described below. CD20_T12_FLAG pIRES puro3 described above was double digested with PasI (Fermentas, catalog number: ER1861) and NotI (New England Biolabs, Beverly, Mass., U.S.A.) and ligated into pGEX-6P-1 (Amersham; catalog number 27-4597-01) previously digested with the same enzymes. After digestion, DNAs were loaded onto a 1% agarose gel as described above. The expected band size was excised and extracted from the gel using QiaQuick™ Gel Extraction kit (Qiagen, catalog number: 28707). The digested DNA was ligated into pGEX-6P-1 vector previously digested with the same enzymes, using the LigaFast™ Rapid DNA Ligation System (Promega, catalog number: M8221). The resulting DNAs were transformed into competent E. coli bacteria DH5a (RBC Bioscience, Taipei, Taiwan, catalog number: RH816) according to manufacturer's instructions, then plated on LB-ampicillin agar plates for selection of recombinant plasmids, and incubated overnight at 37° C. The following day, a number of colonies that grew on the selective plates were taken for further analysis by streak-plating on another selective plate and by PCR using GoTaq ReadyMix (Promega, catalog number: M7122). Screening positive clones was performed by PCR using pGEX-6P-1 vector specific primer and gene specific primer (data not shown). After completion of all PCR cycles, half of the reaction was analyzed using 1% agarose gel as described above. After verification of expected band size, two positive colonies were grown in 5 ml Terrific Broth supplemented with 100 μg/ml ampicillin, with shaking overnight at 37° C. Plasmid DNA was isolated from bacterial cultures using Qiaprep™ Spin Miniprep Kit (Qiagen, catalog number: 27106). Accurate cloning was verified by sequencing the inserts (Weizmann Institute, Rehovot, Israel). Upon verification of an error-free colony (i.e. no mutations within the ORF), recombinant plasmids were processed for further analyses.

The DNA sequence of the resulting GST_CD20_T12 (amino acids 66-109)_FLAG (SEQ ID NO:75) is shown in FIG. 16; gene specific sequence corresponding to CD20_T12 (amino acids 66-109) sequence is marked in bold faced, GST sequence is in italics and underlined and FLAG sequence is in italics.

The amino acid sequence of GST_CD20_P5 (amino acids 66-109)_FLAG (SEQ ID NO:76) is shown in FIG. 17; amino acid sequences corresponding to CD20_P5 (amino acids 66-109) sequence is marked in bold faced, GST sequence is in italics and underlined and FLAG sequence is in italics.

### Example 3_{—}4

### Determining Cell Localization of CD20 Proteins

In order to determine CD20_P5 cellular localization, C20 T12 P5 (SEQ ID NO:31) was cloned in frame to FLAG tag, as described above. Protein localization was observed upon transient transfection (Chen et al., Molecular Vision 2002; 8; 372-388) using confocal microscopy. 48 hours following transfection, the cells were stained with anti FLAG antibodies conjugated to Cy-3 fluorophore and were observed for the presence of fluorescent signal. CD20_T12 P5_FLAG pIRESpuro3 (SEQ ID NO:73) construct was transiently transfected into HEK-293T cells as follows: HEK-293T (ATCC, CRL-11268) cells were plated on sterile glass coverslips, 13 mm diameter (Marienfeld, catalog number: 01 115 30), which were placed in a 6 well plate, using 2 ml pre-warmed DMEM [Dulbecco's modified Eagle's Media, Biological Industries (Beit Ha'Emek, Israel), catalog number: 01-055-1A]+10% FBS [Fetal Bovine Serum, Biological Industries (Beit Ha'Emek, Israel), catalog number: 04-001-1A]+4 mM L-Glutamine [Biological Industries (Beit Ha'Emek, Israel), catalog number: 03-020-1A]. 500,000 cells per well were transfected with 2 μg of DNA construct using 6 μl FuGENE 6 reagent (Roche, catalog number: 11-814-443-001) diluted into 94 μl DMEM. The mixture was incubated at room temperature for 15 minutes. The complex mixture was added dropwise to the cells and swirled. Cells were placed in incubator maintained at 37° C. with 5% CO₂ content. 48 hours post transient transfection, cells on coverslip were further processed for immunostaining and analysis by confocal microscopy. The cover slip was washed in phosphate buffered saline (PBS), then fixed for 15 minutes with a solution of 3.7% paraformaldehyde (PFA) (Sigma, catalog number: P-6148)/3% glucose (Sigma, catalog number: G5767) (diluted in PBS). Quenching of PFA was done by a 5 minute incubation in 3 mM glycine (Sigma, catalog number: G7126) (diluted in PBS). After two 5-minute washes in PBS, cells were permeabilized with 0.1% triton-X100 (diluted in PBS) for 5 minutes. After two 5-minute washes in PBS, blocking of non-specific regions was done with 5% bovine serum albumin (BSA) (Sigma, catalog number: A4503) (diluted in PBS) for 20 minutes. The coverslip was then incubated, in a humid chamber for 1 hour, with mouse anti FLAG-Cy3 antibodies (Sigma, catalog number: A9594), diluted 1:100 in 5% BSA in PBS, followed by three 5-minute washes in PBS. The coverslip was then mounted on a slide with Gel Mount Aqueous medium (Sigma, catalog number: G0918) and cells were observed for the presence of fluorescent product using confocal microscopy.

In this experiment, ectopic expression of the variant CD20_P5_FLAG (SEQ ID NO:74) HEK 293T cells was mainly detected in the cell cytosol (data not shown).

### Example 3_{—}5

### Production of Polyclonal Antibodies Specific to CD20 PROTEINS

All polyclonal antibodies production procedure, including peptide synthesis, peptide conjugation, animal care, animal immunizations, bleeding and antibody purification were performed at Sigma-Aldrich (Israel). Two pairs of rabbits were injected to prepare antibodies for CD20_P5 (rabbit numbers 5347 and 5358, 5359 and 5360 respectively).

Peptides which were used for rabbit immunization were as follows: MTTPRNSVNGTFPAEPMKG CD20_SV1 (SEQ ID NO:77) a sequence taken from the N′ terminus corresponding to amino acids residues 1-19 of HSCD20B_{—}1_P5 (SEQ ID NO:33) (also referred herein as CD20_P5) protein, in which Cystein was added to the C′ terminus of the peptide for KLH conjugation. This peptide sequence is common to CD20_P5 (SEQ ID NO:108) and to wild type CD20 protein (SEQ ID NO:32). The second peptide sequence was: MPECEKRKMSNSHHHFL CD20_SV95 (SEQ ID NO:78), a sequence specific to CD20_P5 only, corresponding to amino acids residues 95-109 of HSCD20B_{—}1_P5 (SEQ ID NO:33) protein. 25 mg of each peptide were synthesized with 95% purity of which 10 mg were conjugated to KLH carrier. Each pair of rabbits was immunized with the corresponding conjugated peptide as follows: rabbits 5347 and 5358 were immunized with CD20_SV1 (SEQ ID NO:77) peptide, and rabbits 5359 and 5360 were immunized with CD20_SV95 (SEQ ID NO:78) peptide. Animals were immunized every two weeks. 100 ml production bleeds from each rabbit were collected and affinity purification was performed with the peptide against which the respective antibodies were raised. The purified antibodies were analyzed by ELISA.

### Characterization of Purified CD20_SV95 (SEQ ID NO:78) Antibodies

The specificity of anti CD20_SV95 (SEQ ID NO:78) antibodies purified from rabbits 5359 and 5360 described above was determined using Western blot analysis on bacterial cell extracts as described below. E. coli bacteria DH5α (RBC Bioscience, Taipei, Taiwan, catalog number: RH816) transformed with GST_CD20_P5 (amino acids 66-109)_FLAG pGEX-6P-1 described above, or with empty pGEX-P6-1 vector, were grown at 37° C. over-night in the presence of 100 μg/ml ampicillin. The next day, 10 ml of LB containing 100 μg/ml ampicillin were inoculated with 0.2 ml of the over-night culture. The culture was grown for two hours at 37° C. to optical density (OD)₆₀₀ of 0.4-0.6. At this point, a sample of 2 ml bacteria was taken (termed T=0) and span down at 10,000 rpm for 1 minute and the pellet was frozen at −20° C. In order to induce expression of GST_CD20_P5 (amino acids 66-109)_FLAG, 1 mM of IPTG (Roche, catalog number 10724815001) was added to the rest 8 ml of bacterial cultures. The cultures were further incubated for 3 hours. At the end of incubation, OD was measured again and a sample of 2 ml bacteria was taken (termed T=3), span down at 10,000 rpm for 1 minute and the bacteria pellet were processed as following: The bacteria pellets were normalized based on the OD₆₀₀ at harvest and re-suspended in NuPAGE® LDS sample buffer (Invitrogen, catalog number: NP0007) containing 1,4-Dithiothreitol (DTT; a reducing agent) at a final concentration of 100 mM. The samples were then incubated at 100° C. for 3 minutes, followed by a 1 minute spin at 14,000 rpm. 5 μl of each sample were loaded on a 12% NuPAGE® Bis-Tris gels (Invitrogen, catalog number: NP0341), and gels were run in 1×MOPS SDS running buffer (Invitrogen, catalog number: NP0001), using the XCell SureLock™ Mini-Cell (Invitrogen, catalog number: E10001), according to manufacturer's instructions. The separated proteins were transferred into nitrocellulose membranes (Schleicher & Schuell, catalog number: 401385) using the XCell™ II blotting apparatus (Invitrogen, catalog number E19051), according to manufacturer's instructions. Non-specific regions of the membrane were blocked by incubation in 10% skim-milk diluted in Tris buffered saline (TBS) supplemented with 0.05% Tween-20 (TBST) for 1 hour at room temperature (all subsequent incubations occur for 1 hour at room temperature). Blocking solution was then replaced with primary antibody solution: purified antibodies of rabbits 5359 and 5360 anti-CD20 antibodies described above diluted 1:250 in blocking solution. After three 10-minute washes, secondary antibody was applied: goat anti-rabbit conjugated to horse radish-peroxidase (Jackson ImmunoResearch, catalog number: 111-035-144) diluted 1:10,000 in blocking solution for one hour. After three 10-minute washes, ECL substrate (GE-Amersham, catalog number: RPN2209) was applied for 1 minute, followed by exposure to X-ray film (Fuji, catalog number: 100NIF).

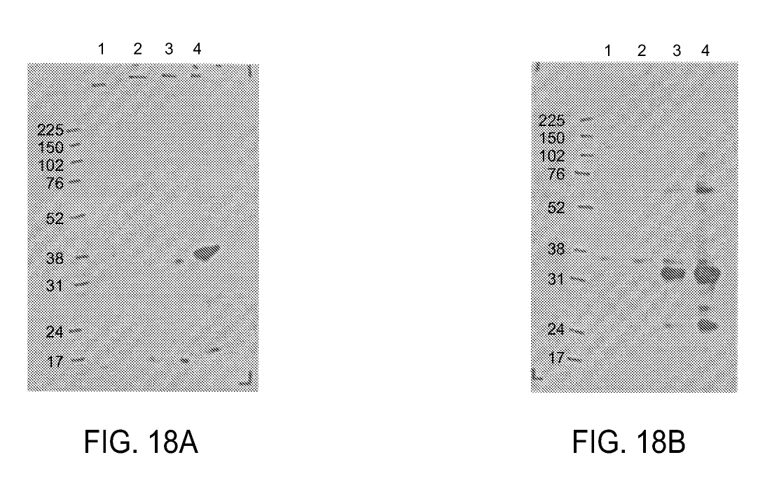

FIGS. 18A-B demonstrate that anti CD20_SV95 (SEQ ID NO:78) from both rabbit 5359 and rabbit 5360 recognize GST_CD20_P5 (a.a 66-109)_FLAG (SEQ ID NO:76) expressed in bacteria. E. coli bacteria DH5α were transformed with GST_CD20_P5 (a.a 66-109)_FLAG pGEX-6P-1 or with the empty vector pGEX-P6-1. Cell lysate were analyzed by Western blot analysis using anti CD20_SV95 (SEQ ID NO:78) antibodies from rabbit 5359 (FIG. 18A) or rabbit 5360 (FIG. 18B) Lane 1: pGEX-6P-1, T0; Lane 2: pGEX-6P-1, T3, Lane 3: GST_CD20_P5, T0; Lane 4: GST_CD20_P5, T3,

Purified antibodies from rabbits 5359 and 5360 were further tested by immune-staining of HEK-293T transiently transfected with CD20_T12 P5 pIRESpuro described above. In this experiment no specific staining was obtained (data not shown).

### Example 4

### Example 4_{—}1

### Description for Cluster HUMDAF

CD55, also named decay-accelerating factor (DAF), is a membrane bound (GPI-anchored) protein which belongs to the group of membrane-associated complement regulatory proteins (CRPs) (Kim and Song, 2006, Clinical Immunology 118: 127-136). It protects cells from bystander injury (complement-mediated lysis) when complement is activated. As its name implies, DAF (CD55) acts by accelerating the decay of complement components, C3 and C5 convertases, preventing the formation of the membrane attack complex. CD55 is composed of four N-terminal short consensus repeats (SCRs) (also named complement control protein domains, CCPs), a heavily glycosylated serine, threonine and proline (STP)— rich domain, and a C-terminal GPI-anchored portion.

Several isoforms have been reported for CD55. In rodents, alternative splice variants have been identified that produce GPI-anchored, transmembrane and soluble forms (Harris et al, 1999, Biochem J. 341: 821-829; Nonaka et al, 1995, J. Immunol. 155: 3037-3048; Miwa et al, 2000, Immunogenetics 51: 129-137). In humans, several GPI-anchored and soluble isoforms have been reported (Moran et al 1992, J. Immunol. 149: 1736-1743; Osuka et al 2006, Genomics 88: 316-322). The wild type GPI-anchored form is the major form, and is expressed on the plasma membranes of all blood cells and almost all other cell types that are in immediate contact with plasma complement, such as endothelial and epithelial cells. Soluble isoforms are expressed at lower levels and were detected in bodily fluids and extracellular matrix. The soluble isoforms are generated by alternative usage of an optional exon and lack the GPI-anchored portion at the C-terminal (Caras et al 1987, Nature 325: 545-549; Osuka et al 2006, Genomics 88: 316-322).

The importance of CD55 is demonstrated by its increase in tumor and inflammatory environments, indicating that its expression is associated with the alteration of cells under these pathological circumstances.

CD55 is overexpressed on a wide range of solid tumors. CD55 is also known to be deposited within tumor stroma, by cleavage from the cell membrane and/or by secretion of an active soluble form. Like other CRPs, CD55 has been detected in various malignancies such as CLL, CML, ALL, AML, colorectal cancer, gastric cancer, thyroid cancer, medullary thyroid cancer, malignant glioma, breast cancer, renal cancer, non-small cell lung cancer, ovarian cancer, cervical cancer and in cell lines derived from those tumor types. CD55 expression on tumor cells provides a means of evasion from complement attack. The expression of CD55 in gastric and colorectal carcinomas is associated with invasion and metastasis, and with poor prognosis (Durrant et al 2003, 52: 638-642). In addition, CD55 is frequently detectable within the stools of patients with colorectal carcinomas and might contribute to the early diagnosis of this disease (reviewed in Mikesch et al 2006, Biochim. Biophys. Acta 1766: 42-52).

Malignant tumors express this and other CRPs at high levels to protect the cancerous cells from complement mediated tumor cell lysis (Mikesch et al 2006, Cell. Oncol. 28: 223-232). CD55 also decreases cell adhesion which might play a role in invasive tumor growth and formation of metastases. Adhesion of T-lymphocytes to human leukemic cells is also decreased by CD55. Furthermore, CD55 also has an inhibiting effect on NK cells, which could promote tumor initiation and primary growth. Other pro-tumorigenic functions exerted by CD55 are autocrine loops for cell rescue and evasion of apoptosis, and neoangiogenesis (reviewed in Mikesch et al 2006, Cell. Oncol. 28: 223-232; Mikesch et al 2006, Biochim. Biophys. Acta 1766: 42-52).

CD55 is target for anti-cancer therapy, and monoclonal antibodies targeting this molecule are in various stages of clinical trials. Onyvax-105, an anti-CD55 mAb developed by Onyvax, is in Phase II for colorectal cancer and osteosarcoma, and in preclinical development for prostate cancer. SC-1, another anti-CD55 mAb developed by Cambridge Antibody Technology, is in Phase I/II for gastric cancer. A human monoclonal anti-idiotypic antibody, 105AD7, which targets CD55, has been used in clinical trials as a form of active specific immunotherapy or cancer vaccine that aims to stimulate specific T-cells to target tumor specific antigens. Results indicate that the 105AD7 is capable of stimulating T-cells to target tumor specific antigens, which then become activated, and kill tumor cells by apoptosis. 105AD7 was used in conjunction to myelosuppressive chemotherapy in a clinical trial of osteosarcoma (Pritchard-Jones et al 2005, Br. J. Cancer 92:1358-1365), and in adjuvant clinical trials in patients with colorectal cancer (Maxwell-Armstrong, 2002 Ann R. Coll. Surg. Engl. 84: 314-318; Ullenhag et al 2006, Clin. Cancer Res. 12: 7389-7396).

Overexpression of CRPs, including CD55, by tumor cells restricts the anti-tumor effect of complement-dependent cytotoxicity (CDC) induced by therapeutic antibodies targeted to cancer cells, such as rituximab (Macor et al 2007). In vivo studies indeed showed that anti-CD55 mAbs enhance the anti-tumor activity of Rituximab, by enhancement of CDC (Macor et al 2007, Cancer Res. 67: 10556-10563). These findings indicate that drugs targeting CD55 could be combined with various antibodies and other agents, to enhance their therapeutic effect.

By protecting autologous cells and tissues from complement-mediated damage, various CRPs including DAF (CD55) can play a role in preventing or modulating autoimmune disease and inflammation (Lublin 2005, Immunohematol. 21: 39-47). Indeed, in settings of acute or chronic inflammation, membrane CRPs are up-regulated in order to offer extra protection of the vascular wall from complement injury. Accordingly, a number of inflammatory cytokines as well as C-reactive protein (a marker of chronic inflammation) have been shown to induce DAF expression on endothelial cells.

Complement activation is evident in various inflammatory diseases, including rheumatoid arthritis (RA), systemic lupus erythematosus (SLE), lupus nephtirits and multiple sclerosis (MS). In RA, soluble products of complement activation are present in the synovial fluid of affected joints. While complement itself is not always the primary cause of these diverse diseases, it acts to sustain the pro-inflammatory cycle and perpetuate tissue damage. By removing the transmembrane domains or GPI anchors, soluble recombinant regulatory proteins have been engineered, which can be given systemically. Recombinant soluble complement inhibitors (including soluble DAF) have been shown to be effective for the treatment of inflammatory disease in various rodent models. The use of recombinant DAF for modulation of autoimmune diseases and inflammation is being actively investigated (Lublin 2005, Immunohematol. 21: 39-47). For example, soluble recombinant DAF fused to Fc portion of immunoglobulin, was caused a sustained reduction in plasma complement activity, and reduced severity of disease in a rat model of arthritis (Harris et al 2002, Clin. Exp. Immunol. 129: 198-207).

Complement activation also occurs in a number of ischemia-reperfusion (IR) injury settings and is responsible, at least partially, for initiating and/or propagating the inflammatory response associated with IR (Arumugam et al, 2004, Shock 21: 401-409). The compelling evidence for complement activation in such disorders has driven the search for therapeutic reagents capable of inhibiting the complement cascade. Such reagents are currently in clinical trials for treatment of acute inflammatory disorders, such as acute respiratory distress syndrome (ARDS) or IR injury. A soluble form of CD55 afforded protection in animal models of IR injury (Weeks et al 2007, Clin. Immunol. 124: 311-327).

Besides its importance as a regulator of the complement system, CD55 is also known to be a ligand for the T cell early activation antigen CD97, and their interaction has been shown to inhibit the proliferation of activated T cells (Spendlove et al 2006, Cancer Immunol. Immunother. 55: 987-995). Furthermore, CD55 also seems to inhibit NK cells, and to serve as a receptor for certain viruses and other microorganisms.

In addition, CD55 involvement in acute and chronic inflammation may stem from its ability to directly interact with CD97, a receptor which is constitutively expressed on granulocytes and monocytes and rapidly up-regulated on T and B cells upon activation. Direct stimulation of CD55 on T cells using a crosslinking mAb or CD97 can enhance T cell activation (Capasso et al 2006, J. Immunol. 177: 1070-1077). The interaction of CD55 with CD97 has also been shown to play an important role in the migration of neutrophils in models of inflammatory bowel disease (IBD) and pneumonia (Leemans et al 2004, J. Immunol. 172: 1125-1131). Furthermore, synovial tissue of RA patients is characterized by an influx and retention of CD97+ inflammatory cells. CD55 is expressed abundantly in the synovial tissue, predominantly on fibroblast-like synoviocytes, endothelium and extracellular matrix. Blocking of CD97 with an anti-CD97 mAb in an animal model of RA resulted in significant reduction of several symptoms (Kop et al 2006, Arthritis Res. Ther. 8: R155), suggesting that use of agents preventing CD97-CD55 interaction might be beneficial in RA therapy.

DAF can influence the outcome of a T cell response to a given antigen by processes independent of complement activation (Longhi et al 2006, Trends in Immunol. 27: 102-108). Downregulation of DAF could represent a strategy for tumor immunotherapy, for instance by enhancing the survival and proliferative capacity of anti-tumor T cell responses before re-infusion.

Deficiency of the DAF gene in mice enhanced T cell responses to active immunization. Such mice also displayed exacerbated disease progression and pathology of EAE, an animal model of MS (Liu et al 2005, J. Exp. Med. 201: 567-577). These findings indicate that DAF is implicated in T cell immunity in vivo, and that it is a promising target for organ transplantation, tumor evasion and vaccine development.

Another use of DAF is in the field of xenotranplantation. The limited and inadequate availability of organs from human donors has resulted in the utilization of xenografts as an alternative tool. Nevertheless, hyperacute rejection following xenograft determines the loss of the transplanted organ. The “primum movens” is the activation of the complement pathway mediated by the binding of natural xenogenic antibodies to the endothelium of the graft, followed by the lysis of the endothelial cells with subsequent edema, thrombosis and necrosis of the transplanted organ. Various molecular approaches, such as the development of transgenic animals expressing human CRPs such as hCD59 or hCD55, and the use of their organs in xenotransplantation in order to downregulate complement activation, and prevent hyperacute and acute graft rejection (Ghebremariam et al 2005 Ann N Y Acad. Sci. 1056: 123-143).

WO2005/071058, US Patent Application Publication No. 2006/0068405, and European Patent application Publication No. 1713900, all assigned to the applicants of the present invention, filed on Jan. 27 2005 (earliest priority from a U.S. provisional application No. 60/539,129, filed on Jan. 27 2004), disclose three of the CD55 splice variants, referred herein as HUMDAF_P14 (SEQ ID NO:51), HUMDAF_P15 (SEQ ID NO:52) and HUMDAF_P26 (SEQ ID NO:54). The sequences disclosed therein contain a known SNP at position 455 of HUMDAF_P14 (SEQ ID NO:51) and at position 455 of HUMDAF_P15 (SEQ ID NO:52).

The WO2005/071058, US Patent Application Publication No. 2006/0068405, and the European Patent application Publication No. 1713900 disclose overexpression of the CD55 variants corresponding to HUMDAF_P14 (SEQ ID NO:51) and HUMDAF_P15 (SEQ ID NO:52), based on the source of the corresponding ESTs, in several cancerous tissues, such as in cancers of the lung and bone-marrow tumor, as well as in immunological disorders, particularly in rheumatoid arthritis, transplant rejection and reperfusion injury. These applications further disclose involvement of CD55 variants corresponding to HUMDAF_P14 (SEQ ID NO:51) and HUMDAF_P15 (SEQ ID NO:52) in complement factor 1 stimulation or inhibition. These applications further disclose the use of the novel CD55 variants as monoclonal antibody targets for treatment of cancer, specifically lung tumor and bone marrow-tumor, as well as immunosuppressant, antiarthritic, for treatment of immunological disorders, rheumatoid arthritis, transplant rejection, cardiovascular disorders, and reperfusion injury. However, none of these applications neither teach nor suggest the use of the ectodomains of CD55 splice variants in immunotherapy and for treatment of cancer, immune related indications, autoimmune diseases, inflammation of the respiratory tract, ischemia-reperfusion injury related disorders, transplant rejection, therapy of disease states in which complement activation and deposition is involved in pathogenesis or use of CD55 variant-transgenic animals for xenotransplantation. In addition, none of these applications neither teaches nor suggests the use of CD55 splice variants as targets for cancer therapy, and drug development for immunotherapy and for treatment of cancer other than lung cancer and bone marrow-tumor, particularly wherein the cancer is selected from colorectal cancer, prostate cancer, pancreas cancer, ovarian cancer, gastric cancer, liver cancer, and wherein the cancer is non-metastatic, invasive or metastatic, for therapy of inflammation of the respiratory tract disorders, therapy of disease states in which complement activation and deposition is involved in pathogenesis or use of CD55 variant-transgenic animals for xenotransplantation.

US Patent Application Publication No. 2004-0142325, assigned to the applicants of the present invention, and WO2004/023973, assigned to INCYTE CO., disclose among thousands of other transcripts, one of the CD55 splice variants, referred herein as HUMDAF_P26 (SEQ ID NO:54). The WO2004/023973 generally states that all the disclosed sequences are useful in diagnosing a condition, disease or disorder associated with these molecules, e.g. autoimmune or inflammatory disorders, in gene therapy or in gene mapping. US Patent Application Publication No. 2004-0142325 predicts overexpression of the CD55 variant, based on the source of its ESTs, in several cancerous tissues, such as in cancers of the brain, placenta, cervix, lung, blood, bone marrow, thyroid, salivary gland, uterus, lymph node, and colon. None of these applications neither teaches nor suggests the use of the ectodomains of CD55 splice variants for in immunotherapy and for treatment of cancer, immune related indications, autoimmune diseases, inflammation of the respiratory tract, ischemia-reperfusion injury related disorders, transplant rejection, therapy of disease states in which complement activation and deposition is involved in pathogenesis or use of CD55 variant-transgenic animals for xenotransplantation. In addition, none of these applications neither teaches nor suggests the use of CD55 splice variants as targets for immunotherapy, cancer therapy, and drug development for immunotherapy and for treatment of cancer, immune related indications, autoimmune diseases, inflammation of the respiratory tract, ischemia-reperfusion injury related disorders, transplant rejection, therapy of disease states in which complement activation and deposition is involved in pathogenesis or use of CD55 variant-transgenic animals for xenotransplantation.

CD55 variants corresponding to polypeptides referred herein as HUMDAF_P14 (SEQ ID NO:51) and HUMDAF_P15 (SEQ ID NO:52), appear in a later published paper by Osuka F. et al., Genomics 88, 2006, 316-322. Osuka F. et al., Genomics 88, 2006, 316-322 discloses ubiquitous expression of CD55 variants corresponding to HUMDAF_P14 (SEQ ID NO:51) and HUMDAF_P15, and assign them defense activities of the host cells from autologous complement attack. However, Osuka F. et al. paper neither teaches nor suggests the use of CD55 splice variants as targets for immunotherapy, cancer therapy, and drug development for immunotherapy and for treatment of cancer, immune related indications, autoimmune diseases, inflammation of the respiratory tract, ischemia-reperfusion injury related disorders, transplant rejection, therapy of disease states in which complement activation and deposition is involved in pathogenesis or use of CD55 variant-transgenic animals for xenotransplantation. Osuka F. et al. paper also neither teaches nor suggests the use of the ectodomains of CD55 splice variants for in immunotherapy and for treatment of cancer, immune related indications, autoimmune diseases, inflammation of the respiratory tract, ischemia-reperfusion injury related disorders, transplant rejection, therapy of disease states in which complement activation and deposition is involved in pathogenesis or use of CD55 variant-transgenic animals for xenotransplantation.

Cluster HUMDAF (internal ID 69838490) features 8 transcripts of interest, the names for which are given in Table 52. The selected protein variants are given in table 53.

TABLE 52
Transcripts of interest
Transcript Name

HUMDAF_T10 (SEQ ID NO: 34)
HUMDAF_T11 (SEQ ID NO: 35)
HUMDAF_T17 (SEQ ID NO: 36)
HUMDAF_T19 (SEQ ID NO: 37)
HUMDAF_T24 (SEQ ID NO: 38)
HUMDAF_T30 (SEQ ID NO: 39)
HUMDAF_T31 (SEQ ID NO: 40)
HUMDAF_T32 (SEQ ID NO: 41)

TABLE 53
Proteins of interest

| | Corresponding |
|---|---|
| Protein Name | Transcript(s) |
| HUMDAF_P14 (SEQ ID NO: 51) | HUMDAF_T10 |
| | (SEQ ID NO: 34) |
| HUMDAF_P15 (SEQ ID NO: 52) | HUMDAF_T11 |
| | (SEQ ID NO: 35); |
| | HUMDAF_T19 |
| | (SEQ ID NO: 37) |
| HUMDAF_P20 (SEQ ID NO: 53) | HUMDAF_T17 |
| | (SEQ ID NO: 36) |
| HUMDAF_P26 (SEQ ID NO: 54) | HUMDAF_T24 |
| | (SEQ ID NO: 38) |
| HUMDAF_P29 (SEQ ID NO: 55) | HUMDAF_T30 |
| | (SEQ ID NO: 39) |
| HUMDAF_P30 (SEQ ID NO: 56) | HUMDAF_T31 |
| | (SEQ ID NO: 40) |
| HUMDAF_P31 (SEQ ID NO: 57) | HUMDAF_T32 |
| | (SEQ ID NO: 41) |

These sequences are variants of the known protein Complement decay-accelerating factor precursor (SwissProt accession identifier DAF_HUMAN (SEQ ID NO:42); known also according to the synonyms CD55 antigen), referred to herein as the previously known protein.

Protein Complement decay-accelerating factor precursor (SEQ ID NO:42) is known or believed to have the following function(s): This protein recognizes C4b and C3b fragments that condense with cell-surface hydroxyl or amino groups when nascent C4b and C3b are locally generated during C4 and c3 activation. Interaction of daf with cell-associated C4b and C3b polypeptides interferes with their ability to catalyze the conversion of C2 and factor B to enzymatically active C2a and Bb and thereby prevents the formation of C4b2a and C3bBb, the amplification convertases of the complement cascade; Also acts as the receptor for echovirus 7 and related viruses (echoviruses 13, 21, 29 and 33). Known polymorphisms for this sequence are as shown in Table 54.

TABLE 54
Amino acid mutations for Known Protein

| SNP position(s) on | |
|---|---|
| amino acid sequence | Comment |
| 82 | L −> R (in WES(a) antigen). /FTId = VAR_001999 |
| 227 | A −> P (in Cr(a−) antigen). /FTId = VAR_002001 |
| 52 | R −> L (in Tc(b) antigen). /FTId = VAR_001997 |
| 80 | I −> T |
| 85 | S −> M |
| 187 | S −> T |
| 199 | S −> L (in Dr(a−) antigen). /FTId = VAR_002000 |
| 38 | L −> G |
| 240 | R −> H (in GUTI(−) antigen). /FTId = |
| | VAR_015884 |
| 52 | R −> P (in Tc(c) antigen). /FTId = VAR_001998 |
| 297 | Q −> H |

Protein Complement decay-accelerating factor precursor (SEQ ID NO:42) localization is believed to be Isoform 2: Cell membrane; lipid-anchor; GPI-anchor.

The previously known protein also has the following indication(s) and/or potential therapeutic use(s): Reperfusion injury; Transplant rejection, general; Arthritis, rheumatoid. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Complement factor 1 stimulant; Complement factor inhibitor; CD59 agonist. A therapeutic role for a protein represented by the cluster has been predicted. The cluster was assigned this field because there was information in the drug database or the public databases (e.g., described herein above) that this protein, or part thereof, is used or optionally may be used for a potential therapeutic indication: Anticancer, immunological; Cytokine; Antiarthritic, immunological; Recombinant, other; Immunosuppressant; Cardiovascular.

As noted above, cluster HUMDAF features 8 transcript(s), which were listed in Table 52 above. These transcript(s) encode for protein(s) which are variant(s) of protein Complement decay-accelerating factor precursor (SEQ ID NO:42). A description of each variant protein according to the present invention is now provided.

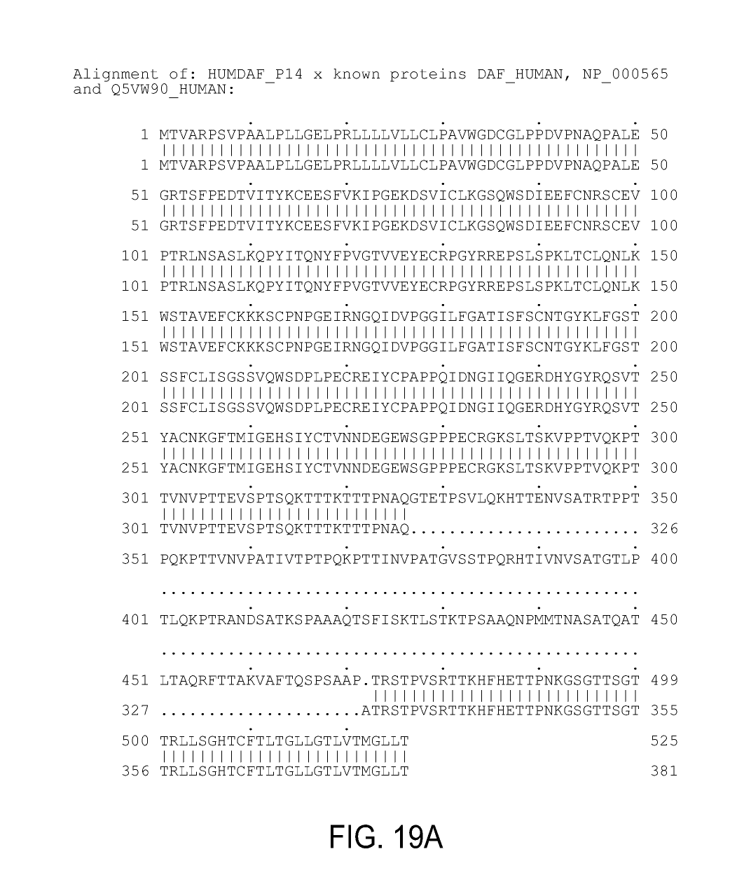

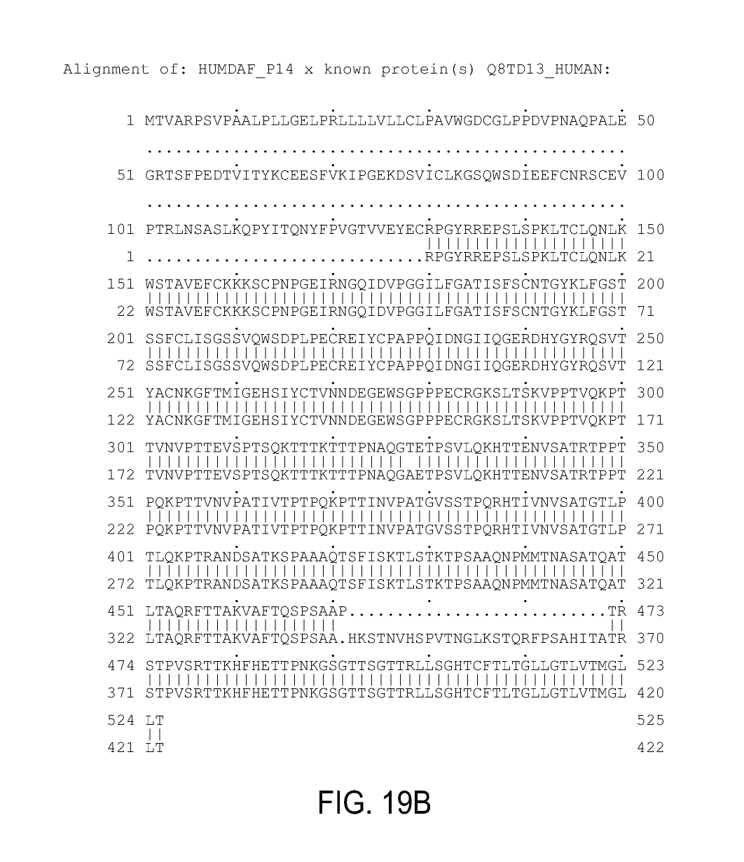

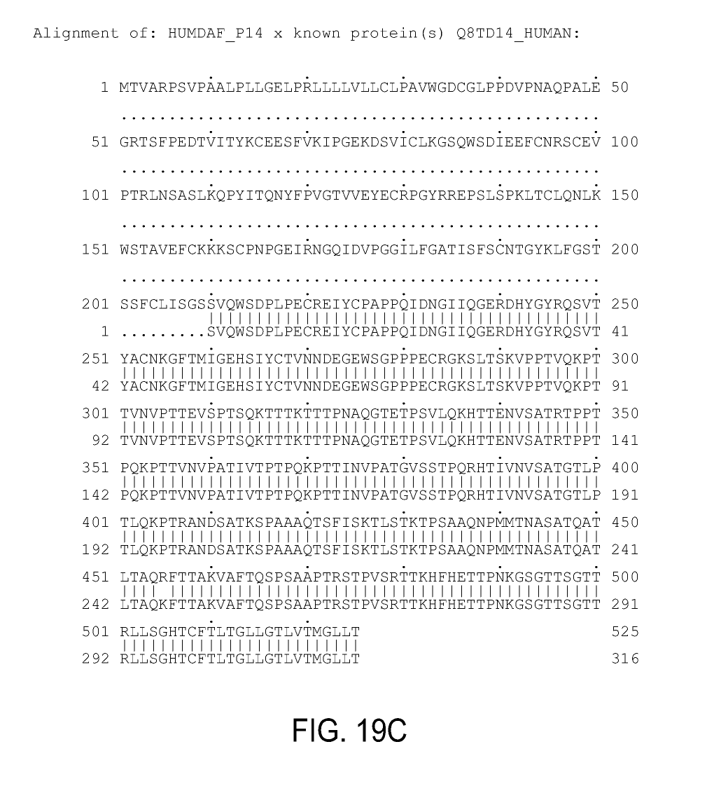

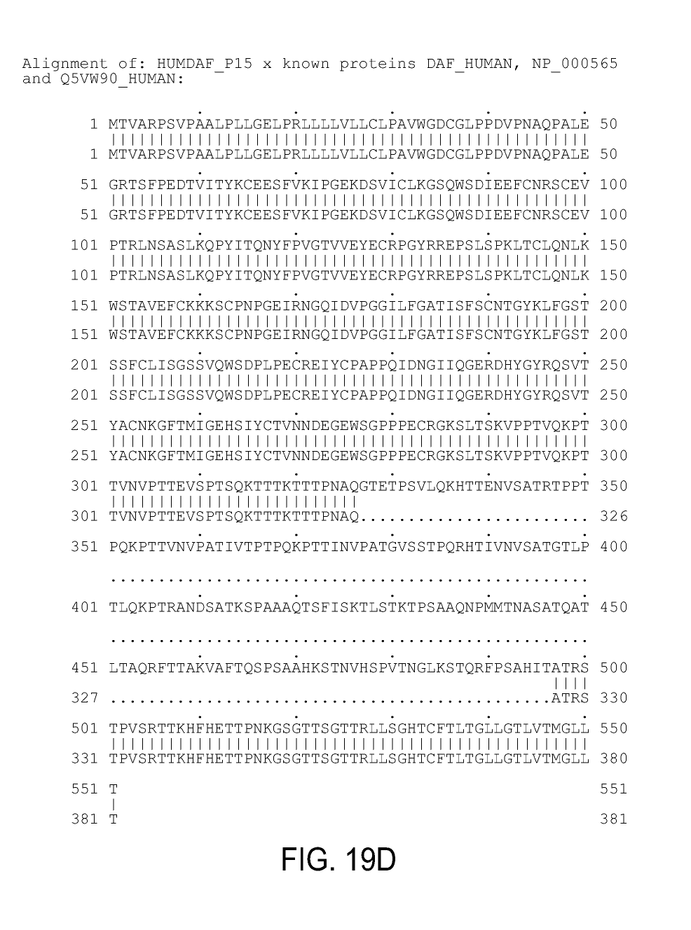

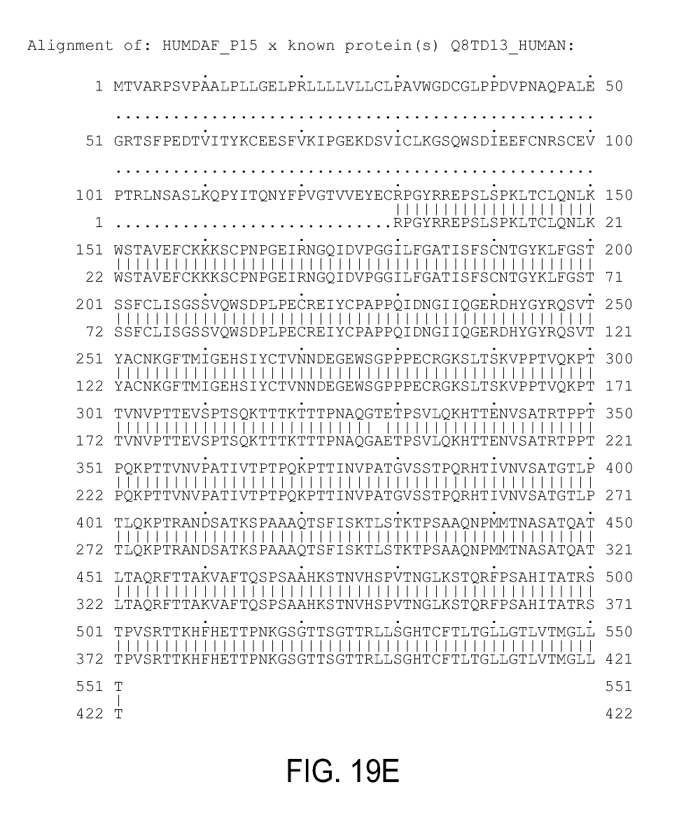

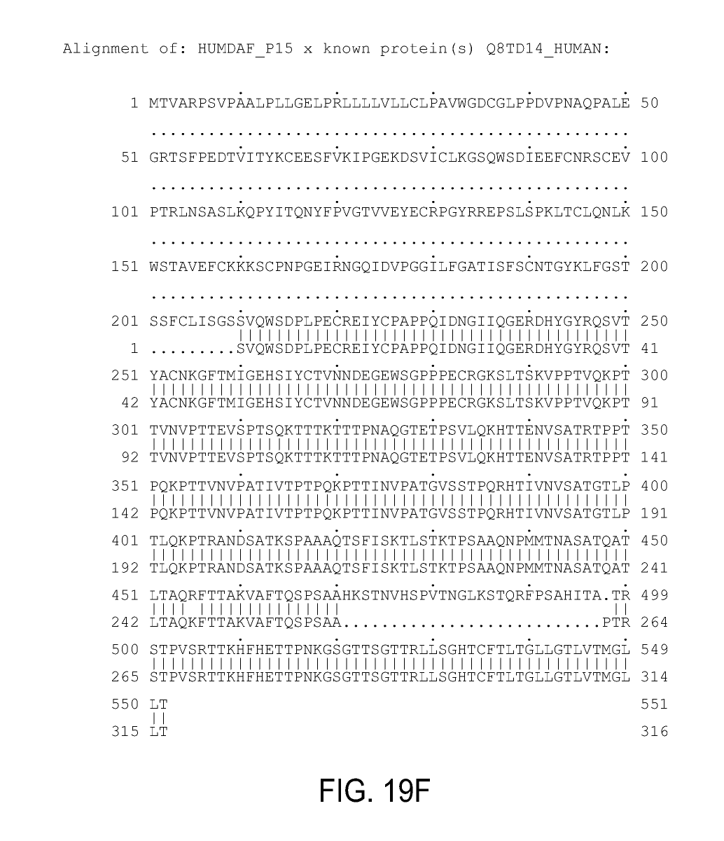

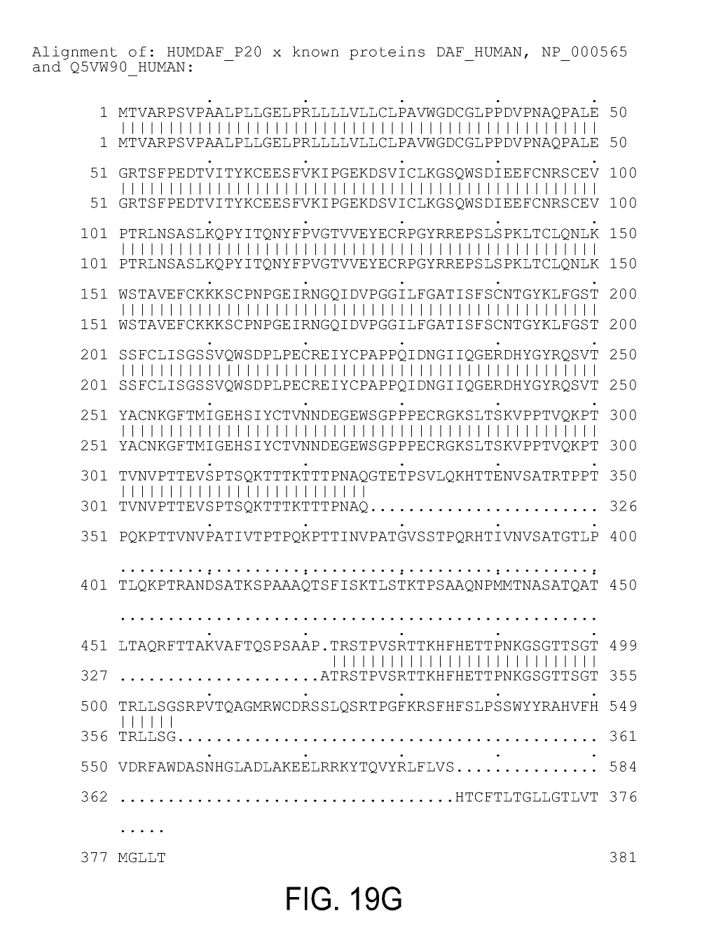

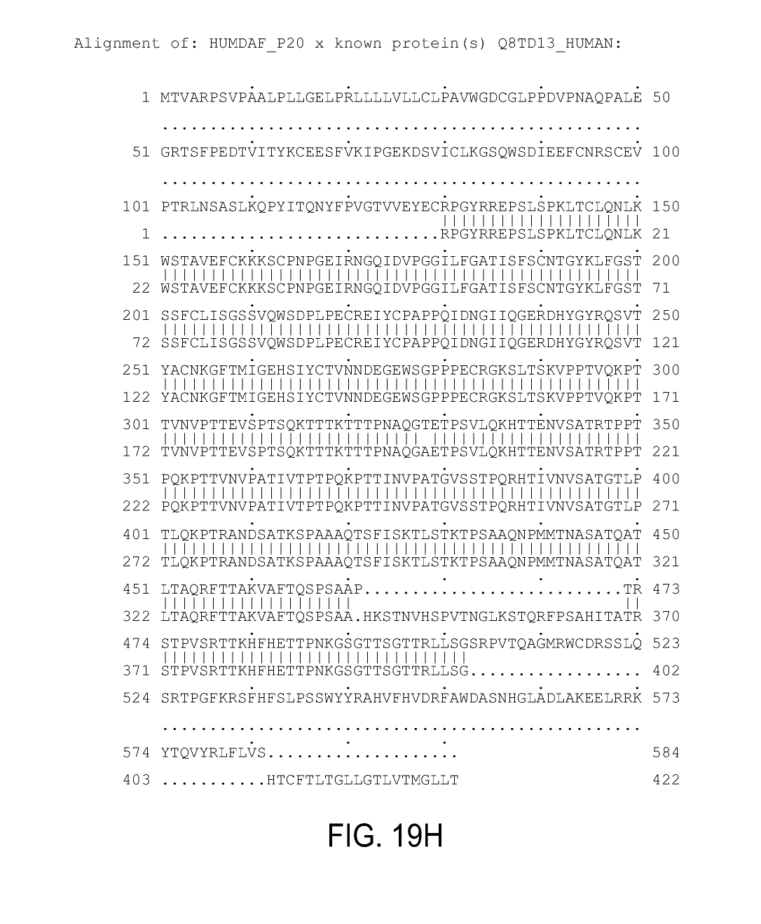

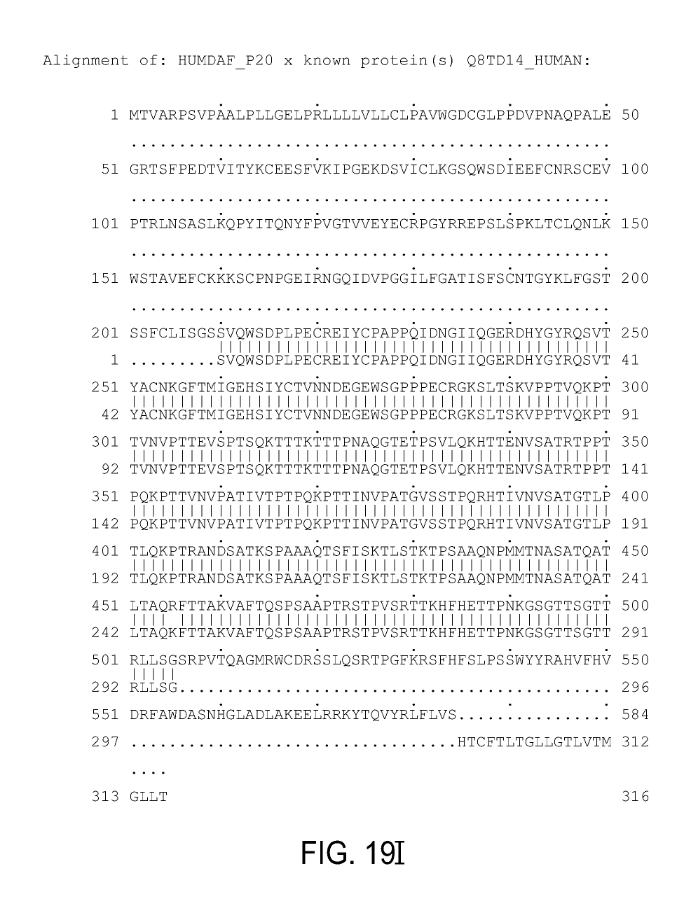

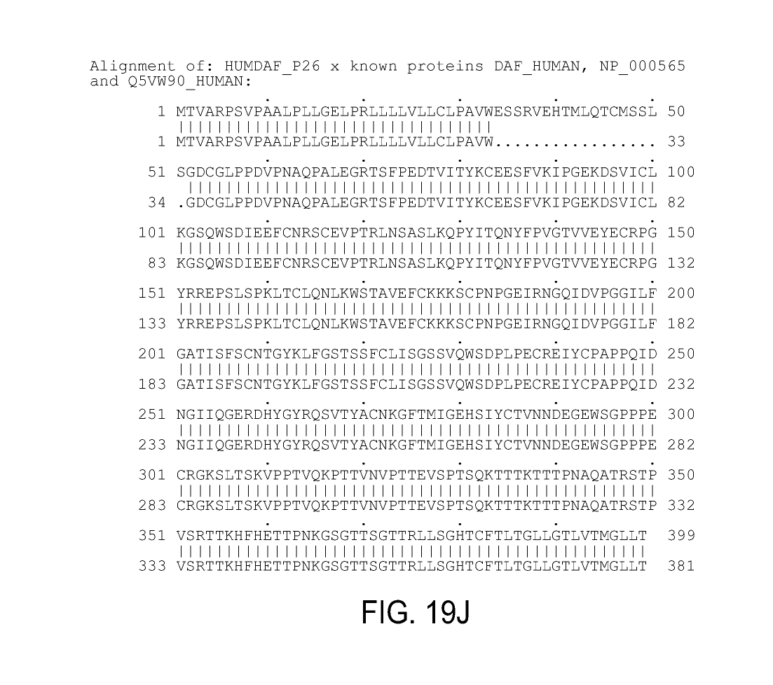

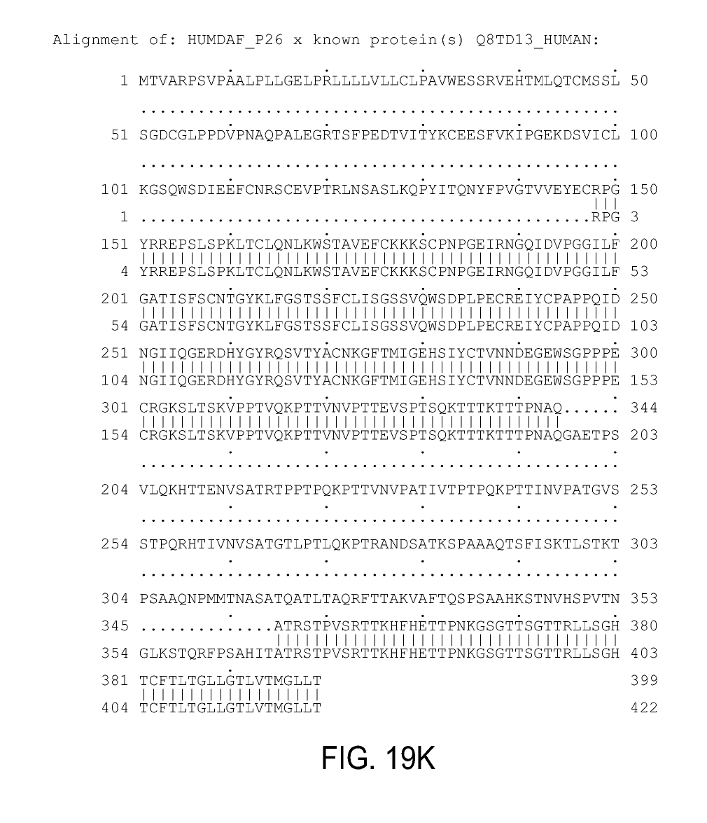

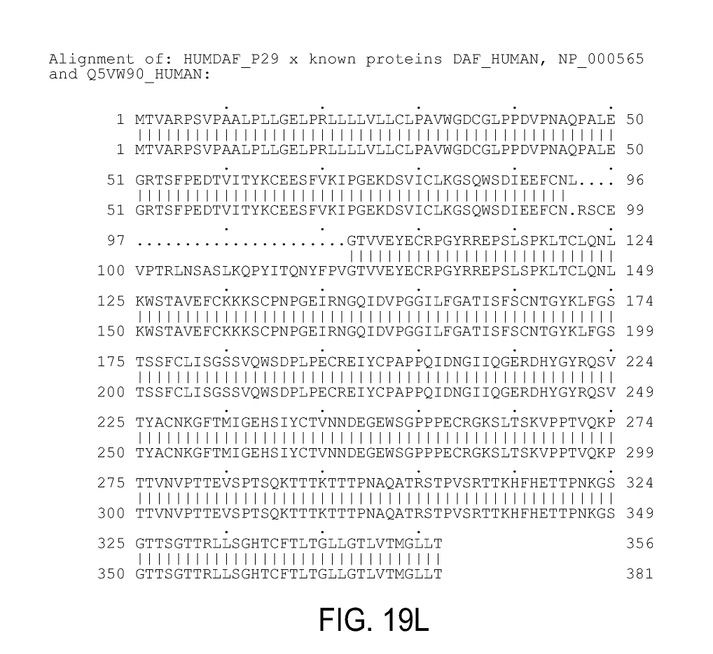

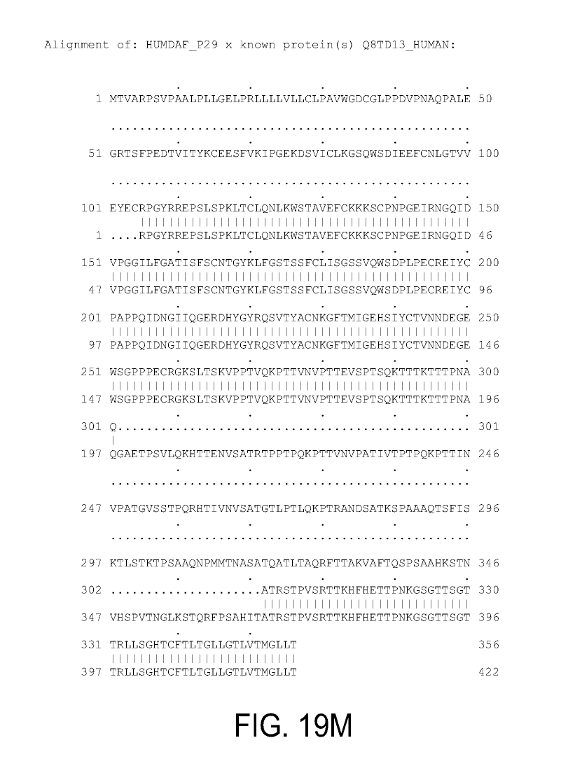

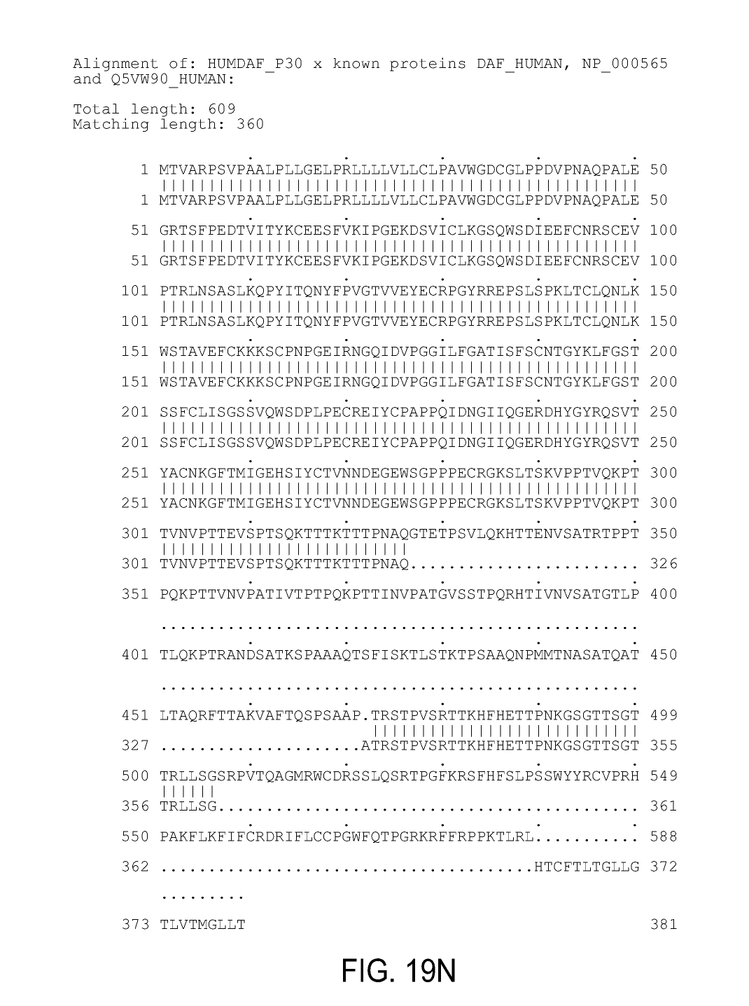

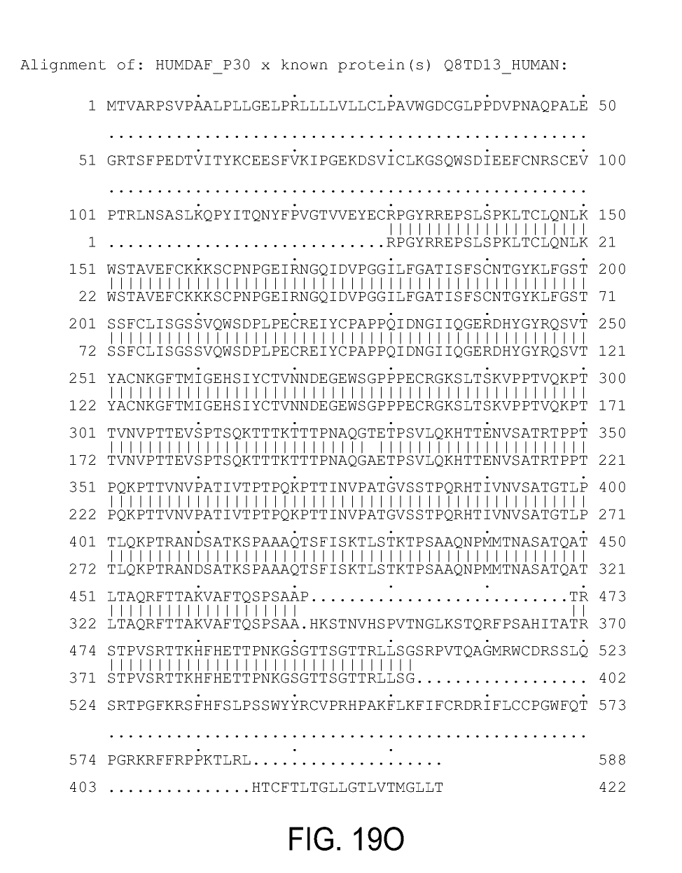

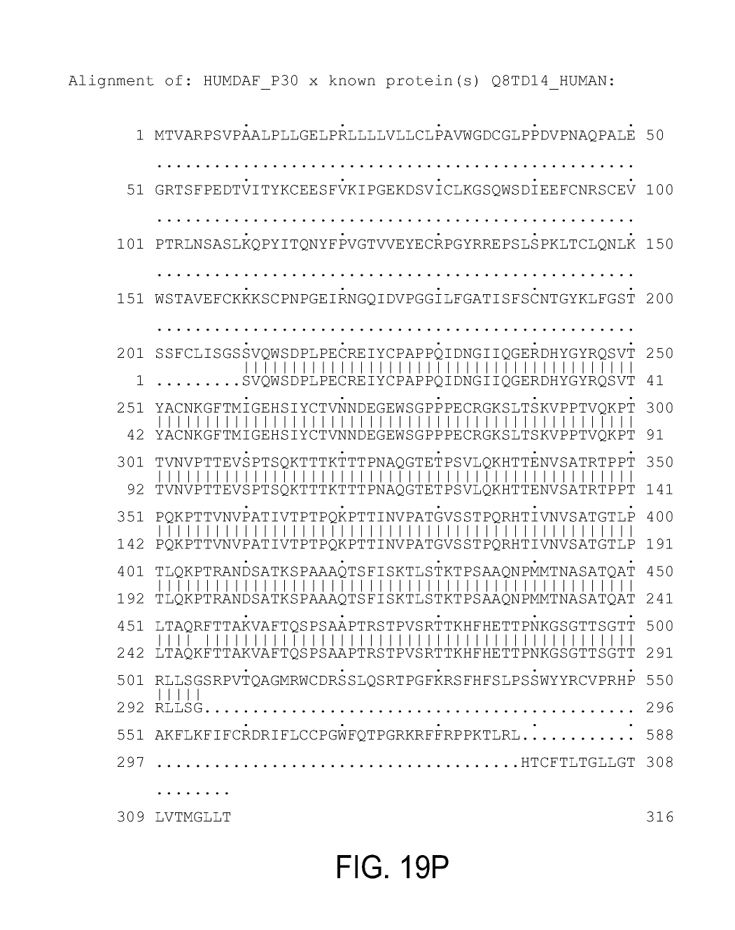

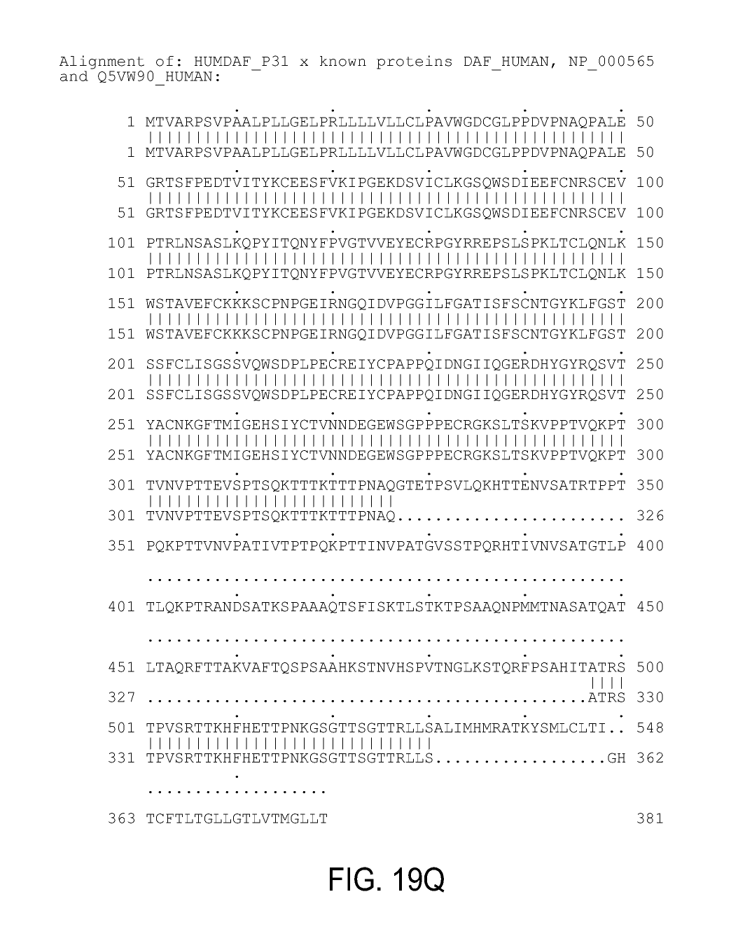

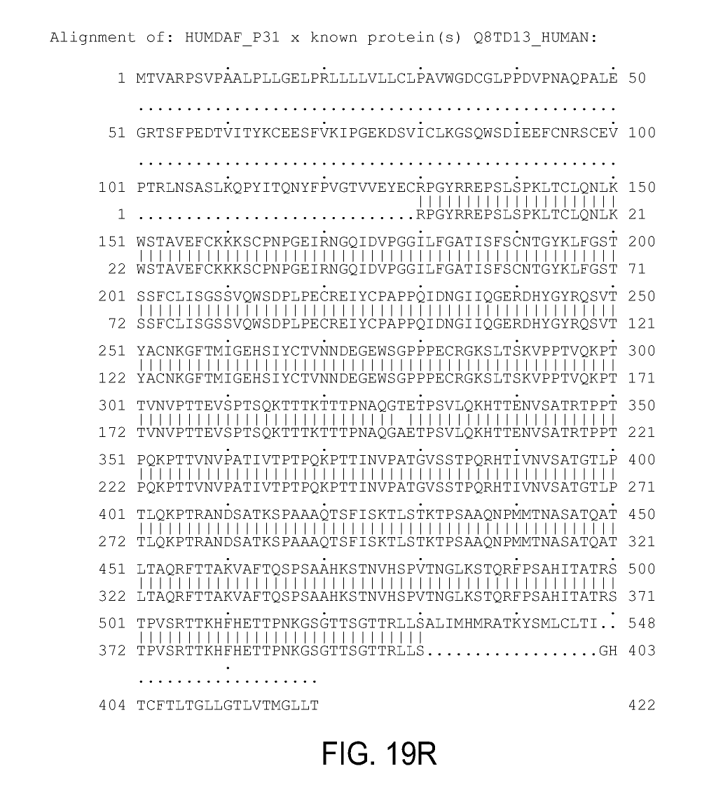

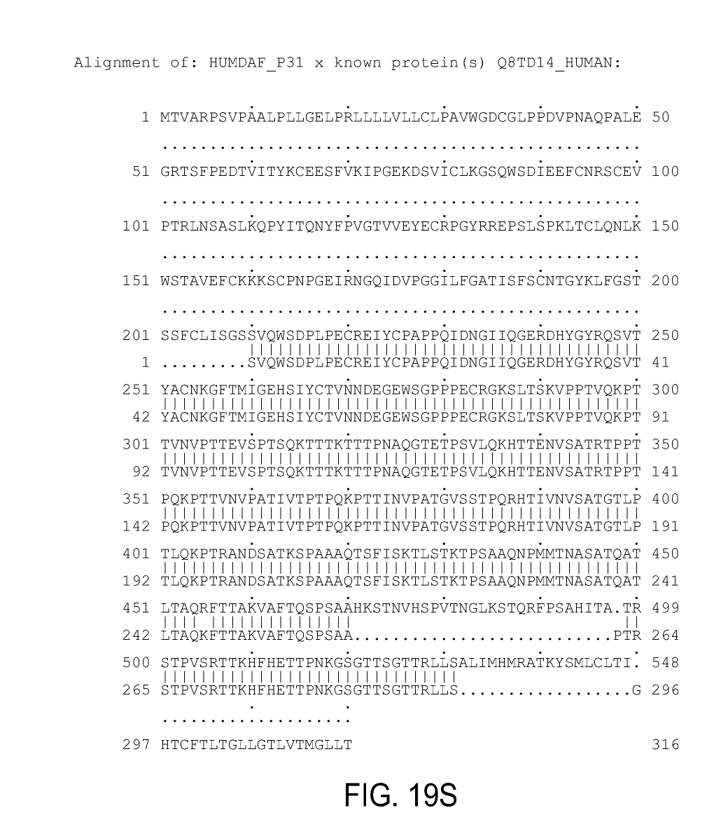

Variant protein HUMDAF_P14 (SEQ ID NO:51) according to the present invention has an amino acid sequence encoded by transcript HUMDAF_T10 (SEQ ID NO:34). One or more alignments to one or more previously published protein sequences are shown in FIGS. 19A, 19B and 19C. A brief description of the relationship of the variant protein according to the present invention to each such aligned protein is as follows:

1. Comparison report between HUMDAF_P14 (SEQ ID NO:51) and known protein DAF_HUMAN (SEQ ID NO:42) (FIG. 19A):

A. An isolated chimeric polypeptide encoding for HUMDAF_P14 (SEQ ID NO:51), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGSSVQWSDPLPECREIYCPAPPQIDNGIIQG ERDHYGYRQSVTYACNKGFTMIGEHSIYCTVNNDEGEWSGPPPECRGKSLTSKVPPT VQKPTTVNVPTTEVSPTSQKTTTKTTTPNAQ corresponding to amino acids 1-326 of known protein DAF_HUMAN (SEQ ID NO:42), which also corresponds to amino acids 1-326 of HUMDAF_P14 (SEQ ID NO:51), a second amino acid sequence being at least 70%, optionally at least 80%, preferably at least 85%, more preferably at least 90% and most preferably at least 95% homologous to a polypeptide having the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP corresponding to amino acids 327-471 of HUMDAF_P14 (SEQ ID NO:51), and a third amino acid sequence being at least 90% homologous to TRSTPVSRTTKHFHETTPNKGSGTTSGTTRLLSGHTCFTLTGLLGTLVTMGLLT corresponding to amino acids 328-381 of known protein DAF_HUMAN (SEQ ID NO:42), which also corresponds to amino acids 472-525 of HUMDAF_P14 (SEQ ID NO:51), wherein said first amino acid sequence, second amino acid sequence and third amino acid sequence are contiguous and in a sequential order.

B. An isolated polypeptide encoding for an edge portion of HUMDAF_P14 (SEQ ID NO:51), comprising an amino acid sequence being at least 70%, optionally at least about 80%, preferably at least about 85%, more preferably at least about 90% and most preferably at least about 95% homologous to the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP of HUMDAF_P14 (SEQ ID NO:51).

2. Comparison report between HUMDAF_P14 (SEQ ID NO:51) and known protein Q8TD13_HUMAN (SEQ ID NO:50) (FIG. 19B):

A. An isolated chimeric polypeptide encoding for HUMDAF_P14 (SEQ ID NO:51), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC corresponding to amino acids 1-129 of HUMDAF_P14 (SEQ ID NO:51), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQG corresponding to amino acids 1-198 of known protein(s) Q8TD13_HUMAN (SEQ ID NO:50), which also corresponds to amino acids 130-327 of HUMDAF_P14 (SEQ ID NO:51), a bridging amino acid T corresponding to amino acid 328 of HUMDAF_P14 (SEQ ID NO:51), a third amino acid sequence being at least 90% homologous to ETPSVLQKHTTENVSATRTPPTPQKPTTVNVPATIVTPTPQKPTTINVPATGVSSTPQR HTIVNVSATGTLPTLQKPTRANDSATKSPAAAQTSFISKTLSTKTPSAAQNPMMTNAS ATQATLTAQRFTTAKVAFTQSPSAA corresponding to amino acids 200-341 of known protein(s) Q8TD13_HUMAN (SEQ ID NO:50), which also corresponds to amino acids 329-470 of HUMDAF_P14 (SEQ ID NO:51), a fourth bridging amino acid sequence comprising of P, and a fifth amino acid sequence being at least 90% homologous to TRSTPVSRTTKHFHETTPNKGSGTTSGTTRLLSGHTCFTLTGLLGTLVTMGLLT corresponding to amino acids 369-422 of known protein(s) Q8TD13_HUMAN (SEQ ID NO:50), which also corresponds to amino acids 472-525 of HUMDAF_P14 (SEQ ID NO:51), wherein said first amino acid sequence, second amino acid sequence, bridging amino acid, third amino acid sequence, fourth amino acid sequence and fifth amino acid sequence are contiguous and in a sequential order.

B. An isolated polypeptide encoding for a head of HUMDAF_P14 (SEQ ID NO:51), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC of HUMDAF_P14 (SEQ ID NO:51).

C. An isolated polypeptide encoding for an edge portion of HUMDAF_P14 (SEQ ID NO:51), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise APT having a structure as follows (numbering according to HUMDAF_P14 (SEQ ID NO:51)): a sequence starting from any of amino acid numbers 470−x to 470; and ending at any of amino acid numbers 472+((n−3)−x), in which x varies from 0 to n−3.

6. Comparison report between HUMDAF_P14 (SEQ ID NO:51) and known protein Q8TD14 HUMAN (SEQ ID NO:48) (FIG. 19C)

A. An isolated chimeric polypeptide encoding for HUMDAF_P14 (SEQ ID NO:51), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS corresponding to amino acids 1-209 of HUMDAF_P14 (SEQ ID NO:51), a second amino acid sequence being at least 90% homologous to SVQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQ corresponding to amino acids 1-245 of known protein(s) Q8TD14 HUMAN (SEQ ID NO:48), which also corresponds to amino acids 210-454 of HUMDAF_P14 (SEQ ID NO:51), a bridging amino acid R corresponding to amino acid 455 of HUMDAF_P14 (SEQ ID NO:51), and a third amino acid sequence being at least 90% homologous to FTTAKVAFTQSPSAAPTRSTPVSRTTKHFHETTPNKGSGTTSGTTRLLSGHTCFTLTGL LGTLVTMGLLT corresponding to amino acids 247-316 of known protein(s) Q8TD14 HUMAN (SEQ ID NO:48), which also corresponds to amino acids 456-525 of HUMDAF_P14 (SEQ ID NO:51), wherein said first amino acid sequence, second amino acid sequence, bridging amino acid and third amino acid sequence are contiguous and in a sequential order.

B. An isolated polypeptide encoding for a head of HUMDAF_P14 (SEQ ID NO:51), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS of HUMDAF_P14 (SEQ ID NO:51).

The localization of the variant protein was determined according to results from a number of different software programs and analyses, including analyses from SignalP and other specialized programs. The variant protein is believed to be located as follows with regard to the cell: membrane.

Variant protein HUMDAF_P14 (SEQ ID NO:51) also has the following non-silent SNPs (Single Nucleotide Polymorphisms) as listed in Table 55, (given according to their position(s) on the amino acid sequence, with the alternative amino acid(s) listed; the last column indicates whether the SNP is known or not; the presence of known SNPs in variant protein HUMDAF_P14 (SEQ ID NO:51) sequence provides support for the deduced sequence of this variant protein according to the present invention).

TABLE 55
Amino acid mutations

| SNP position(s) on | | |
|---|---|---|
| amino acid sequence | Alternative amino acid(s) | Previously known SNP? |
| 11 | A −> | No |
| 159 | K −> T | No |
| 185 | T −> | No |
| 232 | D −> G | No |
| 233 | N −> H | No |
| 328 | T −> A | No |
| 369 | Q −> * | No |
| 455 | R −> K | No |

The glycosylation sites of variant protein HUMDAF_P14 (SEQ ID NO:51), as compared to the known protein Complement decay-accelerating factor precursor (SEQ ID NO:42), are described in Table 56 (given according to their position(s) on the amino acid sequence in the first column; the second column indicates whether the glycosylation site is present in the variant protein; and the last column indicates whether the position is different on the variant protein).

TABLE 56
Glycosylation site(s)

| Position(s) on known | Present in variant | Position(s) on variant |
|---|---|---|
| amino acid sequence | protein? | protein |
| 95 | Yes | 95 |

Variant protein HUMDAF_P14 (SEQ ID NO:51) is encoded by the transcript HUMDAF_T10 (SEQ ID NO:34), for which the coding portion starts at position 329 and ends at position 1903. The transcript also has the following SNPs as listed in Table 57 (given according to their position on the nucleotide sequence, with the alternative nucleic acid listed).

TABLE 57
Nucleic acid SNPs

| Polymorphism | SNP position(s) on nucleotide sequence |
|---|---|
| G −> A | 25, 1692 |
| A −> C | 130, 804, 1025, 2315, 2587, 2693 |
| A −> G | 130, 1023, 1310, 1426, 2168 |
| C −> | 277, 308, 883, 2404 |
| −> A | 349 |
| −> T | 349, 2232 |
| G −> | 359 |
| A −> | 881, 2168 |
| C −> T | 1433, 2404 |
| T −> | 2183, 2226, 2343 |
| T −> C | 2183 |
| T −> G | 2343 |

Variant protein HUMDAF_P15 (SEQ ID NO:52) according to the present invention has an amino acid sequence encoded by transcripts HUMDAF_T11 (SEQ ID NO:35) and HUMDAF_T19 (SEQ ID NO:37). One or more alignments to one or more previously published protein sequences are shown in FIGS. 19D, 19E and 19F. A brief description of the relationship of the variant protein according to the present invention to each such aligned protein is as follows:

1. Comparison report between HUMDAF_P15 (SEQ ID NO:52) and known protein DAF_HUMAN (SEQ ID NO:42) (FIG. 19D):

A. An isolated chimeric polypeptide encoding for HUMDAF_P15 (SEQ ID NO:52), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGSSVQWSDPLPECREIYCPAPPQIDNGIIQG ERDHYGYRQSVTYACNKGFTMIGEHSIYCTVNNDEGEWSGPPPECRGKSLTSKVPPT VQKPTTVNVPTTEVSPTSQKTTTKTTTPNAQ corresponding to amino acids 1-326 of known protein DAF_HUMAN (SEQ ID NO:42), which also corresponds to amino acids 1-326 of HUMDAF_P15 (SEQ ID NO:52), a second amino acid sequence being at least 70%, optionally at least 80%, preferably at least 85%, more preferably at least 90% and most preferably at least 95% homologous to a polypeptide having the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAHKSTNVHSPVTNGLKSTQRFPSAHIT corresponding to amino acids 327-496 of HUMDAF_P15 (SEQ ID NO:52), and a third amino acid sequence being at least 90% homologous to ATRSTPVSRTTKHFHETTPNKGSGTTSGTTRLLSGHTCFTLTGLLGTLVTMGLLT corresponding to amino acids 327-381 of known protein DAF_HUMAN (SEQ ID NO:42), which also corresponds to amino acids 497-551 of HUMDAF_P15 (SEQ ID NO:52), wherein said first amino acid sequence, second amino acid sequence and third amino acid sequence are contiguous and in a sequential order.

B. An isolated polypeptide encoding for an edge portion of HUMDAF_P15 (SEQ ID NO:52), comprising an amino acid sequence being at least 70%, optionally at least about 80%, preferably at least about 85%, more preferably at least about 90% and most preferably at least about 95% homologous to the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAHKSTNVHSPVTNGLKSTQRFPSAHIT of HUMDAF_P15 (SEQ ID NO:52).

2. Comparison report between HUMDAF_P15 (SEQ ID NO:52) and known protein Q8TD13_HUMAN (SEQ ID NO:50) (FIG. 19E):

A. An isolated chimeric polypeptide encoding for HUMDAF_P15 (SEQ ID NO:52), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC corresponding to amino acids 1-129 of HUMDAF_P15 (SEQ ID NO:52), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQG corresponding to amino acids 1-198 of known protein(s) Q8TD13_HUMAN (SEQ ID NO:50), which also corresponds to amino acids 130-327 of HUMDAF_P15 (SEQ ID NO:52), a bridging amino acid T corresponding to amino acid 328 of HUMDAF_P15 (SEQ ID NO:52), and a third amino acid sequence being at least 90% homologous to ETPSVLQKHTTENVSATRTPPTPQKPTTVNVPATIVTPTPQKPTTINVPATGVSSTPQR HTIVNVSATGTLPTLQKPTRANDSATKSPAAAQTSFISKTLSTKTPSAAQNPMMTNAS ATQATLTAQRFTTAKVAFTQSPSAAHKSTNVHSPVTNGLKSTQRFPSAHITATRSTPV SRTTKHFHETTPNKGSGTTSGTTRLLSGHTCFTLTGLLGTLVTMGLLT corresponding to amino acids 200-422 of known protein(s) Q8TD13_HUMAN (SEQ ID NO:50), which also corresponds to amino acids 329-551 of HUMDAF_P15 (SEQ ID NO:52), wherein said first amino acid sequence, second amino acid sequence, bridging amino acid and third amino acid sequence are contiguous and in a sequential order.

B. An isolated polypeptide encoding for a head of HUMDAF_P15 (SEQ ID NO:52), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC of HUMDAF_P15 (SEQ ID NO:52).

6. Comparison report between HUMDAF_P15 (SEQ ID NO:52) and known protein Q8TD14 HUMAN (SEQ ID NO:48) (FIG. 19F:)

A. An isolated chimeric polypeptide encoding for HUMDAF_P15 (SEQ ID NO:52), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS corresponding to amino acids 1-209 of HUMDAF_P15 (SEQ ID NO:52), a second amino acid sequence being at least 90% homologous to SVQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQ corresponding to amino acids 1-245 of known protein(s) Q8TD14 HUMAN (SEQ ID NO:48), which also corresponds to amino acids 210-454 of HUMDAF_P15 (SEQ ID NO:52), a bridging amino acid R corresponding to amino acid 455 of HUMDAF_P15 (SEQ ID NO:52), a third amino acid sequence being at least 90% homologous to FTTAKVAFTQSPSAA corresponding to amino acids 247-261 of known protein(s) Q8TD14 HUMAN (SEQ ID NO:48), which also corresponds to amino acids 456-470 of HUMDAF_P15 (SEQ ID NO:52), a fourth amino acid sequence being at least 70%, optionally at least 80%, preferably at least 85%, more preferably at least 90% and most preferably at least 95% homologous to a polypeptide having the sequence HKSTNVHSPVTNGLKSTQRFPSAHITA corresponding to amino acids 471-497 of HUMDAF_P15 (SEQ ID NO:52), and a fifth amino acid sequence being at least 90% homologous to TRSTPVSRTTKHFHETTPNKGSGTTSGTTRLLSGHTCFTLTGLLGTLVTMGLLT corresponding to amino acids 263-316 of known protein(s) Q8TD14 HUMAN (SEQ ID NO:48), which also corresponds to amino acids 498-551 of HUMDAF_P15 (SEQ ID NO:52), wherein said first amino acid sequence, second amino acid sequence, bridging amino acid, third amino acid sequence, fourth amino acid sequence and fifth amino acid sequence are contiguous and in a sequential order.

B. An isolated polypeptide encoding for a head of HUMDAF_P15 (SEQ ID NO:52), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS of HUMDAF_P15 (SEQ ID NO:52).

C. An isolated polypeptide encoding for an edge portion of HUMDAF_P15 (SEQ ID NO:52), comprising an amino acid sequence being at least 70%, optionally at least about 80%, preferably at least about 85%, more preferably at least about 90% and most preferably at least about 95% homologous to the sequence HKSTNVHSPVTNGLKSTQRFPSAHITA of HUMDAF_P15 (SEQ ID NO:52).

The localization of the variant protein was determined according to results from a number of different software programs and analyses, including analyses from SignalP and other specialized programs. The variant protein is believed to be located as follows with regard to the cell: membrane.

Variant protein HUMDAF_P15 (SEQ ID NO:52) also has the following non-silent SNPs (Single Nucleotide Polymorphisms) as listed in Table 58, (given according to their position(s) on the amino acid sequence, with the alternative amino acid(s) listed; the last column indicates whether the SNP is known or not; the presence of known SNPs in variant protein HUMDAF_P15 (SEQ ID NO:52) sequence provides support for the deduced sequence of this variant protein according to the present invention).

TABLE 58
Amino acid mutations

| SNP position(s) on | | |
|---|---|---|
| amino acid sequence | Alternative amino acid(s) | Previously known SNP? |
| 11 | A −> | No |
| 159 | K −> T | No |
| 185 | T −> | No |
| 232 | D −> G | No |
| 233 | N −> H | No |
| 328 | T −> A | No |
| 369 | Q −> * | No |
| 455 | R −> K | No |
| 471 | H −> R | No |

The glycosylation sites of variant protein HUMDAF_P15 (SEQ ID NO:52), as compared to the known protein Complement decay-accelerating factor precursor (SEQ ID NO:42), are described in Table 59 (given according to their position(s) on the amino acid sequence in the first column; the second column indicates whether the glycosylation site is present in the variant protein; and the last column indicates whether the position is different on the variant protein).

TABLE 59
Glycosylation site(s)

| Position(s) on known | Present in variant | Position(s) on variant |
|---|---|---|
| amino acid sequence | protein? | protein |
| 95 | Yes | 95 |

Variant protein HUMDAF_P15 (SEQ ID NO:52) is encoded by the following transcripts: HUMDAF_T11 (SEQ ID NO:35) and HUMDAF_T19 (SEQ ID NO:37).

The coding portion of transcript HUMDAF_T11 (SEQ ID NO:35) starts at position 329 and ends at position 1981. The transcript also has the following SNPs as listed in Table 60 (given according to their position on the nucleotide sequence, with the alternative nucleic acid listed).

TABLE 60
Nucleic acid SNPs

| Polymorphism | SNP position(s) on nucleotide sequence |
|---|---|
| G −> A | 25, 1692 |
| A −> C | 130, 804, 1025, 2393, 2665, 2771 |
| A −> G | 130, 1023, 1310, 1426, 1740, 2246 |
| C −> | 277, 308, 883, 2482 |
| −> A | 349 |
| −> T | 349, 2310 |
| G −> | 359 |
| A −> | 881, 2246 |
| C −> T | 1433, 2482 |
| T −> | 2261, 2304, 2421 |
| T −> C | 2261 |
| T −> G | 2421 |

The coding portion of transcript HUMDAF_T19 (SEQ ID NO:37) starts at position 329 and ends at position 1981. The transcript also has the following SNPs as listed in Table 61 (given according to their position on the nucleotide sequence, with the alternative nucleic acid listed).

TABLE 61
Nucleic acid SNPs

| Polymorphism | SNP position(s) on nucleotide sequence |
|---|---|
| G −> A | 25, 1692 |
| A −> C | 130, 804, 1025 |
| A −> G | 130, 1023, 1310, 1426, 1740 |
| C −> | 277, 308, 883 |
| −> A | 349 |
| −> T | 349 |
| G −> | 359 |
| A −> | 881 |

C->T 1433

Variant protein HUMDAF_P20 (SEQ ID NO:53) according to the present invention has an amino acid sequence encoded by transcript HUMDAF_T17 (SEQ ID NO:36). One or more alignments to one or more previously published protein sequences are shown in FIGS. 19G, 19H and 19I. A brief description of the relationship of the variant protein according to the present invention to each such aligned protein is as follows:

1. Comparison report between HUMDAF_P20 (SEQ ID NO:53) and known protein DAF_HUMAN (SEQ ID NO:42) (FIG. 19G):

A. An isolated chimeric polypeptide encoding for HUMDAF_P20 (SEQ ID NO:53), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGSSVQWSDPLPECREIYCPAPPQIDNGIIQG ERDHYGYRQSVTYACNKGFTMIGEHSIYCTVNNDEGEWSGPPPECRGKSLTSKVPPT VQKPTTVNVPTTEVSPTSQKTTTKTTTPNAQ corresponding to amino acids 1-326 of known protein DAF_HUMAN (SEQ ID NO:42), which also corresponds to amino acids 1-326 of HUMDAF_P20 (SEQ ID NO:53), a second amino acid sequence being at least 70%, optionally at least 80%, preferably at least 85%, more preferably at least 90% and most preferably at least 95% homologous to a polypeptide having the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP corresponding to amino acids 327-471 of HUMDAF_P20 (SEQ ID NO:53), a third amino acid sequence being at least 90% homologous to TRSTPVSRTTKHFHETTPNKGSGTTSGTTRLLSG corresponding to amino acids 328-361 of known protein DAF_HUMAN (SEQ ID NO:42), which also corresponds to amino acids 472-505 of HUMDAF_P20 (SEQ ID NO:53), and a fourth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRAHVFHVDRFAWDASNHG LADLAKEELRRKYTQVYRLFLVS corresponding to amino acids 506-584 of HUMDAF_P20 (SEQ ID NO:53), wherein said first amino acid sequence, second amino acid sequence, third amino acid sequence and fourth amino acid sequence are contiguous and in a sequential order.

B. An isolated polypeptide encoding for an edge portion of HUMDAF_P20 (SEQ ID NO:53), comprising an amino acid sequence being at least 70%, optionally at least about 80%, preferably at least about 85%, more preferably at least about 90% and most preferably at least about 95% homologous to the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP of HUMDAF_P20 (SEQ ID NO:53).

C. An isolated polypeptide encoding for an edge portion of HUMDAF_P20 (SEQ ID NO:53), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRAHVFHVDRFAWDASNHG LADLAKEELRRKYTQVYRLFLVS of HUMDAF_P20 (SEQ ID NO:5).

2. Comparison report between HUMDAF_P20 (SEQ ID NO:53) and known protein Q8TD13_HUMAN (SEQ ID NO:50) (FIG. 19H):

A. An isolated chimeric polypeptide encoding for HUMDAF_P20 (SEQ ID NO:53), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC corresponding to amino acids 1-129 of HUMDAF_P20 (SEQ ID NO:53), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQG corresponding to amino acids 1-198 of known protein(s) Q8TD13_HUMAN (SEQ ID NO:50), which also corresponds to amino acids 130-327 of HUMDAF_P20 (SEQ ID NO:53), a bridging amino acid T corresponding to amino acid 328 of HUMDAF_P20 (SEQ ID NO:53), a third amino acid sequence being at least 90% homologous to ETPSVLQKHTTENVSATRTPPTPQKPTTVNVPATIVTPTPQKPTTINVPATGVSSTPQR HTIVNVSATGTLPTLQKPTRANDSATKSPAAAQTSFISKTLSTKTPSAAQNPMMTNAS ATQATLTAQRFTTAKVAFTQSPSAA corresponding to amino acids 200-341 of known protein(s) Q8TD13_HUMAN (SEQ ID NO:50), which also corresponds to amino acids 329-470 of HUMDAF_P20 (SEQ ID NO:53), a fourth bridging amino acid sequence comprising of P, a fifth amino acid sequence being at least 90% homologous to TRSTPVSRTTKHFHETTPNKGSGTTSGTTRLLSG corresponding to amino acids 369-402 of known protein(s) Q8TD13_HUMAN (SEQ ID NO:50), which also corresponds to amino acids 472-505 of HUMDAF_P20 (SEQ ID NO:53), and a sixth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRAHVFHVDRFAWDASNHG LADLAKEELRRKYTQVYRLFLVS corresponding to amino acids 506-584 of HUMDAF_P20 (SEQ ID NO:53), wherein said first amino acid sequence, second amino acid sequence, bridging amino acid, third amino acid sequence, fourth amino acid sequence, fifth amino acid sequence and sixth amino acid sequence are contiguous and in a sequential order.

B. An isolated polypeptide encoding for a head of HUMDAF_P20 (SEQ ID NO:53), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC of HUMDAF_P20 (SEQ ID NO:53).

C. An isolated polypeptide encoding for an edge portion of HUMDAF_P20 (SEQ ID NO:53), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise APT having a structure as follows (numbering according to HUMDAF_P20 (SEQ ID NO:53)): a sequence starting from any of amino acid numbers 470−x to 470; and ending at any of amino acid numbers 472+((n−3)−x), in which x varies from 0 to n−3.

D. An isolated polypeptide encoding for an edge portion of HUMDAF_P20 (SEQ ID NO:53), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRAHVFHVDRFAWDASNHG LADLAKEELRRKYTQVYRLFLVS of HUMDAF_P20 (SEQ ID NO:53).

3. Comparison report between HUMDAF_P20 (SEQ ID NO:53) and known protein(s) Q8TD14 HUMAN (SEQ ID NO:48) (FIG. 19I):

A. An isolated chimeric polypeptide encoding for HUMDAF_P20 (SEQ ID NO:53), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS corresponding to amino acids 1-209 of HUMDAF_P20 (SEQ ID NO:53), a second amino acid sequence being at least 90% homologous to SVQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQ corresponding to amino acids 1-245 of known protein(s) Q8TD14 HUMAN (SEQ ID NO:48), which also corresponds to amino acids 210-454 of HUMDAF_P20 (SEQ ID NO:53), a bridging amino acid R corresponding to amino acid 455 of HUMDAF_P20 (SEQ ID NO:53), a third amino acid sequence being at least 90% homologous to FTTAKVAFTQSPSAAPTRSTPVSRTTKHFHETTPNKGSGTTSGTTRLLSG corresponding to amino acids 247-296 of known protein(s) Q8TD14 HUMAN (SEQ ID NO:48), which also corresponds to amino acids 456-505 of HUMDAF_P20 (SEQ ID NO:53), and a fourth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRAHVFHVDRFAWDASNHG LADLAKEELRRKYTQVYRLFLVS corresponding to amino acids 506-584 of HUMDAF_P20 (SEQ ID NO:53), wherein said first amino acid sequence, second amino acid sequence, bridging amino acid, third amino acid sequence and fourth amino acid sequence are contiguous and in a sequential order.

B. An isolated polypeptide encoding for a head of HUMDAF_P20 (SEQ ID NO:53), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS of HUMDAF_P20 (SEQ ID NO:53).

C. An isolated polypeptide encoding for an edge portion of HUMDAF_P20 (SEQ ID NO:53), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRAHVFHVDRFAWDASNHG LADLAKEELRRKYTQVYRLFLVS of HUMDAF_P20 (SEQ ID NO:53).

The localization of the variant protein was determined according to results from a number of different software programs and analyses, including analyses from SignalP and other specialized programs. The variant protein is believed to be located as follows with regard to the cell: membrane.

Variant protein HUMDAF_P20 (SEQ ID NO:53) also has the following non-silent SNPs (Single Nucleotide Polymorphisms) as listed in Table 62, (given according to their position(s) on the amino acid sequence, with the alternative amino acid(s) listed; the last column indicates whether the SNP is known or not; the presence of known SNPs in variant protein HUMDAF_P20 (SEQ ID NO:53) sequence provides support for the deduced sequence of this variant protein according to the present invention).

TABLE 62
Amino acid mutations

| SNP position(s) on | | |
|---|---|---|
| amino acid sequence | Alternative amino acid(s) | Previously known SNP? |
| 11 | A −> | No |
| 159 | K −> T | No |
| 185 | T −> | No |
| 232 | D −> G | No |
| 233 | N −> H | No |
| 328 | T −> A | No |
| 369 | Q −> * | No |
| 455 | R −> K | No |

The glycosylation sites of variant protein HUMDAF_P20 (SEQ ID NO:53), as compared to the known protein Complement decay-accelerating factor precursor (SEQ ID NO:42), are described in Table 63 (given according to their position(s) on the amino acid sequence in the first column; the second column indicates whether the glycosylation site is present in the variant protein; and the last column indicates whether the position is different on the variant protein).

### Table 63—Glycosylation Site(s)

Position(s) on known amino Present in variant protein? Position(s) on variant acid sequence protein
95 Yes 95

Variant protein HUMDAF_P20 (SEQ ID NO:53) is encoded by the transcript HUMDAF_T17 (SEQ ID NO:36), for which the coding portion starts at position 329 and ends at position 2080. The transcript also has the following SNPs as listed in Table 64 (given according to their position on the nucleotide sequence, with the alternative nucleic acid listed).

TABLE 64
Nucleic acid SNPs

| Polymorphism | SNP position(s) on nucleotide sequence |
|---|---|
| G −> A | 25, 1692 |
| A −> C | 130, 804, 1025, 2433, 2705, 2811 |
| A −> G | 130, 1023, 1310, 1426, 2286 |
| C −> | 277, 308, 883, 2522 |
| −> A | 349 |
| −> T | 349, 2350 |
| G −> | 359 |
| A −> | 881, 2286 |
| C −> T | 1433, 2522 |
| T −> | 2301, 2344, 2461 |
| T −> C | 2301 |
| T −> G | 2461 |

Variant protein HUMDAF_P26 (SEQ ID NO:54) according to the present invention has an amino acid sequence encoded by transcript HUMDAF_T24 (SEQ ID NO:38). One or more alignments to one or more previously published protein sequences are shown in FIGS. 19J, and 19K. A brief description of the relationship of the variant protein according to the present invention to each such aligned protein is as follows:

1. Comparison report between HUMDAF_P26 (SEQ ID NO:54) and known protein DAF_HUMAN (SEQ ID NO:42) (FIG. 19J):

A. An isolated chimeric polypeptide encoding for HUMDAF_P26 (SEQ ID NO:54), comprising a first amino acid sequence being at least 90% homologous to MTVARPSVPAALPLLGELPRLLLLVLLCLPAVW corresponding to amino acids 1-33 of known protein DAF_HUMAN (SEQ ID NO:42), which also corresponds to amino acids 1-33 of HUMDAF_P26 (SEQ ID NO:54), a second amino acid sequence being at least 70%, optionally at least 80%, preferably at least 85%, more preferably at least 90% and most preferably at least 95% homologous to a polypeptide having the sequence ESSRVEHTMLQTCMSSLS corresponding to amino acids 34-51 of HUMDAF_P26 (SEQ ID NO:54), and a third amino acid sequence being at least 90% homologous to GDCGLPPDVPNAQPALEGRTSFPEDTVITYKCEESFVKIPGEKDSVICLKGSQWSDIEE FCNRSCEVPTRLNSASLKQPYITQNYFPVGTVVEYECRPGYRREPSLSPKLTCLQNLK WSTAVEFCKKKSCPNPGEIRNGQIDVPGGILFGATISFSCNTGYKLFGSTSSFCLISGSS VQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ ATRSTPVSRTTKHFHETTPNKGSGTTSGTTRLLSGHTCFTLTGLLGTLVTMGLLT corresponding to amino acids 34-381 of known protein DAF_HUMAN (SEQ ID NO:42), which also corresponds to amino acids 52-399 of HUMDAF_P26 (SEQ ID NO:54), wherein said first amino acid sequence, second amino acid sequence and third amino acid sequence are contiguous and in a sequential order.

B. An isolated polypeptide encoding for an edge portion of HUMDAF_P26 (SEQ ID NO:54), comprising an amino acid sequence being at least 70%, optionally at least about 80%, preferably at least about 85%, more preferably at least about 90% and most preferably at least about 95% homologous to the sequence ESSRVEHTMLQTCMSSLS of HUMDAF_P26 (SEQ ID NO:54).

2. Comparison report between HUMDAF_P26 (SEQ ID NO:54) and known protein Q8TD13_HUMAN (SEQ ID NO:50) (FIG. 19K):

A. An isolated chimeric polypeptide encoding for HUMDAF_P26 (SEQ ID NO:54), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWESSRVEHTMLQTCMSSLSGDCGLP PDVPNAQPALEGRTSFPEDTVITYKCEESFVKIPGEKDSVICLKGSQWSDIEEFCNRSC EVPTRLNSASLKQPYITQNYFPVGTVVEYEC corresponding to amino acids 1-147 of HUMDAF_P26 (SEQ ID NO:54), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQ corresponding to amino acids 1-197 of known protein(s) Q8TD13_HUMAN (SEQ ID NO:50), which also corresponds to amino acids 148-344 of HUMDAF_P26 (SEQ ID NO:54), and a third amino acid sequence being at least 90% homologous to ATRSTPVSRTTKHFHETTPNKGSGTTSGTTRLLSGHTCFTLTGLLGTLVTMGLLT corresponding to amino acids 368-422 of known protein(s) Q8TD13_HUMAN (SEQ ID NO:50), which also corresponds to amino acids 345-399 of HUMDAF_P26 (SEQ ID NO:54), wherein said first amino acid sequence, second amino acid sequence and third amino acid sequence are contiguous and in a sequential order.

B. An isolated polypeptide encoding for a head of HUMDAF_P26 (SEQ ID NO:54), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWESSRVEHTMLQTCMSSLSGDCGLP PDVPNAQPALEGRTSFPEDTVITYKCEESFVKIPGEKDSVICLKGSQWSDIEEFCNRSC EVPTRLNSASLKQPYITQNYFPVGTVVEYEC of HUMDAF_P26 (SEQ ID NO:54).

C. An isolated chimeric polypeptide encoding for an edge portion of HUMDAF_P26 (SEQ ID NO:54), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QA, having a structure as follows: a sequence starting from any of amino acid numbers 344−x to 344; and ending at any of amino acid numbers 345+((n−2)−x), in which x varies from 0 to n−2.

The localization of the variant protein was determined according to results from a number of different software programs and analyses, including analyses from SignalP and other specialized programs. The variant protein is believed to be located as follows with regard to the cell: membrane.

Variant protein HUMDAF_P26 (SEQ ID NO:54) also has the following non-silent SNPs (Single Nucleotide Polymorphisms) as listed in Table 65, (given according to their position(s) on the amino acid sequence, with the alternative amino acid(s) listed; the last column indicates whether the SNP is known or not; the presence of known SNPs in variant protein HUMDAF_P26 (SEQ ID NO:54) sequence provides support for the deduced sequence of this variant protein according to the present invention).

TABLE 65
Amino acid mutations

| SNP position(s) on | | |
|---|---|---|
| amino acid sequence | Alternative amino acid(s) | Previously known SNP? |
| 11 | A −> | No |
| 177 | K −> T | No |
| 203 | T −> | No |
| 250 | D −> G | No |
| 251 | N −> H | No |

The glycosylation sites of variant protein HUMDAF_P26 (SEQ ID NO:54), as compared to the known protein Complement decay-accelerating factor precursor (SEQ ID NO:42), are described in Table 66 (given according to their position(s) on the amino acid sequence in the first column; the second column indicates whether the glycosylation site is present in the variant protein; and the last column indicates whether the position is different on the variant protein).

TABLE 66
Glycosylation site(s)

| Position(s) on known | Present in variant | Position(s) on variant |
|---|---|---|
| amino acid sequence | protein? | protein |
| 113 | Yes | 113 |

Variant protein HUMDAF_P26 (SEQ ID NO:54) is encoded by the transcript HUMDAF_T24 (SEQ ID NO:38), for which the coding portion starts at position 329 and ends at position 1525. The transcript also has the following SNPs as listed in Table 67 (given according to their position on the nucleotide sequence, with the alternative nucleic acid listed).

TABLE 67
Nucleic acid SNPs

| Polymorphism | SNP position(s) on nucleotide sequence |
|---|---|
| G −> A | 25 |
| A −> C | 130, 858, 1079, 1937, 2209, 2315 |
| A −> G | 130, 1077, 1790 |
| C −> | 277, 308, 937, 2026 |
| −> A | 349 |
| −> T | 349, 1854 |
| G −> | 359 |
| A −> | 935, 1790 |
| T −> | 1805, 1848, 1965 |
| T −> C | 1805 |
| T −> G | 1965 |
| C −> T | 2026 |

Variant protein HUMDAF_P29 (SEQ ID NO:55) according to the present invention has an amino acid sequence encoded by transcript HUMDAF_T30 (SEQ ID NO:39). One or more alignments to one or more previously published protein sequences are shown in FIGS. 19L and 19M. A brief description of the relationship of the variant protein according to the present invention to each such aligned protein is as follows:

1. Comparison report between HUMDAF_P29 (SEQ ID NO:55) and known protein DAF_HUMAN (SEQ ID NO:42) (FIG. 19L):

A. An isolated chimeric polypeptide encoding for HUMDAF_P29 (SEQ ID NO:55), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCN corresponding to amino acids 1-95 of known protein DAF_HUMAN (SEQ ID NO:42), which also corresponds to amino acids 1-95 of HUMDAF_P29 (SEQ ID NO:55), a second bridging amino acid sequence comprising of L, and a third amino acid sequence being at least 90% homologous to GTVVEYECRPGYRREPSLSPKLTCLQNLKWSTAVEFCKKKSCPNPGEIRNGQIDVPGG ILFGATISFSCNTGYKLFGSTSSFCLISGSSVQWSDPLPECREIYCPAPPQIDNGIIQGERD HYGYRQSVTYACNKGFTMIGEHSIYCTVNNDEGEWSGPPPECRGKSLTSKVPPTVQK PTTVNVPTTEVSPTSQKTTTKTTTPNAQATRSTPVSRTTKHFHETTPNKGSGTTSGTTR LLSGHTCFTLTGLLGTLVTMGLLT corresponding to amino acids 122-381 of known protein DAF_HUMAN (SEQ ID NO:42), which also corresponds to amino acids 97-356 of HUMDAF_P29 (SEQ ID NO:55), wherein said first amino acid sequence, second amino acid sequence and third amino acid sequence are contiguous and in a sequential order.

B. An isolated polypeptide encoding for an edge portion of HUMDAF_P29 (SEQ ID NO:55), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise NLG having a structure as follows (numbering according to HUMDAF_P29 (SEQ ID NO:55)): a sequence starting from any of amino acid numbers 95−x to 95; and ending at any of amino acid numbers 97+((n−3)−x), in which x varies from 0 to n−3.

2. Comparison report between HUMDAF_P29 (SEQ ID NO:55) and known proteins Q8TD13_HUMAN (SEQ ID NO:50) (FIG. 19M):

A. An isolated chimeric polypeptide encoding for HUMDAF_P29 (SEQ ID NO:55), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNLGTVVEYEC corresponding to amino acids 1-104 of HUMDAF_P29 (SEQ ID NO:55), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQ corresponding to amino acids 1-197 of known protein(s) Q8TD13_HUMAN (SEQ ID NO:50), which also corresponds to amino acids 105-301 of HUMDAF_P29 (SEQ ID NO:55), and a third amino acid sequence being at least 90% homologous to ATRSTPVSRTTKHFHETTPNKGSGTTSGTTRLLSGHTCFTLTGLLGTLVTMGLLT corresponding to amino acids 368-422 of known protein(s) Q8TD13_HUMAN (SEQ ID NO:50), which also corresponds to amino acids 302-356 of HUMDAF_P29 (SEQ ID NO:55), wherein said first amino acid sequence, second amino acid sequence and third amino acid sequence are contiguous and in a sequential order.

B. An isolated polypeptide encoding for a head of HUMDAF_P29 (SEQ ID NO:55), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNLGTVVEYEC of HUMDAF_P29 (SEQ ID NO:55).

C. An isolated chimeric polypeptide encoding for an edge portion of HUMDAF_P29 (SEQ ID NO:55), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QA, having a structure as follows: a sequence starting from any of amino acid numbers 301−x to 301; and ending at any of amino acid numbers 302+((n−2)−x), in which x varies from 0 to n−2.

The localization of the variant protein was determined according to results from a number of different software programs and analyses, including analyses from SignalP and other specialized programs. The variant protein is believed to be located as follows with regard to the cell: membrane.

Variant protein HUMDAF_P29 (SEQ ID NO:55) also has the following non-silent SNPs (Single Nucleotide Polymorphisms) as listed in Table 68, (given according to their position(s) on the amino acid sequence, with the alternative amino acid(s) listed; the last column indicates whether the SNP is known or not; the presence of known SNPs in variant protein HUMDAF_P29 (SEQ ID NO:55) sequence provides support for the deduced sequence of this variant protein according to the present invention).

TABLE 68
Amino acid mutations

| SNP position(s) on | | |
|---|---|---|
| amino acid sequence | Alternative amino acid(s) | Previously known SNP? |
| 11 | A −> | No |
| 134 | K −> T | No |
| 160 | T −> | No |
| 207 | D −> G | No |
| 208 | N −> H | No |

The glycosylation sites of variant protein HUMDAF_P29 (SEQ ID NO:55), as compared to the known protein Complement decay-accelerating factor precursor (SEQ ID NO:42), are described in Table 69 (given according to their position(s) on the amino acid sequence in the first column; the second column indicates whether the glycosylation site is present in the variant protein; and the last column indicates whether the position is different on the variant protein).

TABLE 69
Glycosylation site(s)

| Position(s) on known | Present in variant | Position(s) on variant |
|---|---|---|
| amino acid sequence | protein? | protein |
| 95 | Yes | 95 |

Variant protein HUMDAF_P29 (SEQ ID NO:55) is encoded by the transcript HUMDAF_T30 (SEQ ID NO:39), for which the coding portion starts at position 329 and ends at position 1396. The transcript also has the following SNPs as listed in Table 70 (given according to their position on the nucleotide sequence, with the alternative nucleic acid listed).

TABLE 70
Nucleic acid SNPs

| Polymorphism | SNP position(s) on nucleotide sequence |
|---|---|
| G −> A | 25 |
| A −> C | 130, 729, 950, 1808, 2080, 2186 |
| A −> G | 130, 948, 1661 |
| C −> | 277, 308, 808, 1897 |
| −> A | 349 |
| −> T | 349, 1725 |
| G −> | 359 |
| A −> | 806, 1661 |
| T −> | 1676, 1719, 1836 |
| T −> C | 1676 |
| T −> G | 1836 |
| C −> T | 1897 |

Variant protein HUMDAF_P30 (SEQ ID NO:56) according to the present invention has an amino acid sequence encoded by transcript HUMDAF_T31 (SEQ ID NO:40). One or more alignments to one or more previously published protein sequences are shown in FIGS. 19N, 19) and 19P. A brief description of the relationship of the variant protein according to the present invention to each such aligned protein is as follows:

1. Comparison report between HUMDAF_P30 (SEQ ID NO:56) and known protein DAF_HUMAN (SEQ ID NO:42) (FIG. 19N):

A. An isolated chimeric polypeptide encoding for HUMDAF_P30 (SEQ ID NO:56), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGSSVQWSDPLPECREIYCPAPPQIDNGIIQG ERDHYGYRQSVTYACNKGFTMIGEHSIYCTVNNDEGEWSGPPPECRGKSLTSKVPPT VQKPTTVNVPTTEVSPTSQKTTTKTTTPNAQ corresponding to amino acids 1-326 of known protein DAF_HUMAN (SEQ ID NO:42), which also corresponds to amino acids 1-326 of HUMDAF_P30 (SEQ ID NO:56), a second amino acid sequence being at least 70%, optionally at least 80%, preferably at least 85%, more preferably at least 90% and most preferably at least 95% homologous to a polypeptide having the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP corresponding to amino acids 327-471 of HUMDAF_P30 (SEQ ID NO:56), a third amino acid sequence being at least 90% homologous to TRSTPVSRTTKHFHETTPNKGSGTTSGTTRLLSG corresponding to amino acids 328-361 of known protein DAF_HUMAN (SEQ ID NO:42), which also corresponds to amino acids 472-505 of HUMDAF_P30 (SEQ ID NO:56), and a fourth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRCVPRHPAKFLKFIFCRDRI FLCCPGWFQTPGRKRFFRPPKTLRL corresponding to amino acids 506-588 of HUMDAF_P30 (SEQ ID NO:56), wherein said first amino acid sequence, second amino acid sequence, third amino acid sequence and fourth amino acid sequence are contiguous and in a sequential order.

B. An isolated polypeptide encoding for an edge portion of HUMDAF_P30 (SEQ ID NO:56), comprising an amino acid sequence being at least 70%, optionally at least about 80%, preferably at least about 85%, more preferably at least about 90% and most preferably at least about 95% homologous to the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAP of HUMDAF_P30 (SEQ ID NO:56).

C. An isolated polypeptide encoding for an edge portion of HUMDAF_P30 (SEQ ID NO:56), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRCVPRHPAKFLKFIFCRDRI FLCCPGWFQTPGRKRFFRPPKTLRL of HUMDAF_P30 (SEQ ID NO:56).

2. Comparison report between HUMDAF_P30 (SEQ ID NO:56) and known protein Q8TD13_HUMAN (SEQ ID NO:50) (FIG. 19O):

A. An isolated chimeric polypeptide encoding for HUMDAF_P30 (SEQ ID NO:56), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC corresponding to amino acids 1-129 of HUMDAF_P30 (SEQ ID NO:56), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQG corresponding to amino acids 1-198 of known protein(s) Q8TD13_HUMAN (SEQ ID NO:50), which also corresponds to amino acids 130-327 of HUMDAF_P30 (SEQ ID NO:56), a bridging amino acid T corresponding to amino acid 328 of HUMDAF_P30 (SEQ ID NO:56), a third amino acid sequence being at least 90% homologous to ETPSVLQKHTTENVSATRTPPTPQKPTTVNVPATIVTPTPQKPTTINVPATGVSSTPQR HTIVNVSATGTLPTLQKPTRANDSATKSPAAAQTSFISKTLSTKTPSAAQNPMMTNAS ATQATLTAQRFTTAKVAFTQSPSAA corresponding to amino acids 200-341 of known protein(s) Q8TD13_HUMAN (SEQ ID NO:50), which also corresponds to amino acids 329-470 of HUMDAF_P30 (SEQ ID NO:56), a fourth bridging amino acid sequence comprising of P, a fifth amino acid sequence being at least 90% homologous to TRSTPVSRTTKHFHETTPNKGSGTTSGTTRLLSG corresponding to amino acids 369-402 of known protein(s) Q8TD13_HUMAN (SEQ ID NO:50), which also corresponds to amino acids 472-505 of HUMDAF_P30 (SEQ ID NO:56), and a sixth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRCVPRHPAKFLKFIFCRDRI FLCCPGWFQTPGRKRFFRPPKTLRL corresponding to amino acids 506-588 of HUMDAF_P30 (SEQ ID NO:56), wherein said first amino acid sequence, second amino acid sequence, bridging amino acid, third amino acid sequence, fourth amino acid sequence, fifth amino acid sequence and sixth amino acid sequence are contiguous and in a sequential order.

B. An isolated polypeptide encoding for a head of HUMDAF_P30 (SEQ ID NO:56), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC of HUMDAF_P30 (SEQ ID NO:56).

C. An isolated polypeptide encoding for an edge portion of HUMDAF_P30 (SEQ ID NO:56), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise APT having a structure as follows (numbering according to HUMDAF_P30 (SEQ ID NO:56)): a sequence starting from any of amino acid numbers 470−x to 470; and ending at any of amino acid numbers 472+((n−3)−x), in which x varies from 0 to n−3.

D. An isolated polypeptide encoding for an edge portion of HUMDAF_P30 (SEQ ID NO:56), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRCVPRHPAKFLKFIFCRDRI FLCCPGWFQTPGRKRFFRPPKTLRL of HUMDAF_P30 (SEQ ID NO:56).

3. Comparison report between HUMDAF_P30 (SEQ ID NO:56) and known protein Q8TD14 HUMAN (SEQ ID NO:48) (FIG. 19P):

A. An isolated chimeric polypeptide encoding for HUMDAF_P30 (SEQ ID NO:56), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS corresponding to amino acids 1-209 of HUMDAF_P30 (SEQ ID NO:56), a second amino acid sequence being at least 90% homologous to SVQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQ corresponding to amino acids 1-245 of known protein(s) Q8TD14 HUMAN (SEQ ID NO:48), which also corresponds to amino acids 210-454 of HUMDAF_P30 (SEQ ID NO:56), a bridging amino acid R corresponding to amino acid 455 of HUMDAF_P30 (SEQ ID NO:56), a third amino acid sequence being at least 90% homologous to FTTAKVAFTQSPSAAPTRSTPVSRTTKHFHETTPNKGSGTTSGTTRLLSG corresponding to amino acids 247-296 of known protein(s) Q8TD14 HUMAN (SEQ ID NO:48), which also corresponds to amino acids 456-505 of HUMDAF_P30 (SEQ ID NO:56), and a fourth amino acid sequence being at least 70%, optionally at least 80%, preferably at least 85%, more preferably at least 90% and most preferably at least 95% homologous to a polypeptide having the sequence SRPVTQAGMRCDRSSLQSRTPGFKRSFHFSLPSSWYYRCVPRHPAKFLKFIFCRDRI FLCCPGWFQTPGRKRFFRPPKTLRL corresponding to amino acids 506-588 of HUMDAF_P30 (SEQ ID NO:56), wherein said first amino acid sequence, second amino acid sequence, bridging amino acid, third amino acid sequence and fourth amino acid sequence are contiguous and in a sequential order.

B. An isolated polypeptide encoding for a head of HUMDAF_P30 (SEQ ID NO:56), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS of HUMDAF_P30 (SEQ ID NO:56).

C. An isolated polypeptide encoding for an edge portion of HUMDAF_P30 (SEQ ID NO:56), comprising an amino acid sequence being at least 70%, optionally at least about 80%, preferably at least about 85%, more preferably at least about 90% and most preferably at least about 95% homologous to the sequence SRPVTQAGMRCDRSSLQSRTPGFKRSFHFSLPSSWYYRCVPRHPAKFLKFIFCRDRI FLCCPGWFQTPGRKRFFRPPKTLRL of HUMDAF_P30 (SEQ ID NO:56).

The localization of the variant protein was determined according to results from a number of different software programs and analyses, including analyses from SignalP and other specialized programs. The variant protein is believed to be located as follows with regard to the cell: membrane.

Variant protein HUMDAF_P30 (SEQ ID NO:56) also has the following non-silent SNPs (Single Nucleotide Polymorphisms) as listed in Table 71, (given according to their position(s) on the amino acid sequence, with the alternative amino acid(s) listed; the last column indicates whether the SNP is known or not; the presence of known SNPs in variant protein HUMDAF_P30 (SEQ ID NO:56) sequence provides support for the deduced sequence of this variant protein according to the present invention).

TABLE 71
Amino acid mutations

| SNP position(s) on | | |
|---|---|---|
| amino acid sequence | Alternative amino acid(s) | Previously known SNP? |
| 11 | A −> | No |
| 159 | K −> T | No |
| 185 | T −> | No |
| 232 | D −> G | No |
| 233 | N −> H | No |
| 328 | T −> A | No |
| 369 | Q −> * | No |
| 455 | R −> K | No |

The glycosylation sites of variant protein HUMDAF_P30 (SEQ ID NO:56), as compared to the known protein Complement decay-accelerating factor precursor (SEQ ID NO:42), are described in Table 72 (given according to their position(s) on the amino acid sequence in the first column; the second column indicates whether the glycosylation site is present in the variant protein; and the last column indicates whether the position is different on the variant protein).

TABLE 72
Glycosylation site(s)

| Position(s) on known | Present in variant | Position(s) on variant |
|---|---|---|
| amino acid sequence | protein? | protein |
| 95 | Yes | 95 |

Variant protein HUMDAF_P30 (SEQ ID NO:56) is encoded by the transcript HUMDAF_T31 (SEQ ID NO:40), for which the coding portion starts at position 329 and ends at position 2092. The transcript also has the following SNPs as listed in Table 73 (given according to their position on the nucleotide sequence, with the alternative nucleic acid listed).

TABLE 73
Nucleic acid SNPs

| Polymorphism | SNP position(s) on nucleotide sequence |
|---|---|
| G −> A | 25, 1692 |
| A −> C | 130, 804, 1025 |
| A −> G | 130, 1023, 1310, 1426 |
| C −> | 277, 308, 883 |
| −> A | 349 |
| −> T | 349 |
| G −> | 359 |
| A −> | 881 |
| C −> T | 1433 |

Variant protein HUMDAF_P31 (SEQ ID NO:57) according to the present invention has an amino acid sequence is encoded by transcript HUMDAF_T32 (SEQ ID NO:41). One or more alignments to one or more previously published protein sequences are shown in FIGS. 19Q, 19R and 19S. A brief description of the relationship of the variant protein according to the present invention to each such aligned protein is as follows:

1. Comparison report between HUMDAF_P31 (SEQ ID NO:57) and known protein DAF_HUMAN (SEQ ID NO:42) (FIG. 19Q):

A. An isolated chimeric polypeptide encoding for HUMDAF_P31 (SEQ ID NO:57), comprising a first amino acid sequence being at least 90% homologous to MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGSSVQWSDPLPECREIYCPAPPQIDNGIIQG ERDHYGYRQSVTYACNKGFTMIGEHSIYCTVNNDEGEWSGPPPECRGKSLTSKVPPT VQKPTTVNVPTTEVSPTSQKTTTKTTTPNAQ corresponding to amino acids 1-326 of known protein DAF_HUMAN (SEQ ID NO:42), which also corresponds to amino acids 1-326 of HUMDAF_P31 (SEQ ID NO:57), a second amino acid sequence being at least 70%, optionally at least 80%, preferably at least 85%, more preferably at least 90% and most preferably at least 95% homologous to a polypeptide having the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAHKSTNVHSPVTNGLKSTQRFPSAHIT corresponding to amino acids 327-496 of HUMDAF_P31 (SEQ ID NO:57), a third amino acid sequence being at least 90% homologous to ATRSTPVSRTTKHFHETTPNKGSGTTSGTTRLLS corresponding to amino acids 327-360 of known protein DAF_HUMAN (SEQ ID NO:42), which also corresponds to amino acids 497-530 of HUMDAF_P31 (SEQ ID NO:57), and a fourth amino acid sequence being at least 70%, optionally at least 80%, preferably at least 85%, more preferably at least 90% and most preferably at least 95% homologous to a polypeptide having the sequence ALIMHMRATKYSMLCLTI corresponding to amino acids 531-548 of HUMDAF_P31 (SEQ ID NO:57), wherein said first amino acid sequence, second amino acid sequence, third amino acid sequence and fourth amino acid sequence are contiguous and in a sequential order.

B. An isolated polypeptide encoding for an edge portion of HUMDAF_P31 (SEQ ID NO:57), comprising an amino acid sequence being at least 70%, optionally at least about 80%, preferably at least about 85%, more preferably at least about 90% and most preferably at least about 95% homologous to the sequence GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQRFTTAKVAFTQSPSAAHKSTNVHSPVTNGLKSTQRFPSAHIT of HUMDAF_P31 (SEQ ID NO:57).

C. An isolated polypeptide encoding for an edge portion of HUMDAF_P31 (SEQ ID NO:57), comprising an amino acid sequence being at least 70%, optionally at least about 80%, preferably at least about 85%, more preferably at least about 90% and most preferably at least about 95% homologous to the sequence ALIMHMRATKYSMLCLTI of HUMDAF_P31 (SEQ ID NO:57).

2. Comparison report between HUMDAF_P31 (SEQ ID NO:57) and known protein Q8TD13_HUMAN (SEQ ID NO:50) (FIG. 19R):

A. An isolated chimeric polypeptide encoding for HUMDAF_P31 (SEQ ID NO:57), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC corresponding to amino acids 1-129 of HUMDAF_P31 (SEQ ID NO:57), a second amino acid sequence being at least 90% homologous to RPGYRREPSLSPKLTCLQNLKWSTAVEFCKKKSCPNPGEIRNGQIDVPGGILFGATISF SCNTGYKLFGSTSSFCLISGSSVQWSDPLPECREIYCPAPPQIDNGIIQGERDHYGYRQS VTYACNKGFTMIGEHSIYCTVNNDEGEWSGPPPECRGKSLTSKVPPTVQKPTTVNVPT TEVSPTSQKTTTKTTTPNAQG corresponding to amino acids 1-198 of known protein(s) Q8TD13_HUMAN (SEQ ID NO:50), which also corresponds to amino acids 130-327 of HUMDAF_P31 (SEQ ID NO:57), a bridging amino acid T corresponding to amino acid 328 of HUMDAF_P31 (SEQ ID NO:57), a third amino acid sequence being at least 90% homologous to ETPSVLQKHTTENVSATRTPPTPQKPTTVNVPATIVTPTPQKPTTINVPATGVSSTPQR HTIVNVSATGTLPTLQKPTRANDSATKSPAAAQTSFISKTLSTKTPSAAQNPMMTNAS ATQATLTAQRFTTAKVAFTQSPSAAHKSTNVHSPVTNGLKSTQRFPSAHITATRSTPV SRTTKHFHETTPNKGSGTTSGTTRLLS corresponding to amino acids 200-401 of known protein(s) Q8TD13_HUMAN (SEQ ID NO:50), which also corresponds to amino acids 329-530 of HUMDAF_P31 (SEQ ID NO:57), and a fourth amino acid sequence being at least 70%, optionally at least 80%, preferably at least 85%, more preferably at least 90% and most preferably at least 95% homologous to a polypeptide having the sequence ALIMHMRATKYSMLCLTI corresponding to amino acids 531-548 of HUMDAF_P31 (SEQ ID NO:57), wherein said first amino acid sequence, second amino acid sequence, bridging amino acid, third amino acid sequence and fourth amino acid sequence are contiguous and in a sequential order.

B. An isolated polypeptide encoding for a head of HUMDAF_P31 (SEQ ID NO:57), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYEC of HUMDAF_P31 (SEQ ID NO:57).

C. An isolated polypeptide encoding for an edge portion of HUMDAF_P31 (SEQ ID NO:57), comprising an amino acid sequence being at least 70%, optionally at least about 80%, preferably at least about 85%, more preferably at least about 90% and most preferably at least about 95% homologous to the sequence ALIMHMRATKYSMLCLTI of HUMDAF_P31 (SEQ ID NO:57).

3. Comparison report between HUMDAF_P31 (SEQ ID NO:57) and known protein Q8TD14 HUMAN (SEQ ID NO:48) (FIG. 19S):

A. An isolated chimeric polypeptide encoding for HUMDAF_P31 (SEQ ID NO:57), comprising a first amino acid sequence being at least 70%, optionally at least 80%, preferably at least 85%, more preferably at least 90% and most preferably at least 95%, homologous to a polypeptide having the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS corresponding to amino acids 1-209 of HUMDAF_P31 (SEQ ID NO:57), a second amino acid sequence being at least 90% homologous to SVQWSDPLPECREIYCPAPPQIDNGIIQGERDHYGYRQSVTYACNKGFTMIGEHSIYCT VNNDEGEWSGPPPECRGKSLTSKVPPTVQKPTTVNVPTTEVSPTSQKTTTKTTTPNAQ GTETPSVLQKHTTENVSATRTPPTPQKPTTVNVPATIVTPTPQKPTTINVPATGVSSTP QRHTIVNVSATGTLPTLQKPTRANDSATKSPAAAQTSFISKTLSTKTPSAAQNPMMTN ASATQATLTAQ corresponding to amino acids 1-245 of known protein(s) Q8TD14 HUMAN (SEQ ID NO:48), which also corresponds to amino acids 210-454 of HUMDAF_P31 (SEQ ID NO:57), a bridging amino acid R corresponding to amino acid 455 of HUMDAF_P31 (SEQ ID NO:57), a third amino acid sequence being at least 90% homologous to FTTAKVAFTQSPSAA corresponding to amino acids 247-261 of known protein(s) Q8TD14 HUMAN (SEQ ID NO:48), which also corresponds to amino acids 456-470 of HUMDAF_P31 (SEQ ID NO:57), a fourth amino acid sequence being at least 70%, optionally at least 80%, preferably at least 85%, more preferably at least 90% and most preferably at least 95% homologous to a polypeptide having the sequence HKSTNVHSPVTNGLKSTQRFPSAHITA corresponding to amino acids 471-497 of HUMDAF_P31 (SEQ ID NO:57), a fifth amino acid sequence being at least 90% homologous to TRSTPVSRTTKHFHETTPNKGSGTTSGTTRLLS corresponding to amino acids 263-295 of known protein(s) Q8TD14 HUMAN (SEQ ID NO:48), which also corresponds to amino acids 498-530 of HUMDAF_P31 (SEQ ID NO:57), and a sixth amino acid sequence being at least 70%, optionally at least 80%, preferably at least 85%, more preferably at least 90% and most preferably at least 95% homologous to a polypeptide having the sequence ALIMHMRATKYSMLCLTI corresponding to amino acids 531-548 of HUMDAF_P31 (SEQ ID NO:57), wherein said first amino acid sequence, second amino acid sequence, bridging amino acid, third amino acid sequence, fourth amino acid sequence, fifth amino acid sequence and sixth amino acid sequence are contiguous and in a sequential order.

B. An isolated polypeptide encoding for a head of HUMDAF_P31 (SEQ ID NO:57), comprising a polypeptide being at least 70%, optionally at least about 80%, preferably at least about 85%, more preferably at least about 90% and most preferably at least about 95% homologous to the sequence MTVARPSVPAALPLLGELPRLLLLVLLCLPAVWGDCGLPPDVPNAQPALEGRTSFPED TVITYKCEESFVKIPGEKDSVICLKGSQWSDIEEFCNRSCEVPTRLNSASLKQPYITQNY FPVGTVVEYECRPGYRREPSLSPKLTCLQNLKWSTAVEFCKKKSCPNPGEIRNGQIDV PGGILFGATISFSCNTGYKLFGSTSSFCLISGS of HUMDAF_P31 (SEQ ID NO:57).

C. An isolated polypeptide encoding for an edge portion of HUMDAF_P31 (SEQ ID NO:57), comprising an amino acid sequence being at least 70%, optionally at least about 80%, preferably at least about 85%, more preferably at least about 90% and most preferably at least about 95% homologous to the sequence HKSTNVHSPVTNGLKSTQRFPSAHITA of HUMDAF_P31 (SEQ ID NO:57).

D. An isolated polypeptide encoding for an edge portion of HUMDAF_P31 (SEQ ID NO:57), comprising an amino acid sequence being at least 70%, optionally at least about 80%, preferably at least about 85%, more preferably at least about 90% and most preferably at least about 95% homologous to the sequence ALIMHMRATKYSMLCLTI of HUMDAF_P31 (SEQ ID NO:57).

The localization of the variant protein was determined according to results from a number of different software programs and analyses, including analyses from SignalP and other specialized programs. The variant protein is believed to be located as follows with regard to the cell: membrane.

Variant protein HUMDAF_P31 (SEQ ID NO:57) also has the following non-silent SNPs (Single Nucleotide Polymorphisms) as listed in Table 74, (given according to their position(s) on the amino acid sequence, with the alternative amino acid(s) listed; the last column indicates whether the SNP is known or not; the presence of known SNPs in variant protein HUMDAF_P31 (SEQ ID NO:57) sequence provides support for the deduced sequence of this variant protein according to the present invention).

TABLE 74
Amino acid mutations

| SNP position(s) on | | |
|---|---|---|
| amino acid sequence | Alternative amino acid(s) | Previously known SNP? |
| 11 | A −> | No |
| 159 | K −> T | No |
| 185 | T −> | No |
| 232 | D −> G | No |
| 233 | N −> H | No |
| 328 | T −> A | No |
| 369 | Q −> * | No |
| 455 | R −> K | No |
| 471 | H −> R | No |

The glycosylation sites of variant protein HUMDAF_P31 (SEQ ID NO:57), as compared to the known protein Complement decay-accelerating factor precursor (SEQ ID NO:42), are described in Table 75 (given according to their position(s) on the amino acid sequence in the first column; the second column indicates whether the glycosylation site is present in the variant protein; and the last column indicates whether the position is different on the variant protein).

TABLE 75
Glycosylation site(s)

| Position(s) on known | Present in variant | Position(s) on variant |
|---|---|---|
| amino acid sequence | protein? | protein |
| 95 | Yes | 95 |

Variant protein HUMDAF_P31 (SEQ ID NO:57) is encoded by the transcript HUMDAF_T32 (SEQ ID NO:41), for which the coding portion starts at position 329 and ends at position 1972. The transcript also has the following SNPs as listed in Table 76 (given according to their position on the nucleotide sequence, with the alternative nucleic acid listed).

TABLE 76
Nucleic acid SNPs

| Polymorphism | SNP position(s) on nucleotide sequence |
|---|---|
| G −> A | 25, 1692 |
| A −> C | 130, 804, 1025 |
| A −> G | 130, 1023, 1310, 1426, 1740 |
| C −> | 277, 308, 883 |
| −> A | 349 |
| −> T | 349 |
| G −> | 359 |
| A −> | 881 |
| C −> T | 1433 |

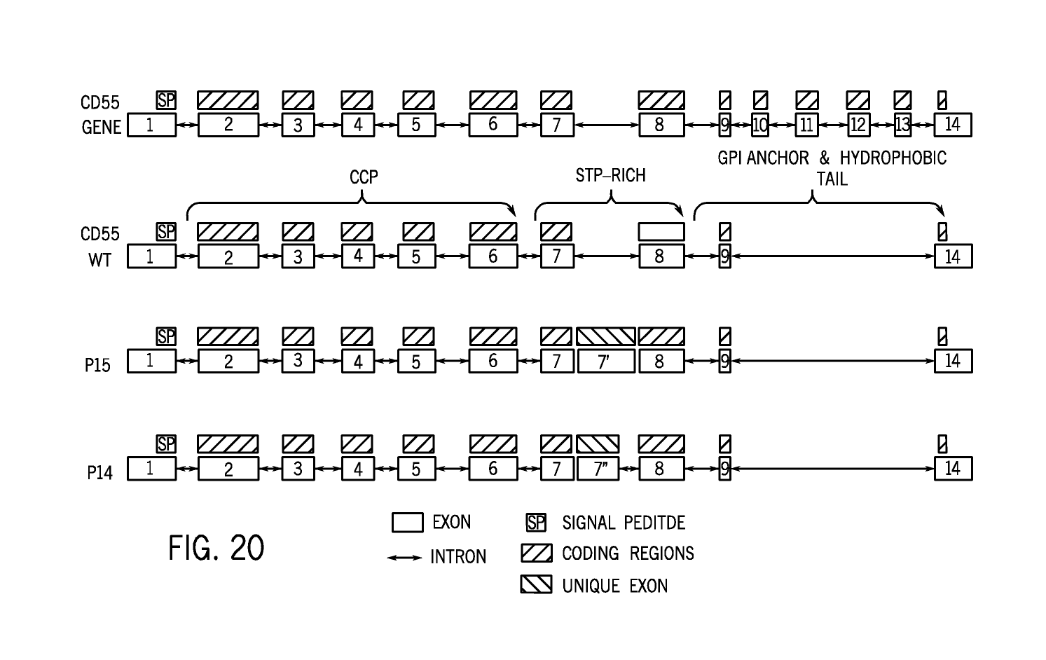

FIG. 20 presents schematic drawing representing CD55 gene structure. The figure presents the exon/intron structure of the wild type CD55 as compared to HUMDAF_P14 (SEQ ID NO:51) (P14) and HUMDAF_P15 (SEQ ID NO:52) (P15) variants. The exons are presented as white rectangles, and appear in numerical order from 1 to 14; the introns are shown as double headed arrays. The signal peptide is marked as SP. The coding sequences are shown as grey rectangles. The unique sequences are indicated accordingly. The GPI anchor and hydrophobic tail are indicated accordingly. CCP stands for complement control protein repeats STP-rich stands for serine-threonine-proline-rich region.

It is expect that P14 and P15 will be powerful molecules executing even higher activity than the wild type CD55, these are highly glycosylated and contain an elongated STP-rich domain These conclusions stem from previous findings showing that high glycosylation of DAF is required for full activity and that a longer STP-rich domain is expected to have higher inhibitory activity of complement.

### Example 4_{—}2

### Expression Analysis of CD55 Transcripts Expression of CD55 Cluster Using MED Discovery Engine

MED discovery engine described in Example 1 herein, was used to assess the expression of HUMDAF transcripts. Expression data for Affymetrix probe sets 201925 s at and 201926_s_at representing CD55 family data is shown in FIGS. 21 and 22. As evident from the scatter plot, presented in FIG. 21, the expression of CD55 transcripts detectable with the above probe sets was higher in liver cancer compared to normal liver samples. As evident from the scatter plot, presented in FIG. 22, the expression of CD55 transcripts detectable with the above probe sets was higher in pancreatic cancer compared to normal pancreas samples. For each group, the median expression is represented by a marker, and the expression values of the different chips in the group are represented by small dashes (“-”). The groups are ordered and marked as follows—“Other” groups (e.g. benign, non-cancer diseases, etc.) with an “x”, Treated cells with a square, Normal with a circle, Matched with a “+”, and Cancer with a diamond.

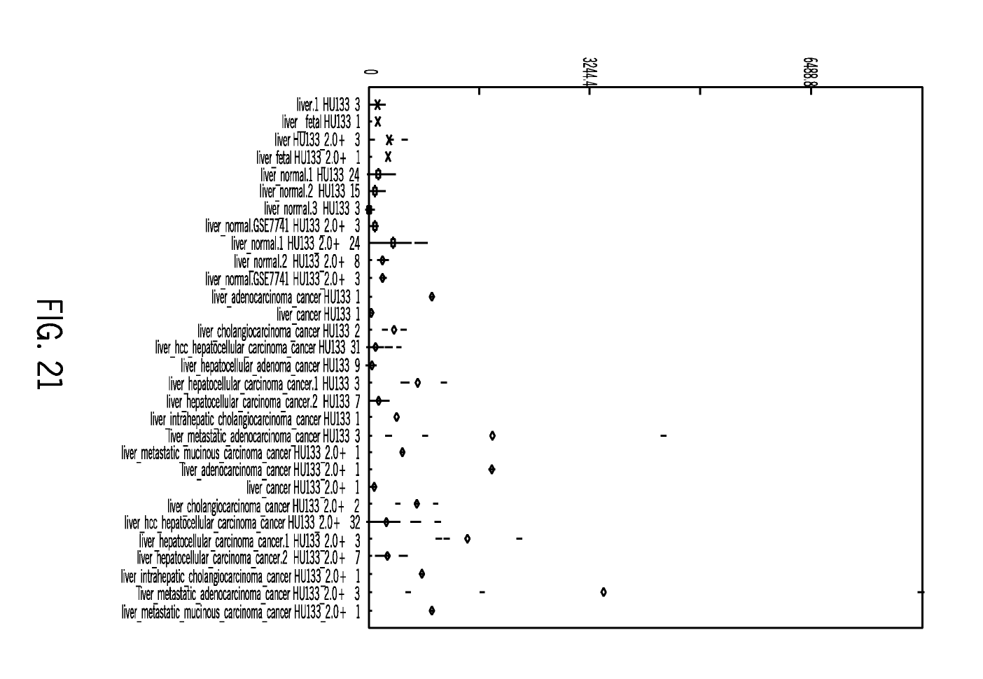

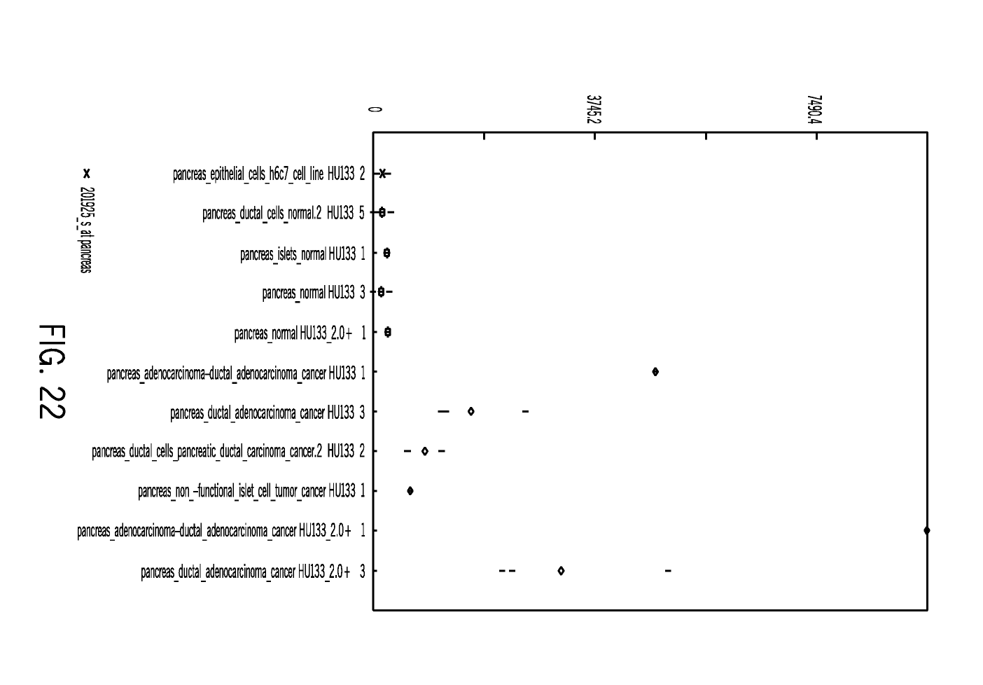

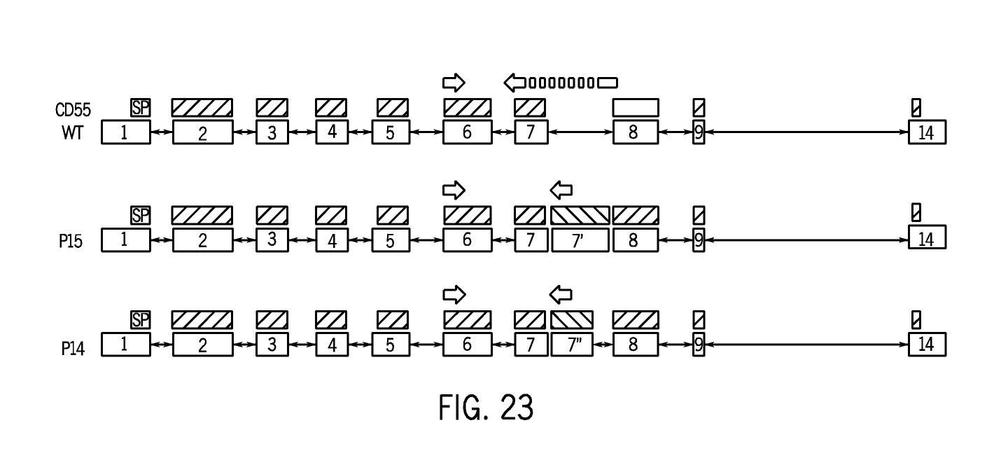

Amplicons specific for CD55 intron 7 retention (variants HUMDAF_P14 (SEQ ID NO:51), HUMDAF_P15 (SEQ ID NO:52) and others) and amplicons specific for wild type CD55 transcripts were used in qRT-PCR analysis on colon panel, containing cancer samples from various stages and from normal colon; healthy panel, containing various normal tissues; and blood panel containing primary immune cells, lymphomas and cell lines. FIG. 23 presents schematic drawing of the primers used for the amplification of the above mentioned specific amplicons. In FIG. 23 the exons are presented as white rectangles, and appear in numerical order from 1 to 14; the introns are shown as double headed arrays. The signal peptide is marked as SP. The coding sequences are shown as grey rectangles. The unique sequences are indicated accordingly. The primers are shown by arrays. For CD55 wt amplicons were designed for the 6^{th} exon and for the junction of the 7^{th} and 8^{th} exon. For the CD55 splice variants, amplicons were designed for the 6^{th} exon and for the intron 7 retention.

### Expression of DAF HUMDAF Transcripts which are Detectable by Amplicon as Depicted in Sequence name HUMDAF_seg24-28F1R1 (SEQ ID NO:90) in Normal and Cancerous Colon Tissues

Expression of DAF transcripts detectable by or according to HUMDAF_seg24-28F1R1 amplicon (SEQ ID NO:90) and primers HUMDAF_seg24-28F1 (SEQ ID NO:88) and HUMDAF_seg24-28R1 (SEQ ID NO:89) (which measure the 6^{th} exon and the intron 7 retention) was measured by real time PCR on colon panel. The samples used are detailed in Table 6 above. For each RT sample, the expression of the above amplicon was normalized to the normalization factor calculated from the expression of these house keeping genes as described in “Materials and Experimental Procedures” herein. The normalized quantity of each RT sample was then divided by the median of the quantities of the normal samples (sample numbers 42-70, Table 6 above), to obtain a value of fold up-regulation for each sample relative to median of the normal samples.

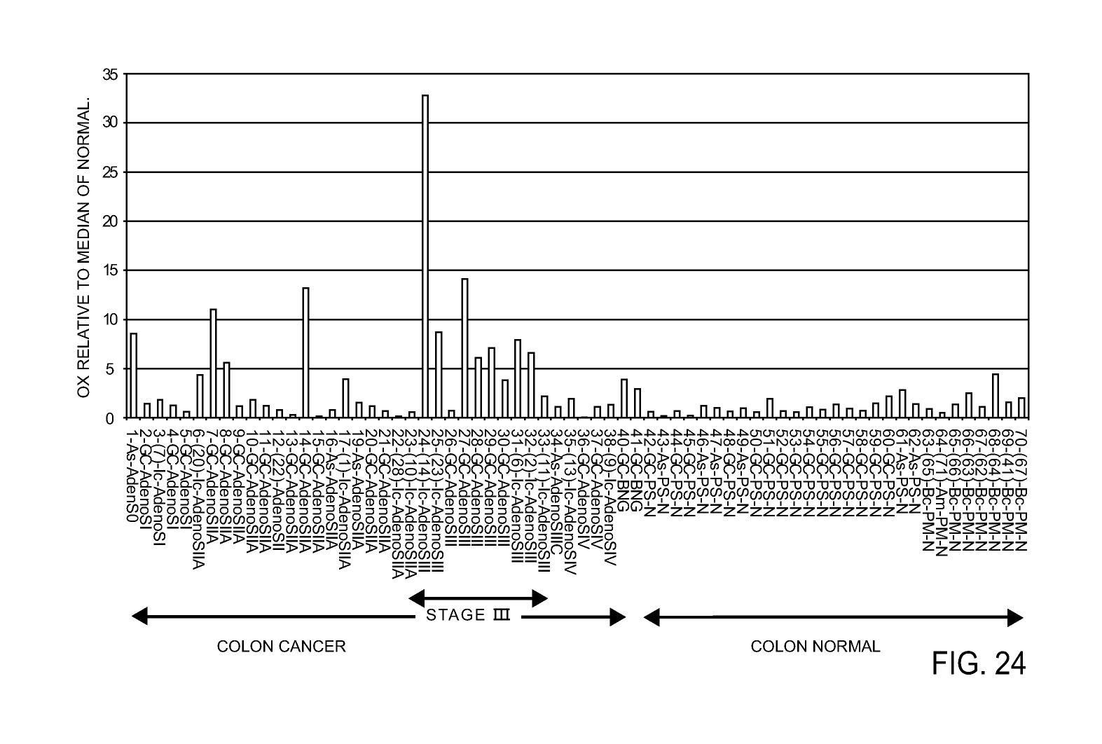

FIG. 24 is a histogram showing over expression of the above-indicated DAF transcripts in cancerous Colon samples relative to the normal samples.

As is evident from FIG. 24, the expression of DAF transcripts detectable by the above amplicon in cancer samples was significantly higher than in the non-cancerous samples (sample numbers 42-70, Table 6 above). Notably an over-expression of at least 5 fold was found in 11 out of 37 adenocarcinoma samples. As is evident from FIG. 24, CD55 variant transcripts detectable by the HUMDAF_seg24-28F1R1 amplicon (SEQ ID NO:90) showed a significant overexpression in stage III colon cancer (>5 fold increase in 7 samples out of 11 colon cancer stage III samples).

Statistical analysis was applied to verify the significance of these results, as described below.

The P value for the difference in the expression levels of DAF transcripts detectable by the above amplicon in Colon cancer samples versus the normal tissue samples was determined by T test as 2.77e-003.

Threshold of 5 fold over expression was found to differentiate between cancer and normal samples with P value of 7.96e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMDAF_seg24-28F1 forward primer (SEQ ID NO:88); and HUMDAF_seg24-28R1 reverse primer (SEQ ID NO:89).

The present invention also preferably encompasses any amplicon obtained through the use of any suitable primer pair; for example, for the above experiment, the following amplicon was obtained as a non-limiting illustrative example only of a suitable amplicon:

### HUMDAF_seg24-28F1R1 (SEQ ID NO:90).

(SEQ ID NO: 88)

Forward primer: >seg24-28F1: GGCCCACCACCTGAATGCAG

(SEQ ID NO: 89)

Reverse primer: >seg24-28R1: TTTCTGAGGAGTTGGTGGGGT
TCTTG
Amplicon: seg24-28F1R1

(SEQ ID NO: 90)

GGCCCACCACCTGAATGCAGAGGAAAATCTCTAACTTCCAAGGTCCCACC
AACAGTTCAGAAACCTACCACAGTAAATGTTCCAACTACAGAAGTCTCAC
CAACTTCTCAGAAAACCACCACAAAAACCACCACACCAAATGCTCAAGGT
ACAGAGACTCCATCAGTTCTTCAAAAACACACCACAGAAAATGTTTCAGC
TACAAGAACCCCACCAACTCCTCAGAAA

Expression of Wild Type DAF HUMDAF Transcripts which are Detectable by Amplicon as Depicted in Sequence Name HUMDAF_seg24junc27-30F2R2 (SEQ ID NO:92) in Normal and Cancerous Colon Tissues

Expression of DAF transcripts detectable by or according to HUMDAF_seg24junc27-30F2R2 amplicon (SEQ ID NO:92) and primers HUMDAF_seg24junc27-30F2 (SEQ ID NO:62) and HUMDAF_seg24junc27-30R2 (SEQ ID NO:91) was measured by real time PCR (which measure the 6^{th} exon and the junction of the 7^{th} and 8^{th} exon) on colon panel. The samples used are detailed in Table 6. For each RT sample, the expression of the above amplicon was normalized to the normalization factor calculated from the expression of these house keeping genes as described in “Materials and Experimental Procedures” herein. The normalized quantity of each RT sample was then divided by the median of the quantities of the normal samples (sample numbers 42-70, Table 6), to obtain a value of fold up-regulation for each sample relative to median of the normal samples.

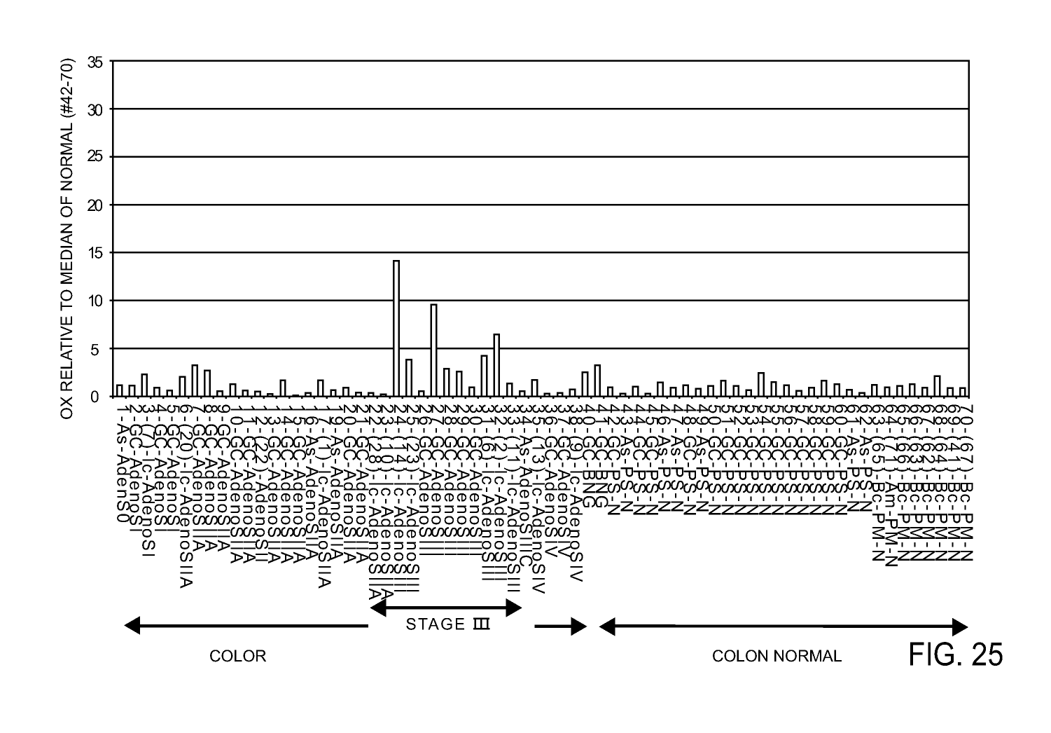

FIG. 25 is a histogram showing over expression of the above-indicated DAF transcripts in cancerous Colon samples relative to the normal samples.

As is evident from FIG. 25, the expression of DAF transcripts detectable by the above amplicon in cancer samples was higher than in the non-cancerous samples (sample numbers 42-70, Table 4 above). Notably an over-expression of at least 5 fold was found in 3 out of 37 adenocarcinoma samples.

Statistical analysis was applied to verify the significance of these results, as described below.

The P value for the difference in the expression levels of DAF transcripts detectable by the above amplicon in Colon cancer samples versus the normal tissue samples was determined by T test as 2.84e-002.

The above values demonstrate statistical significance of the results.

Primer pairs are also optionally and preferably encompassed within the present invention; for example, for the above experiment, the following primer pair was used as a non-limiting illustrative example only of a suitable primer pair: HUMDAF_seg24junc27-30F2 forward primer (SEQ ID NO:62); and HUMDAF_seg24junc27-30R2 reverse primer (SEQ ID NO:91).

The present invention also preferably encompasses any amplicon obtained through the use of any suitable primer pair; for example, for the above experiment, the following amplicon was obtained as a non-limiting illustrative example only of a suitable amplicon:

HUMDAF_seg24junc27-30F2R2 (SEQ ID NO:92).

(SEQ ID NO: 62)

Forward primer: > seg24junc27-30F2: AGTGGCCCACCACC
TGAATG

(SEQ ID NO: 91)

Reverse primer: > seg24junc27-30R2: AGGTGTACTCCGTG
TTGCTTGAG
Amplicon: seg24junc27-30F2R2

(SEQ ID NO: 92)

AGTGGCCCACCACCTGAATGCAGAGGAAAATCTCTAACTTCCAAGGTCCC
ACCAACAGTTCAGAAACCTACCACAGTAAATGTTCCAACTACAGAAGTCT
CACCAACTTCTCAGAAAACCACCACAAAAACCACCACACCAAATGCTCA
AGCAACACGGAGTACACCT

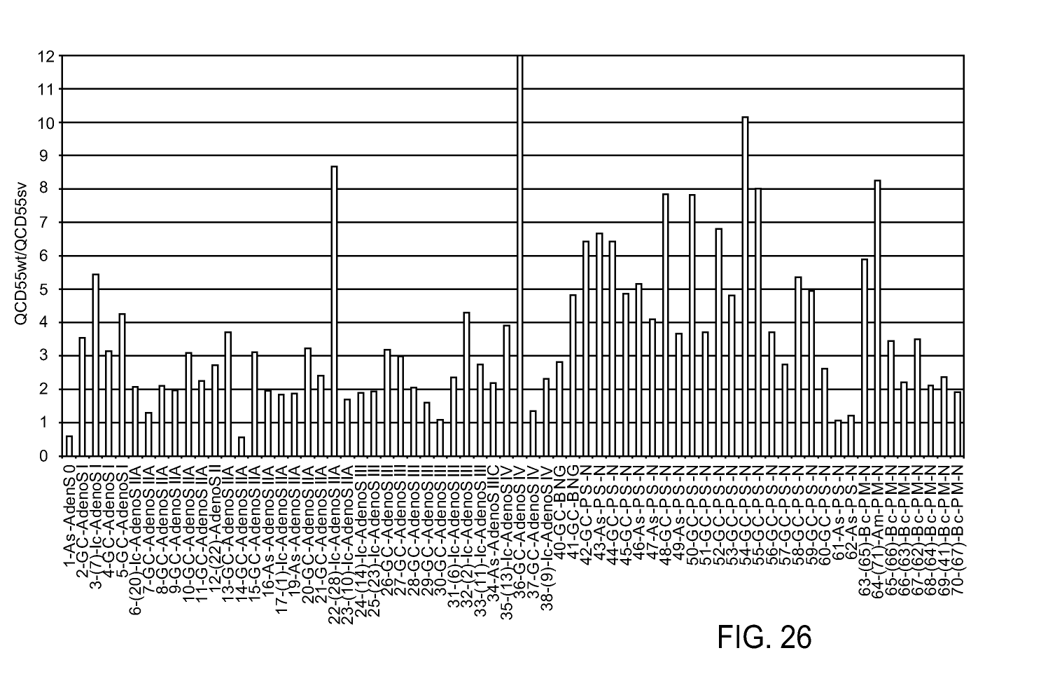

As evident from FIGS. 24 and 25, the CD55 wild type transcripts, detectable by the Amplicon seg24junc27-30F2R2 (SEQ ID NO:92), showed a significantly lower over expression in stage III colon cancer samples than CD55 variant transcripts detectable by the HUMDAF_seg24-28F1R1 amplicon (SEQ ID NO:90) (>5 fold increase in 3 out of 11 colon cancer samples for CD55 wild type transcripts, detectable by the seg24junc27-30F2R2 amplicon (SEQ ID NO:92), as compared to >5 fold increase in 7 out of 11 colon cancer samples for CD55 variant transcripts detectable by the HUMDAF_seg24-28F1R1 amplicon (SEQ ID NO:90)) and lower overexpression in all the colon cancer samples (>5 fold increase in 3 out of 37 colon cancer samples for CD55 wild type transcripts, detectable by the seg24junc27-30F2R2 amplicon (SEQ ID NO:92), as compared to >5 fold increase in 11 out of 37 colon cancer samples for CD55 variant transcripts detectable by the HUMDAF_seg24-28F1R1 amplicon (SEQ ID NO:90)). FIG. 26 presents the ratio of expression of CD55 wild type transcripts, detectable by the seg24junc27-30F2R2 amplicon (SEQ ID NO:92) and the expression of CD55 variant transcripts detectable by the HUMDAF_seg24-28F1R1 amplicon (SEQ ID NO:90) in colon panel. The relative quantity (Q) of the expression for each transcript is calculated based on the efficiency and threshold cycle of the respective amplicon [Q=1/(efficiency^{Ct})]. The ratio of the expression of CD55 wild type transcripts versus CD55 variant transcripts is calculated as Q(WildType) divided by Q(Variant). As evident from FIG. 26, in normal samples, the expression of CD55 wild type transcripts, detectable by the seg24junc27-30F2R2 (SEQ ID NO:92) amplicon is more abundunt than the expression of CD55 variant transcripts detectable by the HUMDAF_seg24-28F1R1 amplicon (SEQ ID NO:90). However, in colon cancer samples, the abundance of CD55 wild type transcripts, detectable by the seg24junc27-30F2R2 amplicon (SEQ ID NO:92) is lower relative to CD55 variant transcripts detectable by the HUMDAF_seg24-28F1R1 amplicon (SEQ ID NO:90), comparable to the normal. Since the expression of CD55 variant transcripts detectable by the HUMDAF_seg24-28F1R1 amplicon (SEQ ID NO:90) is lower in normal colon relative to expression of CD55 wild type transcripts, detectable by the seg24junc27-30F2R2 amplicon (SEQ ID NO:92), the overall overexpression of CD55 variant transcripts in colon cancer is more pronounced.

Expression of DAF HUMDAF Transcripts which are Detectable by Amplicon as Depicted in Sequence Name HUMDAF_seg24-28F1R1 (SEQ ID NO:90) in Different Normal Tissues

Expression of DAF transcripts detectable by or according to HUMDAF_seg24-28F1R1 amplicon (SEQ ID NO:90) and primers HUMDAF_seg24-28F1 (SEQ ID NO:88) and HUMDAF_seg24-28R1 (SEQ ID NO:89) was measured by real time PCR on normal panel. The samples used are detailed in Table 3. Non-detected sample (sample no. 10) was assigned Ct value of 41 and was calculated accordingly. For each RT sample, the expression of the above amplicon was normalized to the normalization factor calculated from the expression of these house keeping genes as described in “Materials and Experimental Procedures” herein.

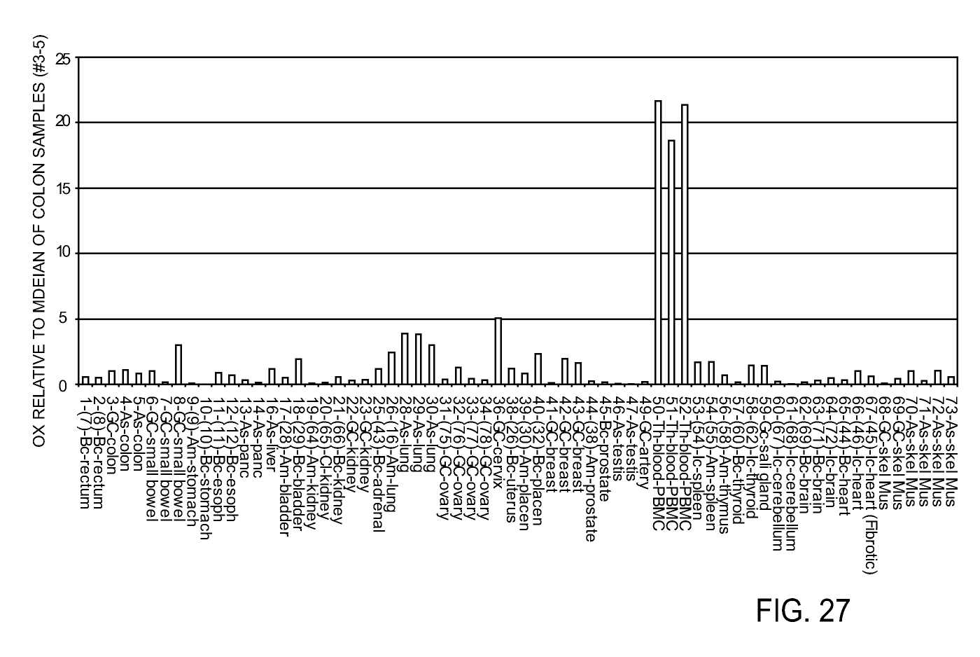

The normalized quantity of each RT sample was then divided by the median of the quantities of the colon samples (sample numbers 3 and 5, Table 3), to obtain a value of relative expression of each sample relative to median of the colon samples. The results presenting the expression of DAF HUMDAF transcripts which are detectable by amplicon as depicted in sequence name HUMDAF_seg24-28F1R1 (SEQ ID NO:90) in different normal tissues relative to median of the colon samples is shown in FIG. 27.

(SEQ ID NO: 88)

Forward primer: >seg24-28F1: GGCCCACCACCTGAATGCAG

(SEQ ID NO: 89)

Reverse primer: >seg24-28R1: TTTCTGAGGAGTTGGTGGGGT
TCTTG
Amplicon: seg24-28F1R1

(SEQ ID NO: 90)

GGCCCACCACCTGAATGCAGAGGAAAATCTCTAACTTCCAAGGTCCCACC
AACAGTTCAGAAACCTACCACAGTAAATGTTCCAACTACAGAAGTCTCAC
CAACTTCTCAGAAAACCACCACAAAAACCACCACACCAAATGCTCAAGGT
ACAGAGACTCCATCAGTTCTTCAAAAACACACCACAGAAAATGTTTCAGC
TACAAGAACCCCACCAACTCCTCAGAAA

Expression of DAF HUMDAF HUMDAF Transcripts which are Detectable by Amplicon as Depicted in Sequence Name HUMDAF_seg24junc27-30F2R2 (SEQ ID NO:92) in Different Normal Tissues

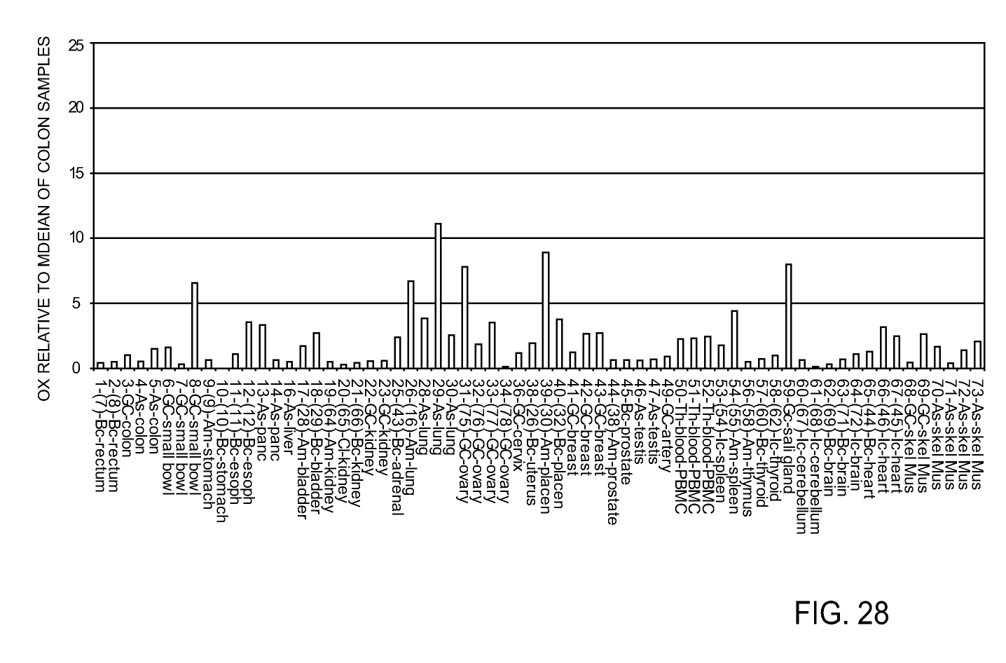

Expression of DAF HUMDAF transcripts detectable by or according to HUMDAF_seg24junc27-30F2R2 amplicon (SEQ ID NO:92) and primers HUMDAF_seg24junc27-30F2 (SEQ ID NO:62) and HUMDAF_seg24junc27-30R2 (SEQ ID NO:91) was measured by real time PCR on normal panel. The samples used are detailed in Table 3. Non-detected sample (sample no. 10) was assigned Ct value of 41 and was calculated accordingly. For each RT sample, the expression of the above amplicon was normalized to the normalization factor calculated from the expression of these house keeping genes as described in “Materials and Experimental Procedures”, herein. The normalized quantity of each RT sample was then divided by the median of the quantities of the colon samples (sample numbers 3-5, Table 3), to obtain a value of relative expression of each sample relative to median of the colon samples. The results presenting the expression of DAF HUMDAF transcripts which are detectable by amplicon as depicted in sequence name HUMDAF_seg24junc27-30F2R2 (SEQ ID NO:92) in different normal tissues relative to median of the colon samples is shown in FIG. 28.

Forward Primer (HUMDAF_seg24junc27-30F2)
(SEQ ID NO: 62): AGTGGCCCACCACCTGAATG
Reverse Primer (HUMDAF_seg24junc27-30R2)
(SEQ ID NO: 91): AGGTGTACTCCGTGTTGCTTGAG
Amplicon (HUMDAF_seg24junc27-30F2R2)
(SEQ ID NO: 92): AGTGGCCCACCACCTGAATGCAGAGGAAAATCT
CTAACTTCCAAGGTCCCACCAACAGTTCAGAAACCTACCACAGTAAATGT
TCCAACTACAGAAGTCTCACCAACTTCTCAGAAAACCACCACAAAAACC
ACCACACCAAATGCTCAAGCAACACGGAGTACACCT

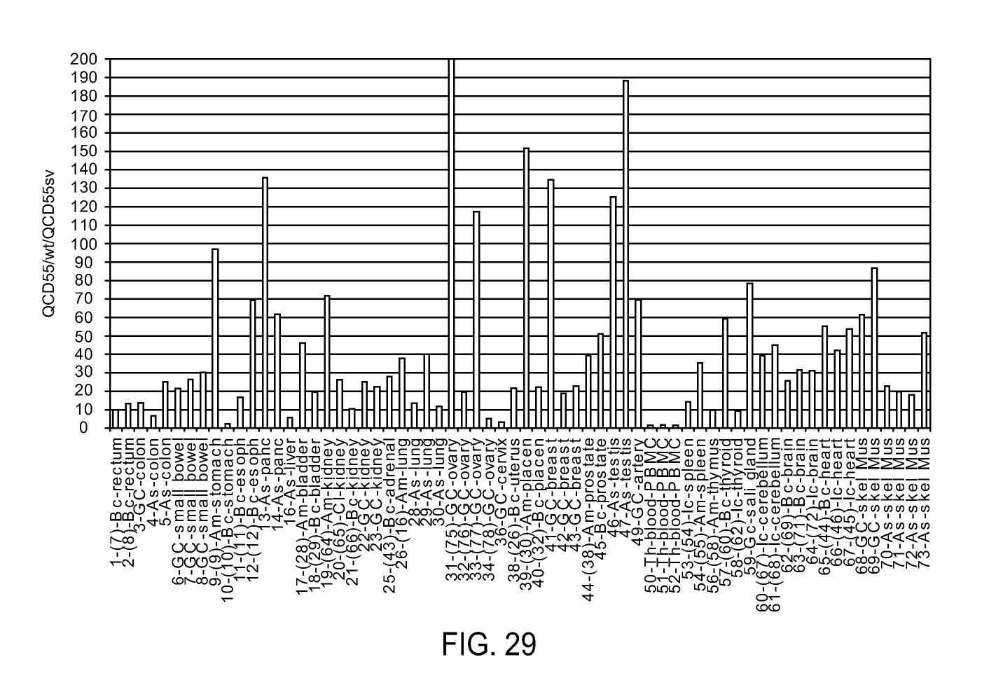

FIG. 29 presents the ratio of expression of the CD55 wild type transcripts detectable by the HUMDAF_seg24junc27-30F2R2 amplicon (SEQ ID NO:92) versus the expression of CD55 variant transcripts detectable by seg24-28F1R1 amplicon (SEQ ID NO:90) in panel of normal healthy samples. As evident from FIG. 29, the expression of CD55 wild type transcripts, detectable by the seg24junc27-30F2R2 amplicon (SEQ ID NO:92) is more abundant than the expression of CD55 variant transcripts detectable by the HUMDAF_seg24-28F1R1 amplicon (SEQ ID NO:90) in the vast majority of healthy tissues checked: in 54 out of 65 tissues the CD55 wild type transcripts are more than 10 fold more abundant than CD55 variant transcripts. As evident from FIGS. 26 and 29, the expression of CD55 variant transcripts detectable by the HUMDAF_seg24-28F1R1 amplicon (SEQ ID NO:90) in normal tissues is much lower than the expression of CD55 wild type transcripts, detectable by the seg24junc27-30F2R2 amplicon (SEQ ID NO:92), but is higher in colon cancer samples. Therefore the CD55 variants might be better targets than the CD55 wild type for anti-cancer therapy.

Expression of DAF HUMDAF Transcripts which are Detectable by Amplicon as Depicted in Sequence Name HUMDAF_seg24-28F1R1 (SEQ ID NO:90) in the Blood-Specific Panel

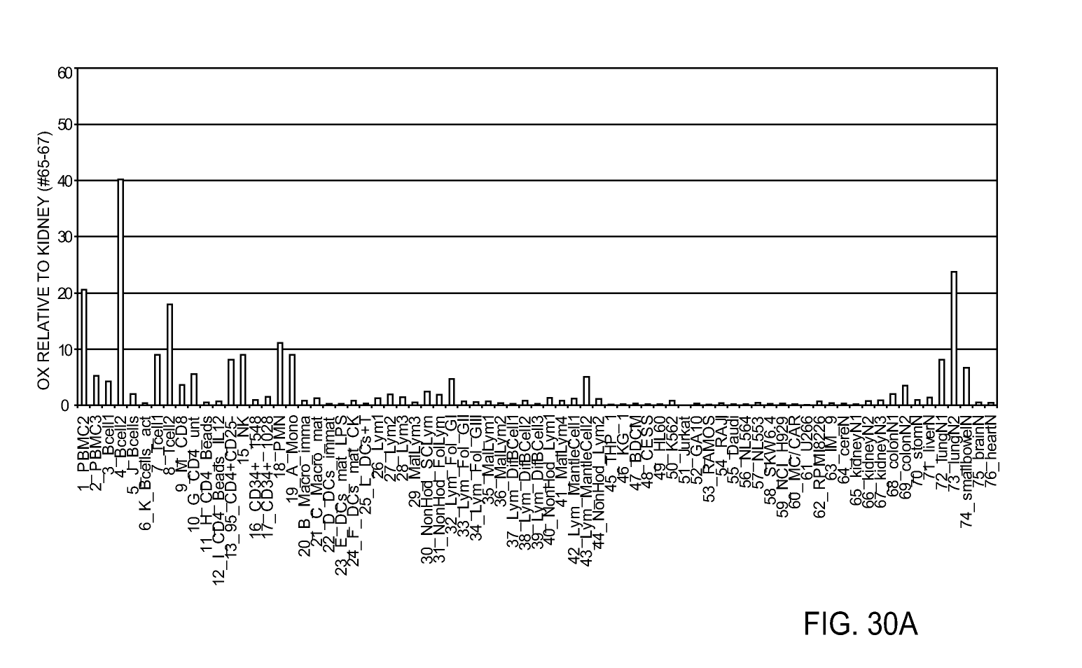

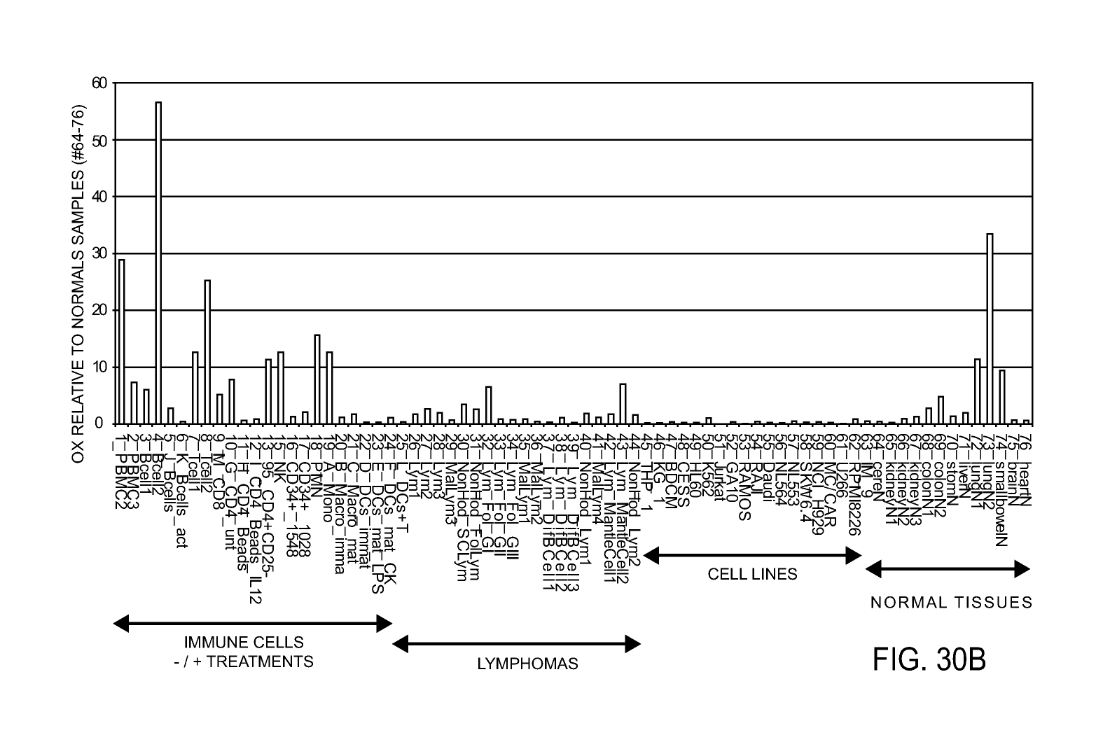

Expression of DAF transcripts, detectable by or according to HUMDAF_seg24-28F1R1 amplicon (SEQ ID NO:90) and primers HUMDAF_seg24-28F1 (SEQ ID NO:88) and HUMDAF_seg24-28R1 (SEQ ID NO:89) was measured by real time PCR on blood panel. The samples used are detailed in Table 2 above. For each RT sample, the expression of the above amplicon was normalized to the normalization factor calculated from the expression of these house keeping genes as described in section “Materials and Experimental Procedures” herein. The normalized quantity of each RT sample was also divided by the median of the quantities of the normal samples (sample numbers 64-76, Table 2), to obtain a value of relative expression of each sample relative to median of the normal samples (FIG. 30A). The normalized quantity of each RT sample was also divided by the median of the quantities of the kidney normal samples (sample numbers 65-67, Table 2), to obtain a value of relative expression of each sample relative to median of the kidney normal samples (FIG. 30B).

The results of this analysis are depicted in the histogram in FIGS. 30A and 30B. Expression of the above-indicated DAF-variant transcript is seen in PBMCs B cells, T cells, NK cells PMNs, monocytes and in normal lung and small bowel tissues.

Primer pairs are also optionally and preferably encompassed within the present invention; for example, for the above experiment, the following primer pair was used as a non-limiting illustrative example only of a suitable primer pair: seg24-28F1 forward primer (SEQ ID NO:88); and seg24-28R1 reverse primer (SEQ ID NO:89).

The present invention also preferably encompasses any amplicon obtained through the use of any suitable primer pair; for example, for the above experiment, the following amplicon was obtained as a non-limiting illustrative example only of a suitable amplicon:

seg24-28F1R1 (SEQ ID NO:90).

(SEQ ID NO: 88)

Forward primer: >seg24-28F1: GGCCCACCACCTGAATGCAG

(SEQ ID NO: 89)

Reverse primer: >seg24-28R1: TTTCTGAGGAGTTGGTGGGGT
TCTTG
Amplicon: seg24-28F1R1

(SEQ ID NO: 90)

GGCCCACCACCTGAATGCAGAGGAAAATCTCTAACTTCCAAGGTCCCACC
AACAGTTCAGAAACCTACCACAGTAAATGTTCCAACTACAGAAGTCTCAC
CAACTTCTCAGAAAACCACCACAAAAACCACCACACCAAATGCTCAAGG
TACAGAGACTCCATCAGTTCTTCAAAAACACACCACAGAAAATGTTTCAG
CTACAAGAACCCCACCAACTCCTCAGAAA

Expression of Wild Type DAF HUMDAF Transcripts which are Detectable by Amplicon as Depicted in Sequence Name HUMDAF_seg24junc27-30F2R2 (SEQ ID NO:92) in the Blood-Specific Panel

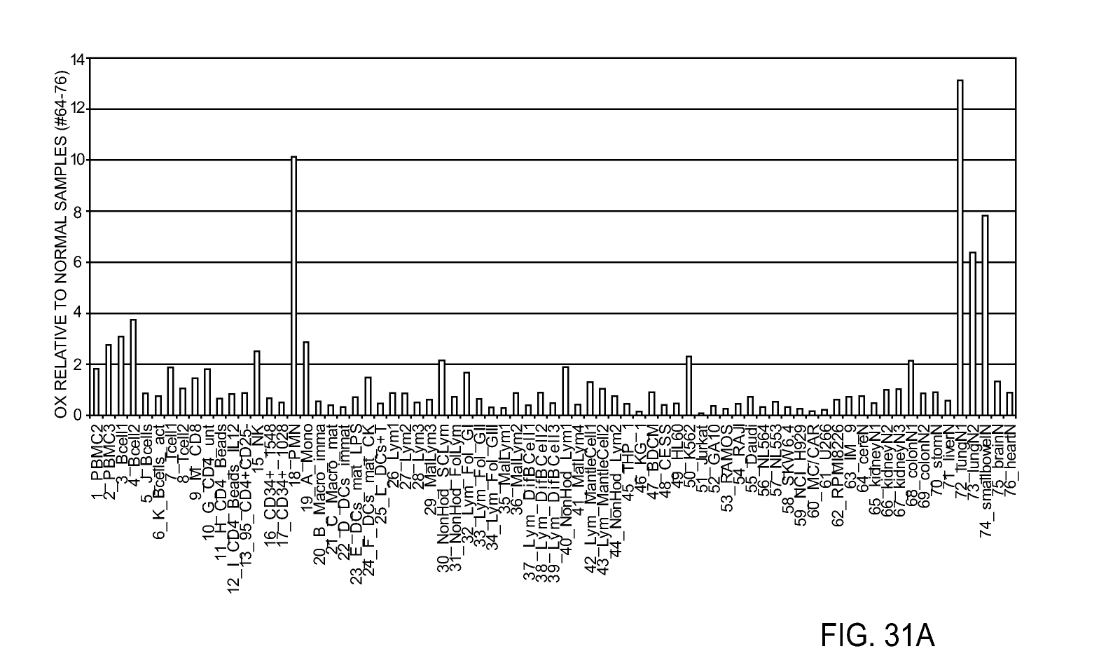

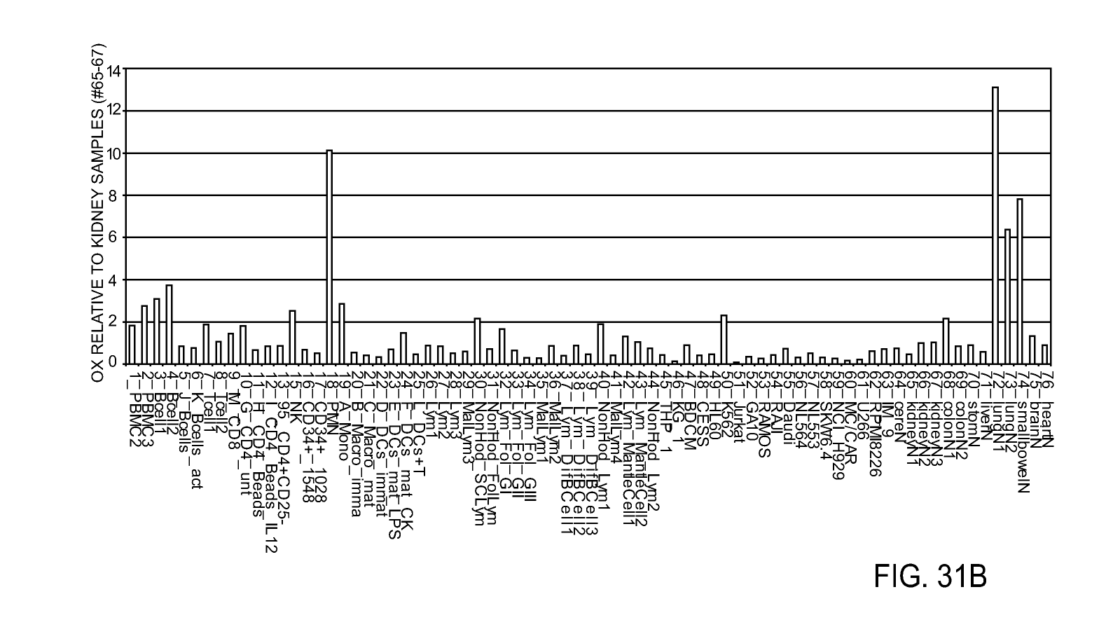

Expression of DAF WT transcripts detectable by or according to HUMDAF_seg24junc27-30F2R2 amplicon (SEQ ID NO:92) and primers HUMDAF_seg24junc27-30F2 (SEQ ID NO:62) and HUMDAF_seg24junc27-30R2 (SEQ ID NO:91) was measured by real time PCR on blood panel. The samples used are detailed in Table 2 above. For each RT sample, the expression of the above amplicon was normalized to the normalization factor calculated from the expression of these house keeping genes as described in section “Materials and Experimental Procedures” above. The normalized quantity of each RT sample was also divided by the median of the quantities of the normal samples (sample numbers 64-76, Table 2), to obtain a value of relative expression of each sample relative to median of the normal samples (FIG. 31A). The normalized quantity of each RT sample was also divided by the median of the quantities of the kidney normal samples (sample numbers 65-67, Table 2), to obtain a value of relative expression of each sample relative to median of the kidney normal samples (FIG. 31B).

The results of this analysis are depicted in histograms in FIGS. 31A and 31B. Expression of the above-indicated DAF transcript is seen in PBMCs B cells, T cells, NK cells, monocytes, while the highest expression is noted in PMNs. High overexpression is also seen normal lung and small bowel.

Primer pairs are also optionally and preferably encompassed within the present invention; for example, for the above experiment, the following primer pair was used as a non-limiting illustrative example only of a suitable primer pair: seg24junc27-30F2 forward primer (SEQ ID NO:62); and seg24junc27-30R2 reverse primer (SEQ ID NO:91).

The present invention also preferably encompasses any amplicon obtained through the use of any suitable primer pair; for example, for the above experiment, the following amplicon was obtained as a non-limiting illustrative example only of a suitable amplicon:

seg24junc27-30F2R2 (SEQ ID NO:92).

(SEQ ID NO: 92)

seg24junc27-30F2R2.

(SEQ ID NO: 62)

Forward primer: > seg24junc27-30F2: AGTGGCCCACCACC
TGAATG

(SEQ ID NO: 91)

Reverse primer: > seg24junc27-30R2: AGGTGTACTCCGTG
TTGCTTGAG
Amplicon: seg24junc27-30F2R2

(SEQ ID NO: 92)

AGTGGCCCACCACCTGAATGCAGAGGAAAATCTCTAACTTCCAAGGTCCC
ACCAACAGTTCAGAAACCTACCACAGTAAATGTTCCAACTACAGAAGTCT
CACCAACTTCTCAGAAAACCACCACAAAAACCACCACACCAAATGCTCAA
GCAACACGGAGTACACCT

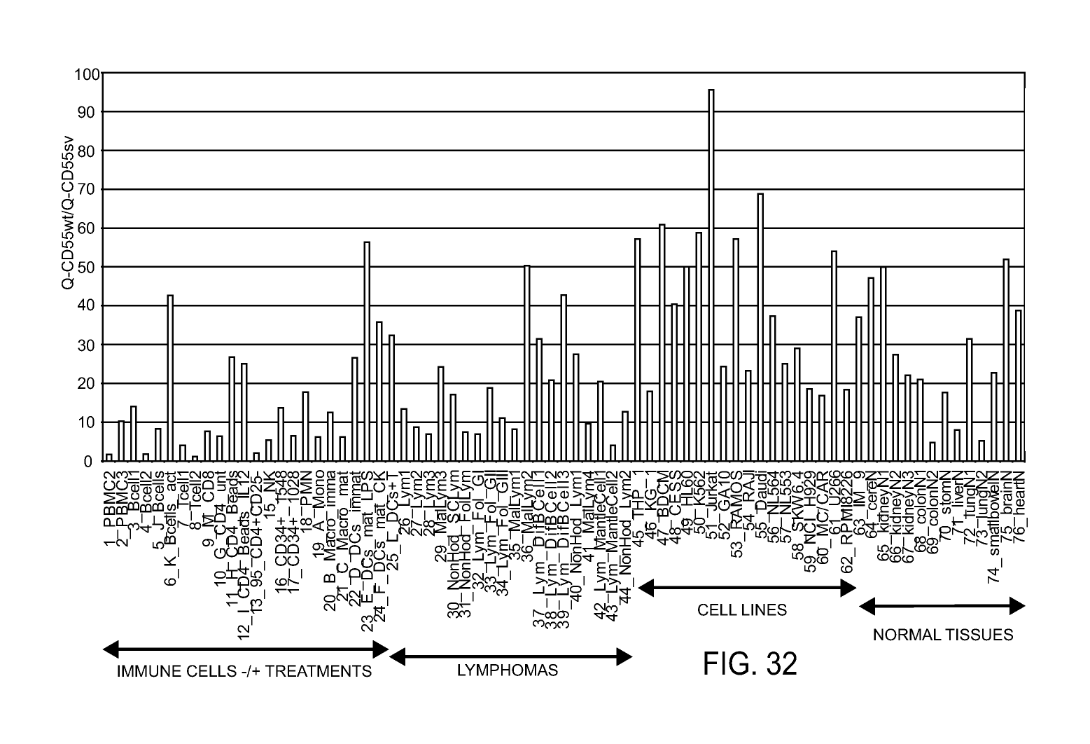

FIG. 32 presents the ratio of the expression quantity of CD55 wild type transcripts detectable by HUMDAF_seg24junc27-30F2R2 amplicon (SEQ ID NO:92) versus the expression of the CD55 variant transcripts detectable by HUMDAF_seg24-28F1R1 amplicon (SEQ ID NO:90) in panel of immune cells and normal tissues. As evident from FIG. 32, the expression quantity of the CD55 variant transcripts detectable by HUMDAF_seg24-28F1R1 amplicon (SEQ ID NO:90) is relatively low compared to CD55 wild type transcripts in most immune cells and normal tissues. In vast majority of the immune-derived samples CD55 wild type transcripts detectable by HUMDAF_seg24junc27-30F2R2 (SEQ ID NO:92) are more abundant than CD55 variant transcripts detectable by HUMDAF_seg24-28F1R1 amplicon (SEQ ID NO:90).

In one experiment that was carried out with primers HUMDAF_seg24-28F (SEQ ID NO:88) and HUMDAF_seg24-28R (SEQ ID NO:89) no differential expression was observed in the breast cancerous samples, lung cancerous samples, and ovary cancerous samples, relative to the corresponding normal samples.

### Example 4_{—}3

### Validation Analysis of CD55 Transcripts

In order to validate the most abundant transcript of CD55, RT-PCR validation was carried out using specific primers directed to the sequences of the HUMDAF_T10 (SEQ ID NO:34), HUMDAF_T11 (SEQ ID NO:35), HUMDAF_T17 (SEQ ID NO:36), HUMDAF_T19 (SEQ ID NO:37) or HUMDAF_T32 (SEQ ID NO:41), as described below. 1. A reverse transcription reaction was carried out as follows: 10 μg of purified ovary, lung or colon cancer RNA were mixed with 150 ng Random Hexamer primers (Invitrogen, Carlsbad, Calif., USA, catalog number: 48190-011) and 500 μM dNTPs in a total volume of 156 μl. The mixture was incubated for 5 min at 65° C. and then quickly chilled on ice. Thereafter, 50 ml of 5× SuperscriptII first strand buffer (Invitrogen, catalog number: 18064-014, part number: Y00146), 24 μl 0.1M DTT and 400 units RNasin (Promega, Milwaukee, Wis., U.S.A., catalog number: N2511) were added, and the mixture was incubated for 10 min at 25° C., followed by further incubation at 42° C. for 2 min. Then, 10 μl (2000 units) of SuperscriptII (Invitrogen, catalog number: 18064-014) was added and the reaction (final volume of 2500 was incubated for 50 min at 42° C. and then inactivated at 70° C. for 15 min. The resulting cDNA was diluted 1:20 in TE buffer (10 mM Tris, 1 mM EDTA pH 8).

2. PCR was done using GoTaq ReadyMix (Promega, catalog number M122) under the following conditions: 5 μl cDNA from the above; 1 μl of each primer (10 μM); 5.5 μl H₂O and 12.5 μl ReadyMix in a total reaction volume of 25 μl; with a reaction program of 2 minutes in 94° C.; 30 cycles of: 30 seconds at 94° C., 30 seconds at 51° C., 60 seconds at 72° C.; then 10 minutes at 72° C. The forward primer [100-892 CD55_n29_For (SEQ ID NO:58)] used, was specific to segment 29 (SEQ ID NO:71), which distinguishes between the possible transcript variants and the known wild type CD55 proteins (SEQ ID NOs:42, 48, 50). The reverse primer [100-895 CD55 n50 Rev (SEQ ID NO:59)] was directed to segment 50 (SEQ ID NO:72), and includes the 3′ of the ORF. The predicted transcripts that could be identified using the above primer set and the expected PCR products are described in Table 77 below. Specific information regarding the cDNA sample used is given in Table 78.

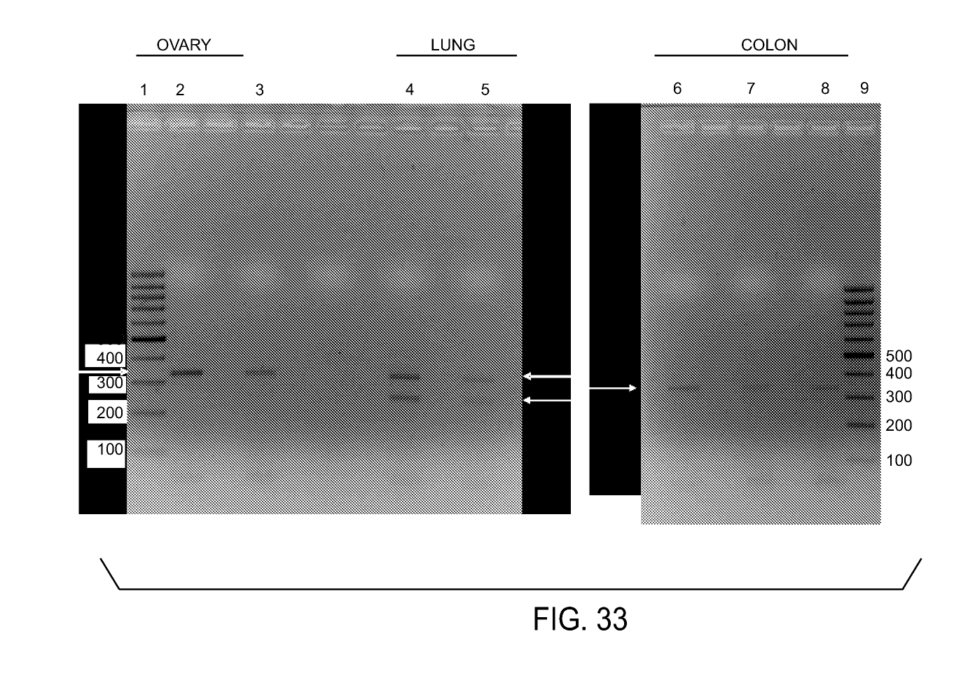

25 μl of the PCR products described above were loaded onto a 2% agarose gel stained with ethidium bromide, electrophoresed in 1×TAE solution at 100V, and visualized with UV light. The results are shown in FIG. 33. The expected band sizes of 332 bp was detected in the ovary colon and lung samples. An additional band of 254 bp was detected in the lung sample.

FIG. 33 demonstrates the gel analysis of the PCR product described above. Lanes 1 and 9 represent 100 bp DNA marker (Fermentas, Catalog number SM0244) lanes 2-8 represent the PCR products as follows, lane 2—Ovary borderline tumor 38-GC-SIA-BRD; lane 3—Ovary cancer 30-GC-SIC-MUC; lane 4—Lung cancer 17-(89)-Bc-Adeno; lane 5—Lung cancer 18—(76)-Bc-Adeno; lane 6—Colon cancer 24-(14)-Ic-AdenoSIII; lane 7—Colon cancer 25-(23)-Ic-AdenoSIII; lane 8—Colon cancer 27-GC-AdenoSIII.

PCR products from lanes 2; 3; 4; 6 and 8 of FIG. 33 were excised and extracted from the gel using QiaQuick™ Gel Extraction kit (Qiagen, catalog number: 28707) and sequenced. The 332 by fragments derived from lanes 2; 3; 4; 6 and 8 were verified as the predicted HUMDAF_T11 (SEQ ID NO:35), while the 254 by fragment excised from lane 4 was verified as the predicted HUMDAF_T10 (SEQ ID NO:34). This analysis demonstrates that T11 is the most abundant transcript out of the six transcripts tested.

TABLE 77
possible products using forward primer 100-892 (SEQ ID
NO: 58) and a reverse primer 100-895 (SEQ ID NO: 59)

| Transcript | Protein | Expected product size |
|---|---|---|
| HUMDAF_T10 | HUMDAF_P14 | 254 bp |
| HUMDAF_T11 | HUMDAF_P15 | 332 bp |
| HUMDAF_T17 | HUMDAF_P20 | 372 bp |
| HUMDAF_T32 | HUMDAF_P31 | 438 bp |
| HUMDAF_T19 | HUMDAF_P15 | 332 bp |

TABLE 78
tissue cDNA samples used as templates to identify CD55 transcripts

| | | | sample_id | | |
|---|---|---|---|---|---|
| | | | (GCI)/case id | TISSUE_ID | |
| | | | (Asterand)/ | (GCI)/ | |
| | Source/ | sample | lot no.(old | specimen | |
| Tissue | Delivery | name | samples) | ID (Asterand) | DIAGNOSIS |
| colon_cancer | Ichilov | 24-(14)-Ic- | CG-222 (2) | | |
| | | AdenoSIII | | | |
| colon_cancer | Ichilov | 25-(23)-Ic- | CG-282 | | |
| | | AdenoSIII | | | |
| colon_cancer | GCI- | 27-GC- | IG9NK | IG9NKAD3 | |
| | 3rd_del | AdenoSIII | | | |
| ovary_borderline_tumor | GCI- | 38-GC-SIA- | SC656 | SC656AKT | MUCINOUS |
| | 3rd_del | BRD | | | BORDERLINE |
| | | | | | TUMOR |
| ovary_cancer | GCI- | 30-GC-SIC- | IMDA1 | IMDA1ANG | MUCINOUS |
| | 1st_del | MUC | | | ADENOCARCINOMA |
| lung_cancer | Biochain | 17-(89)-Bc- | A609077 | | ADENOCARCINOMA |
| | | Adeno | | | |
| lung_cancer | Biochain | 18-(76)-Bc- | A609218 | | ADENOCARCINOMA |
| | | Adeno | | | |

### Example 4_{—}4

### Production of Polyclonal Antibodies Specific to CD55 Variant Proteins (SEQ ID NOS: 51, 52, 53, 56, 57)

All polyclonal Abs production procedure, including peptides synthesis, peptides conjugation, animal immunizations, bleeding and antibodies purification were performed at Sigma-Aldrich (Israel).

One pair of New Zealand White rabbits were injected with peptide described below to prepare antibodies specific for CD55 variant proteins (SEQ ID NOs: 51, 52, 53, 56, 57) rabbit numbers 5619 and 5620. All animal care, handling and injections were performed by Sigma (Israel). The peptide (SEQ ID NO: 70), used for rabbit immunization, derived from the unique protein region of CD55 variant proteins (SEQ ID NOs: 51, 52, 53, 56, 57), corresponding to amino acids residues 328-347 (SEQ ID NO:70) of the CD55 variant (SEQ ID NOs: 51, 52, 53, 56, 57) proteins. Cystein was added to the peptide's C′ terminus for KLH conjugation. Rabbits 5619 and 5620 were immunized with the CD55 variants specific peptide (SEQ ID NO:70). Animals were immunized every two weeks. Three test bleeds were collected and analyzed by ELISA. 100 ml production bleeds from each rabbit were collected and antibodies are affinity purified against the immunized peptide. The purified antibodies are analyzed by ELISA and Western Blot analysis by methods known in the art.

### Example 4_{—}4_{—}1

### Cloning of CD55 Transcripts Cloning of CD55 Transcript HUMDAF_T0_P0 Fused to FLAG

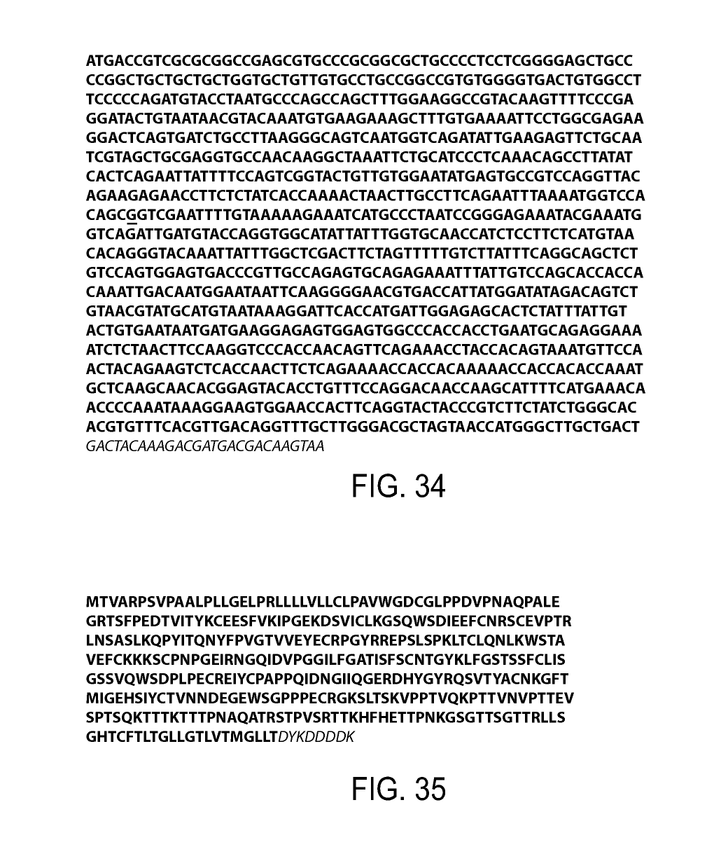

In order to test the specificity of the affinity purified antibodies, known wild type CD55 (SEQ ID NO:42) and CD55 variant of the invention HUMDAF_P15 (SEQ ID NO:52) were cloned. The cloning of CD55 HUMDAF TO (also referred herein as CD55_T0) (SEQ ID NO:66) was done as follows. The 5′ region of CD55HUMDAF TO was amplified using ovary borderline tumor cDNA as a template and PCR primers #100-901 (SEQ ID NO:60) and #100-907 (SEQ ID NO: 65). PCR was done using GoTaq ReadyMix (Promega, catalog number M122) under the following conditions: 5 μl cDNA; 1 μl of each primer (10 μM); 5.5 μl H₂O and 12.5 μl ReadyMix in a total reaction volume of 25 μl; with a reaction program of 2 minutes in 94° C.; 30 cycles of: 30 seconds at 94° C., 30 seconds at 52° C., 2.5 minutes at 72° C.; then 10 minutes at 72° C. The PCR product was then digested with NheI and PpuMI. Next, a second PCR was done amplifying the 3′ region of CD55_T0 and partially overlapping with the PCR fragment from above. PCR was done using ovary borderline tumor cDNA as a template and PCR primers #100-959 (SEQ ID NO:62) and #100-902 (SEQ ID NO:63), PCR conditions were as described above. Following PCR, the product was digested with PpuMI and AgeI. The two digested PCR fragments were loaded onto a 1% agarose gel stained with ethidium bromide, electrophoresed in 1×TAE solution at 100V, and visualized with UV light. After verification of expected band size, the PCR products were excised and extracted from the gel using QiaQuick™ Gel Extraction kit (Qiagen, catalog number: 28707). The digested DNA fragments were ligated to each other and to pIRESpuro3 vector (Clontech, catalog number 631619) previously digested with NheI and AgeI using the LigaFast™ Rapid DNA Ligation System (Promega, catalog number: M8221). The resulting DNA was transformed into competent E. coli bacteria DH5a (RBC Bioscience, Taipei, Taiwan, catalog number: RH816) according to manufacturer's instructions, then plated on LB-ampicillin agar plates for selection of recombinant plasmids, and incubated overnight at 37° C. The following day, a number of colonies from each transformation that grew on the selective plates were taken for further analysis by streak-plating on another selective plate and by PCR using GoTaq ReadyMix (Promega, catalog number: M7122). Screening positive clones was performed by PCR using pIRESpuro3 vector specific primer and gene specific primer (data not shown). After completion of all PCR cycles, half of the reaction was analyzed using 1% agarose gel as described above. After verification of expected band size, 2 positive colonies from each ligation reactions were grown in 5 ml Terrific Broth supplemented with 100 μg/ml ampicillin, with shaking overnight at 37° C. Plasmid DNA was isolated from bacterial cultures using Qiaprep™ Spin Miniprep Kit (Qiagen, catalog number: 27106). Accurate cloning was verified by sequencing the inserts (Weizmann Institute, Rehovot, Israel). Upon verification of a colony that has one silent mutation in the ORF, the recombinant plasmid was processed for further analyses. The DNA sequence of the resulting CD55 transcript HUMDAF TO FLAG (SEQ ID NO:66) is shown in FIG. 34. Gene specific sequence corresponding to CD55_T0 ORF sequence is marked in bold faced, FLAG tag sequence is in italics, silent mutation is underlined. The amino acid sequence of HUMDAF PO FLAG (also referred herein as CD55_P0_FLAG) protein (SEQ ID NO:67) is shown in FIG. 35; amino acid sequence corresponding to CD55 ORF is marked in bold faced, FLAG sequence is in italics.

### Cloning f CD55 Variant HUMDAF_T11_P15(1-523) Fused to FLAG

Cloning of CD55 HUMDAF_T11 (SEQ ID NO:35) partial open reading frame (ORF) amino acids 1-523 of the CD55 HUMDAF_P15 protein (SEQ ID NO: 52) fused to FLAG was carried out as described below.

The 5′ region of CD55 HUMDAF_T11 (SEQ ID NO:35) (also referred herein as CD55_T11) was amplified using ovary borderline tumor cDNA as a template and PCR primers #100-901 (SEQ ID NO:60) and #100-893 (SEQ ID NO:61). PCR was done using GoTaq ReadyMix (Promega, catalog number M122) under the following conditions: 5 μl cDNA; 1 μl of each primer (10 μM); 5.5 μl H₂O and 12.5 μl ReadyMix in a total reaction volume of 25 μl; with a reaction program of 2 minutes in 94° C.; 30 cycles of: 30 seconds at 94° C., 30 seconds at 52° C., 2.5 minutes at 72° C.; then 10 minutes at 72° C. Next, a second PCR was done amplifying the 3′ region of CD55_T11 and partially overlapping with the PCR fragment from above. PCR was done using ovary borderline tumor cDNA as a template and PCR primers #100-892 (SEQ ID NO:58) and #100-950 (SEQ ID NO:64), PCR conditions were as described above. Following PCR, the two PCR DNA fragments were pooled into one tube and used as template for a third PCR using primers #100-901 (SEQ ID NO:60) and #100-950 (SEQ ID NO:64)). The PCR product was then digested with NheI and AgeI and ligated into pIRESpuro3 as described above. DNA was transformed into competent E. coli bacteria DH5α (RBC Bioscience, Taipei, Taiwan, catalog number: RH816) as described above. Screening of positive clones was performed as described above. Accurate cloning was verified by sequencing the inserts (Weizmann Institute, Rehovot, Israel).

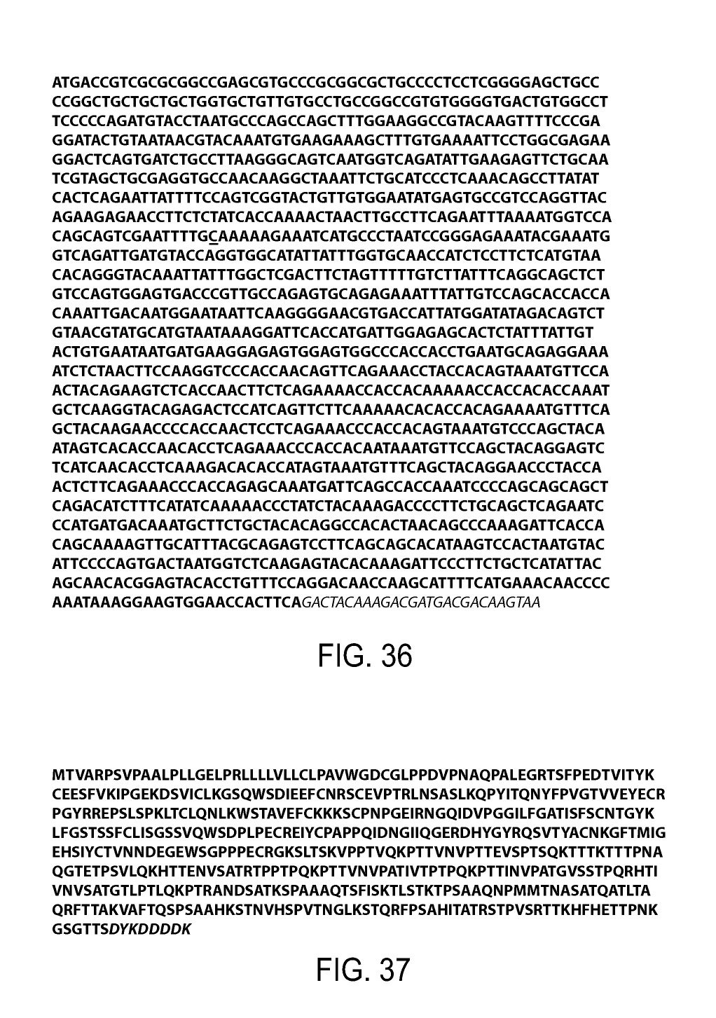

The DNA sequence of the resulting CD55_T11_P15(1-523)_FLAG (SEQ ID NO: 68) is shown in FIG. 36; gene specific sequence corresponding to CD55_T11 ORF sequence is marked in bold faced, FLAG tag sequence is in italics, point mutation is underlined.

The amino acid sequence of CD55_T11_P15(1-523)_FLAG (SEQ ID NO: 69) (also referred herein as CD55_P15 FLAG or CD55 HUMDAF_T11_P15(1-523)_FLAG) is shown in FIG. 37; amino acid sequence corresponding to CD55 HUMDAF_P15 (SEQ ID NO:52) ORF is marked in bold faced, FLAG sequence is in italics.

### Example 4_{—}4_{—}2

### Characterization of Test Bleed CD55 Variants Specific Antibodies by Immuno-Precipitation and Western Blot Using CD55 Transfected Cells Lysates

In order to verify the specificity of antibodies raised against the selected peptide of CD55 variant s (SEQ ID NO: 70), immuno-precipitation followed by western blot analysis was done using non purified serum from rabbits 5619 and 5620 described above, and CHO-K1 (ATCC, CCL-61) stable transfectants cell lysates of CD55 HUMDAF TO FLAG (SEQ ID NO:66) or CD55 HUMDAF_T11_P15(1-523)_FLAG (SEQ ID NO:68) as described below.

CHO-K1 (ATCC, CCL-61) cells were plated in a sterile 6 well plate suitable for tissue culture, using 2 ml pre-warmed of complete media, F12 Nutrient Mixture (HAM), (Gibco, catalog number: 21765-029)+10% FBS [Fetal Bovine Serum, Biological Industries (Beit Ha'Emek, Israel), catalog number: 04-001-1A]+4 mM L-Glutamine [Biological Industries (Beit Ha'Emek, Israel), catalog number: 03-020-1A]. 300,000 cells per well were transfected with 2 μg of DNA construct using 6 μl FuGENE 6 reagent (Roche, catalog number: 11-814-443-001) diluted into 94 μl F12 medium. The mixture was incubated at room temperature for 15 minutes. The complex mixture was added dropwise to the cells and swirled. Cells were placed in incubator maintained at 37° C. with 5% CO₂ content. 48 hours following transfection, transfected cells were transferred to a 75 cm² tissue culture flask containing 15 ml of selection media: complete media supplemented with 10 μg\ml puromycin (Sigma, catalog number P8833). Cells were placed in incubator, and media was changed every 3-4 days, until clone formation observed. Upon sufficient quantities of cells passing through selection, 3-5 million cells were harvested. As a control, the same amount of CHO-K1 un-transfected cells were also harvested and treated the same way as the transfected cell. CD55_P0_FLAG (SEQ ID NO:67); CD55_T11_P15 1-523FLAG (SEQ ID NO:69) and untransfected cell lysates were Immuno-precipitated using anti CD55 antibody NaM16-4D3 (Santa Cruz Biotechnology, catalog number: SC-51733), this commercial antibody recognizes an epitope common to wild type CD55_P0 (SEQ ID NO:42) and to CD55 variants (SEQ ID NOs: 51, 52, 53, 56, 57). Immuno-precipitation was done as follows: Cells were lysed in 400 μl RIPA buffer (50 mM Tris HCl pH 8, 150 mM NaCl, 1% NP-40, 0.5% sodium Deoxycholate, 0.1% SDS) supplemented with protease inhibitors (Roche, catalog number: 11873580001), for 1.5 hrs at 4° C. Following centrifugation at 4° C. for 15 minutes at 20,000×g, the clear supernatants were transferred to clean tubes. The supernatants were incubated for 30 minutes at 4° C. with 50 μl protein A sepharose beads (Amersham, catalog number: 17-5280-04) which were pre-washed and diluted 1:1 with RIPA buffer. After spinning (20 seconds at 4000 rpm, Eppendorf centrifuge) the cleared supernatants were transferred to clean tubes and incubated with 1 μg commercial mouse anti CD55 antibody NaM16-4D3 (Santa Cruz Biotechnology, catalog number: SC-51733). Following an overnight incubation at 4° C., 50 μl of, pre-washed and diluted 1:1 with RIPA buffer, protein A sepharose beads were added and incubated for 45 minutes at 4° C. The beads-antibody complex was then washed 3 times with 1 ml cold RIPA buffer, by spinning at 4000 rpm for 20 seconds. The proteins were eluted by addition of 80 μl 4× NuPAGE® LDS sample buffer (Invitrogen, catalog number: NP0007) diluted to 1× with 100 mM citrate phosphate buffer pH3.5. In addition, 1,4-Dithiothreitol (DTT; a reducing agent) was added to a final concentration of 100 mM. The samples were then incubated at 100° C. for 3 minutes, followed by a 20 seconds spin at 4000 rpm. SDS-PAGE (Laemmli, U.K., Nature 1970; 227; 680-685) was performed upon loading of 20 μl of sample per lane into a 4-12% NuPAGE® Bis-Tris gels (Invitrogen, catalog number: NP0322), and gels were run in 1×MOPS SDS running buffer (Invitrogen, catalog number: NP0001), using the XCell SureLock™ Mini-Cell (Invitrogen, catalog number: E10001), according to manufacturer's instructions. The separated proteins were transferred to nitrocellulose membranes (Schleicher & Schuell, catalog number: 401385) using the XCell™ II blotting apparatus (Invitrogen, catalog number E19051), according to manufacturer's instructions.

The membranes containing blotted proteins were processed for antibody detection as follows:

Non-specific regions of the membrane were blocked by incubation with 10% skim-milk diluted in Tris buffered saline supplemented with 0.05% Tween-20 (Sigma cat: P5927) for 1 hour at room temperature (all subsequent incubations occur for 1 hour at room temperature). Blocking solution was then replaced with primary antibody solution: 3^{rd} bleed (before purification) from rabbits 5619 and 5620 described above diluted 1:500 in blocking solution. As a control, the membrane was incubated with commercial mouse anti CD55 antibody (Abcam, catalog number: ab54595). This commercial antibody recognizes an epitope common to wild type CD55_P0 (SEQ ID NO:42) and to CD55 variants (SEQ ID NOs: 51, 52, 53, 56, 57). After 3 10-minute washes, secondary antibody was applied: goat anti-rabbit conjugated to horse radish-peroxidase (Jackson ImmunoResearch, catalog number: 111-035-144) diluted 1:10,000 in blocking solution or goat anti-mouse conjugated to horse radish-peroxidase (Jackson ImmunoResearch, catalog number: 115-035-062) diluted 1:10,000 in blocking solution. After three 10-minutes washes, ECL substrate (GE-Amersham, catalog number: RPN2209) was applied for 1 minute, followed by exposure to X-ray film (Fuji, catalog number: 100NIF).

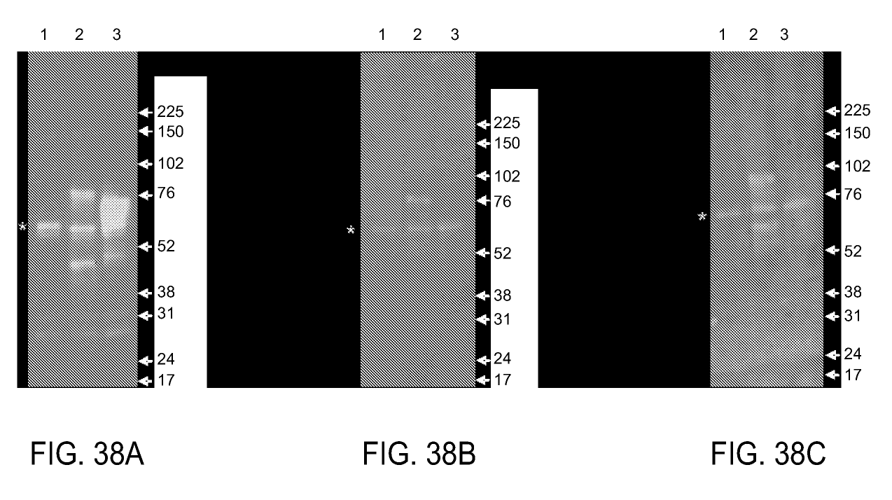

FIG. 38A-C demonstrates that both serum 5619 and 5620 specifically recognize an epitope located within CD55 variant proteins (SEQ ID NOs: 51, 52, 53, 56, 57), but do not detect the wild type CD55_P0 protein (SEQ ID NO:42). However, the commercial mouse anti CD55 recognizes both the wild type CD55 PO (SEQ ID NO:42) and the variant CD55_P15 (SEQ ID NO:52). Un-transfected CHO-K1 cells (lane 1) or CHO-K1 stably transfected with either CD55_P15 1-523FLAG pIRESpuro3 (SEQ ID NO:68) (lane 2) or CD55_P0_FLAG (SEQ ID NO:67) were subjected to immuno-precipitation with mouse anti CD55 (NaM16-4D3) antibody, followed by western blot with commercial mouse anti CD55 (FIG. 38A) or rabbit anti CD55_P15 sera (FIGS. 38B and 38C). A cross reactive band is marked by *.

### Example 4_{—}5

### Immuno-Staining of Colon Cell-Lines Using Antibodies Specific to CD55 Variants

Antibody-Protein interaction was demonstrated using immuno-fluorescence analysis on colon cells listed in Table 79, as described below.

TABLE 79

| Cell lines | Organ, desease |
|---|---|
| HCT116 (ATCC, CCL-247) | Colon, colorectal carcinoma |
| HT29 (ATCC, HTB38) | Colon, colorectal adenocarcinoma |
| SW480 (ATCC, CCL-228) | Colon, colorectal adenocarcinoma |

250,000 cells per well were plated on sterile glass coverslips, 13 mm diameter (Marienfeld, catalog number: 01 115 30), which were placed in a 6 well plate, using 2 ml pre-warmed DMEM [Dulbecco's modified Eagle's Media, Biological Industries (Beit Ha'Emek, Israel), catalog number: 01-055-1A]+5% FBS [Fetal Bovine Serum, Biological Industries (Beit Ha'Emek, Israel), catalog number: 04-001-1A]+4 mM L-Glutamine [Biological Industries (Beit Ha'Emek, Israel), catalog number: 03-020-1A]+ PEN-STREP solution ((Beit Ha'Emek, Israel), catalog number: 03-031-1B) diluted 1:100 (100units/ml PENICILIN 0.1 mg/ml streptomycin).

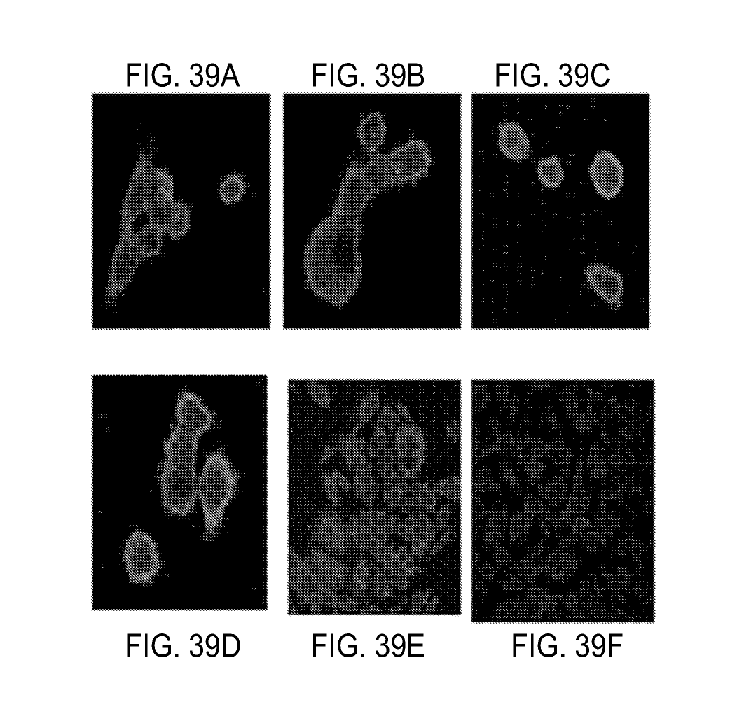

24 hours post plating the cells on coverslips cells were further processed for immunostaining and analysis by confocal microscopy. The cover slips were washed in phosphate buffered saline (PBS), then fixed for 15 minutes with a solution of 3.7% paraformaldehyde (PFA) (Sigma, catalog number: P-6148) and 3% glucose (Sigma, catalog number: G5767), followed by 5 minutes incubation with 3 mM glycine (Sigma, catalog number: G7126). After 1 wash in PBS, the cells were permeabilized by incubation with 0.1% triton X-100/PBS solution for 5 minutes. After two 5-minute washes in PBS, blocking of non-specific regions was done with 5% bovine serum albumin (BSA) (Sigma, catalog number: A4503) (diluted in PBS) for 20 minutes. The coverslips were then incubated, in a humid chamber for 1 hour, with purified rabbit anti-CD55 antibodies described above (RB 5619 and 5620) 1 mg/ml diluted 1:000 in 5% BSA in PBS. The antibodies were washed 3 times for 5-minutes in PBS. The coverslips were then incubated, in a humid chamber for 1 hour, with secondary antibody: donkey anti-rabbit conjugated to Cy-3 fluorophore (Jackson ImmunoResearch, catalog number: 711-165-152), diluted 1:200 in 3% BSA in PBS. After three 5-minute washes in PBS, the fixed coverslips were mounted on slides with Gel Mount Aqueous medium (Sigma, catalog number: G0918) and cells were observed for the presence of fluorescent product using confocal microscopy. FIG. 39 presents the results, demonstrating the specific binding of the antibodies 5619 and 5620, raised against a peptide shown in SEQ ID NO:70, to CD55 variant proteins (SEQ ID NOs: 51, 52, 53, 56, 57) in colon cells.

FIGS. 39A and 39B demonstrate by red fluorescence of antibodies 5619 and 5620 respectively, conjugated to Cy3 fluorophore, that one or more of CD55 splice variant proteins (SEQ ID NOs: 51, 52, 53, 56, 57) is expressed in the cell membrane of HCT116 cell line and is recognized by the specific antibody raised against CD55 variants specific peptide (SEQ ID NO. 70). FIGS. 39C and 39D demonstrate by red fluorescence of antibodies 5619 and 5620 respectively conjugated to Cy3 fluorophore that one or more of CD55 splice variant proteins (SEQ ID NOs: 51, 52, 53, 56, 57) is expressed in the cell membrane of HT29 cell line and is recognized by the specific antibody raised against CD55 variants specific peptide (SEQ ID NO70) from rabbits 5619 and 5620 respectively.

FIGS. 39E and 39F demonstrate that the red fluorescence signal is non specific, in SW480 cells. The image was obtained using the 40× objective of the confocal microscope.

### Example 5

### Development of Fully Human Anti-KIAA0746, Anti-CD20 and Anti-CD55 Antibodies

Generation Of Human Monoclonal Antibodies Against KIAA0746, CD20 and CD55 Antigen

Fusion proteins composed of the extracellular domain of the KIAA0746, CD20 and CD55 linked to an IgG2 Fc polypeptide are generated by standard recombinant methods and used as antigen for immunization.

Transgenic HuMab Mouse.

Fully human monoclonal antibodies to KIAA0746, CD20 and CD55 are prepared using mice from the HCo7 strain of the transgenic HuMab Mouse®, which expresses human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851, and a human heavy chain transgene, HCo7, as described in U.S. Pat. Nos. 5,545,806; 5,625,825; and 5,545,807.

HuMab Immunizations:

To generate fully human monoclonal antibodies to KIAA0746, CD20 and CD55, mice of the HCo7 HuMab Mouse® (strain can be immunized with purified recombinant KIAA0746, CD20 and CD55 fusion protein derived from mammalian cells that are transfected with an expression vector containing the gene encoding the fusion protein. General immunization schemes for the HuMab Mouse® are described in Lonberg, N. et al (1994) Nature 368(6474): 856-859; Fishwild, D. et al. (1996) Nature Biotechnology 14: 845-851 and PCT Publication WO 98/24884. The mice are 6-16 weeks of age upon the first infusion of antigen. A purified recombinant KIAA0746, CD20 and CD55 antigen preparation (5-50.mu.g, purified from transfected mammalian cells expressing KIAA0746, CD20 and CD55 fusion protein) is used to immunize the HuMab mice intraperitoneally.

Transgenic mice are immunized twice with antigen in complete Freund's adjuvant or Ribi adjuvant IP, followed by 3-21 days IP (up to a total of 11 immunizations) with the antigen in incomplete Freund's or Ribi adjuvant. The immune response is monitored by retroorbital bleeds. The plasma is screened by ELISA (as described below), and mice with sufficient titers of anti-KIAA0746, anti-CD20 or anti-CD55 human immunoglobulin are used for fusions. Mice are boosted intravenously with antigen 3 days before sacrifice and removal of the spleen.

Selection of HuMab Mice™ Producing Anti-KIAA0746, Anti-CD20 or Anti-CD55 Antibodies:

To select HuMab Mice™ producing antibodies that bind KIAA0746, CD20 and CD55 sera from immunized mice is tested by a modified ELISA as originally described by Fishwild, D. et al. (1996). Briefly, microtiter plates are coated with purified recombinant KIAA0746, CD20 and CD55 fusion protein at 1-2.mu.g/ml in PBS, 50.mu.l/wells incubated 4 degrees C. overnight then blocked with 200.mu.l/well of 5% BSA in PBS. Dilutions of plasma from KIAA0746, CD20 and CD55-immunized mice are added to each well and incubated for 1-2 hours at ambient temperature. The plates are washed with PBS/Tween and then incubated with a goat-anti-human kappa light chain polyclonal antibody conjugated with alkaline phosphatase for 1 hour at room temperature. After washing, the plates are developed with pNPP substrate and analyzed by spectrophotometer at OD 415-650. Mice that developed the highest titers of anti-KIAA0746, anti-CD20 or anti-CD55 antibodies are used for fusions. Fusions are performed as described below and hybridoma supernatants are tested for anti-KIAA0746, anti-CD20 or anti-CD55 activity by ELISA.

Generation of Hybridomas Producing Human Monoclonal Antibodies to KIAA0746, CD20 or CD55

The mouse splenocytes, isolated from the HuMab mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of P3X63 Ag8.6.53 (ATCC CRL 1580) nonsecreting mouse myeloma cells with 50% PEG (Sigma). Cells are plated at approximately 1×10−5/well in flat bottom microtiter plate, followed by about two week incubation in selective medium containing 10% fetal calf serum, supplemented with origen (IGEN) in RPMI, L-glutamine, sodium pyruvate, HEPES, penicillin, streptamycin, gentamycin, 1×HAT, and beta-mercaptoethanol. After 1-2 weeks, cells are cultured in medium in which the HAT is replaced with HT. Individual wells are then screened by ELISA (described above) for human anti-KIAA0746, anti-CD20 or anti-CD55 monoclonal IgG antibodies. Once extensive hybridoma growth occurred, medium is monitored usually after 10-14 days. The antibody secreting hybridomas are replated, screened again and, if still positive for human IgG, anti-KIAA0746, anti-CD20 or anti-CD55 monoclonal antibodies are subcloned at least twice by limiting dilution. The stable subclones are then cultured in vitro to generate small amounts of antibody in tissue culture medium for further characterization.

Hybridoma clones are selected for further analysis.

Structural Characterization of Desired Anti Anti-KIAA0746, Anti-CD20 or Anti-CD55 Human Monoclonal Antibodies

The cDNA sequences encoding the heavy and light chain variable regions of the obtained anti-KIAA0746, anti-CD20 or anti-CD55 monoclonal antibodies are obtained from the resultant hybridomas, respectively, using standard PCR techniques and are sequenced using standard DNA sequencing techniques.

The nucleotide and amino acid sequences of the heavy chain variable region and of the light chain variable region are identified. These sequences may be compared to known human germline immunoglobulin light and heavy chain sequences and the CDRs of each heavy and light of the obtained anti-KIAA0746, anti-CD20 or anti-CD55 sequences identified.

Characterization of Binding Specificity and Binding Kinetics of Anti-KIAA0746, Anti-CD20 or Anti-CD55 Human Monoclonal Antibodies

The binding affinity, binding kinetics, binding specificity, and cross-competition of anti-KIAA0746, anti-CD20 or anti-CD55 antibodies are examined by Biacore analysis. Also, binding specificity is examined by flow cytometry.

Binding Affinity and Kinetics

Anti-KIAA0746, anti-CD20 or anti-CD55 antibodies produced according to the invention are characterized for affinities and binding kinetics by Biacore analysis (Biacore AB, Uppsala, Sweden). Purified recombinant human KIAA0746, CD20 or CD55 fusion protein is covalently linked to a CM5 chip (carboxy methyl dextran coated chip) via primary amines, using standard amine coupling chemistry and kit provided by Biacore. Binding is measured by flowing the antibodies in HBS EP buffer (provided by BIAcore AB) at a concentration of 267 nM at a flow rate of 50.mu.l/min. The antigen-association antibodies association kinetics is followed for 3 minutes and the dissociation kinetics is followed for 7 minutes. The association and dissociation curves are fit to a 1:1 Langmuir binding model using BIAevaluation software (Biacore AB). To minimize the effects of avidity in the estimation of the binding constants, only the initial segment of data corresponding to association and dissociation phases are used for fitting.

Epitope Mapping of Obtained Anti-KIAA0746, Anti-CD20 or Anti-CD55 Antibodies

Biacore is used to determine epitope grouping of anti-KIAA0746, anti-CD20 or anti-CD55 antibodies are used to map their epitopes on the KIAA0746, CD20 or CD55 antigen, respectively. These different antibodies are coated on three different surfaces of the same chip to 8000 RUs each. Dilutions of each of the mAbs are made, starting at 10 mu.g/mL and is incubated with Fc fused KIAA0746, CD20 or CD55 (50 nM) for one hour. The incubated complex is injected over all the three surfaces (and a blank surface) at the same time for 1.5 minutes at a flow rate of 20.mu.L/min. Signal from each surface at end of 1.5 minutes, after subtraction of appropriate blanks, has been plotted against concentration of mAb in the complex. Upon analysis of the data, the anti-KIAA0746, anti-CD20 or anti-CD55 antibodies are categorized into different epitope groups depending on the epitope mapping results. The functional properties thereof are also compared.

Chinese hamster ovary (CHO) cell lines that express KIAA0746, CD20 or CD55 protein at the cell surface are developed and used to determine the specificity of the KIAA0746, CD20 or CD55 HuMAbs by flow cytometry. CHO cells are transfected with expression plasmids containing full length cDNA encoding a transmembrane forms of KIAA0746, CD20 or CD55 antigen or a variant thereof. The transfected proteins contained an epitope tag at the N-terminus are used for detection by an antibody specific for the epitope. Binding of an anti-KIAA0746, anti-CD20 or anti-CD55 MAb is assessed by incubating the transfected cells with each of the KIAA0746, CD20 or CD55 Abs at a concentration of 10 mu.g/ml. The cells are washed and binding is detected with a FITC-labeled anti-human IgG Ab. A murine anti-epitope tag Ab, followed by labeled anti-murine IgG, is used as the positive control. Non-specific human and murine Abs are used as negative controls. The obtained data is used to assess the specificity of the HuMAbs for the KIAA0746, CD20 or CD55 antigen target.

These antibodies and other antibodies specific to KIAA0746, CD20 or CD55 may be used in the afore-described anti-KIAA0746, anti-CD20 or anti-CD55 related therapies such as treatment of cancers wherein KIAA0746, CD20 or CD55 antigen is differentially expressed, such as ovarian cancer, lung cancer, breast cancer, kidney cancer, liver cancer, pancreatic cancer, prostate cancer, melanoma and hematological malignancies such as Multiple Myeloma, lymphoma, Non-Hodgkin's lymphoma, leukemia and T cell leukemia, involving the KIAA0746, CD20 or CD55 antigen, such as in the treatment of cancers and inflammatory or autoimmune diseases wherein such antibodies will e.g., prevent negative stimulation of T cell activity against desired target cancer cells or prevent the positive stimulation of T cell activity thereby eliciting a desired anti-autoimmune effect.

The invention has been described and embodiments provided relating to manufacture and selection of desired anti-KIAA0746, anti-CD20 or anti-CD55 antibodies for use as therapeutics and diagnostic methods wherein the disease or condition is associated with KIAA0746, CD20 or CD55 antigen. The descriptions given are intended to exemplify, but not limit, the scope of the invention. The invention is now further described by the claims which follow.

1-165. (canceled)

166. An isolated polypeptide having an amino acid sequence as set forth in HUMDAF_P30 (SEQ ID NO:56), or a fragment or variant thereof that possesses at least 95% sequence identity therewith.

167. The polypeptide of claim 166, wherein said fragment comprises an ectodomain of SEQ ID NO:56.

168. An isolated polypeptide having an amino acid sequence as set forth in SEQ ID NO:108, or a conjugate thereof.

169. The polypeptide of claim 166, comprising the amino acid sequence as set forth in SEQ ID NO:70 or an immunogenic fragment thereof.

170. The polypeptide of claim 166, comprising an amino acid sequence having at least 80% homology to the sequence as that set forth in SEQ ID NO: 196, or an immunogenic fragment thereof.

171. A monoclonal or polyclonal antibody or an antigen binding fragment thereof comprising an antigen binding site that binds specifically to any one of SEQ ID NOs:56, 108, 70, 196.

172. A pharmaceutical composition, comprising the antibody of claim 171 in a pharmaceutically acceptable carrier, adapted for treatment or prevention of disorder selected from cancer and immune related conditions.

173. The pharmaceutical composition of claim 172, wherein the antibody comprises an antigen binding site that binds specifically to any one of SEQ ID NOs:56, 108, 70, wherein the cancer is selected from the group consisting of tumors of breast, prostate, lung, colon, colorectal, ovary, spleen, kidney, bladder, head and neck, uterus, testicles, stomach, gastric, cervix, liver, bone, skin, pancreas, brain, and hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and wherein the cancer is non-metastatic, invasive or metastatic.

174. The pharmaceutical composition of claim 172, wherein said antibody comprises an antigen binding site that binds specifically to SEQ ID NO:196, wherein the cancer is selected from the group consisting of tumors of breast, prostate, colon, colorectal, ovary, spleen, kidney, bladder, head and neck, uterus, testicles, stomach, gastric, cervix, liver, bone, skin, pancreas, brain, and wherein the cancer is non-metastatic, invasive or metastatic.

175. The pharmaceutical composition of claim 172, wherein said antibody comprises an antigen binding site that binds specifically to any one of SEQ ID NOs:56, 108, 70, and wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

176. The pharmaceutical composition of claim 172, wherein said antibody comprises an antigen binding site that binds specifically to SEQ ID NO:196, and wherein the immune related condition is selected from the group consisting of systemic lupus erythematosus (SLE), lupus nephtirits and multiple sclerosis (MS), inflammatory bowel disease (IBD), ulcerative colitis, psoriasis, acute and chronic rejection of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

177. The antibody or fragment according to claim 171, wherein the antibody is a fully human antibody, a chimeric antibody, a humanized or primatized antibody.

178. The antibody or fragment according to claim 171, wherein the antibody is selected from the group consisting of Fab, Fab′, F(ab′)₂, F(ab′), F(ab), Fv or scFv fragment and minimal recognition unit.

179. The antibody or fragment according to claim 171, wherein the antibody is coupled to a detectable marker, selected from any one of radioisotope, a metal chelator, an enzyme, a fluorescent compound, a bioluminescent compound or a chemiluminescent compound, or to an effector moiety, selected from any one of an enzyme, a toxin, a therapeutic agent, or a chemotherapeutic agent.

180. A method of treating or preventing a disorder selected from any one of cancer and immune related condition in a patient, comprising administering to the patient an effective amount of the antibody or fragment according to claim 171 or the pharmaceutical composition according to claim 172.

181. The method of claim 180, used in combination therapy with other treatment methods known in the art selected from the group consisting of radiation therapy, antibody therapy, chemotherapy, surgery, or in combination therapy with conventional drugs, anti-cancer agents, immunosuppressants, cytotoxic drugs for cancer, chemotherapeutic agents, or in combination with therapeutic agents targeting other complement regulatory proteins (CRPs), or wherein the treatment is combined with a moiety useful for treating immune related conditions, selected from any one of a cytokine antibody, cytokine receptor antibody, drug, or another immunomodulatory agent.

182. The method of claim 180, wherein the cancer is selected from the group consisting of and tumors of breast, prostate, lung, colon, colorectal, ovary, spleen, kidney, bladder, head and neck, uterus, testicles, stomach, gastric, cervix, liver, bone, skin, pancreas, brain, and hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and wherein the cancer is non-metastatic, invasive or metastatic.

183. The method of claim 180, wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

184. An assay for detecting the presence of polypeptide comprising the amino acid sequence as set forth in any one of SEQ ID NOs:56, 108, 70, 196, or a fragment or variant thereof in a biological sample comprising contacting the sample with an antibody specific to any one of SEQ ID NOs:56, 108, 70, 196, and detecting the specific binding of said antibody to any one of SEQ ID NOs:56, 108, 70, 196, or a fragment or variant thereof in the sample.

185. A method for any one of screening for a disease, detecting a presence or a severity of a disease, diagnosing a disease, prognosis of a disease, monitoring disease progression or treatment efficacy or relapse of a disease, or selecting a therapy for a disease, comprising detecting in a subject or in a sample obtained from the subject a polypeptide having a sequence at least 95% homologous to an amino acid sequence selected from those set forth in any one of SEQ ID NOs:56, 108, 70, 196.

186. The method of claim 185, wherein detecting the polypeptide is performed in vivo or in vitro.

187. The method of claim 186 wherein the disease is selected from any one of cancer and immune related condition.

188. The method of claim 187, comprising detecting in a subject or in a sample obtained from the subject a polypeptide having a sequence at least 95% homologous to an amino acid sequence selected from those set forth in any one of SEQ ID NOs:56, 108, 70, wherein the cancer is selected from the group consisting of tumors of breast, prostate, lung, colon, colorectal, ovary, spleen, kidney, bladder, head and neck, uterus, testicles, stomach, gastric, cervix, liver, bone, skin, pancreas, brain, and hematological malignancies such as acute lymphocytic leukemia, chronic lymphocytic leukemia, acute myelogenous leukemia, chronic myelogenous leukemia, multiple myeloma, Hodgkin's lymphoma, Non-Hodgkin's lymphoma, and wherein the cancer is non-metastatic, invasive or metastatic.

189. The method of claim 187, comprising detecting in a subject or in a sample obtained from the subject a polypeptide having a sequence at least 95% homologous to an amino acid sequence set forth in SEQ ID NO:196, wherein the cancer is selected from the group consisting of tumors of breast, prostate, colon, colorectal, ovary, spleen, kidney, bladder, head and neck, uterus, testicles, stomach, gastric, cervix, liver, bone, skin, pancreas, brain, and wherein the cancer is non-metastatic, invasive or metastatic.

190. The method of claim 186, comprising detecting in a subject or in a sample obtained from the subject a polypeptide having a sequence at least 95% homologous to an amino acid sequence selected from those set forth in any one of SEQ ID NOs:56, 108, 70, wherein the immune related condition is selected from the group consisting of rheumatoid arthritis (RA), systemic lupus erythematosus (SLE), lupus nephtirits and multiple sclerosis (MS), inflammatory bowel disease (IBD), ulcerative colitis, psoriasis, acute and chronic rejection of organ transplantation and of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

191. The method of claim 186, comprising detecting in a subject or in a sample obtained from the subject a polypeptide having a sequence at least 95% homologous to an amino acid sequence set forth in SEQ ID NO:196, wherein the immune related condition is selected from the group consisting of, systemic lupus erythematosus (SLE), lupus nephtirits and multiple sclerosis (MS), inflammatory bowel disease (IBD), ulcerative colitis, psoriasis, acute and chronic rejection of allogeneic stem cell transplantation, autologous stem cell transplantation, bone marrow transplantation, treatment of Graft Versus Host Disease (GVHD), rejection in xenotransplantation, and disease states in which complement activation and deposition is involved in pathogenesis.

192. The method of claim 184 wherein the detection is conducted by immunoassay.


SEQUENCE LISTING (219 sequences)

SEQ ID NO: 1 - DNA, 4553 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 2 - DNA, 4393 bp, Homo sapiens
aaagagcgcagtggtggcggtggcggcggcaccacacaaaaccctcgggaaacatccagaacgtgccgcgaaccagccggcagggtggggcgcagcccggctgcaaacatgccagcaaggaggcagtcccaaccccgcaggtggacaggtggagaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 3 - DNA, 4815 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggccacagaaagtccagaatttctaccttgttcctagcaagaagagagatcagtgtttaaggttcaggccacctcttccatagatgttttactgcatctgccatgactaggagcctgggattgttggaagtccactcctctaagcgaggatgcaagaaggattcaagaactgaaggctagtagacaactccccagccaacatccgacatgcccttcagccagtagaggtagctgttgcatcaactagctccccagccttgtctgcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 4 - DNA, 3982 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 5 - DNA, 4469 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtgcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 6 - DNA, 3649 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactgga

SEQ ID NO: 7 - DNA, 3383 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcaggtaaagatttataaaaagcgaaagcaatatattaaaaaaaaaaaaagcaggg

SEQ ID NO: 8 - DNA, 4165 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaacatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 9 - DNA, 3627 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtatgggcaaaacatgtagctgagaaaaatggctacttgggccatgtcatccgcaaaggcctcaatgcctacctggaaggttcatggcatgaagctttgctgtattatgttttagcagcagaaactggaattgaagtgtcacagacaaatttagcacacatctgtgaggagaggccagacctggccaggagatacttgggtgttaactgtgtttggagatactataatttctctgtttttcaaatcgatgctccttcctttgcatatttgaagatgggagacctttactactatggccaccaaaaccagtcacaagacctggagttgtctgtgcagatgtacgcccaagccgccctggatggagactcccagggattttttaacctggccctgctaatcgaggaaggtacgataatcccacaccatatcttggatttcttggaaattgactcaactctccattctaataacatctccattctccaggaactgtacgaaagatctactttctgggaacctttctgctatccatattgatcgcctggactgtgcagtatttccagtctgtctcagacccccaggaacaaaatataacagagtagtgtaaaagtttgtcctctccagcaatctcatggcaaaaaggctcgaaagcacaactgtgcaaacacatttgaagacgtccatcatgtcacttccactgagatcccactggcgaaagcaagtcacatggggcgcggtggctcacacctgtaatctcagcaccttgggaggctgaagcaggcagatcacttaaggccagaagttcaagaccagcctgggcaaaatggagaaaccccatctcgactaaaaaatacaaaaattagccgggcatggtggtgcatgcctgtaatcc

SEQ ID NO: 10 - DNA, 3807 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcctccccagacactgccacgtccactgcaagtccagctgtgactccagctgcagatgcctctgaccaagaccagcccacagtaactaataacccggagccacgtgggtgaactgtgcactccagttctctccagatgagagagaatcttttcaacagctggtattgggaagctggggccagggcatgatcctgataaacaccttaaatgtcttgtcaactggatgcaaattttgcaattggtgtcattttttttaaagtcaaattacaaggaagtacccagatcaggcagtggtaataccaaaggtcatcaaacacatacaaggaacatcttgatcatagggcatgtggggaagtttactgggccatcacagacttttgttctagtgattgtatgtattaggagtcatagcatgccctacggcagatctggattcttatacactaagatgtgtcttaagaatcacagtgcgtgcttcatccctttattgaagaacagaaaattatgactactctacaaggtggataatattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaactgaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactactttatttacagtagtaaataaaattatttttatacaattgactaccagaaa

SEQ ID NO: 11 - DNA, 4552 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtgtaagactctgtcaaacgttgcatttgaagggaaagccatatcctatattctctgtgcctgagcattttttaagttgagttctttatttttaccagttgtacatgcatgtagttttaaaagtcatgtaatttgacaagacaaaattaaaacttttaattaaaaatttaaaaataatttgacaaaggctaaacaagaaatccttgcctacctcacttcatcccacccctagactctattttctgctccctagaatgaatcactttcaatcttttttgaaagatttattttattttatttattttttatttgtctctatttctaaataacatgcttataccactatttcttggtattttcatccgaggcattgtctaatgatgtcccactgcgaaggataaagatgtagttttctttgactctgccacctcccactactcagctcactcatacttcctgccatctttcatcttcccaataagtatatcattttggttacattagtatcagggtttacattattatgaccatgtaaatgctatttctaactgagccatgtagtatactctgattacttttcctttcttgcacaactttttcttttctatggattgctacttattttttattgtttatttgctaagctttctgtatacttatcattttctatgtatttgatctccaaattctcctccaggtgcctgaatttcctcttggtatgtccagacctatctaaatattatattaatttaaccttcttggtgacatccatcctggagtctttgttcaggacaatgctgtcatgctgagattaactgtcatcattatgggtatttactttccctccatctgtgtcttttttggttctcttctttgtcagacccctttctttctctttcttggtctgcacttaaattttggtggagcacatccaatagtaggttcctgaggtatggtgaatgggaggcacatttttgaggtcttgcagatctgaaaatgttttacaggagttgtcaaaccatgacccatagatgaaatgtagcttggtacctgttttgtatggctccaagagctaagaatgctttttacattcttgaggagttataaaacaaataaagaagaatatgcaacagagactatatgtggcccacaaagcttaagatatttactatcctgctctttacagaaaaagtttgtcgacctctgttttaggccaccctcacactgataccctggctctccaaaagattgttcttcataacacatttgggttcaaattcaacctgacctgaccttggtgataaacttgacttagactgacttgaagttttttatcattgttatttagtatatttgacatgaatattcatgttttgtggattttctgttatcctcttgttaatgatttctagcttaatttcactgtgatcagagaatatattctgaatgattccaatcctttgaaatttgtcaaagtgtgctttatgattggtatatggtcaattttggtatatgttttatggctttttgaaaagagtgtactttctgaacttgctggatcaacatctacatatatatcagttacatcaagtttgttaattacattattcagagcctccccatcctttctgatgtttctttttctgcttgttagttcagttactgaaagaagtatattaaaatctccaactatatttgtggacttacctacctctctttttagttctgtcaattttgcattatatatcttgaatctatgctattagatcacatagatttagaattgttttgtctttgtagttgatccctttatcatt

SEQ ID NO: 12 - DNA, 3368 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaagggtcaaggagtaaaaaagaacagacggcttgccttagagctgatgaagaaagcagcttccaagggattgcatcaggcagtcaatggcctgggatggtattaccacaaattcaagaaaaattacgccaaagcagcaaagtactggttaaaagcagaagaaatggggaacccagatgcgtcatacaatcttggagtcctgcatttggatggcatcttccctggagttcctggaaggaatcaaactttagctggtgaatatttccataaggctgcgcaaggtggacacatggaagggaccttgtggtgttctctctactatatcacaggcaacctggagacattccctagagatcctgagaaagctgttgtaaaaagtttgtcgacctctgttttaggccaccctcacactgataccctggctctccaaaagattgttcttcataacacatttgggttcaaattcaacctgacctgaccttggtgataaacttgacttagactgacttgaagttttttatcattgttatttagtatatttgacatgaatattcatgttttgtggattttctgttatcctcttgttaatgatttctagcttaatttcactgtgatcagagaatatattctgaatgattccaatcctttgaaatttgtcaaagtgtgctttatgattggtatatggtcaattttggtatatgttttatggctttttgaaaagagtgtactttctgaacttgctggatcaacatctacatatatatcagttacatcaagtttgttaattacattattcagagcctccccatcctttctgatgtttctttttctgcttgttagttcagttactgaaagaagtatattaaaatctccaactatatttgtggacttacctacctctctttttagttctgtcaattttgcattatatatcttgaatctatgctattagatcacatagatttagaattgttttgtctttgtagttgatccctttatcatt

SEQ ID NO: 13 - DNA, 2670 bp, Homo sapiens
agtggcggcggcggcgggtccggaggtggcggcaggtggccccgcgcgcggcggccggcccggccgggggcgggcgggaaggtggcgcctcgggcgggggccggtccctgcaccaggtgacctgttccggcccggatccgggcggcctcgccatgcagcggcgcggcgcggggctcgggtggccgcggcagcagcagcagcaacccccgccgctcgcggtcggcccccgggccgcagccatggtcccgagtggcggcgtcccccagggcctcggcggccgctctgcctgcgcgctgctcctgctctgctacctgaatgttgtaccatctttgggtaggcagacttccctgacgacatcagtgatacccaaagctgagcagagcgtggcttacaaagactttatttattttactgtctttgaaggaaacgttcgcaacgtttctgaagtctcggttgagtatttatgctctcagccttgtgttgtcaatttggaagcagttgtttcatctgagttcagaagtagcattcccgtgtacaaaaaaaggtggaagaatgagaaacatcttcacaccagcaggacacaaatagtacatgtgaaatttccaagcattatggtttacagagatgattatttcatcagacattccatctctgtatctgcagtgatagtacgcgcctggattactcacaaatacagtggcagagactggaatgttaaatgggaggaaaacttgctccatgctgtagcaaagaattataccctcctgcagaccatcccgccttttgaacgccctttcaaagatcatcaagtgtgccttgagtggaacatgggttatatttggaaccttcgggcaaacaggattccacagtgtcctctggaaaatgatgtggttgccctgcttggctttccttatgcctccagtggagaaaacacaggcattgtcaagaagttcccgaggtttcggaaccgagagctggaggccactcgacgccagaggatggattacccagtgtttactgtttcattgtggctttatttactccattattgcaaggccaacctctgtgggattctgtactttgttgactctaatgagatgtacggcacaccttctgtatttcttacggaagagggctatttgcatattcagatgcatcttgtcaaaggggaagaccttgctgtaaaaactaaattcatcatacctttgaaggagtggtttcgactggatatctcttttaacggaggccagatagtagtaaccactagcattggacaggatttgaaaagctaccacaatcagaccattagcttccgggaggatttccattataatgacacagctgggtacttcattattggagggagcaggtatgtggctggcattgaagggttttttggacccctgaagtactatcgccttcgcagtctgcaccccgcccagatttttaatcccctccttgagaagcaacttgctgaacaaatcaagttatattatgaaaggtgtgctgaggttcaagaaatagtatctgtgtatgcatctgcagcaaagcacgggggcgagagacaagaagcatgccacctccacaactcctacctggacctccagcgcaggtatgggagaccctcgatgtgcagagccttcccctgggagaaggagctgaaagacaaacaccccagcttgttccaggcattgctggagatggatctgctgaccgtgccaaggaaccaaaatgaatctgtatcagaaatcggtgggaagatatttgagaaggctgtaaagagactctctagcattgatggtcttcaccaaattagctctatcgtcccctttctgacggattccagctgctgtggataccataaagcatcctactaccttgcagtcttttatgagactggattaaatgttcctcgggatcagctgcagggcatgttgtatagtttggttggaggccaggggagtgagaggctgtcttcaatgaatcttgggtataaacactaccagggtattgacaactaccccctggactgggaactgtcgtatgcctactacagcaacattgccaccaagacaccccttgaccagcacacactgcaaggagatcaggcatatgttgaaacaattagactaaaagatgatgaaatactcaaggtacaaaccaaagaagatggagatgtctttatgtggttgaagcatgaagctacccgaggcaatgcagcagctcagcaacgattggcccagatgctgttctgggggcagcaaggtgtggccaagaatcccgaagcagcaattgagtggtacgccaagggcgccctggagacggaggatcctgcgttaatctatgactatgccattgtgctattcaaggtaagaatcacttaattcgtgctgaaattgtgcaattcttatcagactcctgagcagtttcctgtgggcgggctggcaaacagggagtcgataactgaagaaatttagtatatgcttctgtcactcttgatggaaaagaatgttggctaaaaaggcagagtcagaagcagttccctgttctcatttttaatcattagtcttatgtttggcacctggattctctgtgctttcgcaagaaccacggcattctggaggagacacccgtattttattgattggcgctcagccgccagcccccaca

SEQ ID NO: 14 - protein, 979 aa, Homo sapiens
MVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEIFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 15 - protein, 702 aa, Homo sapiens
IFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 16 - protein, 1097 aa, Homo sapiens
MAPRPKKQPDKNPLHGRELNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 17 - protein, 1029 aa, Homo sapiens
LCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 18 - protein, 1103 aa, Homo sapiens [sequence omitted]

SEQ ID NO: 19 - protein, 1129 aa, Homo sapiens
KSAVVAVAAAPHKTLGKHPERAANQPAGWGAARLQTCQQGGSPNPAGGQVENVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 20 - protein, 887 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWPQKVQNFYLVPSKKRDQCLRFRPPLP

SEQ ID NO: 21 - protein, 1075 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 22 - protein, 1098 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAIIYFLGTFLLSILIAWTVQYFQSVSASDPPPRPSQASPDTATSTASPAVTPAADASDQDQPTVTNNPEPRG

SEQ ID NO: 23 - protein, 977 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTVRKVLEPQ

SEQ ID NO: 24 - protein, 792 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQHI

SEQ ID NO: 25 - protein, 1010 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERSTFWEPFCYPY

SEQ ID NO: 26 - protein, 839 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGLPRHCHVHCKSSCDSSCRCL

SEQ ID NO: 27 - protein, 833 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVV

SEQ ID NO: 28 - protein, 867 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVKSLSTSVLGHPHTDTLALQKIVLHNTFGFKFNLT

SEQ ID NO: 29 - protein, 714 aa, Homo sapiens
MVPSGGVPQGLGGRSACALLLLCYLNVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKVRIT

SEQ ID NO: 30 - protein, 850 aa, Homo sapiens
MDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 31 - DNA, 1263 bp, Homo sapiens
gtgttaaaccaaagtaattggagcgaagcccaaggtagcagaagctactgatttcctgtcacctgatgtctatcagcgatttcatcttcaggcctggactacaccactcaccctcccagtgtgcttgagaaacaaactgcacccactgaactccgcagctagcatccaaatcagcccttgagatttgaggccttggagactcagatcctgaacaagagagaacaaaatctctactttgatggaacttccattctgtggggaagagactgacaataagcaattaaataaataagaactcagcagtaggccttgcctcagatccaaggtcactcggaagaggccatgtctaccctcaatgacactcatggaggaaatgctgagagaagcattcagatgcatgacacaaggtaagactgccaaaaatcttgttcttgctctcctcattttgttatttgttttatttttaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgcctgaatgtgagaaaaggaagatgagcaatagtcatcatcacttcctgtaacagccaatgttttcatggagtgcctgtgccattcaggtcaagtatttccttctgcatcagttcactcttcagagggcatcagagtcatttatgtcactgtgaaccccaaagggcagttccacaagttaaaaacaaagaaaaactagaaataaaacttttaaatttatggtatgagtattaattgatgaggaaatttgagttctgtctctttggtcttactatattcctagtcacagatccccagatgattgagtaaaaggcatgaatttagtgtcactgagcctgaataaaggaggaatatgacagctgaaaaatgaatacaactgataaaaatgggtggatggttgtgtgaaagttgctgaaagtgtaggcttctttctgaccagttatcaatgttaaaaagtgatctccctctctcctctatctcctgtcttgcccaccccctctcca

SEQ ID NO: 32 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 33 - protein, 109 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMPECEKRKMSNSHHHFL

SEQ ID NO: 34 - DNA, 2798 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcaccaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 35 - DNA, 2876 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcacataagtccactaatgtacattccccagtgactaatggtctcaagagtacacaaagattcccttctgctcatattacagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 36 - DNA, 2916 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcaccaacacggagtacacctgtttccaggacaaccaagcattttcatgaaacaaccccaaataaaggaagtggaaccacttcaggtactacccgtcttctatctggttctcgtcctgtcacccaggctggtatgcggtggtgtgatcgtagctcactgcagtctcgaactcctgggttcaagcgatccttccacttcagcctcccaagtagctggtactacagggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 37 - DNA, 2087 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcacataagtccactaatgtacattccccagtgactaatggtctcaagagtacacaaagattcccttctgctcatattacagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttca

SEQ ID NO: 38 - DNA, 2420 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtgggagtccagccgtgtggagcacacgatgctgcaaacttgcatgtcatctctttcaggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 39 - DNA, 2291 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatctcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 40 - DNA, 2217 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcaccaacacggagtacacctgtttccaggacaaccaagcattttcatgaaacaaccccaaataaaggaagtggaaccacttcaggtactacccgtcttctatctggttctcgtcctgtcacccaggctggtatgcggtggtgtgatcgtagctcactgcagtctcgaactcctgggttcaagcgatccttccacttcagcctcccaagtagctggtactacaggtgtgtgccacgacacccggctaagtttttgaaatttattttttgtagagacaggattttcctatgttgcccaggctggtttcaaactcctggccgtaagcgatttttccggcctcccaaaacgttgcgattataagtgtgagccactgcacctggccccacattttctttatccatttgtacattgatggacacttaagatgattccatatctttgctattgtgaatagtgcttcaataaatatgtgaatgcacata

SEQ ID NO: 41 - DNA, 2193 bp, Homo sapiens
gcagtggagagagtgagtcccagagggtgttgccagcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcacataagtccactaatgtacattccccagtgactaatggtctcaagagtacacaaagattcccttctgctcatattacagcaacacggagtacacctgtttccaggacaaccaagcattttcatgaaacaaccccaaataaaggaagtggaaccacttcaggtactacccgtcttctatctgctcttataatgcacatgagagcaacaaagtactcaatgttgtgtttgaccatttaagtgtgactggtggtacctcagaaataagactttctggtaaattataaaagggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttca

SEQ ID NO: 42 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 43 - protein, 293 aa, Homo sapiens
MLQTCMSSLSGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKT

SEQ ID NO: 44 - protein, 419 aa, Homo sapiens
RPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGAETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHITATRSTPVSRTTKHFHETTPNKGSGTTSGTTRLLSALIMHMRATKYSMLCLTI

SEQ ID NO: 45 - protein, 440 aa, Homo sapiens
MTVARPSVPAALPLLGELPRLLLLVLLCLPAVWGDCGLPPDVPNAQPALEGRTSFPEDTVITYKCEESFVKIPGEKDSVTCLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 46 - protein, 55 aa, Homo sapiens
KVPPTVQKPTTVNVPTTEVSPTSQKTTTKTTTPNAQATRSTPVSRTTKHFHETTP

SEQ ID NO: 47 - protein, 444 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRCVPRHPAKFLKFIFCRDRIFLCCPGWFQTPGRKRFFRPPKTLRL

SEQ ID NO: 48 - protein, 316 aa, Homo sapiens
SVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQKFTTAKVAFTQSPSAAPTRSTPVSRTTKHFHETTPNKGSGTTSGTTRLLSGHTCFTLTGLLGTLVTMGLLT

SEQ ID NO: 49 - protein, 265 aa, Homo sapiens
RPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGQLTLFRFTEYGSNVLWWKYELDQDCRIKWSLIYCGQGFSY

SEQ ID NO: 50 - protein, 422 aa, Homo sapiens
RPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGAETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHITATRSTPVSRTTKHFHETTPNKGSGTTSGTTRLLSGHTCFTLTGLLGTLVTMGLLT

SEQ ID NO: 51 - protein, 525 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAPTRSTPVSRTTKHFHETTPNKGSGTTSGTTRLLSGHTCFTLTGLLGTLVTMGLLT

SEQ ID NO: 52 - protein, 551 aa, Homo sapiens [sequence omitted]

SEQ ID NO: 53 - protein, 584 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAPTRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 54 - protein, 399 aa, Homo sapiens
MTVARPSVPAALPLLGELPRLLLLVLLCLPAVWESSRVEHTMLQTCMSSLSGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 55 - protein, 356 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNLGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 56 - protein, 588 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAPTRSTPVSRTTKHFHETTPNKGSGTTSGTTRLLSGSRPVTQAGMRWCDRSSLQSRTPGFKRSFHFSLPSSWYYRCVPRHPAKFLKFIFCRDRIFLCCPGWFQTPGRKRFFRPPKTLRL

SEQ ID NO: 57 - protein, 548 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHITATRSTPVSRTTKHFHETTPNKGSGTTSGTTRLLSALIMHMRATKYSMLCLTI

SEQ ID NO: 58 - DNA, 20 bp, Artificial Sequence
  Synthetic oligonucleotide
gctacacaggccacactaac

SEQ ID NO: 59 - DNA, 20 bp, Artificial Sequence
  Synthetic oligonucleotide
ctcttctttggctaagtcag

SEQ ID NO: 60 - DNA, 34 bp, Artificial Sequence
  Synthetic oligonucleotide
cctagctagccaccatgaccgtcgcgcggccgag

SEQ ID NO: 61 - DNA, 20 bp, Artificial Sequence
  Synthetic oligonucleotide
gttagtgtggcctgtgtagc

SEQ ID NO: 62 - DNA, 20 bp, Artificial Sequence
  Synthetic oligonucleotide
agtggcccaccacctgaatg

SEQ ID NO: 63 - DNA, 62 bp, Artificial Sequence
  Synthetic oligonucleotide
gcgaccggtttacttgtcgtcatcgtctttgtagtcagtcagcaagcccatggttactagcg

SEQ ID NO: 64 - DNA, 62 bp, Artificial Sequence
  Synthetic oligonucleotide
cgcaccggtttacttgtcgtcatcgtctttgtagtctgaagtggttccacttcctttatttg

SEQ ID NO: 65 - DNA, 20 bp, Artificial Sequence
  Synthetic oligonucleotide
tgggaccttggaagttagag

SEQ ID NO: 66 - DNA, 1170 bp, Artificial Sequence
  Synthetic oligonucleotide
atgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcggtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgactgactacaaagacgatgacgacaagtaa

SEQ ID NO: 67 - protein, 389 aa, Artificial Sequence
  Synthetic peptide
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLTDYKDDDDK

SEQ ID NO: 68 - DNA, 1596 bp, Artificial Sequence
  Synthetic oligonucleotide
atgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgcaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcacataagtccactaatgtacattccccagtgactaatggtctcaagagtacacaaagattcccttctgctcatattacagcaacacggagtacacctgtttccaggacaaccaagcattttcatgaaacaaccccaaataaaggaagtggaaccacttcagactacaaagacgatgacgacaagtaa

SEQ ID NO: 69 - protein, 531 aa, Artificial Sequence
  Synthetic peptide
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHITATRSTPVSRTTKHFHETTPNKGSGTTSDYKDDDDK

SEQ ID NO: 70 - protein, 20 aa, Artificial Sequence
  Synthetic peptide
TETPSVLQKHTTENVSATRT

SEQ ID NO: 71 - DNA, 432 bp, Artificial Sequence
  Synthetic oligonucleotide
gtacagagactccatcagttcttcaaaaacacaccacagaaaatgtttcagctacaagaaccccaccaactcctcagaaacccaccacagtaaatgtcccagctacaatagtcacaccaacacctcagaaacccaccacaataaatgttccagctacaggagtctcatcaacacctcaaagacacaccatagtaaatgtttcagctacaggaaccctaccaactcttcagaaacccaccagagcaaatgattcagccaccaaatccccagcagcagctcagacatctttcatatcaaaaaccctatctacaaagaccccttctgcagctcagaatcccatgatgacaaatgcttctgctacacaggccacactaacagcccaaagattcaccacagcaaaagttgcatttacgcagagtccttcagcagcac

SEQ ID NO: 72 - DNA, 957 bp, Artificial Sequence
  Synthetic oligonucleotide
ggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggta

SEQ ID NO: 73 - DNA, 354 bp, Artificial Sequence
  Synthetic oligonucleotide
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgcctgaatgtgagaaaaggaagatgagcaatagtcatcatcacttcctggactacaaagacgatgacgacaagtaa

SEQ ID NO: 74 - protein, 117 aa, Artificial Sequence
  Synthetic peptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMPECEKRKMSNSHHHFLDYKDDDDK

SEQ ID NO: 75 - DNA, 843 bp, Artificial Sequence
  Synthetic oligonucleotide
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctggggggtcttctgatgatcccagcagggatctatgcacccatctgtgtgactgtgtggtaccctctctggggaggcattatgcctgaatgtgagaaaaggaagatgagcaatagtcatcatcacttcctggactacaaagacgatgacgacaagtaa

SEQ ID NO: 76 - protein, 280 aa, Artificial Sequence
  Synthetic peptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGGLLMIPAGIYAPICVTVWYPLWGGIMPECEKRKMSNSHHHFLDYKDDDDK

SEQ ID NO: 77 - protein, 19 aa, Artificial Sequence
  Synthetic peptide
MTTPRNSVNGTFPAEPMKG

SEQ ID NO: 78 - protein, 17 aa, Artificial Sequence
  Synthetic peptide
MPECEKRKMSNSHHHFL

SEQ ID NO: 79 - DNA, 20 bp, Artificial Sequence
  Synthetic oligonucleotide
cctttctgacggattccagc

SEQ ID NO: 80 - DNA, 24 bp, Artificial Sequence
  Synthetic oligonucleotide
tccaaccaaactatacaacatgcc

SEQ ID NO: 81 - DNA, 125 bp, Artificial Sequence
  Synthetic oligonucleotide
cctttctgacggattccagctgctgtggataccataaagcatcctactaccttgcagtcttttatgagactggattaaatgttcctcgggatcagctgcagggcatgttgtatagtttggttgga

SEQ ID NO: 82 - DNA, 19 bp, Artificial Sequence
  Synthetic oligonucleotide
tgacggattccagctgctg

SEQ ID NO: 83 - DNA, 19 bp, Artificial Sequence
  Synthetic oligonucleotide
cctggcctccaaccaaact

SEQ ID NO: 84 - DNA, 126 bp, Artificial Sequence
  Synthetic oligonucleotide
tgacggattccagctgctgtggataccataaagcatcctactaccttgcagtcttttatgagactggattaaatgttcctcgggatcagctgcagggcatgttgtatagtttggttggaggccagg

SEQ ID NO: 85 - DNA, 24 bp, Artificial Sequence
  Synthetic oligonucleotide
cccatctgtgtgactgtgtggtac

SEQ ID NO: 86 - DNA, 26 bp, Artificial Sequence
  Synthetic oligonucleotide
tctgatgccctctgaagagtgaactg

SEQ ID NO: 87 - DNA, 179 bp, Artificial Sequence
  Synthetic oligonucleotide
cccatctgtgtgactgtgtggtaccctctctggggaggcattatgcctgaatgtgagaaaaggaagatgagcaatagtcatcatcacttcctgtaacagccaatgttttcatggagtgcctgtgccattcaggtcaagtatttccttctgcatcagttcactcttcagagggcatcaga

SEQ ID NO: 88 - DNA, 20 bp, Artificial Sequence
  Synthetic oligonucleotide
ggcccaccacctgaatgcag

SEQ ID NO: 89 - DNA, 26 bp, Artificial Sequence
  Synthetic oligonucleotide
tttctgaggagttggtggggttcttg

SEQ ID NO: 90 - DNA, 228 bp, Artificial Sequence
  Synthetic oligonucleotide
ggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaaggtacagagactccatcagttcttcaaaaacacaccacagaaaatgtttcagctacaagaaccccaccaactcctcagaaa

SEQ ID NO: 91 - DNA, 23 bp, Artificial Sequence
  Synthetic oligonucleotide
aggtgtactccgtgttgcttgag

SEQ ID NO: 92 - DNA, 168 bp, Artificial Sequence
  Synthetic oligonucleotide
agtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacct

SEQ ID NO: 93 - protein, 991 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRL

SEQ ID NO: 94 - protein, 1033 aa, Homo sapiens
KHPERAANQPAGWGAARLQTCQQGGSPNPAGGQVENVVPSLGRQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRL

SEQ ID NO: 95 - protein, 855 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWPQKVQNFYLVPSKKRDQCLRFRPPLP

SEQ ID NO: 96 - protein, 963 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRL

SEQ ID NO: 97 - protein, 990 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLR

SEQ ID NO: 98 - protein, 945 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTVRKVLEPQ

SEQ ID NO: 99 - protein, 760 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQHI

SEQ ID NO: 100 - protein, 978 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERSTFWEPFCYPY

SEQ ID NO: 101 - protein, 807 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGLPRHCHVHCKSSCDSSCRCL

SEQ ID NO: 102 - protein, 801 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVV

SEQ ID NO: 103 - protein, 835 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVKSLSTSVLGHPHTDTLALQKIVLHNTFGFKFNLT

SEQ ID NO: 104 - protein, 682 aa, Homo sapiens
RQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKVRIT

SEQ ID NO: 105 - protein, 750 aa, Homo sapiens
NLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRL

SEQ ID NO: 106 - protein, 23 aa, Artificial Sequence
  Synthetic peptide
PLWGGIMPECEKRKMSNSHHHFL

SEQ ID NO: 107 - protein, 63 aa, Artificial Sequence
  Synthetic peptide
MTTPRNSVNGTFPAEPMKGPIAMQSGPKPLFRRMSSLVGPTQSFFMRESKTLGAVQIMNGLFH

SEQ ID NO: 108 - protein, 463 aa, Artificial Sequence
  Synthetic peptide
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAPTRSTPVSRTTKHFHETTPNKGSGTTS

SEQ ID NO: 109 - protein, 489 aa, Artificial Sequence
  Synthetic peptide
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHITATRSTPVSRTTKHFHETTPNKGSGTTS

SEQ ID NO: 110 - protein, 336 aa, Artificial Sequence
  Synthetic peptide
SRVEHTMLQTCMSSLSGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 111 - protein, 294 aa, Artificial Sequence
  Synthetic peptide
DCGLPPDVPNAQPALEGRTSFPEDTVITYKCEESFVKIPGEKDSVICLKGSQWSDIEEFCNLGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 112 - protein, 489 aa, Artificial Sequence; identical to SEQ ID NO: 109
  Synthetic peptide

SEQ ID NO: 113 - DNA, 35 bp, Artificial Sequence
  Synthetic oligonucleotide
ctagctagccaccatgacaacacccagaaattcag

SEQ ID NO: 114 - DNA, 60 bp, Artificial Sequence
  Synthetic oligonucleotide
ccgaattcttattacttgtcgtcatcgtctttgtagtccaggaagtgatgatgactattg

SEQ ID NO: 115 - DNA, 23 bp, Artificial Sequence
  Synthetic oligonucleotide
aatacgactcactatagggagac

SEQ ID NO: 116 - DNA, 27 bp, Artificial Sequence
  Synthetic oligonucleotide
aaggatcccatctggatgtgcaggtag

SEQ ID NO: 117 - DNA, 29 bp, Artificial Sequence
  Synthetic oligonucleotide
tattcgaacacctggtgaagggcgaggac

SEQ ID NO: 118 - DNA, 30 bp, Artificial Sequence
  Synthetic oligonucleotide
taggatccaatcttgccgccgatctcggac

SEQ ID NO: 119 - DNA, 27 bp, Artificial Sequence
  Synthetic oligonucleotide
taggatccgcccatttcctcggccttc

SEQ ID NO: 120 - DNA, 26 bp, Artificial Sequence
  Synthetic oligonucleotide
tattcgaaaaccccgacgcctcctac

SEQ ID NO: 121 - DNA, 20 bp, Artificial Sequence
  Synthetic oligonucleotide
cagcacgtccttgatcttgg

SEQ ID NO: 122 - DNA, 1647 bp, Artificial Sequence
  Synthetic oligonucleotide
atgaacagcttcagcaccagcgccttcggccctgtggcctttagcctgggcctgctgctggtgctgcctgccgcctttcctgctcctgtgcctcctcagacaagcttgaccacctccgtgatccccaaggccgagcagagcgtggcctacaaggacttcatctacttcaccgtgttcgagggcaacgtgcggaacgtgtccgaggtgtccgtggagtacctgtgcagccagccctgcgtggtgaacctggaagccgtggtgtccagcgagttccggtccagcatccccgtgtacaagaagcggtggaagaacgagaagcacctgcacaccagccggacccagatcgtgcacgtgaagttccccagcatcatggtgtaccgggacgactacttcatccggcacagcatcagcgtgtccgccgtgatcgtgcgggcctggatcacccacaagtacagcggcagggactggaacgtgaagtgggaggaaaacctgctgcacgccgtggccaagaactacaccctgctgcagaccatcccccccttcgagcggcccttcaaggaccaccaggtctgcctggaatggaacatgggctacatctggaacctgcgggccaacagaatcccccagtgccccctggaaaacgacgtggtggccctgctgggctttccttacgccagcagcggcgagaacaccggcatcgtgaagaagttcccccggttccggaacagagagctggaagccaccaggcggcagaggatggactaccccgtgttcaccgtgtccctgtggctgtatctgctgcactactgcaaggccaacctgtgcggcatcctgtacttcgtggacagcaacgagatgtacggcacccccagcgtgtttctgaccgaggaaggctacctgcacatccagatgggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaagtga

SEQ ID NO: 123 - DNA, 1446 bp, Artificial Sequence
  Synthetic oligonucleotide
atgaacagcttcagcaccagcgccttcggccccgtggccttcagcctgggcctgctgctggtgctgcctgccgccttccctgcccccgtgccccccttcgaacacctggtgaagggcgaggacctggccgtgaaaaccaagttcatcatccccctgaaagagtggttccggctggacatcagcttcaacggcggccagatcgtggtgaccacaagcatcggccaggacctgaagagctaccacaaccagaccatcagcttccgggaggacttccactacaacgacaccgccggctacttcatcatcggcggcagcagatacgtggccggcatcgagggctttttcggccccctgaagtactaccggctgagatctctgcaccccgcccagattttcaaccccctgctggaaaagcagctggccgaacagatcaagctgtactacgagagatgcgccgaggtgcaggaaattgtctccgtctacgcctctgccgccaagcacggcggcgagagacaggaagcctgccacctgcacaactcctacctggacctgcagcggagatacggcagacccagcatgtgccgggccttcccttgggagaaagagctgaaggacaagcaccccagcctgttccaggctctgctggaaatggacctgctgaccgtgccccggaaccagaacgagagcgtgtccgagatcggcggcaagattggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaagtga

SEQ ID NO: 124 - DNA, 1602 bp, Artificial Sequence
  Synthetic oligonucleotide
atgaacagcttcagcaccagcgccttcggccccgtggccttcagcctgggcctgctgctggtgctgcctgccgccttccctgcccccgtgccccccttcgaaaaggccgtgaagcggctgtccagcatcgacggcctgcaccagatcagcagcatcgtgccctttctgacagactccagctgctgcggctaccacaaggccagctactatctggccgtgttctacgagacaggcctgaacgtgcccagggaccagctgcagggcatgctgtacagcctggtgggcggccagggcagcgagagactgagcagcatgaacctgggctacaagcactaccagggcatcgacaactaccccctggactgggagctgtcctacgcctactacagcaatatcgccaccaagacccccctggaccagcacacactgcagggcgaccaggcctacgtggagacaatccggctgaaggacgacgagatcctgaaggtgcagaccaaggaagatggcgacgtgttcatgtggctgaagcacgaggccaccagaggaaatgccgctgcccagcagagactggcccagatgctgttctggggacagcagggcgtggccaaaaaccctgaggccgccatcgagtggtatgccaagggcgccctggaaacagaggaccccgccctgatctacgactacgccatcgtgctgttcaagggccagggcgtgaagaagaaccggcggctggccctggaactgatgaagaaggccgccagcaagggactgcaccaggccgtgaatggcctgggctggtactaccacaagttcaagaagaactacgccaaggccgccaagtactggctgaaggccgaggaaatgggcggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaagtga

SEQ ID NO: 125 - DNA, 1611 bp, Artificial Sequence
  Synthetic oligonucleotide
atgaacagcttcagcaccagcgccttcggccccgtggccttcagcctgggcctgctgctggtgctgcctgccgccttccctgcccccgtgccccccttcgaaaaccccgacgcctcctacaatctgggcgtgctgcacctggatggcatcttccccggcgtgcccggcagaaatcagaccctggccggcgagtactttcacaaggccgcccaggggggccacatggaaggcaccctgtggtgcagcctgtactacatcaccggcaacctggaaaccttccccagggaccccgagaaggccgtggtgtgggccaagcacgtggccgagaagaacggctacctgggccacgtgatcaggaagggcctgaacgcctacctggaaggcagctggcacgaggccctgctgtactatgtgctggccgccgagacaggcatcgaggtgtcccagaccaacctggcccacatctgcgaggaacggcccgacctggccagacgctacctgggagtgaactgcgtgtggcggtactacaacttcagcgtgttccagatcgacgcccccagcttcgcctacctgaagatgggcgacctgtactactacggccaccagaaccagtcccaggatctggaactgtccgtgcagatgtacgcccaggccgctctggatggcgacagccagggcttcttcaacctggctctgctgatcgaagagggcaccatcatccctcaccacatcctggactttctggaaatcgacagcaccctgcacagcaacaacatcagcatcctgcaggaactgtacgagcgctgctggtcccacagcaacgaagagagcttcagcccctgcagcctggcctggctgtacctgcacctgaggctgggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaagtga

SEQ ID NO: 126 - protein, 548 aa, Artificial Sequence
  Synthetic peptide
MNSFSTSAFGPVAFSLGLLLVLPAAFPAPVPPQTSLTTSVIPKAEQSVAYKDFIYFTVFEGNVRNVSEVSVEYLCSQPCVVNLEAVVSSEFRSSIPVYKKRWKNEKHLHTSRTQIVHVKFPSIMVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 127 - protein, 481 aa, Artificial Sequence
  Synthetic peptide
MNSFSTSAFGPVAFSLGLLLVLPAAFPAPVPPFEHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 128 - protein, 533 aa, Artificial Sequence
  Synthetic peptide
MNSFSTSAFGPVAFSLGLLLVLPAAFPAPVPPFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 129 - protein, 536 aa, Artificial Sequence
  Synthetic peptide
MNSFSTSAFGPVAFSLGLLLVLPAAFPAPVPPFENPDASYNLGVLHLDGIFPGVPGRNQTLAGEYFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 130 - DNA, 3096 bp, Artificial Sequence
  Synthetic oligonucleotide
ggtaccgctagcgccaccatgaacagcttcagcaccagcgccttcggccctgtggcctttagcctgggcctgctgctggtgctgcctgccgcctttcctgctcctgtgcctcctcagacaagcttgaccacctccgtgatccccaaggccgagcagagcgtggcctacaaggacttcatctacttcaccgtgttcgagggcaacgtgcggaacgtgtccgaggtgtccgtggagtacctgtgcagccagccctgcgtggtgaacctggaagccgtggtgtccagcgagttccggtccagcatccccgtgtacaagaagcggtggaagaacgagaagcacctgcacaccagccggacccagatcgtgcacgtgaagttccccagcatcatggtgtaccgggacgactacttcatccggcacagcatcagcgtgtccgccgtgatcgtgcgggcctggatcacccacaagtacagcggcagggactggaacgtgaagtgggaggaaaacctgctgcacgccgtggccaagaactacaccctgctgcagaccatcccccccttcgagcggcccttcaaggaccaccaggtctgcctggaatggaacatgggctacatctggaacctgcgggccaacagaatcccccagtgccccctggaaaacgacgtggtggccctgctgggctttccttacgccagcagcggcgagaacaccggcatcgtgaagaagttcccccggttccggaacagagagctggaagccaccaggcggcagaggatggactaccccgtgttcaccgtgtccctgtggctgtatctgctgcactactgcaaggccaacctgtgcggcatcctgtacttcgtggacagcaacgagatgtacggcacccccagcgtgtttctgaccgaggaaggctacctgcacatccagatgcacctggtgaagggcgaggacctggccgtgaaaaccaagttcatcatccccctgaaagagtggttccggctggacatcagcttcaacggcggccagatcgtggtgaccacaagcatcggccaggacctgaagagctaccacaaccagaccatcagcttccgggaggacttccactacaacgacaccgccggctacttcatcatcggcggcagcagatacgtggccggcatcgagggctttttcggccccctgaagtactaccggctgagatctctgcaccccgcccagattttcaaccccctgctggaaaagcagctggccgaacagatcaagctgtactacgagagatgcgccgaggtgcaggaaattgtctccgtctacgcctctgccgccaagcacggcggcgagagacaggaagcctgccacctgcacaactcctacctggacctgcagcggagatacggcagacccagcatgtgccgggccttcccttgggagaaagagctgaaggacaagcaccccagcctgttccaggctctgctggaaatggacctgctgaccgtgccccggaaccagaacgagagcgtgtccgagatcggcggcaagattttcgaaaaggccgtgaagcggctgtccagcatcgacggcctgcaccagatcagcagcatcgtgccctttctgacagactccagctgctgcggctaccacaaggccagctactatctggccgtgttctacgagacaggcctgaacgtgcccagggaccagctgcagggcatgctgtacagcctggtgggcggccagggcagcgagagactgagcagcatgaacctgggctacaagcactaccagggcatcgacaactaccccctggactgggagctgtcctacgcctactacagcaatatcgccaccaagacccccctggaccagcacacactgcagggcgaccaggcctacgtggagacaatccggctgaaggacgacgagatcctgaaggtgcagaccaaggaagatggcgacgtgttcatgtggctgaagcacgaggccaccagaggaaatgccgctgcccagcagagactggcccagatgctgttctggggacagcagggcgtggccaaaaaccctgaggccgccatcgagtggtatgccaagggcgccctggaaacagaggaccccgccctgatctacgactacgccatcgtgctgttcaagggccagggcgtgaagaagaaccggcggctggccctggaactgatgaagaaggccgccagcaagggactgcaccaggccgtgaatggcctgggctggtactaccacaagttcaagaagaactacgccaaggccgccaagtactggctgaaggccgaggaaatgggcaaccccgacgcctcctacaatctgggcgtgctgcacctggatggcatcttccccggcgtgcccggcagaaatcagaccctggccggcgagtactttcacaaggccgcccaggggggccacatggaaggcaccctgtggtgcagcctgtactacatcaccggcaacctggaaaccttccccagggaccccgagaaggccgtggtgtgggccaagcacgtggccgagaagaacggctacctgggccacgtgatcaggaagggcctgaacgcctacctggaaggcagctggcacgaggccctgctgtactatgtgctggccgccgagacaggcatcgaggtgtcccagaccaacctggcccacatctgcgaggaacggcccgacctggccagacgctacctgggagtgaactgcgtgtggcggtactacaacttcagcgtgttccagatcgacgcccccagcttcgcctacctgaagatgggcgacctgtactactacggccaccagaaccagtcccaggatctggaactgtccgtgcagatgtacgcccaggccgctctggatggcgacagccagggcttcttcaacctggctctgctgatcgaagagggcaccatcatccctcaccacatcctggactttctggaaatcgacagcaccctgcacagcaacaacatcagcatcctgcaggaactgtacgagcgctgctggtcccacagcaacgaagagagcttcagcccctgcagcctggcctggctgtacctgcacctgaggctgggatccgagctc

SEQ ID NO: 131 - DNA, 3801 bp, Artificial Sequence
  Synthetic oligonucleotide
atgaacagcttcagcaccagcgccttcggccctgtggcctttagcctgggcctgctgctggtgctgcctgccgcctttcctgctcctgtgcctcctcagacaagcttgaccacctccgtgatccccaaggccgagcagagcgtggcctacaaggacttcatctacttcaccgtgttcgagggcaacgtgcggaacgtgtccgaggtgtccgtggagtacctgtgcagccagccctgcgtggtgaacctggaagccgtggtgtccagcgagttccggtccagcatccccgtgtacaagaagcggtggaagaacgagaagcacctgcacaccagccggacccagatcgtgcacgtgaagttccccagcatcatggtgtaccgggacgactacttcatccggcacagcatcagcgtgtccgccgtgatcgtgcgggcctggatcacccacaagtacagcggcagggactggaacgtgaagtgggaggaaaacctgctgcacgccgtggccaagaactacaccctgctgcagaccatcccccccttcgagcggcccttcaaggaccaccaggtctgcctggaatggaacatgggctacatctggaacctgcgggccaacagaatcccccagtgccccctggaaaacgacgtggtggccctgctgggctttccttacgccagcagcggcgagaacaccggcatcgtgaagaagttcccccggttccggaacagagagctggaagccaccaggcggcagaggatggactaccccgtgttcaccgtgtccctgtggctgtatctgctgcactactgcaaggccaacctgtgcggcatcctgtacttcgtggacagcaacgagatgtacggcacccccagcgtgtttctgaccgaggaaggctacctgcacatccagatgcacctggtgaagggcgaggacctggccgtgaaaaccaagttcatcatccccctgaaagagtggttccggctggacatcagcttcaacggcggccagatcgtggtgaccacaagcatcggccaggacctgaagagctaccacaaccagaccatcagcttccgggaggacttccactacaacgacaccgccggctacttcatcatcggcggcagcagatacgtggccggcatcgagggctttttcggccccctgaagtactaccggctgagatctctgcaccccgcccagattttcaaccccctgctggaaaagcagctggccgaacagatcaagctgtactacgagagatgcgccgaggtgcaggaaattgtctccgtctacgcctctgccgccaagcacggcggcgagagacaggaagcctgccacctgcacaactcctacctggacctgcagcggagatacggcagacccagcatgtgccgggccttcccttgggagaaagagctgaaggacaagcaccccagcctgttccaggctctgctggaaatggacctgctgaccgtgccccggaaccagaacgagagcgtgtccgagatcggcggcaagattttcgaaaaggccgtgaagcggctgtccagcatcgacggcctgcaccagatcagcagcatcgtgccctttctgacagactccagctgctgcggctaccacaaggccagctactatctggccgtgttctacgagacaggcctgaacgtgcccagggaccagctgcagggcatgctgtacagcctggtgggcggccagggcagcgagagactgagcagcatgaacctgggctacaagcactaccagggcatcgacaactaccccctggactgggagctgtcctacgcctactacagcaatatcgccaccaagacccccctggaccagcacacactgcagggcgaccaggcctacgtggagacaatccggctgaaggacgacgagatcctgaaggtgcagaccaaggaagatggcgacgtgttcatgtggctgaagcacgaggccaccagaggaaatgccgctgcccagcagagactggcccagatgctgttctggggacagcagggcgtggccaaaaaccctgaggccgccatcgagtggtatgccaagggcgccctggaaacagaggaccccgccctgatctacgactacgccatcgtgctgttcaagggccagggcgtgaagaagaaccggcggctggccctggaactgatgaagaaggccgccagcaagggactgcaccaggccgtgaatggcctgggctggtactaccacaagttcaagaagaactacgccaaggccgccaagtactggctgaaggccgaggaaatgggcaaccccgacgcctcctacaatctgggcgtgctgcacctggatggcatcttccccggcgtgcccggcagaaatcagaccctggccggcgagtactttcacaaggccgcccaggggggccacatggaaggcaccctgtggtgcagcctgtactacatcaccggcaacctggaaaccttccccagggaccccgagaaggccgtggtgtgggccaagcacgtggccgagaagaacggctacctgggccacgtgatcaggaagggcctgaacgcctacctggaaggcagctggcacgaggccctgctgtactatgtgctggccgccgagacaggcatcgaggtgtcccagaccaacctggcccacatctgcgaggaacggcccgacctggccagacgctacctgggagtgaactgcgtgtggcggtactacaacttcagcgtgttccagatcgacgcccccagcttcgcctacctgaagatgggcgacctgtactactacggccaccagaaccagtcccaggatctggaactgtccgtgcagatgtacgcccaggccgctctggatggcgacagccagggcttcttcaacctggctctgctgatcgaagagggcaccatcatccctcaccacatcctggactttctggaaatcgacagcaccctgcacagcaacaacatcagcatcctgcaggaactgtacgagcgctgctggtcccacagcaacgaagagagcttcagcccctgcagcctggcctggctgtacctgcacctgaggctgggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaagtga

SEQ ID NO: 132 - protein, 684 aa, Homo sapiens
MARHRNVRGYNYDEDFEDDDLYGQSVEDDYCISPSTAAQFIYSRRDKPSVEPVEEYDYEDLKESSNSVSNHQLSGFDQARLYSCLDHMREVLGDAVPDEILIEAVLKNKFDVQKALSGVLEQDRVQSLKDKNEATVSTGKIAKGKPVDSQTSRSESEIVPKVAKMTVSGKKQTMGFEVPGVSSEENGHSFHTPQKGPPIEDAIASSDVLETASKSANPPHTIQASEEQSSTPAPVKKSGKLRQQIDVKAELEKRQGGKQLLNLVVIGHVDAGKSTLMGHMLYLLGNINKRTMHKYEQESKKAGKASFAYAWVLDETGEERERGVTMDVGMTKFETTTKVITLMDAPGHKDFIPNMITGAAQADVAVLVVDASRGEFEAGFETGGQTREHGLLVRSLGVTQLAVAVNKMDQVNWQQERFQEITGKLGHFLKQAGFKESDVGFIPTSGLSGENLITRSQSSELTKWYKGLCLLEQIDSFKPPQRSIDKPFRLCVSDVFKDQGSGFCITGKIEAGYIQTGDRLLAMPPNETCTVKGITLHDEPVDWAAAGDHVSLTLVGMDIIKINVGCIFCGPKVPIKACTRFRARILIFNIEIPITKGFPVLLHYQTVSEPAVIKRLISVLNKSTGEVTKKKPKFLTKGQNALVELQTQRPIALELYKDFKELGRFMLRYGGSTIAAGVVTEIKE

SEQ ID NO: 133 - DNA, 21 bp, Artificial Sequence
  Synthetic oligonucleotide
gctccaggccataaggacttc

SEQ ID NO: 134 - DNA, 21 bp, Artificial Sequence
  Synthetic oligonucleotide
cagcttcaaactctcccctgc

SEQ ID NO: 135 - DNA, 103 bp, Artificial Sequence
  Synthetic oligonucleotide
gctccaggccataaggacttcattccaaatatgattacaggagcagcccaggcggatgtagctgttttagttgtagatgccagcaggggagagtttgaagctg

SEQ ID NO: 136 - protein, 664 aa, Homo sapiens
MSGVRGLSRLLSARRLALAKAWPTVLQTGTRGFHFTVDGNKRASAKVSDSISAQYPVVDHEFDAVVVGAGGAGLRAAFGLSEAGFNTACVTKLFPTRSHTVAAQGGINAALGNMEEDNWRWHFYDTVKGSDWLGDQDAIHYMTEQAPAAVVELENYGMPFSRTEDGKIYQRAFGGQSLKFGKGGQAHRCCCVADRTGHSLLHTLYGRSLRYDTSYFVEYFALDLLMENGECRGVIALCIEDGSIHRIRAKNTVVATGGYGRTYFSCTSAHTSTGDGTAMITRAGLPCQDLEFVQFHPTGIYGAGCLITEGCRGEGGILINSQGERFMERYAPVAKDLASRDVVSRSMTLEIREGRGCGPEKDHVYLQLHHLPPEQLATRLPGISETAMIFAGVDVTKEPIPVLPTVHYNMGGIPTNYKGQVLRHVNGQDQIVPGLYACGEAACASVHGANRLGANSLLDLVVFGRACALSIEESCRPGDKVPPIKPNAGEESVMNLDKLRFADGSIRTSELRLSMQKSMQNHAAVFRVGSVLQEGCGKISKLYGDLKHLKTFDRGMVWNTDLVETLELQNLMLCALQTIYGAEARKESRGAHAREDYKVRIDEYDYSKPIQGQQKKPFEEHWRKHTLSYVDVGTGKVTLEYRPVIDKTLNEADCATVPPAIRSY

SEQ ID NO: 137 - DNA, 20 bp, Artificial Sequence
  Synthetic oligonucleotide
ttccttgccaggacctagag

SEQ ID NO: 138 - DNA, 20 bp, Artificial Sequence
  Synthetic oligonucleotide
cataaacctttcgccttgac

SEQ ID NO: 139 - DNA, 128 bp, Artificial Sequence
  Synthetic oligonucleotide
ttccttgccaggacctagagtttgttcagttccaccccacaggcatatatggtgctggttgtctcattacggaaggatgtcgtggagagggaggcattctcattaacagtcaaggcgaaaggtttatg

SEQ ID NO: 140 - protein, 494 aa, Homo sapiens
MPRVYIGRLSYQARERDVERFFKGYGKILEVDLKNGYGFVEFDDLRDADDAVYELNGKDLCGERVIVEHARGPRRDGSYGSGRSGYGYRRSGRDKYGPPTRTEYRLIVENLSSRCSWQDLKDYMRQAGEVTYADAHKGRKNEGVIEFVSYSDMKRALEKLDGTEVNGRKIRLVEDKPGSRRRRSYSRSRSHSRSRSRSRHSRKSRSRSGSSKSSHSKSRSRSRSGSRSRSKSRSRSQSRSRSKKEKSRSPSKEKSRSRSHSAGKSRSKSKDQAEEKIQNNDNVGKPKSRSPSRHKSKSKSRSRSQERRVEEEKRGSVSRGRSQEKSLRQSRSRSRSKGGSRSRSRSRSKSKDKRKGRKRSREESRSRSRSRSKSERSRKRGSKRDSKAGSSKKKKKEDTDRSQSRSPSRSVSKEREHAKSESSQREGRGESENAGTNQETRSRSRSNSKSKPNLPSESRSRSKSASKTRSRSKSRSRSASRSPSRSRSRSHSRS

SEQ ID NO: 141 - DNA, 21 bp, Artificial Sequence
  Synthetic oligonucleotide
aatttgtcaagtcggtgcagc

SEQ ID NO: 142 - DNA, 20 bp, Artificial Sequence
  Synthetic oligonucleotide
tcaccccttcatttttgcgt

SEQ ID NO: 143 - DNA, 106 bp, Artificial Sequence
  Synthetic oligonucleotide
aatttgtcaagtcggtgcagctggcaagacctaaaggattatatgcgtcaggcaggagaagtgacttatgcagatgctcacaagggacgcaaaaatgaaggggtga

SEQ ID NO: 144 - protein, 361 aa, Homo sapiens
MFSSVAHLARANPFNTPHLQLVHDGLGDLRSSSPGPTGQPRRPRNLAAAAVEEYSCEFGSAKYYALCGFGGVLSCGLTHTAVVPLDLVKCRMQVDPQKYKGIFNGFSVTLKEDGVRGLAKGWAPTFLGYSMQGLCKFGFYEVFKVLYSNMLGEENTYLWRTSLYLAASASAEFFADIALAPMEAAKVRIQTQPGYANTLRDAAPKMYKEEGLKAFYKGVAPLWMRQIPYTMMKFACFERTVEALYKFVVPKPRSECSKPEQLVVTFVAGYIAGVFCAIVSHPADSVVSVLNKEKGSSASLVLKRLGFKGVWKGLFARIIMIGTLTALQWFIYDSVKVYFRLPRPPPPEMPESLKKKLGLTQ

SEQ ID NO: 145 - DNA, 20 bp, Artificial Sequence
  Synthetic oligonucleotide
cagccaggttatgccaacac

SEQ ID NO: 146 - DNA, 21 bp, Artificial Sequence
  Synthetic oligonucleotide
tcaaagcaggcgaacttcatc

SEQ ID NO: 147 - DNA, 140 bp, Artificial Sequence
  Synthetic oligonucleotide
cagccaggttatgccaacactttgagggatgcagctcccaaaatgtataaggaagaaggcctaaaagcattctacaagggggttgctcctctctggatgagacagataccatacaccatgatgaagttcgcctgctttga

SEQ ID NO: 148 - DNA, 2405 bp, Homo sapiens
tccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaaaaaaaaaaaaaaaaa

SEQ ID NO: 149 - DNA, 20 bp, Artificial Sequence
  Synthetic oligonucleotide
tgggaacaagagggcatctg

SEQ ID NO: 150 - DNA, 22 bp, Artificial Sequence
  Synthetic oligonucleotide
ccaccactgcatcaaattcatg

SEQ ID NO: 151 - DNA, 86 bp, Artificial Sequence
  Synthetic oligonucleotide
tgggaacaagagggcatctgctaaagtttcagattccatttctgctcagtatccagtagtggatcatgaatttgatgcagtggtgg

SEQ ID NO: 152 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 153 - DNA, 21 bp, Artificial Sequence
  Synthetic oligonucleotide
tgacactggcaaaacaatgca

SEQ ID NO: 154 - DNA, 21 bp, Artificial Sequence
  Synthetic oligonucleotide
ggtccttttcaccagcaagct

SEQ ID NO: 155 - DNA, 94 bp, Artificial Sequence
  Synthetic oligonucleotide
tgacactggcaaaacaatgcagactttgctttccttggtcaggcagtataatccaaagatggtcaaggtcgcaagcttgctggtgaaaaggacc

SEQ ID NO: 156 - DNA, 2395 bp, Homo sapiens
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcc

SEQ ID NO: 157 - DNA, 20 bp, Artificial Sequence
  Synthetic oligonucleotide
gaggccgtcaccaagaacat

SEQ ID NO: 158 - DNA, 19 bp, Artificial Sequence
  Synthetic oligonucleotide
ggacagccggtcagagctc

SEQ ID NO: 159 - DNA, 111 bp, Artificial Sequence
  Synthetic oligonucleotide
gaggccgtcaccaagaacattcacgagtcctgcatgagccagataggctggaaccgcatcatcgtggagaagcccttcgggagggacctgcagagctctgaccggctgtcc

SEQ ID NO: 160 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 161 - DNA, 20 bp, Artificial Sequence
  Synthetic oligonucleotide
cggtttgctgcggtaatcat

SEQ ID NO: 162 - DNA, 21 bp, Artificial Sequence
  Synthetic oligonucleotide
tttcttgctgccagtctggac

SEQ ID NO: 163 - DNA, 122 bp, Artificial Sequence
  Synthetic oligonucleotide
cggtttgctgcggtaatcatgaggataagagagccacgaaccacggcactgattttcagttctgggaaaatggtgtgcacaggagccaagagtgaagaacagtccagactggcagcaagaaa

SEQ ID NO: 164 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 165 - DNA, 19 bp, Artificial Sequence
  Synthetic oligonucleotide
atttgggtcgcggttcttg

SEQ ID NO: 166 - DNA, 21 bp, Artificial Sequence
  Synthetic oligonucleotide
tgccttgacattctcgatggt

SEQ ID NO: 167 - DNA, 133 bp, Artificial Sequence
  Synthetic oligonucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 168 - DNA, 1536 bp, Homo sapiens
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 169 - DNA, 19 bp, Artificial Sequence
  Synthetic oligonucleotide
tgagagtgattcgcgtggg

SEQ ID NO: 170 - DNA, 21 bp, Artificial Sequence
  Synthetic oligonucleotide
ccagggtacgaggctttcaat

SEQ ID NO: 171 - DNA, 91 bp, Artificial Sequence
  Synthetic oligonucleotide
tgagagtgattcgcgtgggtacccgcaagagccagcttgctcgcatacagacggacagtgtggtggcaacattgaaagcctcgtaccctgg

SEQ ID NO: 172 - protein, 339 aa, Homo sapiens
MDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 173 - DNA, 24 bp, Artificial Sequence
  Synthetic oligonucleotide
aacatcatggatcagaacaacagc

SEQ ID NO: 174 - DNA, 26 bp, Artificial Sequence
  Synthetic oligonucleotide
atcattggactaaagatagggattcc

SEQ ID NO: 175 - DNA, 101 bp, Artificial Sequence
  Synthetic oligonucleotide
aacatcatggatcagaacaacagcctgccaccttacgctcagggcttggcctcccctcagggtgccatgactcccggaatccctatctttagtccaatgat

SEQ ID NO: 176 - protein, 24 aa, Artificial Sequence
  Synthetic peptide
MVPSGGVPQGLGGRSACALLLLCY

SEQ ID NO: 177 - protein, 100 aa, Artificial Sequence
  Synthetic peptide
KSAVVAVAAAPHKTLGKHPERAANQPAGWGAARLQTCQQGGSPNPAGGQVENVVPSLGRQTSLTTSVIPKAEQSVAYKDFIYFTVFEGNVRNVSEVSVEY

SEQ ID NO: 178 - protein, 24 aa, Artificial Sequence
  Synthetic peptide
MVPSGGVPQGLGGRSACALLLLCY

SEQ ID NO: 179 - protein, 26 aa, Artificial Sequence
  Synthetic peptide
PQKVQNFYLVPSKKRDQCLRFRPPLP

SEQ ID NO: 180 - protein, 24 aa, Artificial Sequence
  Synthetic peptide
MVPSGGVPQGLGGRSACALLLLCY

SEQ ID NO: 181 - protein, 24 aa, Artificial Sequence
  Synthetic peptide
MVPSGGVPQGLGGRSACALLLLCY

SEQ ID NO: 182 - protein, 24 aa, Artificial Sequence
  Synthetic peptide
MVPSGGVPQGLGGRSACALLLLCY

SEQ ID NO: 183 - protein, 8 aa, Artificial Sequence
  Synthetic peptide
VRKVLEPQ

SEQ ID NO: 184 - protein, 24 aa, Artificial Sequence
  Synthetic peptide
MVPSGGVPQGLGGRSACALLLLCY

SEQ ID NO: 185 - protein, 2 aa, Artificial Sequence
  Synthetic peptide
HI

SEQ ID NO: 186 - protein, 24 aa, Artificial Sequence
  Synthetic peptide
MVPSGGVPQGLGGRSACALLLLCY

SEQ ID NO: 187 - protein, 11 aa, Artificial Sequence
  Synthetic peptide
STFWEPFCYPY

SEQ ID NO: 188 - protein, 24 aa, Artificial Sequence
  Synthetic peptide
MVPSGGVPQGLGGRSACALLLLCY

SEQ ID NO: 189 - protein, 20 aa, Artificial Sequence
  Synthetic peptide
LPRHCHVHCKSSCDSSCRCL

SEQ ID NO: 190 - protein, 24 aa, Artificial Sequence
  Synthetic peptide
MVPSGGVPQGLGGRSACALLLLCY

SEQ ID NO: 191 - protein, 24 aa, Artificial Sequence
  Synthetic peptide
MVPSGGVPQGLGGRSACALLLLCY

SEQ ID NO: 192 - protein, 34 aa, Artificial Sequence
  Synthetic peptide
KSLSTSVLGHPHTDTLALQKIVLHNTFGFKFNLT

SEQ ID NO: 193 - protein, 24 aa, Artificial Sequence
  Synthetic peptide
MVPSGGVPQGLGGRSACALLLLCY

SEQ ID NO: 194 - protein, 4 aa, Artificial Sequence
  Synthetic peptide
VRIT

SEQ ID NO: 195 - protein, 16 aa, Artificial Sequence
  Synthetic peptide
PECEKRKMSNSHHHFL

SEQ ID NO: 196 - protein, 145 aa, Artificial Sequence
  Synthetic peptide
GTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAP

SEQ ID NO: 197 - protein, 129 aa, Artificial Sequence
  Synthetic peptide
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYEC

SEQ ID NO: 198 - protein, 209 aa, Artificial Sequence
  Synthetic peptide
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGS

SEQ ID NO: 199 - protein, 170 aa, Artificial Sequence
  Synthetic peptide
GTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQRFTTAKVAFTQSPSAAHKSTNVHSPVTNGLKSTQRFPSAHIT

SEQ ID NO: 200 - protein, 129 aa, Artificial Sequence; identical to SEQ ID NO: 197
  Synthetic peptide

SEQ ID NO: 201 - protein, 209 aa, Artificial Sequence; identical to SEQ ID NO: 198
  Synthetic peptide

SEQ ID NO: 202 - protein, 27 aa, Artificial Sequence
  Synthetic peptide
HKSTNVHSPVTNGLKSTQRFPSAHITA

SEQ ID NO: 203 - protein, 145 aa, Artificial Sequence; identical to SEQ ID NO: 196
  Synthetic peptide

SEQ ID NO: 204 - protein, 79 aa, Artificial Sequence
  Synthetic peptide
SRPVTQAGMRWCDRSSLQSRTPGFKRSFHFSLPSSWYYRAHVFHVDRFAWDASNHGLADLAKEELRRKYTQVYRLFLVS

SEQ ID NO: 205 - protein, 129 aa, Artificial Sequence; identical to SEQ ID NO: 197
  Synthetic peptide

SEQ ID NO: 206 - protein, 209 aa, Artificial Sequence; identical to SEQ ID NO: 198
  Synthetic peptide

SEQ ID NO: 207 - protein, 18 aa, Artificial Sequence
  Synthetic peptide
ESSRVEHTMLQTCMSSLS

SEQ ID NO: 208 - protein, 147 aa, Artificial Sequence
  Synthetic peptide
MTVARPSVPAALPLLGELPRLLLLVLLCLPAVWESSRVEHTMLQTCMSSLSGDCGLPPDVPNAQPALEGRTSFPEDTVITYKCEESFVKIPGEKDSVICLKGSQWSDIEEFCNRSCEVPTRLNSASLKQPYITQNYFPVGTVVEYEC

SEQ ID NO: 209 - protein, 104 aa, Artificial Sequence
  Synthetic peptide
MTVARPSVPAALPLLGELPRLLLLVLLCLPAVWGDCGLPPDVPNAQPALEGRTSFPEDTVITYKCEESFVKIPGEKDSVICLKGSQWSDIEEFCNLGTVVEYEC

SEQ ID NO: 210 - protein, 145 aa, Artificial Sequence; identical to SEQ ID NO: 196
  Synthetic peptide

SEQ ID NO: 211 - protein, 83 aa, Artificial Sequence
  Synthetic peptide
SRPVTQAGMRWCDRSSLQSRTPGFKRSFHFSLPSSWYYRCVPRHPAKFLKFIFCRDRIFLCCPGWFQTPGRKRFFRPPKTLRL

SEQ ID NO: 212 - protein, 129 aa, Artificial Sequence; identical to SEQ ID NO: 197
  Synthetic peptide

SEQ ID NO: 213 - protein, 209 aa, Artificial Sequence; identical to SEQ ID NO: 198
  Synthetic peptide

SEQ ID NO: 214 - protein, 170 aa, Artificial Sequence; identical to SEQ ID NO: 199
  Synthetic peptide

SEQ ID NO: 215 - protein, 18 aa, Artificial Sequence
  Synthetic peptide
ALIMHMRATKYSMLCLTI

SEQ ID NO: 216 - protein, 129 aa, Artificial Sequence; identical to SEQ ID NO: 197
  Synthetic peptide

SEQ ID NO: 217 - protein, 209 aa, Artificial Sequence; identical to SEQ ID NO: 198
  Synthetic peptide

SEQ ID NO: 218 - protein, 27 aa, Artificial Sequence
  Synthetic peptide
HKSTNVHSPVTNGLKSTQRFPSAHITA

SEQ ID NO: 219 - DNA, 732 bp, Artificial Sequence
  Synthetic oligonucleotide
ggatccgagaacctgtactttcagggcagcggcgagcccagaggccccaccatcaagccctgccccccctgcaagtgcccagcccctaacctgctgggcggacccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggactacaacagcaccctgcgggtggtgtccgccctgcccatccagcaccaggactggatgagcggcaaagaattcaagtgcaaggtgaacaacaaggacctgcctgcccccatcgagcggaccatcagcaagcccaagggcagcgtgagagccccccaggtgtacgtgctgccccctcccgaggaagagatgaccaagaaacaggtgaccctgacctgcatggtgaccgacttcatgcccgaggacatctacgtggagtggaccaacaacggcaagaccgagctgaactacaagaacaccgagcccgtgctggacagcgacggcagctacttcatgtatagcaagctgagagtcgagaagaaaaactgggtggagcggaacagctacagctgcagcgtggtgcacgagggcctgcacaaccaccacaccaccaagagcttcagccggacccccggcaag